(19) United States
(12) Patent Application Publication
(10) Pub. No.: US 2019/0161803 A1
(43) Pub. Date: May 30, 2019

(54) POLYMORPHISMS TOLL LIKE RECEPTOR GENES PREDICTS CLINICAL OUTCOMES OF COLORECTAL CANCER PATIENTS

(71) Applicant: University of Southern California, Los Angeles, CA (US)

(72) Inventor: Heinz-Josef Lenz, Los Angeles, CA (US)

(21) Appl. No.: 16/092,748

(22) PCT Filed: Apr. 21, 2017
(86) PCT No.: PCT/US17/28998

§ 371 Date: Oct. 10, 2018

Related U.S. Application Data
(60) Provisional application No. 62/326,621, filed on Apr. 22, 2016.

(51) Int. Cl. C12Q 1/6886; C07K 16/28; A61K 39/395; A61K 31/519; A61K 31/513; C07K 16/22; A61K 31/4745

(52) U.S. Cl. CPC C12Q 1/6886; C07K 16/2863; A61K 39/3955; A61K 31/519; A61K 31/513; C07K 2317/24; A61K 31/4745; C12Q 2600/156; C12Q 2600/106; C12Q 2600/118; C07K 16/22

(57) ABSTRACT

Methods are provided for identifying the clinical outcome of cancer patients following a therapy comprising cetuximab or irinotecan plus bevacizumab. The methods entail screening a cell or tissue sample isolated from the patient for an rs3853839 or rs5743618 polymorphism. After determining if a patient is likely to be successfully treated, the disclosure also provides methods for treating the patient.

### CROSS REFERENCE TO RELATED APPLICATIONS

This application claims priority under 35 U.S.C. § 119(e) to U.S. Provisional Application No. 62/326,621, filed Apr. 22, 2016, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 18, 2017, is named 064189-9045 SL.txt and is 126,494 bytes in size.

### BACKGROUND

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response.

In recent years, improved anti-cancer therapies have led to significantly improved clinical outcome of patients with colorectal cancer. However, as not all patients benefit from a chemotherapy regimen, predictive and prognostic biomarkers are critical in identifying patients who most benefit from the chemotherapy. Ras testing is the only validated predictive biomarker of EGFR-inhibitor efficacy (1, 2), and there has been a lot of effort in identifying the biomarker of anti VEGF therapies (3-5). To date, several studies have suggested that inflammatory and immune responses within the tumor microenvironment may play a role in the efficacy of cytotoxic chemotherapy and targeted antibodies. Most recently, it was shown that colorectal tumors with high MSI (microsatellite instability) likely respond to immune checkpoint inhibitors (6). Key genes involved in immune regulation and inflammatory response may be novel biomarkers predicting clinical responses and tumor prognosis in patients with metastatic colorectal cancer (mCRC) patients.

Toll like receptors (TLRs) play a crucial role in the intestinal mucosal innate and acquired immunity to maintain gut homeostasis (7). TLR, one of the pattern recognition receptors, recognizes not only exogenous pathogen-associated molecular patterns (PAMPs) but also endogenous molecules, termed damage-associated molecular patterns (DAMPs), which are released from injured or dying cells (8). Activated TLRs, by PAMPs or DAMPs, initiate signal cascades, such as nuclear factor-kappa B (NFκB), mitogen-associated protein kinase (MAPK), and interferon (IFN) pathways, and promote the secretion of cytokines and chemokines, which regulate immune and inflammatory responses against microbial infection or tissue injury (9). Within the tumor microenvironment, TLRs are expressed not only on immune cells but also on tumor cells and stromal cells (10). Individual TLR signaling in each cell initiates divergent pathways that can influence either tumor promotion (e.g. pro-inflammation, angiogenesis, and anti-apoptosis) or anti-tumor immunity (11). These biological responses mediated by TLRs in the tumor microenvironment are thought to influence clinical prognosis, as well as response to anti-cancer therapy (12).

Although considerable research correlating gene expression and/or polymorphisms has been reported, much work remains to be done. This disclosure supplements the existing body of knowledge and provides related advantages as well.

### SUMMARY

Toll-like receptor signaling pathways are implicated in the regulation of immune system through type I interferon induction. Immune responses within the tumor microenvironment may influence the efficacy of chemotherapy. TLR7 and TLR9 agonists showed promising results in preclinical and/or clinical trials for cancer patients, in particular in association with cetuximab (cet). It is described herein that genetic variations in TLR7 are associated with clinical outcome in mCRC patients receiving an anti-EGFR based chemotherapy such as cetuximab. It is also described herein that genetic variations in TLR1 are associated with clinical outcome in mCRC patients receiving irinotecan and bevacizumab-based chemotherapy.

This disclosure relates to methods and kits for one or more of: detecting a polymorphism in a cancer patient or a patient suspected of having cancer, selecting a cancer patient for therapy, classifying a cancer patient as eligible for therapy, identifying a cancer patient that is likely to experience longer or shorter progression free survival following therapy, treating a cancer patient, and increasing progression-free and/or overall survival of a cancer patient. In some aspects, the cancer is GI cancer, colon cancer, rectal cancer, or colorectal cancer.

This disclosure provides an in vitro method of detecting a polymorphism in a patient with cancer or a patient suspected of having cancer, e.g., GI cancer, colon cancer, rectal cancer, or colorectal cancer, the method comprising, or alternatively consisting essentially of, or yet further consisting of screening a biological sample from the patient to detect the genotype of (G/G), (C/G), or (C/C) for rs3853839. In one aspect, the patient is known to have the cancer. In another aspect, the patient is suspected of having the cancer.

Also provided by this disclosure is a method for selecting a cancer patient, e.g. a GI cancer patient, a colon cancer patient, a rectal cancer patient, or a colorectal cancer patient, for a therapy comprising, or alternatively consisting essentially of, or yet further consisting of administration of an effective amount of cetuximab, the method comprising, or alternatively consisting essentially of, or yet further consisting of screening a biological sample isolated from the patient for an rs3853839 polymorphism, and selecting the patient for the therapy if the genotype of (G/G) for rs3853839 is present in the sample. In one aspect, the patient is known to have the cancer. In another aspect, the patient is suspected of having the cancer. The cetuximab therapy can be first line, second line, third line, fourth line, or fifth line therapy and can be administered alone or in combination with other therapies, e.g., surgical resection, radiation therapy, or other chemical or biological based therapies.

Further provided by this disclosure is a method for classifying a cancer patient, e.g. a GI cancer patient, a colon cancer patient, a rectal cancer patient, or a colorectal cancer patient, as eligible for a therapy comprising, or alternatively consisting essentially of, or yet further consisting of administration of an effective amount of cetuximab, the method comprising, or alternatively consisting essentially of, or yet further consisting of screening a biological sample isolated from the patient for an rs3853839 polymorphism, and classifying the patient as eligible for the therapy if the genotype of (G/G) for rs3853839 is present in the sample. In one aspect, the patient is known to have the cancer. In another aspect, the patient is suspected of having the cancer. The cetuximab therapy can be first line, second line, third line, fourth line, or fifth line therapy and can be administered alone or in combination with other therapies, e.g., surgical resection, radiation therapy, or other chemical or biological based therapies.

Also provided herein is a method for identifying whether a cancer patient, e.g. a GI cancer patient, a colon cancer patient, a rectal cancer patient, or a colorectal cancer patient, is likely to experience a relatively longer or shorter progression free survival following a therapy comprising, or alternatively consisting essentially of, or yet further consisting of administration of an effective amount of cetuximab, the method comprising, or alternatively consisting essentially of, or yet further consisting of screening a biological sample isolated from the patient for an rs3853839 polymorphism, and identifying that the patient is likely to experience a longer progression free survival if the genotype of (G/G) for rs3853839 is present in the sample, relative to a corresponding cancer patient not having the genotype. In one aspect, the patient is known to have the cancer. In another aspect, the patient is suspected of having the cancer. The cetuximab therapy can be first line, second line, third line, fourth line, or fifth line therapy and can be administered alone or in combination with other therapies, e.g., surgical resection, radiation therapy, or other chemical or biological based therapies.

Yet further provided is a method for treating a cancer patient, e.g. a GI cancer patient, a colon cancer patient, a rectal cancer patient, or a colorectal cancer patient, selected for treatment based on the presence of the genotype of (G/G) for rs3853839 in a biological sample from the patient, the method comprising, or alternatively consisting essentially of, or yet further consisting of administering to the patient a therapy comprising a therapeutically effective amount of cetuximab or an equivalent thereof. In one aspect, the patient is known to have the cancer. In another aspect, the patient is suspected of having the cancer. The cetuximab therapy can be first line, second line, third line, fourth line, or fifth line therapy and can be administered alone or in combination with other therapies, e.g., surgical resection, radiation therapy, or other chemical or biological based therapies.

Also provided herein is a method for increasing the progression-free and/or overall survival of a cancer patient, e.g. a GI cancer patient, a colon cancer patient, a rectal cancer patient, or a colorectal cancer patient, the method comprising, or alternatively consisting essentially of, or yet further consisting of screening a biological sample isolated from the patient for an rs3853839 polymorphism, and classifying the patient as eligible for the therapy with cetuximab if the genotype of (G/G) for rs3853839 is present in the sample or not eligible for the therapy comprising cetuximab if the genotype of (G/G) for rs3853839 is not present in the sample. In one aspect, the patient is known to have the cancer. In another aspect, the patient is suspected of having the cancer. The cetuximab therapy can be first line, second line, third line, fourth line, or fifth line therapy and can be administered alone or in combination with other therapies, e.g., surgical resection, radiation therapy, or other chemical or biological based therapies.

This disclosure also provides an in vitro method of detecting a polymorphism in a patient with cancer or a patient suspected of having cancer, e.g., GI cancer, colon cancer, rectal cancer, or colorectal cancer, the method comprising, or alternatively consisting essentially of, or yet further consisting of screening a biological sample from the patient to detect the genotype of (G/T) or (G/G) for rs5743618. In one aspect, the patient is known to have the cancer. In another aspect, the patient is suspected of having the cancer.

Further provided is a method for selecting a cancer patient, e.g. a GI cancer patient, a colon cancer patient, a rectal cancer patient, or a colorectal cancer patient, for a therapy comprising, or alternatively consisting essentially of, or yet further consisting of administration of an effective amount of irinotecan and/or bevacizumab, the method comprising, or alternatively consisting essentially of, or yet further consisting of screening a biological sample isolated from the patient for an rs5743618 polymorphism, and selecting the patient for the therapy if the genotype of (G/T) or (G/G) for rs5743618 is present in the sample. In one aspect, the patient is known to have the cancer. In another aspect, the patient is suspected of having the cancer. The irinotecan and/or bevacizumab therapy can be first line, second line, third line, fourth line, or fifth line therapy and can be administered alone or in combination with other therapies, e.g., surgical resection, radiation therapy, or other chemical or biological based therapies.

Also provided is a method for classifying a cancer patient, e.g. a GI cancer patient, a colon cancer patient, a rectal cancer patient, or a colorectal cancer patient, as eligible for a therapy comprising, or alternatively consisting essentially of, or yet further consisting of administration of an effective amount of irinotecan and/or bevacizumab, the method comprising, or alternatively consisting essentially of, or yet further consisting of screening a biological sample isolated from the patient for an rs5743618 polymorphism, and classifying the patient as eligible for the therapy if the genotype of (G/T) or (G/G) for rs5743618 is present in the sample. In one aspect, the patient is known to have the cancer. In another aspect, the patient is suspected of having the cancer. The irinotecan and/or bevacizumab therapy can be first line, second line, third line, fourth line, or fifth line therapy and can be administered alone or in combination with other therapies, e.g., surgical resection, radiation therapy, or other chemical or biological based therapies.

Yet further provided herein is a method for identifying whether a cancer patient, e.g. a GI cancer patient, a colon cancer patient, a rectal cancer patient, or a colorectal cancer patient, is likely to experience a relatively longer or shorter progression free survival following a therapy comprising, or alternatively consisting essentially of, or yet further consisting of administration of an effective amount of irinotecan and/or bevacizumab, the method comprising, or alternatively consisting essentially of, or yet further consisting of screening a biological sample isolated from the patient for an rs5743618 polymorphism, and identifying that the patient is likely to experience a longer progression free survival if the genotype of (G/T) or (G/G) for rs5743618 is present in the sample, relative to a corresponding cancer patient not having the genotype. In one aspect, the patient is known to have the cancer. In another aspect, the patient is suspected of having the cancer. The irinotecan and/or bevacizumab therapy can be first line, second line, third line, fourth line, or fifth line therapy and can be administered alone or in combination with other therapies, e.g., surgical resection, radiation therapy, or other chemical or biological based therapies.

Also provided is a method for treating a cancer patient, e.g. a GI cancer patient, a colon cancer patient, a rectal cancer patient, or a colorectal cancer patient, selected for treatment based on the presence of the genotype of (G/T) or (G/G) for rs5743618 in a biological sample from the patient, the method comprising, or alternatively consisting essentially of, or yet further consisting of administering to the patient a therapy comprising, or alternatively consisting essentially of, or yet further consisting of a therapeutically effective amount of irinotecan and/or bevacizumab. In one aspect, the patient is known to have the cancer. In another aspect, the patient is suspected of having the cancer. The irinotecan and/or bevacizumab therapy can be first line, second line, third line, fourth line, or fifth line therapy and can be administered alone or in combination with other therapies, e.g., surgical resection, radiation therapy, or other chemical or biological based therapies.

Further provided herein is a method for increasing the progression-free and/or overall survival of a cancer patient, e.g. a GI cancer patient, a colon cancer patient, a rectal cancer patient, or a colorectal cancer patient, the method comprising, or alternatively consisting essentially of, or yet further consisting of screening a biological sample isolated from the patient for an rs5743618 polymorphism, and classifying the patient as eligible for the therapy with irinotecan and/or bevacizumab if the genotype of (G/T) or (G/G) for rs5743618 is present in the sample or not eligible for the therapy comprising irinotecan and/or bevacizumab if the genotype of (G/T) or (G/G) for rs5743618 is not present in the sample. In one aspect, the patient is known to have the cancer. In another aspect, the patient is suspected of having the cancer. The irinotecan and/or bevacizumab therapy can be first line, second line, third line, fourth line, or fifth line therapy and can be administered alone or in combination with other therapies, e.g., surgical resection, radiation therapy, or other chemical or biological based therapies.

Yet further provided are kits for performing the methods described herein, comprising, or alternatively consisting essentially of, or yet further consisting of the reagents to identify or determine the genotype of a sample or a patient and instructions for use.

### BRIEF DESCRIPTION OF THE DRAWINGS

FIG. 1 illustrates the prognostic significance of TLR1 rs5743618 in the TRIBE cohort. The data describe progression free survival (PFS) and overall survival (OS) between the (T/T) (dotted line, n=44) and either the (G/T) or (G/G) (“Any G”, dark line, n=182) genotypes for TLR1 rs5743618 for TRIBE cohort. The x axis depicts months since treatment and the y axis depicts estimated probability of survival.

FIG. 2 illustrates data for progression free survival and overall survival between the (T/T) (dark gray line, n=34) and (T/C) or (C/C) (light gray line, n=9) genotypes for TLR9 s5743836 in patients with a KRAS mutation for FIRE3 cohort with bevacizumab arm. The x axis depicts months since treatment and the y axis depicts estimated probability of survival.

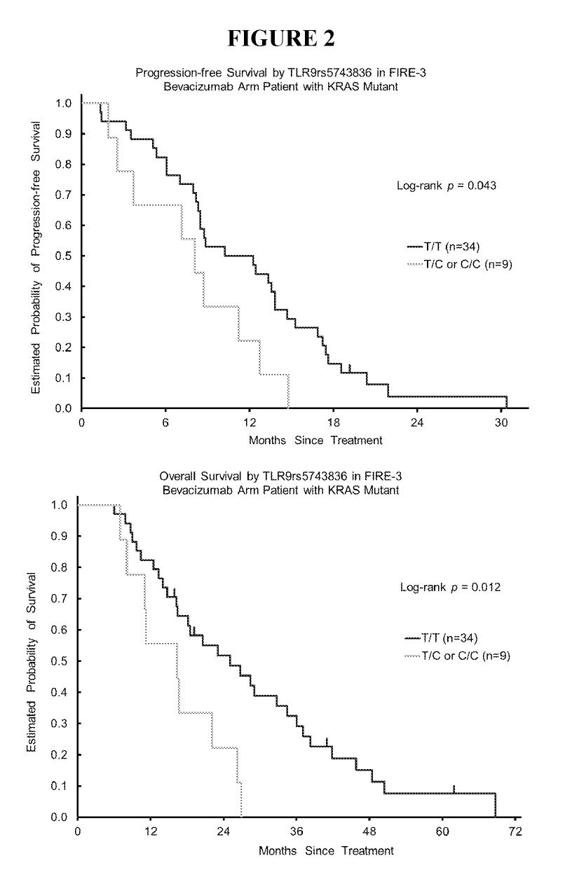

FIG. 3 illustrates data for progression free survival and overall survival between the (T/T) (dark gray line, n=181) and (T/C) or (C/C) (light gray line, n=56) genotypes for TLR9 s5743836 in patients with KRAS wild-type alleles for FIRE3 cohort with bevacizumab arm. The x axis depicts months since treatment and the y axis depicts estimated probability of survival.

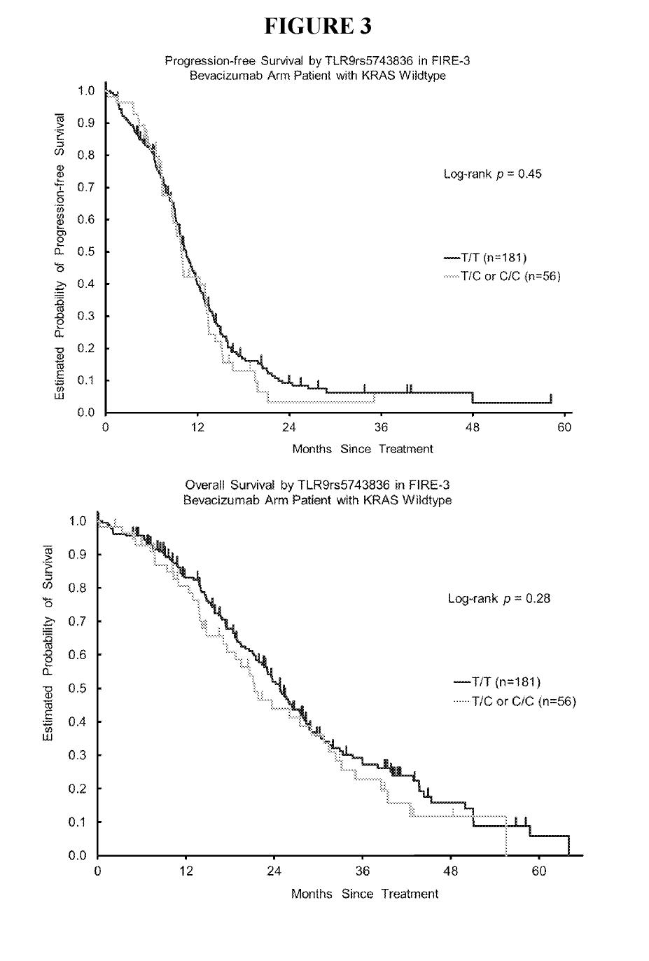

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used herein, the term “cancer” intends a malignant phenotype characterized by the uncontrolled proliferation of malignant cells. The methods and compositions of this disclosure are useful for the treatment and diagnosis of cancer of the gastrointestinal (GI) tract, such as gastric cancer, GI cancer, colon cancer, rectal cancer, and/or colorectal cancer. The cancer can be metastatic, non-metastatic and pre-clinical.

As used herein, the terms “corresponding cancer” when referring to a comparison or relative term indicates that the measured or detected polymorphism is compared to a patient having the same cancer type, e.g., colorectal cancer is compared to colorectal cancer.

The term “chemotherapy” encompasses cancer therapies that employ chemical or biological agents or other therapies, such as radiation therapies, e.g., a small molecule drug or a large molecule, such as antibodies, RNAi and gene therapies. Non-limiting examples of chemotherapies are provided below. Unless specifically excluded, when a chemotherapy is recited, equivalents thereof are intended within the scope of the invention.

An anti-EGFR antibody refers to an antibody or a small molecule inhibiting expression or function of EGFR. In one aspect, anti-EGFR therapy comprises, or alternatively consists essentially of, or yet further, consists of, an antibody or fragment thereof that binds the EGFR antigen. A non-limiting example of such is the antibody cetuximab or equivalents thereof that bind to the same epitope. Another example of such is the antibody panitumumab or equivalents thereof that bind to the same epitope. It can be polyclonal or monoclonal. The antibody may be of any appropriate species such as for example, murine, ovine or human. It can be humanized, chimeric, bispecific, a heteroantibody, a derivative or variant of a polyclonal or monoclonal.

The term “EGFR” or “epidermal growth factor receptor” refers to the human protein having a GenBank Gene ID No. 1956 or any mammal homologue. The epitope that cetuximab binds to is known and is described in, for instance, Li et al., Cancer Cell, 7:301-11 (2005).

Cetuximab (IMC-C225) is marketed under the name Erbitux®. Cetuximab is a chimeric (mouse/human) monoclonal antibody, an epidermal growth factor receptor (EGFR) inhibitor, given by intravenous injection for treatment of metastatic colorectal cancer and head and neck cancer. Cetuximab is manufactured and distributed in North America by ImClone and Bristol-Myers Squibb, while in the rest of the world distribution is by Merck KGaA. In one aspect, an equivalent of cetuximab is an antibody directed to EGFR, or a small molecule targeting EGFR or inhibiting EGFR. In another aspect, an equivalent of cetuximab may also include homologs of cetuximab, mutant cetuximab, recombinant cetuximab that retains substantially the same function of cetuximab. Although not always explicitly stated, when the term cetuximab is used in terms of therapy, Applicant intends not only Erbitux, but also equivalents thereof.

Panitumumab (INN), formerly ABX-EGF, is a fully human monoclonal antibody specific to the epidermal growth factor receptor. Panitumumab is manufactured by Amgen and marketed as Vectibix. In one aspect, an equivalent of panitumumab is an antibody directed to EGFR, or a small molecule targeting EGFR or inhibiting EGFR. In another aspect, an equivalent of panitumumab may also include homologs of panitumumab, mutant panitumumab, recombinant panitumumab that retains substantially the same function of panitumumab. Although not always explicitly stated, when the term panitumumab is used in terms of therapy, Applicant intends not only panitumumab, but also equivalents thereof.

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation. An equivalent of irinotecan is a composition that inhibits a topoisomerase. Non-limiting examples of an equivalent of irinotecan include topotecan, camptothecin and lamellarin D, etoposide, or doxorubicin. Although not always explicitly stated, when the term irinotecan is used in terms of therapy, Applicant intends not only irinotecan, but also equivalents thereof.

Oxaliplatin (trans-/-diaminocyclohexane oxalatoplatinum; L-OHP; CAS No. 61825-94-3) is sold under the trade name of Elotaxin. It is a platinum derivative that causes cell cytotoxicity. Oxaliplatin forms both inter- and intra-strand cross links in DNA, which prevent DNA replication and transcription, causing cell death. Non-limiting examples of an equivalent of oxaliplatin include carboplatin and cisplatin. Although not always explicitly stated, when the term oxaliplatin is used in terms of therapy, Applicant intends not only oxaliplatin, but also equivalents thereof.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. In one aspect, topoisomerase inhibitors include irinotecan, topotecan, camptothecin and lamellarin D, or compounds targeting topoisomerase IA. In another aspect, topoisomerase inhibitors include etoposide, doxorubicin or compounds targeting topoisomerase II.

Pyrimidine antimetabolite includes, without limitation, fluorouracil (5-FU), its equivalents and prodrugs. In one embodiment, a pyrimidine antimetabolite is a chemical that inhibits the use of a pyrimidine. The presence of antimetabolites can have toxic effects on cells, such as halting cell growth and cell division, so these compounds can be used as chemotherapy for cancer.

Fluorouracil (5-FU) belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487. Although not always explicitly stated, when the term 5-FU is used in terms of therapy, Applicant intends not only 5-FU, but also equivalents thereof.

“5-FU based adjuvant therapy” refers to 5-FU alone or alternatively the combination of 5-FU with other treatments, that include, but are not limited to radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

A therapy comprising a pyrimidine antimetabolite includes, without limitation, a pyrimidine antimetabolite alone or alternatively the combination of a pyrimidine antimetabolite with other treatments, that include, but are not limited to, radiation, methyl-CCNU, leucovorin, oxaliplatin, irinotecin, mitomycin, cytarabine, levamisole. Specific treatment adjuvant regimens are known in the art as FOLFOX, FOLFOX4, FOLFOX6, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

FOLFIRI is a chemotherapy regimen for treatment of colorectal cancer. It is made up of the following drugs: FOL—folinic acid (leucovorin), a vitamin B derivative used as a “rescue” drug for high doses of the drug methotrexate and that modulates/potentiates/reduces the side effects of fluorouracil; F—fluorouracil (5-FU), a pyrimidine analog and antimetabolite which incorporates into the DNA molecule and stops synthesis; and IRI—irinotecan (Camptosar), a topoisomerase inhibitor, which prevents DNA from uncoiling and duplicating.

FOLFOX is a chemotherapy regimen for treatment of colorectal cancer. is made up of the following drugs: FOL—folinic acid (leucovorin), F—fluorouracil (5-FU), and OX-oxaliplatin.

FOLFOXFIRI is a chemotherapy regimen for treatment of colorectal cancer. is made up of the following drugs: FOL—folinic acid (leucovorin), F—fluorouracil (5-FU), OX—oxaliplatin and IRI—irinotecan (Camptosar).

Bevacizumab (BV) is sold under the trade name Avastin® by Genentech. It is a humanized monoclonal antibody that binds to and inhibits the biologic activity of human vascular endothelial growth factor (VEGF). Biological equivalent antibodies are identified herein as modified antibodies which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis. Bevacizumab is also in the class of cancer drugs that inhibit angiogenesis (angiogenesis inhibitors). Although not always explicitly stated, when the term bevacizumab is used in terms of therapy, Applicant intends not only bevacizumab but also equivalents thereof.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov. Typically, a patient is given a subsequent chemotherapy regimen because the patient did not shown a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

As used herein, the term “determining the genotype of a cell or tissue sample” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus will be identified as “X/Y” wherein X and Y are two different nucleotides. If the allelic composition of a SNP locus is heterozygous, the genotype of the SNP locus will be identified as “X/X” wherein X identifies the nucleotide that is present at both alleles.

The term “genetic marker” refers to an allelic variant of a polymorphic region of a gene of interest and/or the expression level of a gene of interest.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

The term “genotype” refers to the specific allelic composition of an entire cell or a certain gene and in some aspects a specific polymorphism associated with that gene, whereas the term “phenotype” refers to the detectable outward manifestations of a specific genotype.

The KRAS gene (NM_004985, NM_033360) is a proto-oncogene that encodes a GTPase important in in signal transduction. Mutations in the KRAS gene are found at high rates in cancers, including but not limited to colorectal cancers. Common KRAS mutations in colorectal cancer include but are not limited to mutations at codons 12 or 13 of exon 2 that result in amino acid substitutions in the protein sequence such as Gly12Asp [GGT>GAT] G12D, Gly12Val [GGT>GAC] G12V, Gly12Cys [GGT>TGT] G12C, Gly12Ser [GGT>AGT] G12S, Gly12Ala [GGT>GCT] G12A, Gly12Arg [GGT>CGT] G12R, Gly13Asp [GGC>GAC] G13D. The BRAF gene (NM_004333) is a proto-oncogene that is often mutated in colorectal cancers. The BRAF gene encodes a signal transduction kinase of the Raf family. Common mutations of the BRAF gene that are relevant to cancer result in amino acid substitutions in the protein sequence including but not limited to V600E, R461I, I462S, G463E, G463V, G465A, G465E, G465V, G468A, G468E, N580S, E585K, D593V, F594L, G595R, L596V, T5981, V599D, V599E, V599K, V599R, V600K, A727V. The terms “KRAS wild-type” and “BRAF wild-type” refers to a genotype of a cell or patient in which no mutation is detected in the corresponding gene. In some aspects, no mutation is detected that affects the function or activity of the gene.

The rs3853839 polymorphism is located at chromosome position 10756545 on the X chromosome according to the Genome Reference Consortium Human Build 38 patch release 2 (GRCh38.p2, NCBI). The rs3853839 polymorphism is located within the toll-like receptor 7 (TLR7) gene (SEQ ID NO:31) (mRNA NM_016562). The following nucleotide sequence represents a region of human DNA comprising the rs3853839 polymorphism: TGCTTCAGTGCTTCCTGCTCTTTTT[C/G]CTTGGGCCTGCTTCTGGGTTCCATA (SEQ ID NO:1). Thus, the partial sequence of the (C) allele of rs3853839 is TGCTTCAGTGCTTCCTGCTCTTTTTCCTTGGGCCTGCTTCTGGGTTCCATA (SEQ ID NO:24) and the partial sequence of the (G) allele of rs3853839 is TGCTTCAGTGCTTCCTGCTCTTTTTGCTTGGGCCTGCTTCTGGGTTCCATA (SEQ ID NO:25).

The rs5743618 polymorphism is located at chromosome position 5958091 on chromosome 4 according to the Genome Reference Consortium Human Build 38 patch release 2 (GRCh38.p2, NCBI). The rs5743618 polymorphism is located within the toll-like receptor 1 (TLR1) gene (SEQ ID NO:30), (mRNA NM_003263). The following nucleotide sequence represents a region of human TLR1 DNA comprising the rs5743618 polymorphism: GCTGTGACTGTGACCTCCCTCTGCA[G/T]CTACTTGGATCTGCCCTGGTATCTC (SEQ ID NO:4). Thus, the partial sequence of the (G) allele of TLR1 rs5743618 is GCTGTGACTGTGACCTCCCTCTGCAGCTACTTGGATCTGCCCTGGTATCTC (SEQ ID NO:26) and the partial sequence of the (T) allele of TLR1 rs5743618 is GCTGTGACTGTGACCTCCCTCTGCATCTACTTGGATCTGCCCTGGTATCTC (SEQ ID NO:27)

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs.

“An effective amount” or “therapeutically effect amount” intends to indicate the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

A “patient” as used herein intends an animal patient, a mammal patient or yet further a human patient. For the purpose of illustration only, a mammal includes but is not limited to a simian, a murine, a bovine, an equine, a porcine or an ovine subject.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), objective response rate (RR), toxicity or side effect.

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

Response criteria herein are based on the RECIST criteria (Therasse and Arbuck et al., 2000, New Guidelines to Evaluate Response to Treatment in Solid Tumors, J Natl Cancer Inst, 92:205-16. A “complete response” (CR) to a therapy refers to the clinical status of a patient with evaluable but non-measurable disease, whose tumor and all evidence of disease have disappeared following administration of the therapy. In this context, a “partial response” (PR) refers to a response that is anything less than a complete response. “Stable disease” (SD) indicates that the patient is stable following the therapy. “Progressive disease” (PD) indicates that the tumor has grown (i.e. become larger) or spread (i.e. metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed. “Responder” refers to a patient with either a CR or PR. “Nonresponder” refers to a patient with either SD or PD.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow. Progression-free survival includes the amount of time a patient has experienced a complete response, partial response or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

“Relative Risk” in statistics and mathematical epidemiology, refers to the risk of an event (or of developing a disease) relative to exposure. Relative risk is a ratio of the probability of the event occurring in the exposed group versus a non-exposed group.

“Objective response rate” (RR) refers to the proportion of responders (patients with either a partial (PR) or complete response (CR)) compared to nonresponders (patients with either SD or PD). Response duration can be measured from the time of initial response until documented tumor progression.

The term “identify” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

When a genetic marker or polymorphism “is used as a basis” for identifying or selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

“Having the same cancer” is used when comparing one patient to another or alternatively, one patient population to another patient population. For example, the two patients or patient population will each have or be suffering from colon cancer.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

The term “amplification” or “amplify” as used herein means one or more methods known in the art for copying a target nucleic acid, thereby increasing the number of copies of a selected nucleic acid sequence. Amplification can be exponential or linear. A target nucleic acid can be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods can be used either in place of, or together with, PCR methods.

The term “complement” as used herein means the complementary sequence to a nucleic acid according to standard Watson/Crick base pairing rules. A complement sequence can also be a sequence of RNA complementary to the DNA sequence or its complement sequence, and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

As used herein, the term “hybridize” or “specifically hybridize” refers to a process where two complementary nucleic acid strands anneal to each other under appropriately stringent conditions. Hybridizations are typically conducted with probe-length nucleic acid molecules. Nucleic acid hybridization techniques are well known in the art. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

“Primer” as used herein refers to an oligonucleotide that is capable of acting as a point of initiation of synthesis when placed under conditions in which primer extension is initiated (e.g., primer extension associated with an application such as PCR). The primer is complementary to a target nucleotide sequence and it hybridizes to a substantially complementary sequence in the target and leads to addition of nucleotides to the 3′-end of the primer in the presence of a DNA or RNA polymerase. The 3′-nucleotide of the primer should generally be complementary to the target sequence at a corresponding nucleotide position for optimal expression and amplification. An oligonucleotide “primer” can occur naturally, as in a purified restriction digest or can be produced synthetically. The term “primer” as used herein includes all forms of primers that can be synthesized including, peptide nucleic acid primers, locked nucleic acid primers, phosphorothioate modified primers, labeled primers, and the like.

Primers are typically between about 5 and about 100 nucleotides in length, such as between about 15 and about 60 nucleotides in length, such as between about 20 and about 50 nucleotides in length, such as between about 25 and about 40 nucleotides in length. In some embodiments, primers can be at least 8, at least 12, at least 16, at least 20, at least 25, at least 30, at least 35, at least 40, at least 45, at least 50, at least 55, at least 60 nucleotides in length. An optimal length for a particular primer application can be readily determined in the manner described in H. Erlich, PCR Technology. Principles and Application for DNA Amplification (1989).

“Probe” as used herein refers to nucleic acid that interacts with a target nucleic acid via hybridization. A probe can be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe can specifically hybridize to a target nucleic acid.

Probes can be DNA, RNA or a RNA/DNA hybrid. Probes can be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes can comprise modified nucleobases, modified sugar moieties, and modified internucleotide linkages. A probe can be fully complementary to a target nucleic acid sequence or partially complementary. A probe can be used to detect the presence or absence of a target nucleic acid. Probes are typically at least about 10, 15, 21, 25, 30, 35, 40, 50, 60, 75, 100 nucleotides or more in length.

“Detecting” as used herein refers to determining the presence of a nucleic acid of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, preferably, a human. In an exemplary embodiment, the sample is a biopsy sample.

“Target nucleic acid” as used herein refers to segments of a chromosome, a complete gene with or without intergenic sequence, segments or portions a gene with or without intergenic sequence, or sequence of nucleic acids to which probes or primers are designed. Target nucleic acids can include wild type sequences, nucleic acid sequences containing mutations, deletions or duplications, tandem repeat regions, a gene of interest, a region of a gene of interest or any upstream or downstream region thereof. Target nucleic acids can represent alternative sequences or alleles of a particular gene. Target nucleic acids can be derived from genomic DNA, cDNA, or RNA. As used herein, target nucleic acid can be native DNA or a PCR-amplified product.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segments with a high frequency of complementary base sequences. Exemplary hybridization conditions are as follows. High stringency generally refers to conditions that permit hybridization of only those nucleic acid sequences that form stable hybrids in 0.018 M NaCl at 65° C. High stringency conditions can be provided, for example, by hybridization in 50% formamide, 5×Denhardt's solution, 5×SSC (saline sodium citrate) 0.2% SDS (sodium dodecyl sulfate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1×SSC, 0.2% SDS, at 50° C.

As used herein the term “substantially identical” refers to a polypeptide or nucleic acid exhibiting at least 50%, 75%, 85%, 90%, 95%, or even 99% identity to a reference amino acid or nucleic acid sequence over the region of comparison. For polypeptides, the length of comparison sequences will generally be at least 20, 30, 40, or 50 amino acids or more, or the full length of the polypeptide. For nucleic acids, the length of comparison sequences will generally be at least 10, 15, 20, 25, 30, 40, 50, 75, or 100 nucleotides or more, or the full length of the nucleic acid.

### Descriptive Embodiments

The disclosure further provides diagnostic, prognostic and therapeutic methods, which are based, at least in part, on determination of the identify of a genotype of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome following a clinical procedure such as a therapy or surgery can be expressed in relative terms. For example, a patient having a particular genotype or expression level can experience relatively longer overall survival than a patient or patients not having the genotype or expression level. The patient having the particular genotype or expression level, alternatively, can be considered as likely to survive. Similarly, a patient having a particular genotype or expression level can experience relatively longer progression free survival, or time to tumor progression, than a patient or patients not having the genotype or expression level. The patient having the particular genotype or expression level, alternatively, can be considered as not likely to suffer tumor progression. Further, a patient having a particular genotype or expression level can experience relatively shorter time to tumor recurrence than a patient or patients not having the genotype or expression level. The patient having the particular genotype or expression level, alternatively, can be considered as not likely to suffer tumor recurrence. Yet in another example, a patient having a particular genotype or expression level can experience relatively more complete response or partial response than a patient or patients not having the genotype or expression level. The patient having the particular genotype or expression level, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic assays described herein can be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

It is described herein that cancer patients harboring certain genotypes are likely to experience more desirable clinical outcomes when treated with an anti-EGFR therapy for example, the therapy comprising, consisting essentially of, or yet consisting of, cetuximab, or therapy comprising irinotecan and bevacizumab, as compared to those not having the genotype. More desirable clinical outcomes for a cancer patient following a therapy include, without limitation, higher likelihood to respond to the therapy, relatively longer progression free survival (PFS), relatively longer overall survival (OS), relatively longer time to tumor recurrence (TTR), lower likelihood to experience an adverse effect or toxicity, or relatively milder adverse effect or toxicity.

A summary of some of the findings of the present disclosure is provided in Table 1 below.

TABLE 1

| | | | Favorable | Unfavorable |
|---|---|---|---|---|
| | | | Genotype | Genotype |
| | | | anti-EGFR | anti-EGFR |
| | Nearby | Clinical | Therapy | Therapy |
| Polymorphism | Gene | Endpoint | (Cetuximab) | (Cetuximab) |
| rs3853839 | TLR7 | PFS, OS | G/G | C/G or C/C |
| | | | Favorable | Unfavorable |
| | | | Genotype | Genotype |
| | | | Irinotecan plus | Irinotecan plus |
| | | | Bevacizumab | Bevacizumab |
| rs5743618 | TLR1 | PFS, OS | G/T or G/G | T/T |
| rs3821985 | TLR6 | PFS, OS | A/A | A/C or C/C |
| s5743836 | TLR9 | PFS, OS | T/T | T/C or C/C |

*The genotypes noted here only refer to one DNA strand; for instance, genotype C/G is equivalent to G/C on the opposite strand and should be understood to encompass both strands.

In some embodiments, provided is an in vitro method of detecting a polymorphism in a patient with cancer or a patient suspected of having cancer, e.g., GI cancer, colon cancer, rectal cancer, or colorectal cancer, the method comprising, or alternatively consisting essentially of, or yet further consisting of screening a biological sample from the patient to detect the genotype of (G/G), (C/G), or (C/C) for rs3853839. In some embodiments, the patient is known to have the cancer. In other embodiments, the patient is suspected of having the cancer. In some embodiments, the screening comprises, or alternatively consists essentially of, or yet further consists of detecting the genotype of (G/G) for rs3853839 in the sample. In some embodiments, the screening comprises, or alternatively consists essentially of, or yet further consists of detecting the genotype of (C/G) for rs3853839 in the sample. In some embodiments, the screening comprises, or alternatively consists essentially of, or yet further consists of detecting the genotype of (C/C) for rs3853839 in the sample. In some embodiments, the method further comprises, or alternatively consists essentially of, or yet further consists of, administering a therapy comprising an effective amount of an anti-EGFR therapy, e.g. cetuximab. The therapy can be first line, second line, third line, fourth line of fifth line therapy and is some aspects, can be administered alone or in combination with other therapies, e.g., surgical resection, radiation therapy or other chemical or biological based therapies.

In some embodiments, provided is a method for selecting a cancer patient, e.g. a GI cancer patient, a colon cancer patient, a rectal cancer patient, or a colorectal cancer patient, for an anti-EGFR therapy, for example, comprising, consisting essentially of, or yet consisting of, cetuximab, the method comprising screening a biological sample isolated from the patient for an rs3853839 polymorphism, and selecting the patient for the therapy if the genotype of (G/G) for rs3853839 is present in the sample. In some embodiments, the patient is not selected for a therapy comprising, consisting essentially of, or yet consisting of, a therapeutically effective amount of an anti-EGFR therapy, e.g. cetuximab, if the genotype of (G/G) for rs3853839 is not present in the sample. In some embodiments, the patient is not selected for an anti-EGFR therapy, for example, comprising, consisting essentially of, or yet consisting of, cetuximab, if the genotype of (C/G) or (C/C) for rs3853839 is present in the sample. In some embodiments, the patient is selected for an anti-EGFR-free, or cetuximab-free, therapy if the genotype of (G/G) for rs3853839 is not present in the sample. In some embodiments, the patient is selected for an anti-EGFR-free, or cetuximab-free, therapy if the genotype of (C/G) or (C/C) for rs3853839 is present in the sample. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for classifying a cancer patient, e.g. a GI cancer patient, a colon cancer patient, a rectal cancer patient, or a colorectal cancer patient, as eligible for an anti-EGFR therapy, for example, comprising, consisting essentially of, or yet consisting of, administration of an effective amount of cetuximab, the method comprising screening a biological sample isolated from the patient for an rs3853839 polymorphism, and classifying the patient as eligible for the therapy if the genotype of (G/G) for rs3853839 is present in the sample. In some embodiments, the method comprises, or alternatively consisting essentially, or yet further consisting of, classifying the patient as not eligible for the therapy an anti-EGFR therapy, for example, comprising cetuximab, if the genotype of (G/G) for rs3853839 is not present in the sample. In some embodiments, the patient is classified as not eligible for the therapy comprising, consisting essentially of, or yet consisting of an anti-EGFR therapy, for example, cetuximab, if the genotype of (C/G) or (C/C) for rs3853839 is present in the sample. In some embodiments, the method further comprises, or alternatively consisting essentially of, or yet consisting of, administering a therapy comprising, consisting essentially of, or yet consisting of, a therapeutically effective amount of an anti-EGFR therapy, for example, cetuximab. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for increasing the progression-free and/or overall survival of a cancer patient, e.g. a GI cancer patient, a colon cancer patient, a rectal cancer patient, or a colorectal cancer patient, the method comprising, consisting essentially of, or yet consisting of, screening a biological sample isolated from the patient for an rs3853839 polymorphism, and classifying the patient as eligible for an anti-EGFR therapy, for example, comprising, consisting essentially of, or yet consisting of, cetuximab, if the genotype of (G/G) for rs3853839 is present in the sample or not eligible for the therapy comprising, consisting essentially of, or yet consisting of, an anti-EGFR therapy, for example, comprising, consisting essentially of, or yet consisting of, cetuximab, if the genotype of (G/G) for rs3853839 is not present in the sample. In some embodiments, the patient is classified as not eligible for the therapy comprising, consisting essentially of, or yet consisting of, an anti-EGFR therapy, for example, comprising, consisting essentially of, or yet consisting of, cetuximab, if the genotype of (C/G) or (C/C) for rs3853839 is present in the sample. In some embodiments, the method further comprises, or alternatively consisting essentially of, or yet further consisting of, administering a therapy comprising a therapeutically effective amount of an anti-EGFR therapy, for example, cetuximab, or an anti-EGFR-free or cetuximab-free therapy in accordance with the classification. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for identifying whether a cancer patient, e.g. a GI cancer patient, a colon cancer patient, a rectal cancer patient, or a colorectal cancer patient, is likely to experience a relatively longer or shorter progression free survival (PFS) following a therapy comprising, consisting essentially of, or yet consisting of, a therapeutically effective amount of anti-EGFR therapy, for example, cetuximab, comprising, consisting essentially of, or yet consisting of, screening a biological sample isolated from the patient for an rs3853839 polymorphism, and identifying that the patient is likely to experience a longer progression free survival if the genotype of (G/G) for rs3853839 is present in the sample, relative to a corresponding cancer patient not having the genotype. In some embodiments, the method comprises, or alternatively consisting of, or yet further consisting of, identifying that the patient is likely to experience a shorter progression free survival if the genotype of (G/G) for rs3853839 is not present in the sample, relative to a corresponding cancer patient having the genotype or relative to a corresponding cancer patient having the genotype of (C/G) or (C/C) for rs3853839. In some embodiments, the method comprises, or consists essentially of, or yet further consists of, identifying that the patient is likely to experience a shorter progression free survival if the genotype of (C/G) or (C/C) for rs3853839 is present in the sample, relative to a corresponding cancer patient not having the genotype or relative to a corresponding cancer patient having the genotype of (G/G) for rs3853839. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for treating a cancer patient, e.g. a GI cancer patient, a colon cancer patient, a rectal cancer patient, or a colorectal cancer patient, selected for treatment based on the presence of the genotype of (G/G) for rs3853839 in a biological sample from the patient, the method comprising, consisting essentially of, or yet consisting of, administering to the patient a therapy comprising a therapeutically effective amount of anti-EGFR therapy, for example, cetuximab. Also provided, in some embodiments, is a method for treating a cancer patient selected for treatment based on the absence of the genotype of (C/G) or (C/C) for rs3853839 in a biological sample from the patient, the method comprising, consisting essentially of, or yet consisting of, administering to the patient a therapy comprising, consisting essentially of, or yet consisting of, a therapeutically effective amount of a therapeutically effective amount of anti-EGFR therapy, for example, cetuximab. The therapy can be first line, second line, third line, fourth line of fifth line therapy and is some aspects, can be administered alone or in combination with other therapies, e.g., surgical resection, radiation therapy or other chemical or biological based therapies.

In some embodiments, the method further comprises, or alternatively consisting essentially of, or consisting of, screening a biological sample isolated from the patient for the rs3853839 polymorphism. Thus, also provided, in some embodiments, is a method for treating a cancer patient, e.g. a GI cancer patient, a colon cancer patient, a rectal cancer patient, or a colorectal cancer patient, the method comprising, consisting essentially of, or yet consisting of, screening a biological sample isolated from the patient for the rs3853839 polymorphism and administering to the patient a therapy comprising, consisting essentially of, or yet consisting of, a therapeutically effective amount of anti-EGFR therapy, for example, cetuximab, if the sample has the genotype of (G/G) for rs3853839. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for modifying the treatment of a cancer patient, e.g. a GI cancer patient, a colon cancer patient, a rectal cancer patient, or a colorectal cancer patient, receiving a therapy comprising, consisting essentially of, or yet consisting of, a therapeutically effective amount of anti-EGFR therapy, for example, cetuximab, based on the presence of the genotype of (G/G) for rs3853839 in a biological sample from the patient. For example, provided is a method for modifying the treatment of a cancer patient, e.g. a GI cancer patient, a colon cancer patient, a rectal cancer patient, or a colorectal cancer patient, receiving a therapy comprising, consisting essentially of, or yet consisting of, a therapeutically effective amount of anti-EGFR therapy, for example, cetuximab, the method comprising screening a biological sample isolated from the patient for an rs3853839 polymorphism, and modifying the dosage or frequency of the therapy comprising, consisting essentially of, or yet consisting of, a therapeutically effective amount of anti-EGFR therapy, for example, cetuximab, based on the genotype for rs3853839. In some embodiments, the dosage or frequency of the therapy, or components thereof (e.g., one or more therapeutic agents of the therapy), is increased if the genotype of (G/G) for rs3853839 is not present in the sample. In some embodiments, the dosage or frequency of the therapy, or components thereof, is increased if the genotype of (C/G) or (C/C) for rs3853839 is present in the sample. In some embodiments, the therapy is discontinued if the genotype of (G/G) for rs3853839 is not present in the sample. In some embodiments, the therapy is discontinued if the genotype of (C/G) or (C/C) for rs3853839 is present in the sample. In some embodiments, the therapy is continued if the genotype of (G/G) for rs3853839 is present in the sample. The therapy can be first line, second line, third line, fourth line of fifth line therapy and is some aspects, can be administered alone or in combination with other therapies, e.g., surgical resection, radiation therapy or other chemical or biological based therapies.

In some embodiments, screening a biological sample isolated from the patient for an rs3853839 polymorphism comprises, or alternatively consisting essentially of, or yet consisting of, contacting the biological sample with a nucleic acid probe that specifically binds to nucleic acid containing the rs3853839 polymorphism and overlaps the polymorphic site. For example, in some embodiments, the nucleic acid specifically binds to a nucleic acid having the sequence of SEQ ID NO:1 and overlaps the polymorphic site. In some embodiments, the a nucleic acid is labeled with a detectable moiety, having about 5, about 10, about 15, about 20, about 25, about 30, about 35, or about 40 nucleotides upstream and/or downstream of the polymorphic region. In another aspect, whole genome sequencing can be used to determine the identity of the genome at the site of interest.

In some embodiments, screening a biological sample isolated from the patient for an rs3853839 polymorphism comprises, or consists essentially of, or yet consisting of, amplifying nucleic acid containing the rs3853839 polymorphism. In some embodiments, nucleic acid containing the rs3853839 polymorphism is amplified using a forward primer comprising, consisting essentially of, or yet consisting of, nucleic acid having the sequence of SEQ ID NO:2 and a reverse primer comprising nucleic acid having the sequence of SEQ ID NO:3.

In some aspects, therapy comprising anti-EGFR therapy, for example, cetuximab, further comprises, consisting essentially of, or yet consisting of, a therapeutically effective amount of oxaliplatin. In some aspects, therapy comprising oxaliplatin comprises a therapeutically effective amount of folinic acid and/or a pyrimidine analog. In some aspects, the therapy comprises, or consists essentially of, or yet further consists of, FOLFOX (leucovorin+Fluorouracil (5-FU)+oxaliplatin).

In some aspects, therapy comprising anti-EGFR therapy, for example, cetuximab, further comprises, or consists essentially of, or yet further consists of, a therapeutically effective amount of irinotecan. In some aspects, therapy comprising irinotecan comprises a therapeutically effective amount of folinic acid and/or a pyrimidine analog. In some aspects, the therapy comprises FOLFIRI (leucovorin+Fluorouracil (5-FU)+irinotecan). In some aspects, the therapy further comprises, or consists essentially of, or yet further consists of, therapeutically effective amounts of irinotecan and oxaliplatin. In some aspects, the therapy comprises FOLFOXFIRI (leucovorin+Fluorouracil (5-FU)+oxaliplatin+irinotecan). In some aspects, the therapy further comprises, or consists essentially of, or yet further consists of, a therapeutically effective amount of bevacizumab.

In some aspects, the patient has a wild-type KRAS and/or BRAF gene.

In some aspects, the patient for the methods described herein suffers from colon cancer, non-metastatic colorectal cancer or metastatic colorectal cancer.

In some aspects, the biological sample is a tissue or a cell sample. In some aspects, the sample comprises at least one of a tumor cell, a normal cell adjacent to a tumor, a normal cell corresponding to the tumor tissue type, a blood cell, a peripheral blood lymphocyte, or combinations thereof.

In some aspects, the sample is at least one of blood, plasma, serum, an original sample recently isolated from the patient, a fixed tissue, a previously frozen tissue, a biopsy tissue, a resection tissue, a microdissected tissue, or combinations thereof.

In some aspects, the screening the rs3853839 polymorphism is by a method comprising PCR, RT-PCR, real-time PCR, PCR-RFLP, sequencing, whole genome sequencing, or a nucleic acid probe hybridization in solution or on a solid support, such as a chip or a microarray. In some aspects, the patient is a mammal, such as a human patient.

Also provided, in some embodiments, are kits for screening for selecting a cancer patient, e.g. a GI cancer patient, a colon cancer patient, a rectal cancer patient, or a colorectal cancer patient, for an anti-EGFR therapy, for example, comprising, or consisting essentially of, or yet further consisting of, cetuximab, or for classifying a cancer patient, e.g. a GI cancer patient, a colon cancer patient, a rectal cancer patient, or a colorectal cancer patient, as eligible for an anti-EGFR therapy, for example, comprising, or consisting essentially of, or yet further consisting of, cetuximab. In some embodiments, the kit comprises primer for amplification of nucleic acid containing a rs3853839 polymorphism. For example, in some embodiments, the kit comprises a forward primer comprising nucleic acid having the sequence of SEQ ID NO:2 and a reverse primer comprising, or consisting essentially of, or yet further consisting of, nucleic acid having the sequence of SEQ ID NO:3. In some embodiments, the kit comprises a nucleic acid probe that specifically binds to nucleic acid containing the rs3853839 polymorphism and overlaps the polymorphic site. For example, in some embodiments, the nucleic acid probe specifically binds to a nucleic acid having the sequence of SEQ ID NO:1 and overlaps the polymorphic site. In some embodiments, the nucleic acid probe has about 5, about 10, about 15, about 20, about 25, about 30, about 35 or about 40 or more contiguous nucleotides of SEQ ID NO:1 and overlaps the polymorphic site.

In some embodiments, provided is an in vitro method of detecting a polymorphism in a patient with cancer or a patient suspected of having cancer, e.g., GI cancer, colon cancer, rectal cancer, or colorectal cancer, the method comprising, or alternatively consisting essentially of, or yet further consisting of screening a biological sample from the patient to detect the genotype of (G/T) or (G/G) for rs5743618. In some embodiments, the patient is known to have the cancer. In other embodiments, the patient is suspected of having the cancer. In some embodiments, the screening comprises, or alternatively consists essentially of, or yet further consists of detecting the genotype of (G/T) for rs5743618 in the sample. In some embodiments, the screening comprises, or alternatively consists essentially of, or yet further consists of detecting the genotype of (G/G) for rs5743618 in the sample. In some embodiments, the method further comprises, or alternatively consists essentially of, or yet further consists of, administering a therapy comprising an effective amount of irinotecan and/or bevacizumab. The therapy can be first line, second line, third line, fourth line of fifth line therapy and is some aspects, can be administered alone or in combination with other therapies, e.g., surgical resection, radiation therapy or other chemical or biological based therapies.

In some embodiments, provided is a method for selecting a cancer patient, e.g. a GI cancer patient, a colon cancer patient, a rectal cancer patient, or a colorectal cancer patient, for a therapy comprising, or consisting essentially of, or yet further consisting of, irinotecan and bevacizumab, the method comprising, or consisting essentially of, or yet further consisting of, screening a biological sample isolated from the patient for an rs5743618 polymorphism, and selecting the patient for the therapy if the genotype of (G/T) or (G/G) for rs5743618 is present in the sample. In some embodiments, the patient is not selected for a therapy comprising a therapeutically effective amount of irinotecan and bevacizumab if the genotype of (G/T) or (G/G) for rs5743618 is not present in the sample. In some embodiments, the patient is not selected for a therapy comprising, or consisting essentially of, or yet further consisting of, irinotecan and bevacizumab if the genotype of (T/T) for rs5743618 is present in the sample. In some embodiments, the patient is selected for an irinotecan and bevacizumab-free therapy if the genotype of (G/T) or (G/G) for rs5743618 is not present in the sample. In some embodiments, the patient is selected for an irinotecan and bevacizumab-free therapy if the genotype of (T/T) for rs5743618 is present in the sample. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for classifying a cancer patient, e.g. a GI cancer patient, a colon cancer patient, a rectal cancer patient, or a colorectal cancer patient, as eligible for a therapy comprising irinotecan and bevacizumab, the method comprising, or consisting essentially of, or yet further consisting of, screening a biological sample isolated from the patient for an rs5743618 polymorphism, and classifying the patient as eligible for the therapy if the genotype of (G/T) or (G/G) for rs5743618 is present in the sample. In some embodiments, the method comprises classifying the patient as not eligible for the therapy comprising, or consisting essentially of, or yet further consisting of, irinotecan and bevacizumab if the genotype of (G/T) or (G/G) for rs5743618 is not present in the sample. In some embodiments, the patient is classified as not eligible for the therapy comprising, or consisting essentially of, or yet further consisting of, irinotecan and bevacizumab if the genotype of (T/T) for rs5743618 is present in the sample. In some embodiments, the method further comprises administering a therapy comprising, or consisting essentially of, or yet further consisting of, a therapeutically effective amount of irinotecan and bevacizumab. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for increasing the progression-free and/or overall survival of a cancer patient, e.g. a GI cancer patient, a colon cancer patient, a rectal cancer patient, or a colorectal cancer patient, the method comprising, or consisting essentially of, or yet further consisting of, screening a biological sample isolated from the patient for an rs5743618 polymorphism, and classifying the patient as eligible for the therapy with irinotecan and bevacizumab if the genotype of (G/T) or (G/G) for rs5743618 is present in the sample or not eligible for the therapy comprising, or consisting essentially of, or yet further consisting of, irinotecan and bevacizumab if the genotype of (G/T) or (G/G) for rs5743618 is not present in the sample. In some embodiments, the patient is classified as not eligible for the therapy comprising, or consisting essentially of, or yet further consisting of, irinotecan and bevacizumab if the genotype of (T/T) for rs5743618 is present in the sample. In some embodiments, the method further comprises, or consists essentially of, or consists of, administering a therapy comprising, or consisting essentially of, or yet further consisting of, a therapeutically effective amount of irinotecan and bevacizumab or an irinotecan and bevacizumab-free therapy in accordance with the classification. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for identifying whether a cancer patient, e.g. a GI cancer patient, a colon cancer patient, a rectal cancer patient, or a colorectal cancer patient, is likely to experience a relatively longer or shorter progression free survival (PFS) following a therapy comprising, or consisting essentially of, or yet further consisting of, a therapeutically effective amount of irinotecan and bevacizumab, the method comprising, or consisting essentially of, or yet further consisting of, screening a biological sample isolated from the patient for an rs5743618 polymorphism, and identifying that the patient is likely to experience a longer progression free survival if the genotype of (G/T) or (G/G) for rs5743618 is present in the sample, relative to a corresponding cancer patient not having the genotype. In some embodiments, the method comprises identifying that the patient is likely to experience a shorter progression free survival if the genotype of (G/T) or (G/G) for rs5743618 is not present in the sample, relative to a corresponding cancer patient having the genotype or relative to a corresponding cancer patient having the genotype of (T/T) for rs5743618. In some embodiments, the method comprises, or alternatively consists essentially of, or yet consists of, identifying that the patient is likely to experience a shorter progression free survival if the genotype of (T/T) for rs5743618 is present in the sample, relative to a corresponding cancer patient not having the genotype or relative to a corresponding cancer patient having the genotype of (G/T) or (G/G) for rs5743618. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for treating a cancer patient, e.g. a GI cancer patient, a colon cancer patient, a rectal cancer patient, or a colorectal cancer patient, selected for treatment based on the presence of the genotype of (G/T) or (G/G) for rs5743618 in a biological sample from the patient, the method comprising, or consisting essentially of, or yet further consisting of, administering to the patient a therapy comprising a therapeutically effective amount of irinotecan and bevacizumab. Also provided, in some embodiments, is a method for treating a cancer patient, e.g. a GI cancer patient, a colon cancer patient, a rectal cancer patient, or a colorectal cancer patient, selected for treatment based on the absence of the genotype of (T/T) for rs5743618 in a biological sample from the patient, the method comprising, or consisting essentially of, or yet further consisting of, administering to the patient a therapy comprising, or consisting essentially of, or yet further consisting of, a therapeutically effective amount of a therapeutically effective amount of irinotecan and bevacizumab. The therapy can be first line, second line, third line, fourth line of fifth line therapy and is some aspects, can be administered alone or in combination with other therapies, e.g., surgical resection, radiation therapy or other chemical or biological based therapies.

In some embodiments, the method further comprises screening a biological sample isolated from the patient for the rs5743618 polymorphism. Thus, also provided, in some embodiments, is a method for treating a cancer patient, e.g. a GI cancer patient, a colon cancer patient, a rectal cancer patient, or a colorectal cancer patient, the method comprising, or consisting essentially of, or yet further consisting of, screening a biological sample isolated from the patient for the rs5743618 polymorphism and administering to the patient a therapy comprising, or consisting essentially of, or yet further consisting of, a therapeutically effective amount of a therapeutically effective amount of irinotecan and bevacizumab if the sample has the genotype of (G/T) or (G/G) for rs5743618. The therapy can be first line, second line, third line, fourth line of fifth line therapy and is some aspects, can be administered alone or in combination with other therapies, e.g., surgical resection, radiation therapy or other chemical or biological based therapies.

Also provided, in some embodiments, is a method for modifying the treatment of a cancer patient, e.g. a GI cancer patient, a colon cancer patient, a rectal cancer patient, or a colorectal cancer patient, receiving a therapy comprising, or consisting essentially of, or yet further consisting of, a therapeutically effective amount of irinotecan and bevacizumab based on the presence of the genotype of (G/T) or (G/G) for rs5743618 in a biological sample from the patient. For example, provided is a method for modifying the treatment of patient receiving a therapy comprising, or consisting essentially of, or yet further consisting of, a therapeutically effective amount of irinotecan and bevacizumab, comprising, or consisting essentially of, or yet further consisting of, screening a biological sample isolated from the patient for an rs5743618 polymorphism, and modifying the dosage or frequency of the therapy comprising, or consisting essentially of, or yet further consisting of, a therapeutically effective amount of irinotecan and bevacizumab based on the genotype for rs5743618. In some embodiments, the dosage or frequency of the therapy, or components thereof (e.g., one or more therapeutic agents of the therapy), is increased if the genotype of (G/T) or (G/G) for rs5743618 is not present in the sample. In some embodiments, the dosage or frequency of the therapy, or components thereof, is increased if the genotype of (T/T) for rs5743618 is present in the sample. In some embodiments, the therapy is discontinued if the genotype of (G/T) or (G/G) for rs5743618 is not present in the sample. In some embodiments, the therapy is discontinued if the genotype of (T/T) for rs5743618 is present in the sample. In some embodiments, the therapy is continued if the genotype of (G/T) or (G/G) for rs5743618 is present in the sample. The therapy can be first line, second line, third line, fourth line of fifth line therapy and is some aspects, can be administered alone or in combination with other therapies, e.g., surgical resection, radiation therapy or other chemical or biological based therapies.

In some embodiments, screening a biological sample isolated from the patient for an rs5743618 polymorphism comprises contacting the biological sample with a nucleic acid probe that specifically binds to nucleic acid containing the rs5743618 polymorphism and overlaps the polymorphic site. For example, in some embodiments, the nucleic acid specifically binds to a nucleic acid having the sequence of SEQ ID NO:4 and overlaps the polymorphic site. In some embodiments, the a nucleic acid is labeled with a detectable moiety, having about 5, about 10, about 15, about 20, about 25, about 30, about 35, or about 40 nucleotides upstream and/or downstream of the polymorphic region. In another aspect, whole genome sequencing can be used to determine the identity of the genome at the site of interest. In one aspect, the sample is isolated from a patient suspected of having colon cancer, e.g. or further, having been diagnosed with colon cancer.

In some embodiments, screening a biological sample isolated from the patient for an rs5743618 polymorphism comprises amplifying nucleic acid containing the rs5743618 polymorphism. In some embodiments, nucleic acid containing the rs5743618 polymorphism is amplified using a forward primer comprising nucleic acid having the sequence of SEQ ID NO:5 and a reverse primer comprising nucleic acid having the sequence of SEQ ID NO:6. In one aspect, the sample is isolated from a patient suspected of having colon cancer, e.g. or further, having been diagnosed with colon cancer.

In some aspects, therapy comprising irinotecan and bevacizumab further comprises therapeutically effective amount of folinic acid and/or a pyrimidine analog. In some aspects, the therapy comprises FOLFIRI (leucovorin+Fluorouracil (5-FU)+irinotecan). In some aspects, therapy comprising irinotecan and bevacizumab further comprises a therapeutically effective amount of oxaliplatin. In some aspects, therapy comprising oxaliplatin comprises a therapeutically effective amount of folinic acid and/or a pyrimidine analog. In some aspects, the therapy comprises FOLFOX (leucovorin+Fluorouracil (5-FU)+oxaliplatin). In some aspects, the therapy further comprises therapeutically effective amounts of irinotecan and oxaliplatin. In some aspects, the therapy comprises FOLFOXFIRI (leucovorin+Fluorouracil (5-FU)+oxaliplatin+irinotecan).

In some aspects, the patient has a wild-type KRAS and/or BRAF gene.

In some aspects, the patient for the methods described herein suffers from colon cancer, non-metastatic colorectal cancer or metastatic colorectal cancer.

In some aspects, the biological sample is a tissue or a cell sample. In some aspects, the sample comprises at least one of a tumor cell, a normal cell adjacent to a tumor, a normal cell corresponding to the tumor tissue type, a blood cell, a peripheral blood lymphocyte, or combinations thereof.

In some aspects, the sample is at least one of blood, plasma, serum, an original sample recently isolated from the patient, a fixed tissue, a previously frozen tissue, a biopsy tissue, a resection tissue, a microdissected tissue, or combinations thereof.

In some aspects, the screening the rs5743618 polymorphism is by a method comprising PCR, RT-PCR, real-time PCR, PCR-RFLP, sequencing, whole genome sequencing, or a nucleic acid probe hybridization in solution or on a solid support, such as a chip or a microarray. In some aspects, the patient is a mammal, such as a human patient.

Also provided, in some embodiments, are kits for screening for selecting a cancer patient, e.g. a GI cancer patient, a colon cancer patient, a rectal cancer patient, or a colorectal cancer patient, for a therapy comprising, or consisting essentially of, or yet further consisting of, irinotecan and bevacizumab or for classifying a cancer patient as eligible for a therapy comprising irinotecan and bevacizumab. In some embodiments, the kit comprises primer for amplification of nucleic acid containing a rs5743618 polymorphism. For example, in some embodiments, the kit comprises a forward primer comprising nucleic acid having the sequence of SEQ ID NO:5 and a reverse primer comprising nucleic acid having the sequence of SEQ ID NO:6. In some embodiments, the kit comprises a nucleic acid probe that specifically binds to nucleic acid containing the rs5743618 polymorphism and overlaps the polymorphic site. For example, in some embodiments, the nucleic acid probe specifically binds to a nucleic acid having the sequence of SEQ ID NO:4 and overlaps the polymorphic site. In some embodiments, the nucleic acid probe has about 5, about 10, about 15, about 20, about 25, about 30, about 35 or about 40 or more contiguous nucleotides of SEQ ID NO:4 and overlaps the polymorphic site.

Also provided is a method for provided for screening a biological sample isolated from a patient for other TLR receptor polymorphisms that are associated with progression free survival and overall survival in CRC patients receiving irinotecan and bevacizumab therapy, including, but not limited to TLR6 rs3821985 and TLR9 s5743836.

### Diagnostic Methods

Provided, in one embodiment, is a method for selecting a cancer patient, e.g. a GI cancer patient, a colon cancer patient, a rectal cancer patient, or a colorectal cancer patient, for an anti-EGFR therapy, for example, comprising cetuximab, the method comprising screening a biological sample isolated from the patient for an rs3853839 polymorphism, and selecting the patient for the therapy if the genotype of (G/G) for rs3853839 is present in the sample. In some aspects, the patient is not selected for the therapy if the genotype of (G/G) for rs3853839 is not present in the sample. In some aspects, the patient is not selected for the therapy if the genotype of (C/G) or (C/C) for rs3853839 is present in the sample. In some embodiments, the patient is selected for an cetuximab-free therapy if the genotype of (G/G) for rs3853839 is not present in the sample. In some embodiments, the patient is selected for an cetuximab-free therapy if the genotype of (C/G) or (C/C) for rs3853839 is present in the sample.

Also provided is a method for identifying whether a cancer patient, e.g. a GI cancer patient, a colon cancer patient, a rectal cancer patient, or a colorectal cancer patient, is likely to experience a relatively longer or shorter progression free survival following an anti-EGFR therapy, for example, comprising cetuximab, the method comprising screening a biological sample isolated from the patient for a rs3853839 polymorphism, and identifying that the patient is likely to experience a longer progression free survival if the genotype of (G/G) for rs3853839 is present in the sample, relative to a corresponding cancer patient not having the genotype.

In some aspects, therapy comprising cetuximab further comprises administration of a therapeutically effective amount of oxaliplatin. In some aspects, therapy comprising oxaliplatin comprises a therapeutically effective amount of folinic acid and/or a pyrimidine analog. In some aspects, the therapy comprises FOLFOX (leucovorin+Fluorouracil (5-FU)+oxaliplatin).

In some aspects, therapy comprising cetuximab further comprises administration of a therapeutically effective amount of irinotecan. In some aspects, therapy comprising irinotecan comprises a therapeutically effective amount of folinic acid and/or a pyrimidine analog. In some aspects, the therapy comprises FOLFIRI (leucovorin+Fluorouracil (5-FU)+irinotecan). In some aspects, the therapy further comprises administration of therapeutically effective amounts of irinotecan and oxaliplatin. In some aspects, the therapy comprises FOLFOXFIRI (leucovorin+Fluorouracil (5-FU)+oxaliplatin+irinotecan). In some aspects, the therapy further comprises a therapeutically effective amount of bevacizumab.

Also provided, in one embodiment, is a method for selecting a cancer patient, e.g. a GI cancer patient, a colon cancer patient, a rectal cancer patient, or a colorectal cancer patient, for a therapy comprising irinotecan and bevacizumab, the method comprising screening a biological sample isolated from the patient for an rs5743618 polymorphism, and selecting the patient for the therapy if the genotype of (G/T) or (G/G) for rs5743618 is present in the sample. In some embodiments, the patient is not selected for a therapy comprising a therapeutically effective amount of irinotecan and bevacizumab if the genotype of (G/T) or (G/G) for rs5743618 is not present in the sample. In some embodiments, the patient is not selected for a therapy comprising irinotecan and bevacizumab if the genotype of (T/T) for rs5743618 is present in the sample. In some embodiments, the patient is selected for an irinotecan and bevacizumab-free therapy if the genotype of (G/T) or (G/G) for rs5743618 is not present in the sample. In some embodiments, the patient is selected for an irinotecan and bevacizumab-free therapy if the genotype of (T/T) for rs5743618 is present in the sample.

Also provided is a method for identifying whether a cancer patient, e.g. a GI cancer patient, a colon cancer patient, a rectal cancer patient, or a colorectal cancer patient, is likely to experience a relatively longer or shorter progression free survival following a therapy comprising irinotecan and bevacizumab, the method comprising screening a biological sample isolated from the patient for a rs5743618 polymorphism, and identifying that the patient is likely to experience a longer progression free survival if the genotype of (G/T) or (G/G) for rs5743618 is present in the sample, relative to a corresponding cancer patient not having the genotype.

In some aspects, therapy comprising irinotecan and bevacizumab further comprises therapeutically effective amount of folinic acid and/or a pyrimidine analog. In some aspects, the therapy comprises FOLFIRI (leucovorin+Fluorouracil (5-FU)+irinotecan). In some aspects, therapy comprising irinotecan and bevacizumab further comprises a therapeutically effective amount of oxaliplatin. In some aspects, therapy comprising oxaliplatin comprises a therapeutically effective amount of folinic acid and/or a pyrimidine analog. In some aspects, the therapy comprises FOLFOX (leucovorin+Fluorouracil (5-FU)+oxaliplatin). In some aspects, the therapy further comprises therapeutically effective amounts of irinotecan and oxaliplatin. In some aspects, the therapy comprises FOLFOXFIRI (leucovorin+Fluorouracil (5-FU)+oxaliplatin+irinotecan).

In some aspects, the patient suffers from non-metastatic colorectal cancer or metastatic colorectal cancer. In some aspects, the cancer is metastatic or non-metastatic colon cancer. In some aspects, the cancer is metastatic or non-metastatic rectal cancer.

Any suitable method for identifying the genotype in the patient sample can be used and the disclosures described herein are not to be limited to these methods. For the purpose of illustration only, the genotype is determined by a method comprising, or alternatively consisting essentially of, or yet further consisting of, sequencing, whole genome sequencing, next-generation sequencing, hybridization, nucleic acid amplification, including polymerase chain reaction (PCR), real-time PCR, reverse transcriptase PCR (RT-PCR), nested PCR, ligase chain reaction, or PCR-RFLP, or microarray. These methods as well as equivalents or alternatives thereto are described herein.

The methods are useful in the assistance of an animal, a mammal or yet further a human patient. For the purpose of illustration only, a mammal includes but is not limited to a human, a simian, a murine, a bovine, an equine, a porcine or an ovine subject.

Information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

### Biological Sample Collection and Preparation

The methods and compositions disclosed herein can be used to detect nucleic acids associated with a rs3853839 or rs5743618 polymorphism using a biological sample obtained from a patient. Biological samples can be obtained by standard procedures and can be used immediately or stored, under conditions appropriate for the type of biological sample, for later use. Any liquid or solid biological material obtained from the patient believed to contain nucleic acids comprising the region containing the rs3853839 or rs5743618 polymorphism can be any suitable sample.

Methods of obtaining test samples are known to those of skill in the art and include, but are not limited to, aspirations, tissue sections, swabs, drawing of blood or other fluids, surgical or needle biopsies.

In some aspects, the biological sample is a tissue or a cell sample. Suitable patient samples in the methods include, but are not limited to, blood, plasma, serum, a biopsy tissue, fine needle biopsy sample, amniotic fluid, plasma, pleural fluid, saliva, semen, serum, tissue or tissue homogenates, frozen or paraffin sections of tissue or combinations thereof. In some aspects, the biological sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. In some aspects, the biological sample is an original sample recently isolated from the patient, a fixed tissue, a previously frozen tissue, a resection tissue, or a microdissected tissue. In some aspects, the biological samples are processed, such as by sectioning of tissues, fractionation, purification, nucleic acid isolation, or cellular organelle separation.

In some embodiments, nucleic acid (DNA or RNA) is isolated from the sample according to any methods known to those of skill in the art. In some aspects, genomic DNA is isolated from the biological sample. In some aspects, RNA is isolated from the biological sample. In some aspects, cDNA is generated from mRNA in the sample. In some embodiments, the nucleic acid is not isolated from the biological sample (e.g., the polymorphism is detected directly from the biological sample).

### Detection of Polymorphisms

In some aspects, detection of polymorphisms can be accomplished by molecular cloning of the specified allele and subsequent sequencing of that allele using techniques known in the art, in some aspects, after isolation of a suitable nucleic acid sample. In some aspects, the gene sequences can be amplified directly from a genomic DNA preparation from the biological sample using PCR, and the sequence composition is determined by sequencing the amplified product (i.e., amplicon). Alternatively, the PCR product can be analyzed following digestion with a restriction enzyme, a method known as PCR-RFLP.

In some embodiments, the polymorphism is detected using allele specific hybridization using probes overlapping the polymorphic site. In some aspects, the nucleic acid probes are between 5 and 40 nucleotides in length. In some aspects, the nucleic acid probes are about 5, about 10, about 15, about 20, about 25, about 30, about 35, or about 40 or more nucleotides flanking the polymorphic site. For example, in some embodiments, the nucleic acid specifically binds to a nucleic acid having the sequence of SEQ ID NO:1 or 4 and overlaps the polymorphic site. Exemplary probes include nucleic acid probes having about 5, about 10, about 15, about 20, about 25, about 30, about 35, about 40 or more contiguous nucleotides of SEQ ID NO:1 or 4 and overlaps the polymorphic site.

In another embodiment of the disclosure, several nucleic acid probes capable of hybridizing specifically to the nucleic acid containing the allelic variant are attached to a solid phase support, e.g., a “chip” or “microarray. Such gene chips or microarrays can be used to detect genetic variations by a number of techniques known to one of skill in the art. In one technique, oligonucleotides are arrayed on a gene chip for determining the DNA sequence by the sequencing by hybridization approach. The probes of the disclosure also can be used for fluorescent detection of a genetic sequence. A probe also can be affixed to an electrode surface for the electrochemical detection of nucleic acid sequences.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers can be detectably labeled thereby identifying the polymorphism in the gene(s) of interest. Alternatively, a chemical or biological reaction can be used to identify the probes or primers which hybridized with the DNA or RNA of the gene(s) of interest. The genetic profile of the patient is then determined with the aid of the aforementioned apparatus and methods.

In some aspects, whole genome sequencing, in particular with the “next generation sequencing” techniques, which employ massively parallel sequencing of DNA templates, can be used to obtain genotypes of relevant polymorphisms. Exemplary NGS sequencing platforms for the generation of nucleic acid sequence data include, but are not limited to, Illumina's sequencing by synthesis technology (e.g., Illumina MiSeq or HiSeq System), Life Technologies' Ion Torrent semiconductor sequencing technology (e.g., Ion Torrent PGM or Proton system), the Roche (454 Life Sciences) GS series and Qiagen (Intelligent BioSystems) Gene Reader sequencing platforms.

In some aspects, nucleic acid comprising the polymorphism is amplified to produce an amplicon containing the polymorphism. Nucleic acids can be amplified by various methods known to the skilled artisan. Nucleic acid amplification can be linear or exponential. Amplification is generally carried out using polymerase chain reaction (PCR) technologies. Alternative or modified PCR amplification methods can also be used and include, for example, isothermal amplification methods, rolling circle methods, Hot-start PCR, real-time PCR, Allele-specific PCR, Assembly PCR or Polymerase Cycling Assembly (PCA), Asymmetric PCR, Colony PCR, Emulsion PCR, Fast PCR, Real-Time PCR, nucleic acid ligation, Gap Ligation Chain Reaction (Gap LCR), Ligation-mediated PCR, Multiplex Ligation-dependent Probe Amplification, (MLPA), Gap Extension Ligation PCR (GEXL-PCR), quantitative PCR (Q-PCR), Quantitative real-time PCR (QRT-PCR), multiplex PCR, Helicase-dependent amplification, Intersequence-specific (ISSR) PCR, Inverse PCR, Linear-After-The-Exponential-PCR (LATE-PCR), Methylation-specific PCR (MSP), Nested PCR, Overlap-extension PCR, PAN-AC assay, Reverse Transcription PCR(RT-PCR), Rapid Amplification of cDNA Ends (RACE PCR), Single molecule amplification PCR(SMA PCR), Thermal asymmetric interlaced PCR (TAIL-PCR), Touchdown PCR, long PCR, nucleic acid sequencing (including DNA sequencing and RNA sequencing), transcription, reverse transcription, duplication, DNA or RNA ligation, and other nucleic acid extension reactions known in the art. The skilled artisan will understand that other methods can be used either in place of, or together with, PCR methods, including enzymatic replication reactions developed in the future. See, e.g., Saiki, “Amplification of Genomic DNA” in PCR Protocols, Innis et al., eds., Academic Press, San Diego, Calif., 13-20 (1990); Wharam, et al., 29(11) Nucleic Acids Res, E54-E54 (2001); Hafner, et al., 30(4) Biotechniques, 852-6, 858, 860 passim (2001).

In some aspects, nucleic acid comprising the rs3853839 or rs5743618 polymorphism is amplified to produce an amplicon containing the rs3853839 or rs5743618 polymorphism. For example, in some aspects, nucleic acid comprising SEQ ID NO:1 or 4 is amplified to generate an amplicon comprising SEQ ID NO:1 or 4. In some aspects, nucleic acid containing the rs3853839 or rs5743618 polymorphism is amplified using a forward primer and a reverse primer the flank the rs3853839 or rs5743618 polymorphism. In some aspects, nucleic acid containing the rs3853839 or rs5743618 polymorphism is amplified using a forward primer comprising nucleic acid having the sequence of SEQ ID NO:2 and a reverse primer comprising nucleic acid having the sequence of SEQ ID NO:3 or a forward primer comprising nucleic acid having the sequence of SEQ ID NO:5 and a reverse primer comprising nucleic acid having the sequence of SEQ ID NO:6. In some aspects, the amplicon containing the rs3853839 or rs5743618 polymorphism is detected using a nucleic acid probe. In some aspects, the amplicon containing the rs3853839 or rs5743618 polymorphism is detected by hybridizing a nucleic acid probe containing the rs3853839 or rs5743618 polymorphism or a complement thereof to the corresponding complementary strand of the amplicon and detecting the hybrid formed between the nucleic acid probe and the complementary strand of the amplicon. In some aspects, amplicon containing the rs3853839 or rs5743618 polymorphism is sequenced (e.g., dideoxy chain termination methods (Sanger method and variants thereof), Maxam & Gilbert sequencing, pyrosequencing, exonuclease digestion and next-generation sequencing methods).

In some embodiments, the amplification includes a labeled primer or probe, thereby allowing detection of the amplification products corresponding to that primer or probe. In particular embodiments, the amplification can include a multiplicity of labeled primers or probes; such primers can be distinguishably labeled, allowing the simultaneous detection of multiple amplification products.

In some embodiments, the amplification products are detected by any of a number of methods such as gel electrophoresis, column chromatography, hybridization with a nucleic acid probe, or sequencing the amplicon.

Detectable labels can be used to identify the primer or probe hybridized to a genomic nucleic acid or amplicon. Detectable labels include but are not limited to fluorophores, isotopes (e.g., ³²P, ³³P, ³⁵S, ³H, ¹⁴C, ¹²⁵I, ¹³¹I) electron-dense reagents (e.g., gold, silver), nanoparticles, enzymes commonly used in an ELISA (e.g., horseradish peroxidase, beta-galactosidase, luciferase, alkaline phosphatase), chemiluminiscent compounds, colorimetric labels (e.g., colloidal gold), magnetic labels (e.g., Dynabeads®), biotin, digoxigenin, haptens, proteins for which antisera or monoclonal antibodies are available, ligands, hormones, oligonucleotides capable of forming a complex with the corresponding oligonucleotide complement.

In one embodiment, a primer or probe is labeled with a fluorophore that emits a detectable signal. The term “fluorophore” as used herein refers to a molecule that absorbs light at a particular wavelength (excitation frequency) and subsequently emits light of a longer wavelength (emission frequency). While a suitable reporter dye is a fluorescent dye, any reporter dye that can be attached to a detection reagent such as an oligonucleotide probe or primer is suitable for use in the methods described. Suitable fluorescent moieties include, but are not limited to, the following fluorophores working individually or in combination: 4-acetamido-4′-isothiocyanatostilbene-2,2′disulfonic acid; acridine and derivatives, e,g, acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; and tetramethyl rhodamine isothiocyanate (TRITC).

In some aspects, the primer or probe is further labeled with a quencher dye such as Tamra, Dabcyl, or Black Hole Quencher®(BHQ), especially when the reagent is used as a self-quenching probe such as a TaqMan®(U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

In some aspects, methods for real time PCR use fluorescent primers/probes, such as the TaqMan® primers/probes (Heid, et al., Genome Res 6: 986-994, 1996), molecular beacons, and Scorpion™ primers/probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion®™ and TaqMan® technologies are based on the principle of fluorescence quenching and involve a donor fluorophore and a quenching moiety. The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety. The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® primers/probes while proximal quenching is used in molecular beacon and Scorpion™ type primers/probes.

The detectable label can be incorporated into, associated with or conjugated to a nucleic acid primer or probe. Labels can be attached by spacer arms of various lengths to reduce potential steric hindrance or impact on other useful or desired properties. See, e.g., Mansfield, Mol. Cell. Probes (1995), 9:145-156.

Detectable labels can be incorporated into nucleic acid probes by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3™ or Cy5™ and then incorporated into nucleic acid probes during nucleic acid synthesis or amplification. Nucleic acid probes can thereby be labeled when synthesized using Cy3™- or Cy5™-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu et al., Nat. Biotechnol. (2000), 18:345-348,

Nucleic acid probes can be labeled by non-covalent means known in the art. For example, Kreatech Biotechnology's Universal Linkage System® (ULS®) provides a non-enzymatic labeling technology, wherein a platinum group forms a co-ordinative bond with DNA, RNA or nucleotides by binding to the N7 position of guanosine. This technology can also be used to label proteins by binding to nitrogen and sulfur containing side chains of amino acids. See, e.g., U.S. Pat. Nos. 5,580,990; 5,714,327; and 5,985,566; and European Patent No. 0539466.

Labeling with a detectable label also can include a nucleic acid attached to another biological molecule, such as a nucleic acid, e.g., an oligonucleotide, or a nucleic acid in the form of a stem-loop structure as a “molecular beacon” or an “aptamer beacon”. Molecular beacons as detectable moieties are described; for example, Sokol (Proc. Natl. Acad. Sci. USA (1998), 95:11538-11543) synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (Biochemistry (2001), 40:9387-9395), describing a molecular beacon consist of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi, Anal. Biochem. (2001), 294:126-131; Poddar, Mol. Cell. Probes (2001), 15:161-167; Kaboev, Nucleic Acids Res. (2000), 28:E94. Aptamer beacons can adopt two or more conformations, one of which allows ligand binding. A fluorescence-quenching pair is used to report changes in conformation induced by ligand binding. See also, e.g., Yamamoto et al., Genes Cells (2000), 5:389-396; Smimov et al., Biochemistry (2000), 39:1462-1468.

The nucleic acid primer or probe can be indirectly detectably labeled via a peptide. A peptide can be made detectable by incorporating predetermined polypeptide epitopes recognized by a secondary reporter (e.g., leucine zipper pair sequences, binding sites for secondary antibodies, transcriptional activator polypeptide, metal binding domains, epitope tags). A label can also be attached via a second peptide that interacts with the first peptide (e.g., S-S association).

As readily recognized by one of skill in the art, detection of the complex containing the nucleic acid from a sample hybridized to a labeled probe can be achieved through use of a labeled antibody against the label of the probe. In one example, the probe is labeled with digoxigenin and is detected with a fluorescent labeled anti-digoxigenin antibody. In another example, the probe is labeled with FITC, and detected with fluorescent labeled anti-FITC antibody. These antibodies are readily available commercially. In another example, the probe is labeled with FITC, and detected with anti-FITC antibody primary antibody and a labeled anti-anti FITC secondary antibody.

Nucleic acids can be amplified prior to detection or can be detected directly during an amplification step (i.e., “real-time” methods, such as in TaqMan® and Scorpion™ methods). In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

With regard to the exemplary primers and probes, those skilled in the art will readily recognize that nucleic acid molecules can be double-stranded molecules and that reference to a particular site on one strand refers, as well, to the corresponding site on a complementary strand. In defining a variant position, allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular variant position, allele, or nucleotide sequence. Probes and primers, can be designed to hybridize to either strand and detection methods disclosed herein can generally target either strand.

In some embodiments, the primers and probes comprise additional nucleotides corresponding to sequences of universal primers (e.g., T7, M13, SP6, T3) which add the additional sequence to the amplicon during amplification to permit further amplification and/or prime the amplicon for sequencing.

### Methods of Treatment

The disclosure further provides methods of treating a patient selected by any method of the above embodiments, or identified as likely to experience a more favorable clinical outcome by any of the above methods, following the therapy. In some embodiments, the methods entail administering to the patients such a therapy.

In some embodiments, provided is a method for treating a cancer patient selected for treatment based on the presence of the genotype of (G/G) for rs3853839 in a biological sample from the patient, the method comprising administering to the patient a therapy comprising a therapeutically effective amount of cetuximab. In some embodiments, the patient is treated with an cetuximab-free therapy if the genotype of (G/G) for rs3853839 is not present in the sample. In some embodiments, the patient is treated with an cetuximab-free therapy if the genotype of (C/G) or (C/C) for rs3853839 is present in the sample.

In some embodiments, provided is a method for treating a cancer patient selected for treatment based on the presence of the genotype of (G/T) or (G/G) for rs5743618 in a biological sample from the patient, the method comprising administering to the patient a therapy comprising a therapeutically effective amount of irinotecan and bevacizumab. In some embodiments, the patient is treated with an irinotecan and bevacizumab-free therapy if the genotype of (G/T) or (G/G) for rs5743618 is not present in the sample. In some embodiments, the patient is treated with an irinotecan and bevacizumab-free therapy if the genotype of (T/T) for rs5743618 is present in the sample.

In some aspects, the patient is selected by a method comprising screening a tissue or cell sample isolated from the patient for the rs3853839 or rs5743618 polymorphism. Exemplary methods for screening are described in the diagnostic methods provided above and throughout the present disclosure. Any such diagnostic methods disclosed for the detection of a rs3853839 or rs5743618 polymorphism can be combined with the treatment methods provided herein.

In some aspects, therapy comprising cetuximab further comprises a therapeutically effective amount of oxaliplatin. In some aspects, therapy comprising oxaliplatin comprises a therapeutically effective amount of folinic acid and/or a pyrimidine analog. In some aspects, the therapy comprises FOLFOX (leucovorin+Fluorouracil (5-FU)+oxaliplatin). In some aspects, therapy comprising cetuximab further comprises a therapeutically effective amount of irinotecan. In some aspects, therapy comprising irinotecan comprises a therapeutically effective amount of folinic acid and/or a pyrimidine analog. In some aspects, the therapy comprises FOLFIRI (leucovorin+Fluorouracil (5-FU)+irinotecan). In some aspects, the therapy further comprises therapeutically effective amounts of irinotecan and oxaliplatin. In some aspects, the therapy comprises FOLFOXFIRI (leucovorin+Fluorouracil (5-FU)+oxaliplatin+irinotecan). In some aspects, the therapy further comprises a therapeutically effective amount of bevacizumab.

In some aspects, therapy comprising irinotecan and bevacizumab further comprises therapeutically effective amount of folinic acid and/or a pyrimidine analog. In some aspects, the therapy comprises FOLFIRI (leucovorin+Fluorouracil (5-FU)+irinotecan). In some aspects, therapy comprising irinotecan and bevacizumab further comprises a therapeutically effective amount of oxaliplatin. In some aspects, therapy comprising oxaliplatin comprises a therapeutically effective amount of folinic acid and/or a pyrimidine analog. In some aspects, the therapy comprises FOLFOX (leucovorin+Fluorouracil (5-FU)+oxaliplatin). In some aspects, the therapy further comprises therapeutically effective amounts of irinotecan and oxaliplatin. In some aspects, the therapy comprises FOLFOXFIRI (leucovorin+Fluorouracil (5-FU)+oxaliplatin+irinotecan).

In some aspects, the patient suffers from non-metastatic colorectal cancer or metastatic colorectal cancer. In some aspects, the colorectal cancer is colon cancer. In some aspects, the colorectal cancer is rectal cancer.

Exemplary dosing schedules for the treatment of colorectal cancer with cetuximab include but are not limited 400 mg/m² IV infused over 2 hr and maintenance of 250 mg/m² IV infusion over 60 min every week.

Exemplary dosing schedules for the treatment of colorectal cancer with bevacizumab include but are not limited to 5-10 mg/kg IV every two weeks.

Exemplary dosing schedules for the treatment of colorectal cancer with irinotecan include but are not limited to 125 mg/m² IV infusion over 90 minutes on days 1, 8, 15, 22, then 2 weeks off, then repeat or 350 mg/m² IV infusion over 30-90 minutes once every 3 weeks. Exemplary dosing schedules for the treatment of colorectal cancer with irinotecan as combination therapy include but are not limited to 180 mg/m² IV infusion over 30-90 minutes once on days 1, 15, and 29 IV (infuse over 30-90 min), followed by infusion with leucovorin and 5-fluorouracil; next cycle begins on day 43 (6 week cycle) or 125 mg/m² on days 1, 8, 15, and 22 (infuse over 90 min), followed by bolus doses of leucovorin and 5-fluorouracil.

Exemplary dosing schedules for the treatment of colorectal cancer with Oxaliplatin include but are not limited to Oxaliplatin 75-85 mg/m² IV+leucovorin 200 mg/m² IV infused over 2 hr, then 5-FU 300-400 mg/m² IV bolus over 2-4 minutes, then 5-FU 500-600 mg/m² IV infusion in D5W (500 mL) over 22 hr on day 1 and then repeat on day 2 without oxaliplatin, and then repeat the 2-day regimen every 2 weeks. In some aspect, treatment is provided following tumor resection.

The methods are useful in the assistance of an animal, a mammal or yet further a human patient. For the purpose of illustration only, a mammal includes but is not limited to a human, a simian, a murine, a bovine, an equine, a porcine or an ovine subject. Accordingly, a formulation comprising the necessary therapy or equivalent thereof is further provided herein. The formulation can further comprise one or more preservatives or stabilizers.

The agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates.

Various delivery systems are known and can be used to administer a chemotherapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it can be desirable to administer the pharmaceutical compositions of the disclosure locally to the area in need of treatment; this can be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a therapy or a medicament comprising an effective amount of a chemotherapeutic as described herein for treatment of a human cancer patient having the appropriate expression level of the gene of interest as identified in the experimental examples. Further provided is a therapy comprising a platinum drug, or alternatively a platinum drug therapy, for use in treating a human cancer patient having the appropriate expression level of the gene of interest as identified in the experimental examples.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient and the subject being treated. Single or multiple administrations can be carried out with the dose level and pattern being selected by the treating physician.

### Kits

Kits or panel for use in detecting the rs3853839 and/or rs5743618 polymorphisms in patient biological samples are provided. In some embodiments, a kit comprises at least one reagent necessary to perform the assay. For example, the kit can comprise an enzyme, a buffer or any other necessary reagent (e.g. PCR reagents and buffers). For example, in some aspects, a kit contains, in an amount sufficient for at least one assay, any of the hybridization assay probes, amplification primers, and/or antibodies suitable for detection in a packaging material. In some embodiments, the kit or panel comprises primer and/or probes suitable for screening for the rs3853839 and/or rs5743618 polymorphisms.

The various components of the kit can be provided in a variety of forms. For example, in some aspects, the required enzymes, the nucleotide triphosphates, the probes, primers, and/or antibodies are be provided as a lyophilized reagent. These lyophilized reagents can be pre-mixed before lyophilization so that when reconstituted they form a complete mixture with the proper ratio of each of the components ready for use in the assay. In addition, the kits can contain a reconstitution reagent for reconstituting the lyophilized reagents of the kit. In exemplary kits for amplifying target nucleic acid derived from a cancer patients, the enzymes, nucleotide triphosphates and required cofactors for the enzymes are provided as a single lyophilized reagent that, when reconstituted, forms a proper reagent for use in the present amplification methods.

In some aspects, the kit or panel is for determining the likely clinical outcome of a cancer patient receiving an anti-EGFR therapy, for example, comprising cetuximab, or combination irinotecan and bevacizumab. In some aspects, the kit or panel is for determining the eligibility of a cancer patient for receiving an anti-EGFR therapy, for example, comprising cetuximab, or combination irinotecan and bevacizumab.

Typically, the kits will also include instructions recorded in a tangible form (e.g., contained on paper or an electronic medium) for using the packaged probes, primers, and/or antibodies in a detection assay for determining the presence or amount of the rs3853839 and/or rs5743618 polymorphisms in a test sample.

In some aspects, the kits further comprise a solid support for anchoring the nucleic acid of interest on the solid support. The target nucleic acid can be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, and antigen/antibody.

In one aspect, the kit further comprises an effective amount of the therapy. In one aspect, the therapy comprises, or alternatively consists essentially of, or yet alternatively consisting of, administration of a therapeutically effective amount of cetuximab. In some aspects, therapy comprising cetuximab further comprises a therapeutically effective amount of oxaliplatin. In some aspects, therapy comprising oxaliplatin comprises a therapeutically effective amount of folinic acid and/or a pyrimidine analog. In some aspects, the therapy comprises FOLFOX (leucovorin+Fluorouracil (5-FU)+oxaliplatin). In some aspects, therapy comprising cetuximab further comprises a therapeutically effective amount of irinotecan. In some aspects, therapy comprising irinotecan comprises a therapeutically effective amount of folinic acid and/or a pyrimidine analog. In some aspects, the therapy comprises FOLFIRI (leucovorin+Fluorouracil (5-FU)+irinotecan). In some aspects, the therapy further comprises therapeutically effective amounts of irinotecan and oxaliplatin. In some aspects, the therapy comprises FOLFOXFIRI (leucovorin+Fluorouracil (5-FU)+oxaliplatin+irinotecan). In some aspects, the therapy further comprises a therapeutically effective amount of bevacizumab.

In one aspect, the therapy comprises, or alternatively consists essentially of, or yet alternatively consisting of, administration of a therapeutically effective amount of irinotecan and bevacizumab. In some aspects, therapy comprising irinotecan and bevacizumab further comprises therapeutically effective amount of folinic acid and/or a pyrimidine analog. In some aspects, the therapy comprises FOLFIRI (leucovorin+Fluorouracil (5-FU)+irinotecan). In some aspects, therapy comprising irinotecan and bevacizumab further comprises a therapeutically effective amount of oxaliplatin. In some aspects, therapy comprising oxaliplatin comprises a therapeutically effective amount of folinic acid and/or a pyrimidine analog. In some aspects, the therapy comprises FOLFOX (leucovorin+Fluorouracil (5-FU)+oxaliplatin). In some aspects, the therapy further comprises therapeutically effective amounts of irinotecan and oxaliplatin. In some aspects, the therapy comprises FOLFOXFIRI (leucovorin+Fluorouracil (5-FU)+oxaliplatin+irinotecan).

The kit can comprise at least one probe or primer which is capable of specifically hybridizing to the gene of interest and instructions for use. For example, in some aspects, the kits comprise at least one of the above described nucleic acids. Exemplary kits for amplifying at least a portion of the gene of interest comprise two primers. For example, in some embodiments, the kit comprises a forward primer comprising nucleic acid having the sequence of SEQ ID NO:2 and a reverse primer comprising nucleic acid having the sequence of SEQ ID NO:3 for amplification of the rs5743618 polymorphism or a forward primer comprising nucleic acid having the sequence of SEQ ID NO:5 and a reverse primer comprising nucleic acid having the sequence of SEQ ID NO:6 for amplification of the rs5743618 polymorphism. In some embodiments, the kit further comprises a nucleic acid probe for the detection of the amplicon. In some embodiments, the nucleic acid probe has about 5, about 10, about 15, about 20, or about 25, or about 30, about 35, about 40 or more contiguous nucleotides of SEQ ID NO:1 or 4 and overlaps the polymorphic site. In some aspects, the nucleic acid primers and/or probes are lyophilized.

In some embodiments, at least one of the primers for amplification is capable of hybridizing to the allelic variant sequence. For example, in some embodiments, at least one of the primers for amplification has about 5, about 10, about 15, about 20, or about 25, or about 30, about 35, about 40 or more contiguous nucleotides of SEQ ID NO:1 or 4 and overlaps the polymorphic site. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the surface is silica or glass. In another embodiment, the surface is a metal electrode.

The test samples used in the diagnostic kits include cells, protein or membrane extracts of cells, or biological fluids such as sputum, blood, serum, plasma, or urine. The test samples can also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest or target region.

As amenable, these suggested kit components can be packaged in a manner customary for use by those of skill in the art. For example, these suggested kit components can be provided in solution or as a liquid dispersion or the like.

Typical packaging materials would include solid matrices such as glass, plastic, paper, foil, micro-particles and the like, capable of holding within fixed limits hybridization assay probes, and/or amplification primers. Thus, for example, the packaging materials can include glass vials used to contain sub-milligram (e.g., picogram or nanogram) quantities of a contemplated probe, primer, or antibodies or they can be microtiter plate wells to which probes, primers, or antibodies have been operatively affixed, i.e., linked so as to be capable of participating in an amplification and/or detection methods.

The instructions will typically indicate the reagents and/or concentrations of reagents and at least one assay method parameter which might be, for example, the relative amounts of reagents to use per amount of sample. In addition, such specifics as maintenance, time periods, temperature, and buffer conditions can also be included.

The diagnostic systems contemplate kits having any of the hybridization assay probes, amplification primers, or antibodies described herein, whether provided individually or in one of the combinations described above, for use in determining the presence or amount of rs3853839 and/or rs5743618 polymorphism in a test sample.

The disclosure now being generally described, it will be more readily understood by reference to the following example which is included merely for purposes of illustration of certain aspects and embodiments of the present disclosure, and are not intended to limit the disclosure.

### EXPERIMENTAL EXAMPLE

These examples show that functional significant single nucleotide polymorphisms in genes involved in the degradation pathway predict clinical outcomes of metastatic colorectal cancer treated with cetuximab or combination irinotecan/bevacizumab therapies.

### Example 1

TLR7 (Toll like receptor 7, mRNA: NM_016562) and TLR9 (Toll like receptor 9, mRNA: NM_138688, NM_017442) signaling pathways are implicated in the regulation of immune system through typeI interferon induction. Immune responses within the tumor microenvironment may influence the efficacy of chemotherapy. TLR7 and TLR9 agonists showed promising results in preclinical and/or clinical trials for cancer patients, in particular in association with cetuximab (cet). This study examined whether genetic variations in TLR7 and TLR9, and their downstream molecule IRF5 and IRF7, are associated with outcome in mCRC patients receiving cet-based chemotherapy.

This study included 3 independent cohorts: patients treated with FOLFIRI+cet in the FIRE3 trial as a discovery set (FIRE3Cet cohort, n=299); patients treated with FOLFIRI+bevacizumab in FIRE3 trial as a control set (FIRE3Bev cohort, n=293), patients treated with FOLFOX (or SOX)+cet in JACCROCC05/06 trial as a validation set (Japanese cohort, n=76).

Main patients characteristics' were the following: FIRE3Cet cohort M/F 68/32%, median age 64, Kras status wild/mutant 82/18%; FIRE3Bev cohort M/F 66/34%, median age 64, Kras status wild/mutant 84/16%; Japanese cohort M/F 58/42%, median age 63, all patients were Kras wild type. Median follow up times were 41.8, 40.9, and 24.7 months, respectively.

Genomic DNA was isolated from tissue samples. 6 single nucleotide polymorphisms (SNPs) in TLR7, TLR9, IRF5, and IRF7 were analyzed by PCR and direct sequencing. These SNPs were tested for the association with PFS and OS.

PCR and product sequencing were done using standard procedures. Uni- and multivariate analyses, adjusting for age, gender, rash and racial background, were carried out. Example PCR primers used in the example are provided in the Table 2 below.

TABLE 2

| SNP | Forward primer (SEQ ID NO: 2) | Reverse primer (SEQ ID NO: 3) |
|---|---|---|
| rs3853839 | 5′-ATTGCTTCCGTGTCATCCAG-3′ | 5′-TCCCTATGGAACCCAGAAGC-3′ |

The amplicon generated has the following sequence (S=G or C): 5′ATTGCTTCCGTGTCATCCAGGGCCCCATTCTGTGCAGATTGAGTGTGGGCACCA CACAGGTGGTTGCTGCTTCAGTGCTTCCTGCTCTTTTTSCTTGGGCCTGCTTCTGG GTTCCATAGGGA-3′ (SEQ ID NO:23). Thus, the sequence of the amplicon from the (G) allele is 5′ATTGCTTCCGTGTCATCCAGGGCCCCATTCTGTGCAGATTGAGTGTGGGCACCA CACAGGTGGTTGCTGCTTCAGTGCTTCCTGCTCTTTTTGCTTGGGCCTGCTTCTGG GTTCCATAGGGA-3′ (SEQ ID NO:28) and the sequence of the amplicon from the (C) allele is 5′ATTGCTTCCGTGTCATCCAGGGCCCCATTCTGTGCAGATTGAGTGTGGGCACCA CACAGGTGGTTGCTGCTTCAGTGCTTCCTGCTCTTTTTCCTTGGGCCTGCTTCTGG GTTCCATAGGGA-3′ (SEQ ID NO:29).

Among Kras wild-type patients in the discovery set (FIRE3Cet), patients with TLR7 rs3853839 G/G variant showed a trend toward longer PFS compared to those with any C (median 10.0 vs. 11.8 months, HR 1.39, P=0.092). This preliminary association was confirmed in the Japanese cohort, and patients with G/G genotype showed a PFS benefit compared to those carrying any C (univariate: 9.1 vs. 11.6 months, HR 2.04, P=0.005, multivariate: HR 2.02, 95% CI: 1.143.55, P=0.015). In the control set (FIRE3Bev), this correlation of TLR7rs3853839 with PFS was not observed.

These results suggest that TLR7 rs3853839 polymorphism may predict outcome of cetuximab-based chemotherapy in Kras wild-type patients with metastatic colorectal cancer.

### Example 2

The purpose of this study was to evaluate the clinical significance of single nucleotide polymorphisms in TLR1, TLR2, TLR6, and TAK1 in patients with metastatic colorectal cancer (mCRC).

In recent studies, TLR1 (Toll like receptor 1, mRNA: NM_003263) and TLR6 (Toll like receptor 6, mRNA: NM_006068), each of which forms a heterodimer with TLR2 (Toll like receptor 2, mRNA: NM_003264, NM_001318787, NM_001318789, NM_001318790, NM_001318791), were suggested to be essential in regulating mucosal immune response within the gut (13-16). Applicant herein focused on TLR1, TLR2, TLR6, and their common downstream signal molecule TAK1 (Mitogen-activated protein kinase kinase kinase 7, mRNA: NM_003188, NM_145331, NM_145332, NM_145333), and hypothesized that genetic variations in these genes may cause inter-individual differences of clinical outcome in metastatic colorectal cancer (mCRC) patients treated with chemotherapy. Single nucleotide polymorphisms (SNPs) are substantial germline genetic variations, some of which may alter the gene function and/or activity. In this study, Applicant tested whether SNPs in these genes are associated with clinical outcome in mCRC patients treated with FOLFIRI plus bevacizumab across independently different cohorts.

### Methods

### Patients and Samples

This study enrolled two independent cohorts with mCRC patients who were enrolled in a prospective randomized phase III clinical trial, TRIBE (17) or FIRE-3 (18), and underwent FOLFIRI plus bevacizumab as the 1st-line chemotherapy. TRIBE study consisted a total of 508 patients with untreated mCRC, from 34 Italian centers, were enrolled and randomly assigned to receive either FOLFIRI plus bevacizumab (arm-A) or FOLFOXIRI plus bevacizumab (arm-B). In FIRE-3 study, 752 patients with mCRC, from centers in Germany and Austria, were randomized to receive FOLFIRI plus cetuximab or FOLFIRI plus bevacizumab as the 1st-line chemotherapy. Eligibility criteria of Applicant's study included patients with histologically confirmed colorectal adenocarcinoma, measurable metastatic disease according to Response Evaluation Criteria in Solid Tumors (RECIST) v1.1, and no previous exposure to systemic chemotherapy except for adjuvant chemotherapy, and all patients received irinotecan-based regimen (FOLFIRI). In this study, 228 patients from arm A of TRIBE and 297patients from the bevacizumab arm of FIRE-3 were enrolled as discovery and validation cohorts, respectively.

### Selected Polymorphisms and Genotyping

Candidate SNPs in TLR1, TLR2, TLR6, and TAK1 were selected for analyses when having a minor allele frequency of >10% in Europeans according to the Ensembl database (http://www.ensembl.org/index.html). Among the candidate SNPs, Applicant focused on 9 SNPs which had a biological significance reported in literature reviews or were considered potentially functional according to the F-SNP database (19). The characteristics of the selected polymorphisms are shown in Table 3.

TABLE 3
SNPs and Primers

| | | | | Base change | |
|---|---|---|---|---|---|
| | | | | (Amino | |
| | SNP | Location | MAF* | acid change) | Primer sequence |
| TLR 1 | rs5743618 | exon 4 | 0.25 | T > G | F 5′-TGGCACACCATCCTGAGATAC-3′ |
| | | | | (missense) | (SEQ ID NO: 5) |
| | | | | | R 5′-ACCCGGAAAGTTATAGAGGAACC-3′ |
| | | | | | (SEQ ID NO: 6) |
| | rs5743565 | 5′-UTR | 0.19 | T > C | F 5′-TGGCCTGAGAAACAGAAGGAC-3′ |
| | | | | | (SEQ ID NO: 7) |
| | | | | | R 5′-CCCGCCATTTGTATTCTCTTC-3′ |
| | | | | | (SEQ ID NO: 8) |
| TLR 2 | rs3804099 | Exon 3 | 0.44 | C > T | f 5′-CCTTGAGGAACTTGAGATTGATG-3′ |
| | | | | (synonymous) | (SEQ ID NO: 9) |
| | | | | | R 5′-CCAAACATTCCACGGAACTTG-3′ |
| | | | | | (SEQ ID NO: 10) |
| | rs4696480 | promoter | 0.48 | T > A | F 5′-ATGGTTCTGGAGTCTGGGAAG-3′ |
| | | | | | (SEQ ID NO: 11) |
| | | | | | R 5′-CCAAGGGAGCAGTTTATTGTGAG-3′ |
| | | | | | (SEQ ID NO: 12) |
| TLR 6 | rs3821985 | exon 1 | 0.32 | G > C | F 5′-CCTTCGTCATGAGACCTACTTTG-3′ |
| | | | | (synonymous) | (SEQ ID NO: 13) |
| | | | | | R 5′-CTCATGCACCAAGCACATTC-3′ |
| | | | | | (SEQ ID NO: 14) |
| | rs5743818 | exon 1 | 0.27 | A > C | F 5′-CCCAGGCAGAATCATGTTCAC-3′ |
| | | | | (synonymous) | (SEQ ID NO: 15) |
| | | | | | R 5′-TTGGATCTGCCCTGGTATCTC-3′ |
| | | | | | (SEQ ID NO: 16) |
| TAK1 | rs1145727 | intron | 0.30 | A > G | F 5′-GCTAAGATGAGAGTCAAGACAGAGAC-3′ |
| | | | | | (SEQ ID NO: 17) |
| | | | | | R 5′-GCTGAGTTAATTCTGACAAAAGGAC-3′ |
| | | | | | (SEQ ID NO: 18) |
| | rs157688 | 5′-UTR | 0.31 | T > C | F 5′-TCCTCAAATTAGACAAGGAACAGAG-3′ |
| | | | | | (SEQ ID NO: 19) |
| | | | | | R 5′-AGAAGCCTAGGCCTTAAAGGTG-3′ |
| | | | | | (SEQ ID NO: 20) |
| | rs157432 | intron | 0.30 | T > G | F 5′-AAGAATGGACCCCTGCCTTC-3′ |
| | | | | | (SEQ ID NO: 21) |
| | | | | | R 5′-GCCTTCATCATTAGCCCTTACC-3′ |
| | | | | | (SEQ ID NO: 22) |

5′-UTR: 5′-untranslated region
*MAF, minor allele frequency, According to the Ensembl database (phase 1 of the 1000
Genomes Project) for Europeans.

Genomic DNA was extracted from FFPE (formalin-fixed paraffin-embedded) specimens in patients enrolled in FIRE-3, and from blood in patients enrolled in TRIBE using the QIAamp DNAeasy Kit (Qiagen) according to the manufacturer's instructions (www.qiagen.com). The primers used for polymerase chain reaction analyses are listed in Table 3. DNA sequences were analyzed using the ABI Sequencing Scanner version 1.0 (Applied Biosystems). Investigators involved in SNP analyses were blinded to patients' clinical data.

### Statistical Analysis

The end points of current study were objective response rate (RR), progression-free survival (PFS), and overall survival (OS). Tumor responses based on RECIST were grouped into responders, including complete or partial response, and non-responders, including stable or progressive disease. PFS was defined as the period from the first day of starting first-line chemotherapy to the first day of documented disease progression or death. If progression or death was not observed, PFS was censored on the day of the last CT scan. OS was defined as the period from starting therapy to the date of death or censored on the date of last contact if alive. The differences in baseline patient characteristics between the two cohorts were examined using Chi-square test or the Wilcoxon Rank-Sum test when appropriate. Allelic distribution of polymorphisms by ethnicity was tested for deviation from Hardy-Weinberg equilibrium (HWE) using the exact test. Linkage disequilibrium among selected SNPs was assessed using D′ and r2 values, and the haplotype frequencies were inferred using Haploview version 4.2 (www.broad.mit.edu/mpg/haploview). The power to detect an association between a SNP and PFS would be 80% when the minimum hazard ratio (HR) varied from 1.53 to 2.05 using a two-sided log-rank test at 0.05 significance level in the training cohort (n=228, 174 PFS events). Applicant assumed that the minor allele frequency ranged from 0.05 to 0.4, and the dominant model was considered. The power would be from 88% using the same test to detect the same ranged HRs with the same allele frequencies under the dominant model in the validation cohort (n=297, 252 PFS events). The associations between polymorphisms and PFS, OS and RR were investigated using Kaplan-Meier curves, log-rank test, and Fisher's exact test. A Cox proportional hazards regression model with stratification factors was fitted to re-evaluate the association between SNPs and PFS and OS considering imbalances in the distributions of baseline characteristics among cohorts. The baseline demographic and clinical characteristics that remained significantly associated with endpoints in the multivariable analysis (P<0.1) were included in the final model. All analyses were performed with 2-sided tests at a significance level of 0.05 by using the SAS 9.4 (SAS Institute, Cary, N.C., USA).

### Results

The baseline characteristics of the two cohorts included in this study are summarized in Table 4. Compared to FIRE-3 cohort, TRIBE cohort comprised younger patients and better performance status, fewer patients received primary tumor resection, more patients with synchronous metastasis, and more patients with KRAS mutant. The median PFS, OS and follow-up period were 9.7, 26.1 and 49.3 months in TRIBE cohort; 10.1, 23.8, and 40.8 months in FIRE-3 cohort. Genotyping was successful in at least 90% of cases in each polymorphism analyzed. In failed cases, genotyping was not successful because of limited quantity and/or quality of extracted genomic DNA. The allelic frequencies for all SNPs were within the probability limits of HWE (P>0.001), with exception of TLR6 rs3821985 in FIRE-3 cohort. High linkage disequilibrium was found between TLR6 rs5743818 and TLR6 rs3821985 (D′=0.95, r2=0.53) in TRIBE cohort.

TABLE 4
Baseline clinical characteristics of the TRIBE and FIRE-3 cohorts

| | TRIBE cohort | | FIRE-3 cohort | | |
|---|---|---|---|---|---|
| | (N = 228) | | (N = 297) | | |
| | n (%) | | n (%) | | p^{a} |
| Gender | | | | | |
| Male | 138 | (61) | 195 | (66) | |
| Female | 90 | (39) | 102 | (34) | 0.23 |
| Age | | | | | |
| Median (range) | 60 | (29-75) | 65 | (31-76) | <0.001 |
| <65 | 163 | (71) | 156 | (53) | |
| ≥65 | 65 | (29) | 141 | (47) | <0.001 |
| Performance status | | | | | |
| ECOG 0 | 188 | (83) | 163 | (55) | |
| ECOG 1-2 | 39 | (17) | 134 | (45) | <0.001 |
| Unknown^{b} | 1 | (0.4) | | | |
| Primary tumor site | | | | | |
| Right side | 57 | (25) | 63 | (21) | |
| Left side | 156 | (68) | 180 | (61) | 0.84 |
| Unknown^{b} | 15 | (7) | 54 | (18) | |
| Liver limited disease | | | | | |
| Yes | 72 | (32) | 96 | (32) | |
| No | 156 | (68) | 201 | (68) | 0.86 |
| Number of metastatic sites | | | | | |
| <2 | 99 | (43) | 107 | (36) | |
| ≥2 | 129 | (57) | 142 | (48) | 0.92 |
| Unknown^{b} | | | 48 | (16) | |
| Time to metastasis | | | | | |
| Synchronous | 188 | (82) | 185 | (62) | |
| Metachronous | 40 | (18) | 63 | (21) | 0.038 |
| Unknown^{b} | | | 48 | (16) | |
| Primary tumor resection | | | | | |
| Yes | 144 | (63) | 257 | (87) | |
| No | 84 | (37) | 40 | (13) | <0.001 |
| Adjuvant chemotherapy | | | | | |
| Yes | 28 | (12) | 54 | (18) | |
| No | 200 | (88) | 243 | (82) | 0.065 |
| KRAS status | | | | | |
| Wild-type | 96 | (42) | 249 | (84) | |
| Mutant | 93 | (41) | 48 | (16) | <0.001 |
| Unknown^{b} | 39 | (17) | | | |

^{a}Based on the X² test or the Wilcoxon rank-sum test whenever appropriate.
^{b}Not included in the test.

Association of SNPs with Clinical Outcome

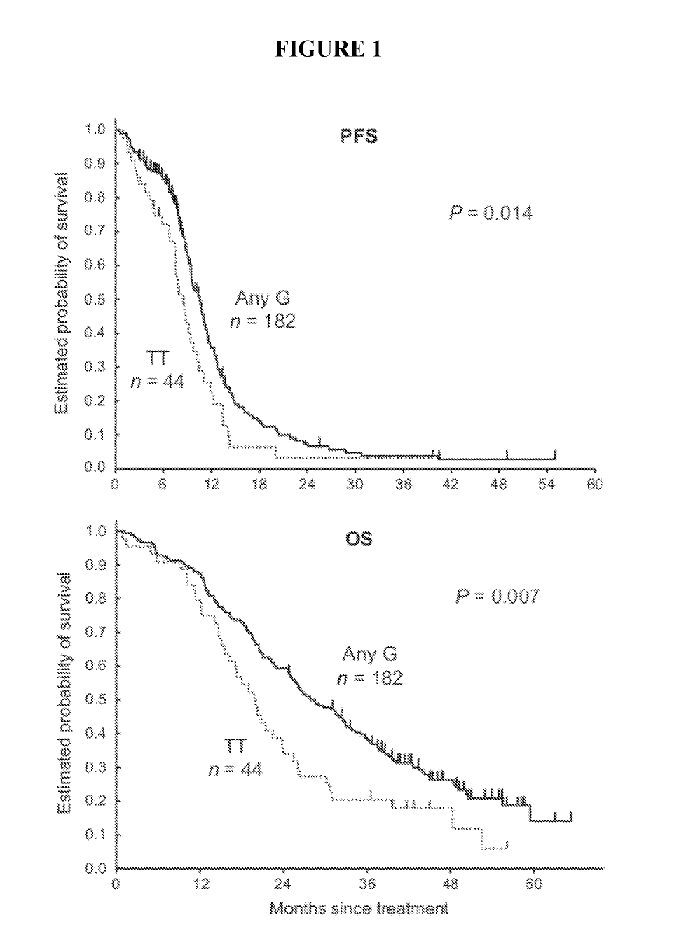

Applicant first evaluated the relation of each SNP to RR, PFS, and OS, using the TRIBE cohort as a discovery study. When there were statistically significant associations between SNP and clinical outcome, a validation study was subsequently performed using the FIRE-3 cohort. The association between TLR1 rs5743618 and clinical outcome is shown in Tables 5-7 below. In TRIBE, the homozygous wild-type T/T genotype was associated with a significantly lower RR compared to other variant T/G and G/G genotypes (43% vs. 62%, P=0.025). In addition, those patients with the T/T genotype showed significantly worse PFS and OS as compared to those with the T/G or G/G genotypes (FIG. 1). The median PFS was 8.2 months for patients with the T/T genotype compared with 10.5 months for patients with other genotypes (HR: 1.57, 95% CI: 1.09-2.28, P=0.014), and the median OS were 19.9 and 27.9 months, respectively (T/T vs. Any G, HR: 1.63, 95% CI: 1.14-2.35, P=0.007). These differences remained statistically significant in multivariate analyses (PFS; HR: 1.50, 95% CI: 1.01-2.22, P=0.046, and OS; HR: 1.53, 95% CI: 1.06-2.23, P=0.025). In FIRE-3, the same association was observed in RR (T/T: 46% vs. Any G: 65%, P=0.021). However, significant differences between genotypes were not observed in PFS and OS. For TLR1 rs5743565, there was no association with clinical outcome.

TABLE 5
PFS with Univariate Analysis

| | | | | PFS | | |
|---|---|---|---|---|---|---|
| | | | | Univariate^{a} | | |
| | | RR | | Median Month | | |
| Genotype | n | n (%) | P | (95% CI) | HR (95% CI) | P |
| TRIBE | | | | | | |
| G/G | 50 | 29 (60%) | | 10.8 (8.8-12.6) | 1 (Reference) | |
| G/T | 132 | 81 (63%) | 0.077 | 10.5 (9.4-11.3) | 0.92 (0.63-1.34) | 0.045 |
| T/T | 44 | 18 (43%) | | 8.2 (7.5-9.7) | 1.48 (0.93-2.35) | |
| Any G | 182 | 110 (62%) | 0.025 | 10.5 (9.5-11.2) | 1 (Reference) | 0.014 |
| T/T | 44 | 18 (43%) | | 8.2 (7.5-9.7) | 1.57 (1.09-2.28) | |
| FIRE-3 | | | | | | |
| G/G | 147 | 95 (68%) | | 10.7 (9.1-12.3) | 1 (Reference) | |
| G/T | 87 | 45 (58%) | 0.020 | 10.4 (9.0-13.2) | 0.94 (0.70-1.26) | 0.466 |
| T/T | 52 | 22 (46%) | | 10.1 (8.5-11.3) | 1.18 (0.84-1.67) | |
| Any G | 234 | 140 (65%) | 0.021 | 10.4 (9.3-11.9) | 1 (Reference) | 0.244 |
| T/T | 52 | 22 (46%) | | 10.1 (0.87-1.68) | 1.21 (0.87-1.68) | |

TABLE 6
PFS with Multivariate Analysis

| | | | | PFS | |
|---|---|---|---|---|---|
| | | RR | | Multivariate^{b} | |
| Genotype | n | n (%) | P | HR (95% CI) | P |
| TRIBE | | | | | |
| G/G | 50 | 29 (60%) | | 1 (Reference) | |
| G/T | 132 | 81 (63%) | 0.077 | 0.89 (0.60-1.34) | 0.120 |
| T/T | 44 | 18 (43%) | | 1.38 (0.85-2.25) | |
| Any G | 182 | 110 (62%) | 0.025 | 1 (Reference) | 0.046 |
| T/T | 44 | 18 (43%) | | 1.50 (1.01-2.22) | |
| FIRE-3 | | | | | |
| G/G | 147 | 95 (68%) | | 1 (Reference) | |
| G/T | 87 | 45 (58%) | 0.020 | 0.98 (0.73-1.31) | 0.559 |
| T/T | 52 | 22 (46%) | | 1.19 (0.84-1.67) | |
| Any G | 234 | 140 (65%) | 0.021 | 1 (Reference) | 0.244 |
| T/T | 52 | 22 (46%) | | 1.20 (0.86-1.66) | |

TABLE 7
Overall Survival

| | | OS | | | | |
|---|---|---|---|---|---|---|
| | | Univariate^{a} | | | | |
| | | Median Month | | | Multivariate^{b} | |
| Genotype | n | (95% CI) | HR (95% CI) | P | HR (95% CI) | P |
| TRIBE | | | | | | |
| G/G | 50 | 23.2 (16.4-37.6) | 1 (Reference) | | 1 (Reference) | |
| G/T | 132 | 28.6 (25.6-33.5) | 0.95 (0.65-1.39) | 0.024 | 0.94 (0.63-1.40) | 0.077 |
| T/T | 44 | 19.9 (15.1-24.0) | 1.57 (0.99-2.49) | | 1.46 (0.91-2.36) | |
| Any G | 182 | 27.9 (25.0-33.0) | 1 (Reference) | 0.007 | 1 (Reference) | 0.025 |
| T/T | 44 | 19.9 (15.1-24.0) | 1.63 (1.14-2.35) | | 1.53 (1.06-2.23) | |
| FIRE-3 | | | | | | |
| G/G | 147 | 24.2 (19.4-27.4) | 1 (Reference) | | 1 (Reference) | |
| G/T | 87 | 26.9 (21.3-31.0) | 0.88 (0.63-1.22) | 0.335 | 0.84 (0.61-1.18) | 0.332 |
| T/T | 52 | 23.1 (15.1-28.0) | 1.19 (0.83-1.72) | | 1.15 (0.79-1.67) | |
| Any G | 234 | 24.8 (21.5-27.6) | 1 (Reference) | 0.205 | 1 (Reference) | 0.265 |
| T/T | 52 | 23.1 (15.1-28.0) | 1.25 (0.88-1.77) | | 1.20 (0.86-1.66) | |

The above Tables 5-7 illustrate data for the objective response rate (RR), progression free survival (PFS), and overall survival (OS) between the (T/T) and (G/T) or (G/G) genotypes for TLR1 rs5743618 for TRIBE and FIRE-3 cohorts. Correlations with P < 0.05 are marked withbold text. The P value was based on Fisher's exact test for tumor response, log-rank test for PFS and OS in the univariable analysis (^{a}) and Wald test in the multivariable Cox proportional hazards regression model adjusting age, ECOG performance status, primary tumor site, number of metastatic sites, resection of the primary tumors, RAS mutation status, adjuvant chemotherapy in TRIBE cohort, adjusting for sex, ECOG performance status, liver limited metastasis, primary tumor resection, and KRAS mutation status in FIRE3 cohort (^{b}).

TLR2: For TLR2 rs3804099 and rs4696480, both polymorphisms significantly associated with PFS in univariate analyses, but these significances were lost when a multivariable testing was applied.

TLR6: In TRIBE, TLR6 rs5743818 A/A genotype was associated with a significantly lower RR compared to the A/C and C/C genotypes. Univariate analysis showed that those patients with the A/A genotype had significantly shorter PFS compared to those with A/C or C/C genotype. However, this significance did not retain statistical significance in multivariate analysis. The association between this SNP and RR was not validated in FIRE-3. For rs3821985, there was no association with clinical outcome.

TAK1: In TRIBE, patients homozygous (A/A) for rs1145727 showed significantly shorter OS compared to those with A/G or G/G genotypes, which retained statistical significance in multivariate analysis. However, these results were not validated in FIRE-3. For rs157688, although the C/C genotype was associated with a significantly longer PFS and OS compared to other genotypes in univariate analyses, these differences did not remain significant in multivariate analyses. For rs157432, no association with clinical outcome was observed.

This data showed for the first time that the SNP in TLR1 was associated with clinical outcome in patients with mCRC. TLR1 rs5743618 was significantly associated with clinical response to chemotherapy FOLFIRI plus bevacizumab, which was validated in an independent cohort. This polymorphism also significantly correlated with PFS and OS in the TRIBE cohort in both univariate and multivariate analyses. These findings indicate that a cellular TLR1 signaling plays a critical role in the efficacy of FOLFIRI plus bevacizumab and may be a novel target for drug development.

TLR1 rs5743618 (base pair change: T1805G, amino acid change: 1602S) is a common non-synonymous SNP lying just at the junction of the transmembrane and cytoplasmic domain of TLR1. Although the mechanism by which 1602S affects the function of TLR1 remains unclear, it has been suggested that a structural change induced by the substitution of a serine (S) for an isoleucine (I) within the transmembrane domain impacts the extracellular ligand-binding domain or the intracellular domain that binds to adaptor proteins (20). Indeed, several studies have shown that 1602S is associated with decreased cytokine responses. Hawn et al. (20) demonstrated that the individuals with a variant genotype (602S) showed significantly decreased IL-6 level compared to those with a wild-type genotype (6021) in a ligand-stimulated whole-blood cytokine assay. In another study, homozygous for 602S allele exhibited significantly lower levels of TNF-α release in response to the ligand (21). Without being bound by theory, Applicant believes that the variant 602S genotype relates to impaired TLR1 signal and subsequent decreased cytokine production.

However, the functional role of the TLR1 signal in the tumor microenvironment is not fully understood. Specific TLRs are known to recognize DAMPs released from stressed or dying tumor cells upon use of chemotherapy agents. The TLR activated by DAMP generates various biological responses including inflammation, immune response, angiogenesis, and anti-apoptosis in the tumor microenvironment, which contribute to create an ideal condition for cancer cell survival and result in chemoresistance (22, 23). Recent studies have described also the biological functions of TLR1, which may lead to cancer promotion and survival.

Without being bound by theory, it is Applicant's belief that TLR1/2 promotes angiogenesis. TLR1/2 heterodimers on endothelial cells recognize a molecular pattern of a lipid oxidation product, w-(2-carboxyethyll)pyrrole (CEP), which is generated as a consequence of oxidative stress (24). TLR1/2 signaling triggered by CEP activates downstream NFκB pathway, and eventually promotes angiogenesis. Notably, the CEP-induced TLR1/2 signal was demonstrated to be independent of the VEGF pathway and have a proangiogenic effect comparable to VEGF in an in vitro study (25). Without being bound by theory, Applicant believes that the TLR1/2 signal acts as an important alternative proangiogenic pathway independent of VEGF. Therefore, oxidative stress induced by chemotherapy may activate the TLR1/2 signal and promote angiogenesis, which results in the resistance to bevacizumab.

Without being bound by theory, it is Applicant's belief that TLR1/2 can function to regulate T-helper 17 (Th17) polarization in the gut. Th17 is known to have a protective immune response against bacteria in the gut, and the TLR1/2 signal in dendritic cells contribute to the Th17 polarization at mucosal surface by inducing interleukin-6 (IL-6) and IL-23 (15). However, there is still controversy over whether Th17 has a tumor-promoting function or has a tumor-suppressing function in the tumor microenvironment. Recent studies have shown that Th17 infiltrate in the tumor microenvironment negatively influenced the prognosis of CRC patients (26, 27). The mechanism underlying this association is considered to be that Th17-related cytokines, such as IL-17, IL-21, and IL-22, stimulate STAT3 and NFκB pathways in cancer cells along with IL-6 and TNF-α, which leads to CRC cell growth and survival (28). Therefore, the TLR1/2 signal may play a key role in inducing Th17 polarization also in the tumor microenvironment.

Without being bound by theory, Applicant's findings suggest that the pro-tumorigenic effects mediated by TLR1/2 were impaired in the individuals with variant T/G or G/G genotypes, which resulted in better responses against FOLFIRI plus bevacizumab.

In conclusion, this is the first study to show the association of genetic variations in TLR1, TLR2, TLR6 and TAK1 with clinical outcome of mCRC patients treated with chemotherapy. Without being bound by theory, Applicant's findings suggest that TLR1 rs5743618 serves as a predictive biomarker of clinical response to FOLFIRI plus bevacizumab. As the long-term goal of pharmacogenetic studies is to use genotype data to predict the efficacy of drugs and to individualize the treatment of patients, this SNP can be helpful in the selection of appropriate patients who would benefit from FOLFIRI plus bevacizumab in mCRC patients.

### Example 3

Genomic DNA was isolated from tissue samples from 561 mCRC patients enrolled in the randomized phase III FIRE3 trial and treated in first-line with either FOLFIRI+bevacizumab (n=283) or FOLFIRI+cetuximab (n=278) was obtained from tissue samples. Two single nucleotide polymorphisms (SNPs) in the TLR6 gene, rs5743818 G/A and rs3821985 G/C, were analyzed by PCR-based direct sequencing. These SNPs were tested for the association with tumor response, progression free survival (PFS) and overall survival (OS). Subgroup analyses by gender, tumor location, and Kras status were also analyzed. In the FOLFIRI+bevacizumab treated patients, TLR6 rs5743818 A/A variant showed a significant association with PFS (10.5 months vs 9.8 months, HR 1.34, log rank p=0.03) and OS (26.4 months vs 21.3 months, HR 1.5, log rank p=0.006) compared to the A/C and C/C variants which remained significant in multivariate analyses (Tables 8-10). In the subgroup analyses patients with rs3821985 G/G with right sided tumors had a significant association with PFS (logrank p, 0.001) and OS (logrank p=0.04′7) however for left sided tumors only OS was significant (logrank p=0.023). In males there was a significant association with PFS (HR 1.42, p=0.039) and OS (HR 1.57, p=0.014) for the A/A allele but not for females (Tables 11-13). In the FOLFIRI+cetuximab treated patients, there was no association with outcome.

Tables 8-10 below illustrate data for progression free survival (PFS) and overall survival (OS) between the (A/A) and (A/C) or (C/C) genotypes for TLR6 rs5743818 for FIRE3 cohort. The data demonstrates the association between TLR6 rs5743818 SNP (synonymous A to C, MAF:0.12) with clinical outcome in FIRE-3 bev arm. The P value was based on Fisher's exact test for tumor response, log-rank test for PFS and OS in the univariable analysis, and Wald test in the multivariable Cox proportional hazards regression model adjusting for sex, ECOG performance status, liver limited metastasis, primary tumor resection, and KRAS mutation status.

TABLE 8
TLR6 rs5743818 Tumor Response

| | Number | Tumor Response | | |
|---|---|---|---|---|
| SNP | in group | Yes | No | P value* |
| TLR6 rs5743818 | | | | 0.665 |
| A/A | 164 | 92 (61%) | 60 (39%) | |
| A/C | 89 | 49 (60%) | 33 (40%) | |
| C/C | 17 | 11 (73%) | 4 (27%) | |
| | | | | 0.894 |
| A/A | 164 | 92 (61%) | 60 (39%) | |
| Any C | 106 | 60 (62%) | 37 (38%) | |

TABLE 9
TLR6 rs5743818 PFS

| | Progression-Free Survival | | | | |
|---|---|---|---|---|---|
| | Media | | | | |
| | (95% CI), | Univarable | | Multivariable | |
| SNP | months | HR (95% CI) | P value* | HR (95% CI) | P value* |
| TLR6 | | | 0.086 | | 0.11 |
| rs5743818 | | | | | |
| A/A | 10.5 | 1 (Reference) | | 1 (Reference) | |
| | (9.7, 12.7) | | | | |
| A/C | 10.1 | 1.32 (0.99, | | 1.31 | |
| | (8.3, 12.0) | 1.75) | | (0.98, 1.74) | |
| C/C | 9.1 | 1.45 (0.86, | | 1.46 | |
| | (7.0, 11.8) | 2.45) | | (0.86, 2.45) | |
| | | | 0.030 | | 0.040 |
| A/A | 10.5 | 1 (Reference) | | 1 (Reference) | |
| | (9.7, 12.7) | | | | |
| Any C | 9.8 | 1.34 (1.02, | | 1.33 | |
| | (8.5, 11.7) | 1.75) | | (1.01, 1.75) | |

TABLE 10
TLR6 rs5743818 OS

| | Overall Survival | | | | |
|---|---|---|---|---|---|
| | Media | | | | |
| | (95% CI), | Univarable | | Multivariable | |
| SNP | months | HR (95% CI) | P value* | HR (95% CI) | P value* |
| TLR6 | | | 0.020 | | 0.059 |
| rs5743818 | | | | | |
| A/A | 26.4 | 1 (Reference) | | 1 (Reference) | |
| | (22.7, 29.0) | | | | |
| A/C | 20.6 | 1.53 | | 1.47 | |
| | (16.7, 24.8) | (1.12, 2.08) | | (1.06, 2.02) | |
| C/C | 25.4 | 1.34 | | 1.33 | |
| | (13.8, 36.0) | (0.72, 2.51) | | (0.71, 2.49) | |
| | | | 0.006 | | 0.018 |
| A/A | 26.4 (22.7, | 1 (Reference) | | 1 (Reference) | |
| | 29.0) | | | | |
| Any C | 21.3 | 1.50 | | 1.44 | |
| | (17.4, 25.6) | (1.12, 2.02) | | (1.06, 1.96) | |

Tables 11-13 below illustrate the association between TLR6 rs3821985 SNP with clinical outcome in FIRE-3 bevacizumab arm. The total n=265. The data relates to the tumor response, progression free survival, and overall survival between the (G/G) and (C/G) or (C/C) genotypes for TLR6 rs3821985 for FIRE3 cohort. The P value was based on Fisher's exact test for tumor response, log-rank test for PFS and OS in the univariable analysis, and Wald test in the multivariable Cox proportional hazards regression model adjusting for sex, ECOG performance status, liver limited metastasis, primary tumor resection, and KRAS mutation status.

TABLE 11
TLR6 rs3821985 Tumor Response

| | Number | Tumor Response | | |
|---|---|---|---|---|
| SNP | in group | Yes | No | P value* |
| TLR6 | | | | 0.357 |
| rs3821985 | | | | |
| G/G | 115 | 66 (62%) | 41 (38%) | |
| C/G | 95 | 50 (56%) | 39 (44%) | |
| C/C | 55 | 33 (69%) | 15 (31%) | |
| | | | | 0.895 |
| G/G | 115 | 66 (62%) | 41 (38%) | |
| Any C | 150 | 83 (61%) | 54 (39%) | |
| | | | | 0.251 |
| Any G | 210 | 116 (59%) | 80 (41%) | |
| C/C | 55 | 33 (69%) | 15 (31%) | |

TABLE 12
TLR6 rs3821985 PFS

| | Progression-Free Survival | | | | |
|---|---|---|---|---|---|
| | Media | | | | |
| | (95% CI), | Univarable | | Multivariable | |
| SNP | months | HR (95% CI) | P value* | HR (95% CI) | P value* |
| TLR6 | | | 0.28 | | 0.43 |
| rs3821985 | | | | | |
| G/G | 10.5 (9.0, | 1 (Reference) | | 1 (Reference) | |
| | 12.8) | | | | |
| C/G | 9.8 (8.1, | 1.25 (0.93, | | 1.20 (0.89, | |
| | 11.7) | 1.68) | | 1.63) | |
| C/C | 10.2 (9.3, | 1.21 (0.85, | | 1.18 (0.83, | |
| | 11.9) | 1.72) | | 1.67) | |
| | | | 0.11 | | 0.19 |
| G/G | 10.5 (9.0, | 1 (Reference) | | 1 (Reference) | |
| | 12.8) | | | | |
| Any C | 10.0 (9.1, | 1.24 (0.95, | | 1.19 (0.91, | |
| | 11.5) | 1.61) | | 1.56) | |
| | | | 0.56 | | 0.62 |
| Any G | 10.1 (9.0, | 1 (Reference) | | 1 (Reference) | |
| | 11.2) | | | | |
| C/C | 10.2 (9.3, | 1.10 (0.80, | | 1.09 (0.79, | |
| | 11.9) | 1.51) | | 1.50) | |

TABLE 13
TLR6 rs3821985 OS

| | Overall Survival | | | | |
|---|---|---|---|---|---|
| | Media | | | | |
| | (95% CI), | Univarable | | Multivariable | |
| SNP | months | HR (95% CI) | P value* | HR (95% CI) | P value* |
| TLR6 | | | 0.20 | | 0.43 |
| rs3821985 | | | | | |
| G/G | 24.7 | 1 (Reference) | | 1 (Reference) | |
| | (21.8, 29.6) | | | | |
| C/G | 21.2 | 1.35 | | 1.25 | |
| | (18.2, 27.5) | (0.97, 1.87) | | (0.89, 1.76) | |
| C/C | 26.5 | 1.18 | | 1.13 | |
| | (18.4, 30.8) | (0.79, 1.76) | | (0.76, 1.69) | |
| | | | 0.096 | | 0.23 |
| G/G | 24.7 | 1 (Reference) | | 1 (Reference) | |
| | (21.8, 29.6) | | | | |
| Any C | 24.8 | 1.28 | | 1.21 | |
| | (19.0, 27.6) | (0.95, 1.74) | | (0.89, 1.64) | |
| | | | 0.89 | | 0.91 |
| Any G | 23.7 | 1 (Reference) | | 1 (Reference) | |
| | (21.2, 26.7) | | | | |
| C/C | 26.5 | 1.03 | | 1.02 | |
| | (18.4, 30.8) | (0.71, 1.47) | | (0.71, 1.47) | |

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

The following articles are referenced in the disclosure hereinabove and are incorporated by reference in their entirety:
1. Amado R G, Wolf M, Peeters M, Van Cutsem E, Siena S, Freeman D J, et al. Wild-type KRAS is required for panitumumab efficacy in patients with metastatic colorectal cancer. J Clin Oncol. 2008; 26:1626-34.
2. Van Cutsem E, Lenz H J, Kohne C H, Heinemann V, Tejpar S, Melezinek I, et al. Fluorouracil, leucovorin, and irinotecan plus cetuximab treatment and RAS mutations in colorectal cancer. J Clin Oncol. 2015; 33:692-700.
3. Ince W L, Jubb A M, Holden S N, Holmgren E B, Tobin P, Sridhar M, et al. Association of k-ras, b-raf, and p53 status with the treatment effect of bevacizumab. J Natl Cancer Inst. 2005; 97:981-9.
4. Kopetz S, Hoff P M, Morris J S, Wolff R A, Eng C, Glover K Y, et al. Phase I I trial of infusional fluorouracil, irinotecan, and bevacizumab for metastatic colorectal cancer: efficacy and circulating angiogenic biomarkers associated with therapeutic resistance. J Clin Oncol. 2010; 28:453-9.
5. Ronzoni M, Manzoni M, Mariucci S, Loupakis F, Brugnatelli S, Bencardino K, et al. Circulating endothelial cells and endothelial progenitors as predictive markers of clinical response to bevacizumab-based first-line treatment in advanced cancer patients. Ann Oncol. 2010; 21:2382-9.
6. Xiao Y, Freeman G J. The microsatellite instable subset of colorectal cancer is a particularly good candidate for checkpoint blockade immunotherapy. Cancer Discov. 2015; 5:16-8.
7. Abreu M T. Toll-like receptor signalling in the intestinal epithelium: how bacterial recognition shapes intestinal function. Nat Rev Immunol. 2010; 10:131-44.
8. Sato Y, Goto Y, Narita N, Hoon D S. Cancer Cells Expressing Toll-like Receptors and the Tumor Microenvironment. Cancer Microenviron. 2009; 2 Suppl 1:205-14.
9. Li T T, Ogino S, Qian Z R. Toll-like receptor signaling in colorectal cancer: carcinogenesis to cancer therapy. World J Gastroenterol. 2014; 20:17699-708.
10. Ridnour L A, Cheng R Y, Switzer C H, Heinecke J L, Ambs S, Glynn S, et al. Molecular pathways: toll-like receptors in the tumor microenvironment—poor prognosis or new therapeutic opportunity. Clin Cancer Res. 2013; 19:1340-6.
11. Pradere J P, Dapito D H, Schwabe R F. The Yin and Yang of Toll-like receptors in cancer. Oncogene. 2014; 33:3485-95.
12. Jinushi M, Yagita H, Yoshiyama H, Tahara H. Putting the brakes on anticancer therapies: suppression of innate immune pathways by tumor-associated myeloid cells. Trends Mol Med. 2013; 19:536-45.
13. Kamdar K, Nguyen V, DePaolo R W. Toll-like receptor signaling and regulation of intestinal immunity. Virulence. 2013; 4:207-12.
14. DePaolo R W, Kamdar K, Khakpour S, Sugiura Y, Wang W, Jabri B. A specific role for TLR1 in protective T(H)17 immunity during mucosal infection. J Exp Med. 2012; 209:1437-44.
15. Sugiura Y, Kamdar K, Khakpour S, Young G, Karpus W J, DePaolo R W. TLR1-induced chemokine production is critical for mucosal immunity against Yersinia enterocolitica. Mucosal Immunol. 2013; 6:1101-9.
16. Morgan M E, Koelink P J, Zheng B, den Brok M H, van de Kant H J, Verspaget H W, et al. Toll-like receptor 6 stimulation promotes T-helper 1 and 17 responses in gastrointestinal-associated lymphoid tissue and modulates murine experimental colitis. Mucosal Immunol. 2014; 7:1266-77.
17. Loupakis F, Cremolini C, Masi G, Lonardi S, Zagonel V, Salvatore L, et al. Initial therapy with FOLFOXIRI and bevacizumab for metastatic colorectal cancer. N Engl J Med. 2014; 371:1609-18.
18. Heinemann V, von Weikersthal L F, Decker T, Kiani A, Vehling-Kaiser U, Al-Batran S E, et al. FOLFIRI plus cetuximab versus FOLFIRI plus bevacizumab as first-line treatment for patients with metastatic colorectal cancer (FIRE-3): a randomised, open-label, phase 3 trial. Lancet Oncol. 2014; 15:1065-75.
19. Lee P H, Shatkay H. F-SNP: computationally predicted functional SNPs for disease association studies. Nucleic Acids Res. 2008; 36:D820-4.
20. Hawn T R, Misch E A, Dunstan S J, Thwaites G E, Lan N T, Quy H T, et al. A common human TLR1 polymorphism regulates the innate immune response to lipopeptides. Eur J Immunol. 2007; 37:2280-9.
21. Johnson C M, Lyle E A, Omueti K O, Stepensky V A, Yegin O, Alpsoy E, et al. Cutting edge: A common polymorphism impairs cell surface trafficking and functional responses of TLR1 but protects against leprosy. J Immunol. 2007; 178:7520-4.
22. Mittal D, Saccheri F, Vénéreau E, Pusterla T, Bianchi M E, Rescigno M. TLR4-mediated skin carcinogenesis is dependent on immune and radioresistant cells. EMBO J. 2010; 29:2242-52.
23. Cherfils-Vicini J, Platonova S, Gillard M, Laurans L, Validire P, Caliandro R, et al. Triggering of TLR7 and TLR8 expressed by human lung cancer cells induces cell survival and chemoresistance. J Clin Invest. 2010; 120:1285-97.
24. Saeed A M, Duffort S, Ivanov D, Wang H, Laird J M, Salomon R G, et al. The oxidative stress product carboxyethylpyrrole potentiates TLR2/TLR1 inflammatory signaling in macrophages. PLoS One. 2014; 9:e106421.
25. West X Z, Malinin N L, Merkulova A A, Tischenko M, Kerr B A, Borden E C, et al. Oxidative stress induces angiogenesis by activating TLR2 with novel endogenous ligands. Nature. 2010; 467:972-6.
26. Fridman W H, Galon J, Pages F, Tartour E, Sautes-Fridman C, Kroemer G. Prognostic and predictive impact of intra- and peritumoral immune infiltrates. Cancer Res. 2011; 71:5601-5.
27. Tosolini M, Kirilovsky A, Mlecnik B, Fredriksen T, Mauger S, Bindea G, et al. Clinical impact of different classes of infiltrating T cytotoxic and helper cells (Th1, th2, treg, th17) in patients with colorectal cancer. Cancer Res. 2011; 71:1263-71.
28. De Simone V, Franz& E, Ronchetti G, Colantoni A, Fantini M C, Di Fusco D, et al. Th17-type cytokines, IL-6 and TNF-α synergistically activate STAT3 and N F-kB to promote colorectal cancer cell growth. Oncogene. 2015; 34:3493-503.

SEQUENCE LISTTNG

```
TLR7 rs3853839 Polymorphism (SEQ ID NO: 1)
TGCTTCAGTGCTTCCTGCTCTTTTT[C/G]CTTGGGCCTGCTTCTGGGTTCCATA
TLR7 rs3853839 Forward Primer (SEQ ID NO: 2)
ATTGCTTCCGTGTCATCCAG
TLR7 rs3853839 Reverse Primer (SEQ ID NO: 3)
TCCCTATGGAACCCAGAAGC
TLR1 rs5743618 Polymorphism (SEQ ID NO: 4)
GCTGTGACTGTGACCTCCCTCTGCA+G/T+CTACTTGGATCTGCCCTGGTATCTC
TLR1 rs5743618 Exon 4 Forward Primer (SEQ ID NO: 5)
TGGCACACCATCCTGAGATAC
TLR1 rs5743618 Exon 4 Reverse Primer (SEQ ID NO: 6)
ACCCGGAAAGTTATAGAGGAACC
TLR1 rs5743565 5′-UTR Forward Primer (SEQ ID NO: 7)
TGGCCTGAGAAACAGAAGGAC
TLR1 rs5743565 5′-UTR Reverse Primer (SEQ ID NO: 8)
CCCGCCATTTGTATTCTCTTC
TLR2 rs3804099 Exon 3 Forward Primer (SEQ ID NO: 9)
CCTTGAGGAACTTGAGATTGATG
TLR2 rs3804099 Exon 3 Reverse Primer (SEQ ID NO: 10)
CCAAACATTCCACGGAACTTG
TLR2 rs4696480 Promoter Forward Primer (SEQ ID NO: 11)
ATGGTTCTGGAGTCTGGGAAG
TLR2 rs4696480 Promoter Reverse Primer (SEQ ID NO: 12)
CCAAGGGAGCAGTTTATTGTGAG
TLR6 rs3821985 Exon 1 Forward Primer (SEQ ID NO: 13)
CCTTCGTCATGAGACCTACTTTG
TLR6 rs3821985 Exon 1 Reverse Primer (SEQ ID NO: 14)
CTCATGCACCAAGCACATTC
TLR6 rs5743818 Exon 1 Forward Primer (SEQ ID NO: 15)
CCCAGGCAGAATCATGTTCAC
TLR6 rs5743818 Exon 1 Reverse Primer (SEQ ID NO: 16)
TTGGATCTGCCCTGGTATCTC
TAK1 rs1145727 Tntron Forward Primer (SEQ ID NO: 17)
GCTAAGATGAGAGTCAAGACAGAGAC
TAK1 rs1145727 Tntron Reverse Primer (SEQ ID NO: 18)
GCTGAGTTATTCTGACAAAAGGAC
TAK1 rs157688 5′-UTR Forward Primer (SEQ ID NO: 19)
TCCTCAAATTAGACAAGGAACAGAG
TAK1 rs157688 5′-UTR Reverse Primer (SEQ ID NO: 20)
AGAAGCCTAGGCCTTAAAGGTG
TAK1 rs157432 Tntron Forward Primer (SEQ ID NO: 21)
AAGAATGGACCCCTGCCTTC
TAK1 rs157432 Tntron Reverse Primer (SEQ ID NO: 22)
GCCTTCATCATTAGCCCTTACC
TLR7 rs3853839 amplicon (S = G or C) (SEQ ID NO: 23)
ATTGCTTCCGTGTCATCCAGGGCCCCATTCTGTGCAGATTGAGTGTGGGCACCACACAGGTGGTTGCTGCTTCAGTGCTT
CCTGCTCTTTTTSCTTGGGCCTGCTTCTGGGTTCCATAGGGA
(C) allele of TLR7 rs3853839 (SEQ ID NO: 24)
TGCTTCAGTGCTTCCTGCTCTTTTTCCTTGGGCCTGCTTCTGGGTTCCATA
(G) allele of TLR7 rs3853839 (SEQ ID NO: 25)
TGCTTCAGTGCTTCCTGCTCTTTTTGCTTGGGCCTGCTTCTGGGTTCCATA
(G) allele of TLR1 rs5743618 (SEQ ID NO: 26)
GCTGTGACTGTGACCTCCCTCTGCAGCTACTTGGATCTGCCCTGGTATCTC
(T) allele of TLR1 rs5743618 (SEQ ID NO: 27)
GCTGTGACTGTGACCTCCCTCTGCATCTACTTGGATCTGCCCTGGTATCTC
(G) amplicon of TLR7 rs3853839 (SEQ ID NO: 28)
ATTGCTTCCGTGTCATCCAGGGCCCCATTCTGTGCAGATTGAGTGTGGGCACCACACAGGTGGTTGCTGCTTCAGTGCTT
CCTGCTCTTTTTGCTTGGGCCTGCTTCTGGGTTCCATAGGGA
(C) amplicon of TLR7 rs3853839 (SEQ ID NO: 29)
ATTGCTTCCGTGTCATCCAGGGCCCCATTCTGTGCAGATTGAGTGTGGGCACCACACAGGTGGTTGCTGCTTCAGTGCTT
CCTGCTCTTTTTCCTTGGGCCTGCTTCTGGGTTCCATAGGGA
TLR1 gene (SEQ ID NO: 30)
>Chromosome: GRCh38:4:38790077:38857417: −1
          >Exon 1: 38,804,791-38,804,754
          >Exon 2: 38,804,382-38,804,306
          >Exon 3: 38,800,948-38,800,857
          >Exon 4: 38,798,898-38,796,257
          >rs5743618: 38,797,027
GTGTTATTTATTATTCCTAAATTAAAATCTAAACCATTTAATAACACCAAATGTTGATCAGAATGTGGGTATGATTGCTC
GTACATATGGCTAAATATGTAGTCATAAATATGTATCAACCAGTAAACTTTCCTTAGAAGTTGATTTTTAAAAAAAAACC
CAAATTGTGGAACTATACTTATTTTATTTTAAACTATCTCATCCAGCTAAAATAATGCTCAAAATTGCTTGATAAGGGAA
ACTGCAATTATTCTTGAAATGTTAAATATGGTGATAATAGAAAACAGGCTGTTTAAAATAACTTTCCACTCAGGTGACAA
ATGTAATAACCAGGCTGCAAACTCTTACAGAGAAATATTACCTTAGAAAAATATTTTTCTTCACATTGAATTGTTTGTAA
GTTGCTTCCTCACACGAATTAGAAAATTGGATTACTGGTTTTGTCTGTAGCCCAGAAAAAACTGCTAACTCACCTCCGGA
CCTTTGAAAGGTAAAAATATACGTAGGAGTAATTCCAGTGTGGGCCAACAGATGGCACCAATCTGTTTCTAAGGTACCGC
TTTACTCAACTTTAAGAACTTGTGTTTGTCAAGCATTTTCAATTGTATTTCCGTTCATTTACAAGTTATTTTCTCTTCTT
CTGAAAAAGAGATCTTGGTAAGTAGAAAGACTTCTGTGGCTTTTTCACATTATGGCACAGGTGCTTCAAGTAAGCAAGTT
TGCTAACCATTCATTATATAGGAAATAGGTAATGTTTCCTTGAAATTTTTGCAAAATGATTTGAAGTGGTTTTTTTTGTA
GATGTTGCAAATGTCCTTTGAATTTCACGAGCAGGATGAAGAGCAGCGTCTTTACAGCTGTCTTGAAAAGATAGAAAACA
ACTAGCGCTTGTGGTGATAATTAAGGAACAAAACTAATGTTATTTATTTCTTCTGATTTGGCCCAGCGAATTTTCTCTTT
TCGTCTTTCTCTAGTTTATTGTTTTCAGTAACATTTAACAGTAATTTTTATATACCACTGATATGGCACCTATTTATTAT
ATGTTTTTACAGCAGACGTGTGTTTGATCTCCCTTGTCGTTTGTCACTGTAACAGTTTGCTGGTCTGTGTCTGTCCCTAA
TTCATAAGCTTTTTGAAAGCAGGAATTATGTCAGTCCTATTCATATTTATATCCCTGGTGCCTAGTGCGTAGCATGGCAC
TGGCAGAAAAATAGACTCTGAATAAACATTTATTAAATGGAGGAAGTGGCCGGGACCAAGCCCCTTCACAAGCTTGAATC
TGGCACAGTGTAGCAATTGCCTTCCTCCAGGTCCTTCCACCTTCAGTTTGACAAATGAAACAATGTTTTTCTTGCCTATT
TGTTAACTTGATTATGTGATCTTTCCTCCACTTATTTCACTTAATTTTTTTTCTGCTCTCTTCTTCCTTGTAGTTTTAAT
TTTCATAGGTCTTGTCTACCCTTTTTTTCTGAAATGTAGCTTATTTCATCTATTTCCTCACCCTTTTTATTTGATGCATT
TAAAAAATCAAGAGCATTTACCTGTTTATTTTTACTTTCTTTAAAAAACTGTGAACTCTCTAATCTTCCACTCTCATCTT
CCCATTTTTCCCTCCTTTTTTCCCAATAAATAGAAAAGTTACTATCTTGTTCATTTAGTGTATTCCCTAGGAACTGCCTC
AGGGTATGAACGCCTCTGGAGGAATATTGCGTTTTTGTGGCAGGATACCATAAGGACAGCTGACCAGCTTTTCCACCTGG
TGACTGATTTTCAGTCCCATGAACCCAGACACCGTGGGTCTGGTTGCCGGGGACCTGTAGGGTAGGTCCTCATCTGTGAA
TGCCAGTTATCTACTATTATCTGAAAGTTCTCCATCTTTATATTTTAATATTCCAAATAAAACAAGCACGTTTCTTTAAA
TATAAAATATTTTCACACAGCTATAAAGACAACTCTTAAATTGCCTTACGATAACTTTTTTGGATTATAAAGGACATTCA
AAATGAGACATTTTAGGGATATCTGAAAGTATAAGAAAGAAAATTAAATTCATCTATAATTCAGAGATTCAGAGAACCAC
TATTATTAGTATTTGGTATATTTCATCACATTGTTTGTCCTCTAGCTTTTACGTATATGTCAGGTTTTAATCATATTTTG
CAAAATTAGAGTCTTACTACATATATAATTTTTTTTTTTTTTTTTTGAGACGGAGTCTTGCTCTGTCGCCCAGGCTGGAG
TGCAGCGGCACGATCTCGGCTCACTGCAAGCTCCGCCTCCCGGGTTCACGCCATTCTCCTGCCTCAGCCTCCCGAGTAGC
TGGGACTACAGGCGCCCGCCACCACGCCCGGCTAATTTTTTTGTATTTTTTTTTAGTAGAGACGGGGTTTCACCGTTAGC
CAGGATGGTCTCGATCTCCTGACCTCGTGATCCACCCGCCTTGGCCTCCCAAAGTGCTGGGATGAGAGGCGTGAGCCACT
GCGCCCGGCCATAATTTTCTATTACTGTATTCATTTAACATTATCATTTTTTCAAAGTACAGTATTTGAGGCTATTATTT
AATTATACATAACTGATTTAATCATTTTCTACACTAGACATTCTGATTTTTTCAATATTTCATTATGTCATGAGTATCCT
TATAGTCATATCTTCTGTGTGTTATTTTTGACTATTTCTTTAGAAAATTCCTTGTAGTGGAATTGCTAAAATAAAGGAAG
GTATATTTTTAAGAAATTTATGTAAATTACCAAATTACCCTTTAGAAAGCTTGTACTAATTTTTCATTCCTGCCTAAGTA
GTAAATGAGGGTTCTTATTTTAGTTTCCACCTACCAACCTGGATATTATATTTTTTAATTTTCCCTATTTGATAGGTGAA
AAAAAAATCTTGCCTTTTAAAAAATCTTTTAAGAAACAAGAGTCCTCCTTTGACCCCCTTCCTCTTTTTATGCTTAAATA
TCTTATAGTAAAAATAACAGGCATTCTGGAAACCTATTTCTAAAAGCTTTATTCTCTATCTCTAAAAAAATTACATTTTC
CTAAAATAGCCAAATAACATTATAAAAAATTACAATTTATTTCTTAATAGCATCTAATGGCTGGTCCATCATAGAATCTG
CCCCTCCTTCTGTTACTGAATATGTTTTATAGCAAGTCTGTCTTAACTGAGATCTAATCCAAGATCATGAGTTTCATCTA
GATGTTATGTGCTTTTTTGATTTAACGTGCTGTTTTATTTTTTGTGAGATTGCACATTATTCATATTATTGGCAATTGAT
TTTTGGCAAATTATTCAGTAAATAGTACAGTAAACTTAATTTTGAATGTTAAAAGTTTGAAAAATATAGATAAAAACTAA
GAGGAAAATAAAGTTCCATTATAATCTTACTGTGCAAAGATAATTACTGTTCATATCTTGGTATGTATCTTATTAATTTT
TCTACTTGCATAGGTACATATTTACTTTGGTACTTTTTAAATAAACTGCTTTTTGTCACATATAGTGAAAATATTTTCAT
GCAATTCGATTTTTATTATTTTTAAAGGCTACATAGTACTATTTAATTATACAGAGATACTAAAATTTATTTGATAACCT
GCTATTTTTGGATAATTGAGGGATTTGTTTTCAAAATAGAATTTATTTTTGCCAGGAGCAGTGGCTTGTACCTGTAACCC
CAGCTCCGTGGGAGGCTGAGGTGAGAGGATGGCATGAGCCCAGGACCTTAAGCTATGATTGTGCCACTGCACTCCAGCCT
GGGCAGTGGGACAAAACCCTGTCTCTTAAAAAGAAAGTGTTTAAGAAAATAGTAATGATAATAATTTATTTTGTGACGGC
CACTTTATTGTAGTCTTTTATTGGTGCTAACATTTTTTCAATGACTTCTCTTTGGCTTTCCACATTGACAGTCTTATAAT
CTAAAAATAATAATGATTTTCTTTCTTCATTTCAGTATTTCTTCCTGTCATTTTTTTTCTTTATTACAGTGGTTAAAACT
TCCGGAAAGAACAATGTGTGCATTCTTTTTTTTTTATTATTATACTTTAAGTTCTAGGGTACATGTGCACAACATGCAGG
TTTGTTATATATGTATACATGTGCCATGTTGGTGTGCTGCACCCATTAACTCGTCATTTACATTACGTATATCTCCTAAT
GCTTTCCCTCCCCCCTCCCCCCACCCCACGACAGGCCCCAGTGTGTTATGTTCCCTTTCCTGTGTCCAAGAGTTCTCATT
GTTCATTTCCCACCTATGAGTGAGAACATGCGGTGTTTGGTTTTTTGTCCTTGCGATAGTTTACGAGAATGATGGTTTCC
AGCTTCATCCATGTCCCTACAAAGGACATGAACTCATCCTTTTTTATGGCTGCATAGTATTCCATGGTGTATATGTGCCA
CATTTTCTTAATCCAGTCTATCATTGATGGACATTTGGGTTGGTTCCAAGTCTTTGCTATTGTGAATAGTGCCACAATAA
ACATGTGTGTGCATGTATCTTTATAGCAGCATGATTTATAATCCTTTGGGTATATACCCAGTAATGGGATAGCTGGGTCA
AATCGTATTTCTAGTTCTAGATCCCTGAGGAATCACCACACTGTCTTCCACAATGGTTGAACTAGTTTACAGTCCCACCA
ACAGTGTAAAAGTGTTCCTATTTCTCCACGTCCTCTCCAGCACCTGTTGTTTCCTGACTTTTTAATGATCGCCATTCTAA
CTGGTGTGAGACGGTATCTCATTGTGGTTTTGATTTGCATTTCTCTGATGGCCAGTGATGATGAGCATTTTTTCATGTGT
CTTTTGGCTGCATAAATGTCTTCTTTTGAGAAGTGTCTGTTCATATCCTTCGCCCACTTTTTGATGGGGTTGTTTATTTT
TTTCTTGTAAATTTGTTTGAGTTCATTGTAGATTCTGGATATTAGCCCTTTGTTGGATGAGTAGATTGCAAAAATTTTCT
CCCATTCTGTAGGTTGCCTATTCATTCTGATGGTAGTTTCTTTTGCTGTGCAGAAGCTCTTTAGTTTAATTAGATCCCAT
TTGTCAATTTTGTCTTTTGTTGCCATTGCTTTTGGTGTTTTAGACTTGAAGTCCTTGCCCATGCCTATGTCCTGAATGGT
ATTGCCTAGGTTTTCTTCTAGAGTTTTTATGGTTTTAAGTCTAACATTTAAGTCTTTAATCCATCTTGAATTAATGTTTG
TATAAGGTGTAAGGAAGGGATCCAGTTTCAGCTTTCTCCATATGGCTAGCCAGTTTTCCCAGCACCATTTATTAAATAGG
GAATCCTTTCCCTATTTCTTGTTTTTGTCAGGCTTGTCAAAGATCAGATGGTTGTAGATGTGTGGTATTACTTCTGAGAG
CTCTGTTCTGTTCCATTGATCTATATCTCTGTTTTGGTACCAGTACCATGCTGTTTTGGTTACTGTAGCCTTGTAGTATA
GTTTGAAATTAGGTAGCATGATGCCTCCAGCTTTGTTCTTTTGGCTTAGGATTGACTTGGCAATATGGGCTCTTTTTTGA
CCCCATATGAACTTTAAAGTAGTTTTTTCCAGTTCTGTGAAGAAAGTCATTGGTAGCTTGATGGGGATGGCATTGAATCT
ATAAATTACTTTGGGCAGTATGGCCATTTTCATGATATTGATTCTTCCTATCCATGAGCATGGAATGTTTGTTTGTGTCC
TCTTTTATTTCATTGAGCAGTGGTTTGTAGTTCTCCTTGAAGAGGTCCTTCACATCCCTTGTAAGTTGGATTCCTAGGTA
TTTTATTCTCTTTGAAGCAATTGTGAATGGGAGTCCACTCATGATTTGGCTCTCTGTTTGTCTGTTATTGATGTATAAGA
ATGCTTGTGATTTTTGTACATTGATTTTGTATCCTGAGACTTTGCTGAAGTTGCTTATCAGCTTAAGGAGATTTTGGGCT
GAGACGATGGGGTTTTCTAAATATACTATCATGTCATCTGCAAAGAGGGACAATTTGACTTCCTCTTTTCCTAATTGAAT
ACCCTTTATTTCTTTCTTTTGCCTGATTGCCCTGGCCAGAACTTCCAACACTATGTTGAATATGAATAGGAGTGGTGAGA
GGGGGCATCTCTGTCATGTGCCAGTTTTCAAAGGGAATGCTTCCAGTTTTTGCCCATTCAGTATGCTATTGGCTGTGGGT
TTGTCATAAATAGCTCTTATTATTTTGAGATACGTCCCATCAATACCTAATTTATTGAGAATTTTTAGCATGAAGAGCTG
TTGAATTTTGTCAAAGGCCTTTTCTGCATCTATTGAGATAATCATGTGGTTTTTGTCTTTGGTTCTGTTTATATGCTGGA
TTACATTTATTGATTTGCGTATGTTGAAACAGCCTTGCATCCCAGGGATGAAGCCCACTTGATCATGGTGGATAAGCTTT
TTGATGTGCTGCTGGATTCAGTTTGCCAGTATTTTATTGAGGATTTTTGTATTAATGTTCATCAGGGATATTGGTCTAAA
ATTCTCTTTTTTTGTTGTGTCTCTGCCAGGCTTTGGTATCAGGATGATGCTGGCCTCATAAAATGAGTTAAGGAGGATTC
CCTCTTTTTCTATTGATTGGAATAGTTTCAGAAGGAATGGTACCAGCTCCTAGTTGTACCTCTGGTAGAATTCAGCTGTG
AATCCGTCTGGTCCTGGACTTTTTTTGGTTGGTAGGCTATTAATTATTGCCTCAATTTCAGAGCCTGTTATTGGCCTATT
CAGGGATTCAACTTCTTCCTGGTTTAGTCTTGGGAGAGTGTAGGTGTCGAGGAATTTATTCACTTCTTCTAGATTTTCTA
GTTTATTTGCATAGAGGTGTTTATAGTATTATCTGATGGTAGTTTGTATTTCTGTGGGATCAGTGGTGATATCCCCTTTA
TCATTTTTTATTGCGTTGATTTGATATTTCTCTCTTTTCTTCTTTATTAGTCTTGCTAGAGGTCTATCAATTTTGTTGAT
CTTTTCAAAAACCCACCTCCTGGATTCATTGATTTTTTGAAGGGTGTTTTGTGTCTCTATCTCCTTCAGTTCTGCTCTGA
TCTTAGTTATTTCTTGCCTTCTGCTAGCTTTTCAATATGTTTGCTCTTGCTTCTCTAGTTCTTTTAATTGTGATGTTAGG
GTGTCAATTTTAGATCTTTCCTGCTTTCTCTTGTGGGCATTTAGTGCTATAAATTTCCCTCTACACACTGCTTTAACTGT
GTCCCTGAGATTGTGGTATGTTGTGTCTTTGTCCTCGCTGGTTTCAAGGAACATCTTTATTTCTGCCTTCATTTCTTTAT
GAACCCAGTAGTCATTCAGGAGCAGGTTGTTCAGTTTCCATGTAGTTGAGTGGTTTTGAGTGAGTTTCTTAATCCTGAGT
TCTAGTTTGATTGCACCATGGTCTGAGAGACAGTTTGTTATAATTTCTGTTCTTTTACATTTGCTGTGGAGTGCTTTACT
TCTAACTATGTTATCAATAAGTGCAGTGTGGTGCTGAGAAAAATATATATTCTGTTGATTTGGGGTGGAGAGTTCTGTAG
ATGTCTATTAGGTTCGCTTGGTGCAGAGCTGAGTTCAATTCCTGGATATCCTTGTTAACTTTCTGTCTCGTTGATCTGTC
TAATGTTGACAGTGGGGTGTTAAAGTCTCCCATTATTATTGTGTGGGAGTCTAAGTCTCTTTGTATTGGGTGCATATATA
TTTAGGATAGTTAGCTCTTCTTGTTGAATTGATCCCTTTACCATTATGCAATGGCCTTCTTTGTCTCTTTTGATTTTTGT
TAGTTTATAGTTTGTTTTATCAGAGACTAGGATTGCAACCCCTGCCTTTTTTTGTTTTCCATTTGCTTGGTAGATCTTCC
TCCATCCCTTTATTTTGAGCCTATGTGTGTCTCTGCACGTGAGATGGGTCTCCTGAATACAGCACACTGGTGGGTCTTGA
CTCTTTATCCAATTTGCCAGTCTGTGTCTTTTAATTGGAGCATTTAGCCCATTTACATTTAAGGTTAATATTATTATGTG
TGAATTTGATCTGTCATTATGATGTTAGCTGGTTATTCTGCTCGTTAGTTGATGCAGTTTCTTCCTAGCATCAATGGTCT
TTACATTTTGGCATGTTGTTGCAGTGGCTGGTACCGGTTCTTCCTTTCCATGTTTAGTGCTTCCTTCAGGAGCTCTTTTA
GGGCAGGCCTGGTGGTGACAAAATCTCTCAGCATTTGCTTGTCTGTAAAGGATTTTATTTCTCCTTCACTTATGAAGCTT
AGTTTGGCTGGATATGAAATTCTGGGTTGAAAATTCTTTTCTTTAAGAACGTTGAATATTGGCCCCCACTCTCTTCTGGC
TTGTAGAGTTTCTGCCGAGAGATCAGCTGTTAGTCTGATGGGCTTCCCTTTGTGGGTAACCCAACCTTTCTCTCTGGCTG
CCCTTAACATTTTTTCCTTCATTCAACTTTGGCGAATCTGACAATTATGTGTCTTGGAGTTGCTCTTCTGGAGGAGTATC
TTTGTGGTGTTCTCTGTATTTCCTGAATTTGAGTGTTGGCCTGCCTTGCTAGGTTGGTGAATTTCTCCTGGATAACATCC
TGCAGAATGTTTTCCAACATGGTTCCATTCTCCCCGTCAATTTCAGGCACACCAATCAGACATAGATTTGGCCTTTTCAC
ATAGTCCCATATTTCTTGGAGGCTTTGTTTGTTTCTTTTTACTCTTTTTCCTCTAAACTTCTCTTCTCACTTCATTTCAT
TCATTTGATCATCAATCACTGATACTCTTTCTTCCAGTTGATCAAATCAGCTACTGAAACTTGTGCATTCGTCACATAGT
TCTAGTGCCATGGTTTTCAGCTCCATCAGGTCCTTTAAGGACTTCTCTACACTGCTTATTCTAGCTAGCCATTCGTCTAA
TCTTTTGTCAAGGTTTTTAGCTTCTTTGCAATGGGTTCGAACTTCCTCCTTTAGTTGGAGAAGTTTGATCGTCTGAAGCC
ATCTCTCAACATGTCAAAGTCATTCTCTCTCCAGCTTTGTTCCGTTGCTGGCGAGGAGCTGCGTTCCTTTGGAGTGGGAG
AGGTGCTCTGATTTTTAGAATTTTCAGCTTTTCTGCTCTGTTTTTTCCCCATCTTTGTGGTTTTATCCACCTTTGGTCTT
TGATGATGGTGACGTACAGATGGGTTTTTGGTGTGGATGTCCTTTCTGTTTGTTAGTTTTCCTTGTAACAGTCAGGACCC
TCAGCTGCAGGTCTGTTGGAGTTTGCTGGAGGTCCACTCCAGACCCTGTTTGACTGGGTATCAGCAGCGGAGGCTGCAGA
ACAGCGAATATTGCTGAACAGCAAATGTTGCTGCCTGATCGTTCCTCTGGAAGCTTTGTCTCAGAGGGGTACCTGGCCAT
GTGAGGTATCAGTCTGCCCCTACTGGGGGGTGCCTCCCAGTTAGGCTACATGGGGGTCAGGGACCCACTTGAGGAGGCAG
TCTGTCCATTCCCAGATCTCAAACTCTGTGCTGGGAGAACCACTACTCTCTTCAAAGCTGTCAGACAGGGACATTTAAGT
CTGCAGAGGTTTCTGCTGCCTTTTGTTGGGCTATGCCCTGCCCCCAGAGGTGGAGTCTACAGAGGCAGGCAGGCCTCCTT
GAGCTGTGGTGGGCTCCACCCAGTTTGAGCTTCCTGGCTGCTTTGTTTACCTACTCAAGCCTCAGCAATGGCTGGCGCCC
CTCCCCCAGCCTCACTGCCACCTTGCAGTTTGAGCTCAGACTGCTGTGCTAGCAATGAGCGAGGCTCCGTGGGTGTGGGA
CCCTCCAAGCCAGGTGCGGGATATAATCTCCTGGTATGCCGTTTGCTAAGACCGTTGGAAAAGCACGGTATTAGGGTGGG
AGTGACCCAATTTTCCAGGTGCCATCTGTCACCCCTTCCTTTGGCTAGGAAAGGGAATTCCTGACCCCTTGTGCTTCCCG
GGTGAGGCAATGCCTCACCGTGCTTCGGCTCACACTCATTGAGCTGCACCCACTGTCCTGCCCCCACTGTCCGACGGGCC
CCAGTGAGATGAACCCGGTACCTCAGCTGGAAATGCAGAAATCACCCATCTTCTGCATCACTTATGCTGAGAGCTATAGA
CTGGAGCTGTTCCTATTTGGCCATCTTGGAACCACCCCCCCTAATGTGTGCATTCTTGATGTTGACTTTAACAGGAATGC
CTTGGGGTGGAATATTGATTAAAGTTAGATATTTGAGGTATTCTTTATAAAGTTTTCAAAGTATTTTTTAAATACCTATA
GTTGTATTTAATCAAAGATGAGGACTGAATTTTATCAAGTGAATTTTTCACCTTTATAGAGATGCTCATCTATTTTTCTT
GTTTGACTTACTCAATTGAATTATTGTTATAGACTTGTTAATGATAGACCATTTCTACAATCTCAGAAACATCCTAATTA
CTCATTATATTTTTTCTTCAGGACACAGCTAGATTGATTGGGGTTTCTTTATTCTGAGCTCTATCCCTTCCCCATAAATG
ACTGAAATTTATCCCTTTTATTTGTATTTTCTCATAATTACTCTTAATTTTATAATATATATACTTACAAATATTTTTCT
AACAACATCAGGAGTTAATTGGCATCTATAGCTTCCTTTCAATTAGACAACTATCTTACATGGTTTTAGTGCACTCTCTC
CCTCCCTACCCCACCTTCAGCCAACTCCCTTCTCCCTTCTTATCATTGGCAGTTTTTAGCTCGAGATTTCTATTAAGATT
TTTGTCATCAATACTTTTTGGTACTTAGCACTAAATTTTACTACTTTTCTTTGCTAAATAATCCTTCTTGTATTTTATTA
TTTTAGAGTTAATTTCTTTTTAAACTCTCCATCCTCTGGGAATTCAGATATGGATCTGTCTTCCATGTCTTTTAGCATTT
TAGTATGTTTTCTGTCTCTTTGGCCTCCCCTGGTGAGTTTGGGAAAATTCTCCTTGGTCTTCTCTATCATTAATTTTCTC
ATTAGTGGTGCTATTTGCAACTCAAGTCATCCAGTGAATTTTTCTTTTCAATAATTGTCTATAATTTCCATGATTCTTAC
TCTATTTTTAAAAGAATCTCTCCTCTTTCTGAAGATATCTATTATAAATGCCTATTCTGCTCTTTTATTTCTGTTTAGTT
GAGTATTAACTTCTTTCATATCTTGAGTTTGGCTCCTTTCTATTATGGTACTTGCTTTCTGCAGTATCATGCTTCTTGAT
TGTGCAACTGTGTTTAAGCCTTCTTGTGAGAGTGCTTGTTGATCTATTTTCATAGCCTGCTTGTTGGGATGCTGGATCAA
ACCAGTCTCTACTAATTTTCTTTTCTTTTCTTTTTTTTTTTTTTTTTTTGAGATGTCTCGCTCTGTGGCCCAGCCTGGAG
TACAGCGGCACAATCTCAGCTCACTGCAACCTCCACCTCCCGGGTTCAAGTGATTCTCCTGCCTCAGCCTCCTGAATAGC
TGGGACTGCAGGCACCCACTGCCACACCCGGCTAATTTTTGTATTTTTAATGGAGACAGGGTTTCACCATGTTGGCCAGG
CTGGTCTCGAACTTCTGACCTGAGGCAATCCATCCACCTCGGCCTCCCAAAGTGCTAGGATTACAGAGGTAAGCCACCAT
GCCTGGCCCCAGTCTCTACTAATTTTCTGTTGATGCTGTAACAAATTACCACAAACGTTGTGGCTTAAAACAATATACAT
TTATTATCTTACAGTTCTGTAGGGTAGGAATCTGACACAGGTCTTGTTGGCTAAAATCAAGGGACTGGCAAGGCTGCATT
CTTTCTGGAGGCTCTAGGAGGGAATCCAGGTACTTGCCTTTTCAAACTTCTAGAGGCTGCTCACATTTCTTGACTCATAG
TCCCCTTCCTTTATCTTCAGGGCCAGCAAGGATGGGCCAAGTTCTTCTCATATCACATCACCCTGACATCCTCTCTGCCT
TCCTCTTCCACTTTTAAGGGTGCTTGTGATTATACTGGGGCCACCCAAGTAATTCAGGATAATCTTCCTATTTTAAGATC
AGTTGATTAGCAACTATAATTTCATCCACAACATTAATTTTCCTTTGCCATGTAACATAGCATATTCACCAAGTTGGGGG
ATTAGAAGTAGACATCTTTGGGAGGCCAGTATTCTGTCATAGGTGAGTCCCTTTCTGGATTTCAGAGGCTTCTCGGTTTC
AGTCCCCTAAAGGTCCCCTTGCCTTTTAGTGTGTTATGGATCTATCAATCGATCAATATAGATAAAACTTTAATAGATAG
ATAGAGATATAACTTAATATTTTTCAAAATATTTGGTCATTTCAGGAATTTAAGGCAAGAAGAGGAAGCTGGAACACATA
CAAGCCACCCTTTTGACTATCATAATTTGTAATGTTTGTATTTGAAAATGTGGTTTTGTCACCCAGGCTGGAGTGCAGTG
GCACAATCTTAGCTCACTGCAACCTCCGCCTCCCAGGTTCGAACGATTCTCTTGCCTCAGCCCCCAAAGTAGCTGGGAAT
ACAGGTGTGTACCACCACGCCTGGCTAATTTTTTTTTTGTTTGTTTAGTAGAGACAGGGTTTCACCATGTTGGCAAGGCT
GGTCTCGAACTCCTTACCTCAGGAGATCCACCCACCTTGGCCTCCCAAAGTGCTGGGATTACAGGTATGAGCCACCAGGC
CAGGTCTCTTAGAACATTTTTAAATGTGATTTTAAAAGTTCCTTTTATAGTCAGAAATATGACTTTTATAATTTCTGTTT
TGGGATACTTAATTTAAGATGTATGTATTTGACCTAAAAAACAATTGATTTTAGTAAATGCTCTATGAACTCTAGAGACT
TAGACATATGATGTTCTCTTTTTGTAGGGTACAGCTTATGTCTCTATTAAATCCTTTATTAATTTCTCATTCAAATTTCC
ATATATGTTTTTATTTTTTGCTACTTGATCTAACAGTGACCCAGAGAAATATGCTAAATACTTTTACTCACAATGTCTCC
ATGTGTTTTGTAACATTTTTTACTTTGTAAATTTTGATGCAATATTACTTGCTGCATAACTGTTCTTATCTGTTACATCT
TCATTGTGAACTATTTGTTTTGACCATATAACCAGTATAAAGTAGTAGCTTCTCCTATCGTTGCCCAACTCTCTAGTACT
TTGACCGATATATACTTTGTATATATTGTGTATAAAAACTGCAATTCCTGATGAACACCGCTACCAGTATTCCAGTCGCT
GTCCATATCGGTAAGTATTGTCTGAGTTACACATCTCAGAATGCGGTGTGCTCCCTAAAGTGGTTCCCTGCCCCATGTCC
AGCTCTTACCTGGTAGTGTCCATCACTAAGTGTAAAGTTTGGGGAGAATGGAGTGTGGCAGTCCGTAGAAGGAAAACTCA
GCATGCTGATCCACTCATGTCAGTACCATTGGGGTAAAAGGGACACTGCCTAGCTACTCATTAAGTACTAGTTGGAACTC
ATCCTCACTGTTTGAATCAGAGACAGAGAGAGGACAAATTTCTTTCTGCCTTTCATCAGGGAGTCTATTTTCCTGAGACA
AAGCTTGAGTGTGGCAGGTTTGCCCTAACACAGTTCCAGTTACATTTTTGTGAGTGAAGATGCTCCTGGATCCTGCCATT
ACATTTCTGTGCTTCTTTGTTTTCAGTGGCCAAGTGGATTTTTCAAATGTATGTGTGTCTCTGTGGGTGTTCTGATGGGC
AGTTGGGAGAAGTTATATGGATCAGTGAAGAGCTTTTAACCATACGCCATTCAGTAATAAAAGTTCCCCAACTCAGAGTT
TTCACTCTTCCAAAGAAAGGTTGGTTGAAACTCTACTCATCTCTTGCCTGTTTTACTATTTCCAATATATCCTCTGATGA
CGAAAAGTGGATCCTTTGTAAATAAAGGACTCATCTCTTTCCTGTTTTACTATTTCCAATATATACCCTGATGGTGAAAA
GTGGACCCTTTGTAAATAAAGGATTCATCTCTTGACTGTTTTACTATTTCCAGTATATCCCCTGATGGTGAAAAGTGGGT
CCTTTTTAAATAAAACTGAGCAATGTGCAGTGCTCATTTTGGTAATCTGCTCTCAATTATCCCTGCAACACAGAGACCTA
CTTTAAAAATGTTGGTGGCTGCTTTTGTCCCCCAGGAGCTCATAGATGCTTTCTTGTGCAGAACAGAGCCTGAAAAGGTT
CTGAGCCAGCCTGGCAGTTCCAGTCCTTTGTCTCAGTCATGAACATGGGGTTTGTGGGCTCATGCACCTGCCGCCCTGCC
TCAGTCAAGTATTCTTTTCTTGTTTGCTCAGCATCATTGTCATGAACGTCAATGTCTATAATGTGTTGTAATGCAACGAG
GCTTTCTGGGCCCTGAGAAAGTAGTTATCTCTTCCATACGGCAGCTTTAGCCTGGCAAACTTTTTCATTTTGTCAGTGTG
TTAGCTGTTAGAACTTGATGTTTTCAGATTATCATGTGCTGAATCCTGTCTCTCTAAATTTGTGGCTTGCAGTCTAATGT
TGGTTGGAAAAATACCCATTGTAATGGGAATTGTGTCAACAATGAGATTCTATAACTAAAACACTCCTGGGATTAAAAAA
AAAAAGCACAGTATATGTGTCATTGCTATGGCCCGAATGTTTGTGTCTCTTCGAAATTCATGTGTTGAAACCTAATTACC
AACGTGATGGTATTAGATGTGTGAGCCTCTGGAAGGTGATTAGGTCATGAGGACAGGCGGCCAGGATGCCCCTGTCGGAG
CTGGGTGGCCATGGAGACTTGTCTGGCCTTGTGTGCTGGCACACAGAGGCATTCTGGCTTTGCCCCCCATGGGGGCTCAC
AGATGCTTTTCTGTGCGGATCAGAGCCTAAGGAGAGTAGACTCCTAAGCATATGTCTGAGTTTCTGAGACTGGCTCCTTG
CTTTCCCTGTAGAATAATTTTAGTCCCACCCCTCTTTCCCAGTCACAAACATGGGATCTGTGGGCTTGTGTGCCTGCTAC
CAGCCCCCAGCAAGGCACAAATGACCACTTTGGCCTGGGTTAATTGCATTTTCTCACTGGCTCCATGAATGGTGATATCA
TTTCTGCCAGGCCACTTATGGCTAGTTTTGGTCAGGTCCAATTCATGAAGGTGGAGGGAGTAGGTAGTTCCTCCTTAAGA
TATAAATATAGCCTCATTCCAGTCCTCATCTGCCCTCCCAAAAGGATTGATCCCTCTCATATTCCACAGGGCTTTGGTCA
TCACATTGCAGGCAGATCCACCTCCTGGCCAGGCCTCAATGTCCTGCCAACCACCTTCCTCCCCTTCCCTTTCTTTTGAT
GTCACCTTGCCCAAATTGGGTTGGGGATCAGAAAGTCGGGTTTGGGTCAGAATCCTGAAAGATGCAATCCCAAGTGCCAT
AATCCTGAATGTTGAAATCCTGGAAGATCAAAATCCTGAAAATATAATTCTGAAAAAAAAATTTTTAAACATTCTTTAAA
AGACATTCATTTACATTTTTGAAAGGCGATTTATTTGAGAAACATATGAAAACATGACAATATTTTATAAATCACTTTAC
AAATAAAATAAACAATAATGACGTACATATTTTTGCAAGCATGAACACTCAGATATGCTAAGGACAGTTGCATGCGTAAA
CACTCAGGAACAGATGAACCGTACTCACAAATAAATGGCTTACGTAACTGCTGCCATCCACACTGTCAAACCATGATGGG
CAACCTAAGTCCTTTGATGCGATTGATCAAAAACTGTGATGGGTCACCACCGCATATATATAGTTGCCCAGAGAGCTGAG
AGCTTGAGAAATTTTATCTTTCACAAATGCAGATGGGCAAAAAGGACTTCTGTTCATTTATTGAGAAAGTTTCCCTTTTT
TCTTCTCTCTTTCTCTCTCTCTCTCCTTTTAATCTCTGCTCACTGCAACTACCTCTGTCTCCTGGGCTCAAGCCATCCTC
CACTTCAGCCTCCCTAGTAGCTGGGACTACAGGCAGGCACCACTATGCCTGGATAATTTTTGTATTTTTTGTAGAGACAG
GGTTTCACTGTGTTGCCCGGGTTGGTCTCAAACCCCAGAGCTCAAGCCATCTGCCCGCTGTGGCCTCCCAAAGTGCTGGG
ATTACAGAGATGAGCCTCTGCTCCCGGCAGAAGTTTCAGTGTTTTCACATACACACACAATGCTTGCACACAAAGTCAAT
GTTGTGATAATGTACTTTTATGGAGTCAACGTTGCATAAAATAAATCAGAACTCTCTAAATAGTCTTTACAAAATTTATA
CCTCCAGTATTGGAAATGATGTGAAGATGAAATACATAGCATAGCAAATTGTAAAAAATAGTGTTGATGATTTAAAATAA
TGGGACAAAGGAAAAGAAAAGCAAAAAAATTAACCATCCACCCCACCCCCCAAAAAAAATTAAGAAGAAAAAGTATATCA
CAGGGACAGATTATGGGCAATTGCATGGAGGTAGTCCATAAGAGCTGGCCAACTTTCACCATTATTAATATTTTAAAGTC
TTGCATCACAACAAATAGCTGATTTATTTCTTTCAGGGCACTGTTCTCCTCAGAGAATACATTCACATTTATTTTCGATG
TGGCACTGCTTGTTTGGAAATGCTTCTCTGCTTCTATTTACATATTTACACCAACATGAGCATTCCTGTTAAATGTTCTC
ATCTTCAGTGCCGTGCTTCTGTGTTGATTTGGGTACATGAAAATCTATTCTGCATGCACTCATATACAGACCACACATTT
GGCAGAAACAATACTGGTGATCAATCAACAACAGCATTGTGTGTCTTCTTTTTTTTTTTTTTTTGAGAGGGAGTCTTGCC
CTGTCACCCAGGCTGGAGTGTAATGGCGCGATCTCGGCTCATTGCAACCTCCACCTCCCGGGTTTAAGCAATTCTCCTGC
CTCAGCCTCCCAAGTAGCTGGGATTACAGGAACCCACCACCACGCCCAGCTAATTCTTGTATTTTTTTAGTAGAGACGGG
GTTTCACCATATTGGCCAGGCAGGTCTCGAACTCCTGACCTCGTGATCCATGGGCCTCAGCCTCCCAAAGTGCTGGAATT
ACAGGTGTGAGCCACCACGCCAGTCCTGTGTCTTCTTATCCTACCATATGCATTATTATTTTCAAATCAGTCAGTAACTT
CACTGGGTGCTTTAGGCAAATGCAACTTTAATTCATTGAGAACTCCTGGTATTTCATCAGCCAGAAGGAATGCCAGTGCA
GACAAATAATGTATTTTTAAACTAAATTTTTCATCATTGCCACATTATGTGGCCAATCCTCTCAACTGAATTTTCTGACA
AATGCATTGGGCTGAATGGAAGAAAACAAACTTTATTGGTAACACCTTGAAATTCCCTTTTAGAAGCCTTGATCACACCT
AATTCCAAATCAAGTGATAGGTCATTATGATTTGAGGATTCAGTTGGACATTAGGGATTTTAGACTTTGGGGACTTTGAT
CTTTTAGGATTTCAGCCTTCAGGATTGTGTCTTTCGGGATTATAATCAGCACTGCTGAAAGTCTAGCTCAGCACTGGCAC
CACTGTCGAGTGCCCATCGGAGGTTTCTCAGGCTCAACTAAACATTCATACCCAAATGCCCCAATAGGCCACACTCAGAA
GCTCAATTTTCTACCTGGTACTTATAGTTCATACACAGTCCTTTCATCCTGGATGCAATTTACCAGTCAGGGATCATTGT
TACAATAAGTAATGTTGGCTGGTACACAGCTGACTTTCTACATGTTTCAGGTTTTCAGAATAGGGCTAGGAAGTTAACTT
GAGGTCAGATCAGTCCATGGAGAAAGAGAGAAATGGATTTGTTAACCCTTCAGCCGTGAAGGGTCACTGGTTGCAGCAGA
TGGCTGAGTTGGTGCCAAACTGGATACTGGTATAAACTACTGAGTATGTCTTGGAGTTTTTGGGACTGGCTTCTTGCCTG
CTTTTTTAAATTATCAAACCGGCTGGTCTAATATTTGGTGGTGGGGAAGGATAAAAGAACTTCTTCTTTAAAAGAGAATA
ACTATTTTTTGAGTACAGCACGTAGTTAACTTTCAATACAGTTGATTTAAAGCCTTTGCCTAGTAAGTCCAATGTCTATG
CTTTTTCCATTTCTATTAATTTTTTTTAAAAAAAAATCAATAGACTTTATTTTTTAGAGCAGTTTTTTTTTGTCTATTTA
GTTTTGCCTTTCCTGGAATATCATATAGTTGAAATAAAATAGTATTTAGCTTTTTCAGACTGACTTCTTTCACCTAGCAA
AATGGATTTAAGGATCTTCCCCCCCTTCCCTCCCCTCCCTCCCTTCCCTCCCTTCCTCCCCTCCCTTTGCCTCCCCTCCC
TCCCTTCCTTCCTTCCTCTTTCTCTCTTTCTATTTCTTTCTCTCTTTTTCTTTCTTTCTCTCTCTTTCTTAACTTTCTTT
CTCTCTCTCCTTCTCTCTCTCCCCCTCTCTCCCTCCTTTCTTTCCTTCCTCCCCTTCCTTCCTTCCTTCCTCCCTTCCTT
CCTTCCTTCCCTCCCTCCCTCCCTTCCTTCCTTCTCTTTTTCTCTTTCTTTCTTCTTTCCTTCCCCACAAAATAATTATA
CATATTTATGGGGTAACTACAATATTTGACACATACATACCGTGTGTCAAATCAGGGTATTTAGGATATCCACCATCTCA
AACATACATCATTTTGTGTGTGGGTGGGGGAGCATTTCAGATCTTCTCTTCTAGCTGTTTTGAAATATACAATAAATTAT
TGTTAGCTATAGTCACCCTACTGTGTTATTGAACACTAGAACTTTTTCTTTTTATCTAACTGTATGTTTTTATCCATTAA
CCAACCTCTCTTCATTCCCTCACTCACCTCTTAGTCTCTGGTAACTATTGTTCTACTCTCTACCCTCCTGAGATGCGTAT
TTTAGCTCCCACAGATGAGTGAGAACACAATATTTGCCTTTCTGTGCCTGGCTTATTTTGCTAATGTAATGACCTCCAGG
TCCATCCATATTGCTGCAAATGACAGGATTTAATTATTTTAAATGGCTGAATGGTATTCCATCGGGTATATATATCACAT
TTTATTTATCCATTCATCTGTTGATGGACTGTTAAGTTGATTCCATATTTTGGCTATTGTGGATAGTGCTGCAATGAACA
TGGAGGTGCAGGTATCCCTTTGATACACTGATTTCTGTTCTTTTAGATAAACATCCAGAAGTGGGGTTGCTGGATTGTAT
GTTAGTTCTATTTTTAGTTTTTTGAGACAACTCTGTACTGTTCTCCATAATGGCTGTACTAATTTACATTCCCACCAACA
GTGTATAAGAGTTCCCTTTTCTCTGCATTCTCACCAGCATTTGCTATTTATTGTCTTTTTGATGACAGCCATTTAAACTA
GAATGAGATGATATCTCGTTGTGGTTTTGATTTGCACTTCCTTGATGATTCATGGTGAGCATTTTTTTCATATAACTCTT
GGCCATTTGTACGTCTTCTTTTGAGAAATGTCTATTCAAATCCTATGCCTACTTTTTCATAGGATTATTATTATTTTTGC
TGTTGAGTTGTTTGAGTTCTTTGTATATTGTGGCTATTAGTCTTTATTGGATGAATAGTTTGCAAATATTTTCGCCCATT
TAACCCTGTTATCTATTCACCCTGTTGATTGTTTCCTGTGCTGTGTAGAAGTTTTTAGTTTAATGTAGTCCCATTTGTCT
ATTCATATTTTCATTGCCTGTACTTTTGAAATTTTAGCCATAAGATCTTTACCTAGACCAGTGTCCTCAAGCATTTCTTC
TGTTTTCTCTTAGTAGTTTTATACTTTCAAGTCTTACATTTAAGTCTGTAATCCATTTTGAATTGATTTTTGTATATGGT
GAGAGATAGGGGTCTAGTTTCATTCTTACAGGGGTCTAGTTTCATTCTTATACATATGAGTGAATATGAAGCCAGCACCA
TTTATTGAAGAGAGTGGTCTTTCCCCAGTGTATGTTGTTGGCATCTTTGTTGAAAATTGATTGGCTGTAAATATGTGGAT
TAATTTCTGGGTTCTGTATTCTGTTCTATTGGTCTATGTATCTGTTTTTGTACCAATACCATGCTACTTTTGCTTTGCAG
TATCGTTTGAAGTCAAGTAGTGTGATCCCTTCAGCTTTGCCCCTTTTCCTCAGTACTGATTTGGCTATTTGGGGTGTTTT
CTGGTTCCATATGAATTTTGAGATTATTTTTTATATTTCTGTAAATAATATCATTGGTATTTTGATAGGGATTGCATTGG
ATCTGTAGATTGCTTTGAGTAGTGTGGTCATTTTAACAATGTTAATTCTTCCAGTCCATGAACATGGGATGTCTTTCCAT
TTTTTTGTGTTCTCAATTTCTTTCATCAGTATTTTGTAGTTTTTGTTGTAGGGGCCTTTTGCCTTCTTGATTAAGTTTAT
TCCTAGGTATTGTAATTGTCTTGTAGCTATGCAAATAAGATTGCTTTCTTGATTTCTTTTTTCTTTTTTTTTTTTTTTGA
GACTGAGTCTCACTCTGTCACCCAGTCTGGAGTGCAGTGGCACAATCTCAGCTCACTACAACCTCTGCCTCCCAGGTTCA
AGTGATTCTCATGCCTCGGCCTCCTGAGTAGCTGGGATTCCAGGTGTCTGCCAACACACCTGGCTAATTTTTTGTATTTT
TAGTAGAGATAAGGTTTCACCATGTTGGCCAGGCTTGTCTTGAACTCCTGACCTCAGGTGATCTGCCCACCTCAGCCTCC
CAAAGTTCTGGGATTACAGGTGTGAGTCACCACACCCAGTCCTTGTCTGGTTTTGGTATCAGGAAAATGCTGGCTTTGTA
GAATGAGTTAGGAGGAATACCTTCATCTTTAATTTTTTTGAATAGTTTGAGAAAACTTTGTGTTAATTGTTTGTTAAAAA
TTCAGCAGTAAAGCCATCCAGTCCTGAGATTTTCTTTGTTGGGAAATGTTTTATTACTGATTCAAACTTATTACTTGTTA
TTGGTCTGTTCAGATTTTTCTATTTCTTCCAGGCTCAAATTTGGTAGGTTGTATATATCCAGAAATTTATACATTTCCTG
TAGGGTTTCCAATTTGTTAACATATAGGTGTTCATAATTGCCTCCAATGATCTTTTATATTTCTGTGGTATCAGTTGTAA
TGTCTCCTTTTTTATTTCTGATTTTATTTATTTTGGTCTTCTTTCTTTTCTTGTCTTGGTTAGTCTAGCTAAAAGTTTAT
TGGTTTTGTTTATCTTTTCAAAAAATAATTTTTTTGTTTTATTGATCTTTTCTATTTTTAAATATCTCTTTTGTTTAGTT
CTCTGAGCTTTAGTATTTCTTTCTTTCTCCTAACATTTGAAAGTGTTTGTTCTTGCTTTTCTATTTGTTCTTGCTTCCTT
GAGGTTCGTTGTTAGGTTGTTTATTTCAAATCTTTCTACTTTTTTGATGTAGGCATTTATTGCTATAAATTTCCATCTTA
GCATTGTTTTTGCTGTGTCTCCTAGGCTTTGGTGTGCTGTATTTGCATTTTAATTTTTTTCAAGAAACTTTTTTACCTCC
TATTTAATTTCTTCATTCACCCAGTGGTTGTTCAGGAGCATGTTGTTTAATTTCCATGTATTTATACAGTTTCAAAAGTT
CATCTTGTTATTGATTTGTAGTTTTATTCCATTGTGATCTGAGAAAATACTTGATATGATTTCAATTTTTAAAACTTTTG
TTGAAACTTGTTTTGTGGCCTAAGATATGGTCTATTCTGAACAATGTTCCATGCACTGATGAGAAGAATGTGTACTCTGC
AGCTATCAGATGAAATGTTCTGTAAATATCTGTTAGGTCCATTTTGTCTAAAGTGCAGTTTAAATCCAATGTTTCTTTGC
TGATATTCTGTCTAAATGATCTGTTCAATGCTGAGAGTGGGATGTTGAAGTTCCCAACCATTATTGTATTGGAGTCTATC
TCTCTCTTTTGATCTAATAATATTTGCTTTATATATCTGGATGTTCCTGTGTGGGGCACATATATACTTAGAATTGTTAT
ATCCTTTTGATGAATTGATCCCTTTAACATTATATAATGGCTTTTTTTGTCTTTTTTAATAGTTTTATACTTAAAGTCTG
TTTTATCTGATAAGTATAGCTATTCTTGCTTGCTTTTGGTTTCCATTAGCAAGGAATATCTTTTTCCTTTCCTTCAATTT
CAGTCTATATGTGTCTTTGAAGGTGGGGTGAATGTCTTGTAGGCAGCATATAGTTGGGTAAGTAAAAAAAATCTATTCAG
CTAGTTTGTATCTTTTAAGTGAGGAAGTTAATCTTTTTACATTCAAGGTTATCACTGATAGGTAACGACTTATTTCTGTC
ATTTGGCTTATTGTTTTCTGGTTGTTTTGTATATCCTTTGTTCCTTTCTTCCTCTCATTGTTTATCATTGAACTTTGGTG
GTTTTCTGTAGTAGTAACTTCAACTCTTTTCTCTTTCATTTATGTATTTGCTCTACTAGTGAGTTTTATACTTTCATGTG
TTTTCATAATGGTAGATATAATCTTTTCACGTCCAGACGTTCTACTCTCTTAAGCATTTCTTGTAGGGCTCGTCTAGTGG
TGATGAATTCCCTCAGTTGTCGCTTGTCTGGGAAAGAATATTTCTTCTTCATTATGGAAGGACAGCTTTGATGGGTATAG
TCTTCTTGGTTGACAGTTTCTTCTTTCAGCACTTTGAGTATATTATCCCATTCTCTCCTGGCCTATAGTTTCTGCTGAAA
AATCTGTTAGTCTGATGGGGAGTCTCTTCTATATGACTTGACTTTTTTTATTTCTTGCTGTTTTTAGGATTCTGTCTTAG
TCTTCAACTTTTGAGAGTTTGACTGTGATCTTCCTCAGAGAGAACATTTTTGAATTAAATCTACTTGGGAATTTTTAAGC
TTCTTGTATCTGGATGTCTATACCTCTTGTAAGACCTGGAAAGTTTTTAGCTATTATTTCATTAAACATGTTTTCTATCC
CTTTGCTCATCTCTTCTGCTTCTGGAACTCCCAAAATGTGAATTTTGTTTGCTTAATGGTGTTCCATAGGCTTTCTTTAT
TCTTTTTTATTCCTTTTTTTTTTTTTTCGCCTCACTGGGTTATTTCAAAAGACCTGTCTTCCAGTTCAGAAATTCTTTAT
TTTGCTCAATCTAGTCTACTGTTTTTTAATTTTTAATTTTATATATATATTTTTTTTTTGAGCTGGAGTTTTGCTCTTGT
CACCCAGGCTGGAGCATAGTGGTGCAATCTCAGCTCGCTGCAACCTCTGCCTCCTGGGTTCAAGCAATTCTCATGCCTCA
GCCTCGCAAGTAGCTGGGATTACAGGTGCCTGCCACCACGCCCAACTAATTTTTGTAATTTTTTTTTTTTTTTAGTACAG
ATATGGTTTCACCATTTTGGCCAGGCTGGTCTCGAACTCCTGACCTCAGGAGATCCACCCACCTCAGCCTCCCAAAGTGC
TGGGATTACAGTTGTGAGCCACTGCACCTGGCCTAGTCTATTGTTTGTACAAGCTCTTGATTATATTTTTTTACTTCATT
TATTGACCTCTGTAGTTCCAAAATTTCTGTTTTGTTCTATTTTTAATATCTATTTCTTTGTTGAATTTCTGACTCAGATC
ATAAATTATTTTTCTGATTTCTTTTATTATTTATCTATGCTCTCTTGTAACTCACTGAGTTTCTTTAGTATCATTATTTT
GAATTACTTTTCAGGCATTTTATAGATTTTCTTTTTGTTGAGATCTGTTATTAGAGAATTATTGTGTTCCTTTGGAGGCT
TCATGTTTCCTTGCACTTTTGTATTTCTTGTGTCCTTATGTTGATATCTATGGATCTGATGTAATAGCTGCTGCTTCCAA
TTTTATGGATTGGCTTTGATAGGGAAAGACTTTTGCCTCTAGGTGTTTTTATAGTGTTAGTTGAGTATAGGGTGCTTTGG
CTTTGATTCTGAGTGGGCACAGCAGTGTAGTTTCTGTATGATTTCTTCACCTGTCATCAGTGTCAGTGGCATTTGGGTTC
CTCAGTGGCTTAGGTTACAGTTGTCAGCAGACACTATGGCAAGGCTTTGCTGGGGATGGGGACACCAGGCAGGCCTGTTC
TCAGGCAACAGTGGTGGCAGCAGTGGCCTAGGCTTGCCGGTTCTCAGGCCCCTGTGTGAAATATGTGGGCACTCGTGGTG
ATGGGTCCTTGAGCCTCCAGGAGATTTAGTTGGATGCTTGCAGTGGCAGTAATGGACCAAGTGGGAGGGCAGGTTCTTGG
GCCCCTGCACAATTTGTGTGGCATTAACTGTAGCAGTAGGAAAAGTGGACCAACCCTCTGGCCCCCAAGTGGTAGACACA
GGCAGCAGCAGGCTGGGCAAGCCAGTCCTCAGGACCCCGTGTGGCATGCATGTGCCACACCCTGTGTGGCACTGCCCTGC
CATGGCAGTGGCAGGCAGGCACTAGCAGCAAAGGGCATGGTGGACCTGTCCTCAGGCCCCTTGGAGGCATGTGTAGATGT
GTCGTAGTCCTGCTGCTAGGGAAGATGGGGTTGCTGTAAGTGGCGGTGGCCCTGGGCAGGTAACTCTGAGGCTCTGGGGA
ATGCACACTTTGACTCCCTTTGTCCCAGGGAAAGCCACTAGGTGCACTGTGCTTCCTGTTTCCTGGGATGCGGGGCATTG
CATGGGCTAGAGTGCTGGGGACCTAGCTGCACTGCTGATCTTAGCTGGCATTACAATGCTGTAGCCCTCTAGGTGGACGT
AGGAGAATATCAGTGGGGCTTCAGGGGTGTGGAGATTCAGGGTTGTTGGGCCCCAGAACAGGATGTGTATGGTGAGGGCT
GGGCTCTTAAAAGAGCCTTATGCTGAAGCTGTTTGGGTCTCAGGGTATGGGGTATGTGGGACCCAATGTGAAGTCCCATT
GTGGAACAATGTGATTGCATATATTCCAGACAGCCCCAGGGCATGTGAAGGCCAAGGAGCTAGCTCATGGCTAGGATTAC
AGGAGTCCATAGTGGAAACATTGACCTCTGAAGATCTCTCAGTTAAACTTTCCCTGGAATGGGAAGTTCCTCCTGGTTCT
GAGCTAGTCCCAGCTGGGCTGGCTGCTTCACTTCCCTCTTCTCCCATGCCTCAGAGGTGCACTGTCACTTCTCCGCTGAA
TTCCAGTGTTCTCTCTTAGATGCTCATTCAATGTGTGATTATCTGCTCACTGTTTTGGTCCTTCTTTGCAGAGGGGGTGA
GGGCTAGGTGCCTCTATTCAGCCATTTTAATGCTTCTCCGGCTCATTTTTTAATTGTTCAATAGTATTTTATTGTGTGAA
TGTACCAGTTTGTTAATATTTTTATCTATTGAGGGATATCTTGTTGCTTCCAATGTTTGACAGTTATGAATAAAGCTTCT
ATAAACATCCACATGGAGGCTTTTATGTGGACATAAATTTTCAGTTCTTTTGGTTAAATACCCAGGAATGTGATTTGCTG
GATTGTGTGATAAGACTATGCTTAGTTTTATAAGAAACTGCATTCCATAGTGGCTGCACCATTTTGCATTCCCACAAACA
GTGAAAGAGAGTTCTTGTTGCTCCACATCCTCACCAACATTTGATGTTGTTTTTGGATTTTACCCATTCTCATAAGTGTA
ATGTAATTTCATTGCTGTATTAATCTGCAAAACCATGATGAAATATGATGTCAAATAGCTATGTATATGTGTATTTGCTC
TTTACATATCTTCTTTGGTGAGGTGTCTGTTTAGATTTTTGTGCCCATTTAAAAACTAGGTTGTTTGTTTTCTTATTGTG
GAGCTTAAGAGTTCTTTGCATAGTTTGGATACAAGTCCCTATCATATATGGGTTTTGCAAATATCTTCTCTGAGTCTGTG
GTTTGTCTTTTTCACTCTCTTGCAGTGTCTTTTGCAAAGCAAAAATTTTAATTTTAATGAAGTCCAACTTAACAGTGTTT
TCTTTCTTGGATTATGTTTTTGATGCGTATCTCAAAAGTCATCACCAAATCCAAAGTCACCTAGATTTTCTCCTATGTTA
TCTTCTGGAAATTTTATAGTTTTGCATTATACATTTAGGTCTATGGTTAATTTTGAGCTGATTTTTGTGAAAACTGTAAG
GTCTGCGTCTAAATTCTTTCTCTTCTTCTTCTTTTTTTTTTTTTGCATGTGGAGGTCTAATTGTTCCAGCACCATTTGTT
GAAAAGACTATCCATTCTCCACTGAGTTGCCTTTGCTCGTTTGTCAAACAGCAGTTGATTATTTTTTTGTGGGTCTATTT
CTAAGCTCTGTATTCTGGTCATTTGACCTATTTGTCTATTCTTTCACCAAAACCACACTGCCTTGATTGCTGTAGCTTTG
TAGCAAGTCTCAATGTTAGGTGGTGTCAGTTATCTGGCTTTGTTCTTTTAATTTCTTTTTTTTTTTTTTCCTGTGAATGA
ACAATACTTTCTGTTTCTTTGCATGGCTAATTTTTTTAAACAAATAAATTAGAGATTCCTTTAATGCCAAGTTACACAAT
CAAAAAGATAACAATCTCTTTCCATTTGCTTTTTGCAAGGTTTGTGTTATTTCAGGGCAAAACGTAGAGCAAGTCCACTG
GCAAATCTGGGGGCTAGTGATTATTTTGTTTTGATTTTTATTTTTTTAAATCAGACATTTTAAACACTACAATGAGGTAA
CTCTAGAAATCAAATTCTGTCCCCCTTTCCCAGAGGTGTTTTATTTATTTATTTATTTATTTTTTGACAGGGTCTCATTC
TGTCACCCAGGCTGGAGTGCAGTGGTGTAATCACAGCTCATTGGAGCCTCGACTTCCTCGGCCCCAAACAATCCTCCCAC
TCAGCCTCCCAAGTAGCTGGGACTATAGGCACACGCCACCATGCCCAGCTAAGTTTTGTATTTTTTGTAGAGATGGAGTT
TCACTATGTTGCGCAGGCTGGCTTTGAACTCCTGGACTCAAGCAATCCACCTGCCTTGCCCTCCCCAAGTTCTGGGACTA
TAGGCTTAAGCCACTGTGCCGGGCCTCCCTTTCCCAGGGTGTATTGTTGCTTCTTGTGGCTTATAGTTGCTTGTTTAATG
ACTTTTCTAAATAATTTTTATAAAGATTATTCTTTGTCATGTGTGGCTATTACATTTTCTGTCCAGTTAGCTTAGTGGTC
AGCCAGTAACTTGAGATTTCTTTAAACATCTGGAGCAAAAACAAAAACCAAATAAACTAGCAAAAGCAAAAACAAAAACA
CCCCTGATCTTTGCAGGTTGGCTGTGTTGGATCACTTTCTCAATGCTTAGCCAGTCTATTCACAGCTCTGCTTTAGCCTT
CACTTTCTGCATGCATGGAAGCTAAAGATGAGCCAGAGGTGAAGTTTAGGATTTTCTCAGGTCTCCTCTGAACATGCGTC
CAGCCTTGGGCACGTGAGTGGACTTCTGGACTCCCTGGTATATACAGAGATTTCCTCAGCCTTTTTTCTCCCACATATCT
TTCTCCATATATCTTTGCATATTTCCCAGACTTTTTGATGTGTCTGCTGCTTGCCCCCTCTGTTAGCCCTTGCTCAGGTG
GCTGCAGCTAGTATGTTTGCCTTTAATTGTTTACAGCAAACACCAGCCAGAAGGCTGTTCCAGCCCTGAGAAAGTTCTAA
GGTGTACAAAACAAAGGCAAGCCCCTGAGTGTGAATCCCTCAGGCAGCAACCAGACAGGTCAAAACACACAACCACAATT
CTTTGATAACAAGGCCTATATTGCCCCTTCTGGCATGAGCAAGCTATATAACTGGTTATAAAAAGTATTACATTAAATTA
TGGGGACAGAAATGACTCTACTAGTGAATGAAGAAATCATTTAGATTTTATGGTTAGTAAGTGGAGGTTTGAGAGTAACC
ATCCGATCTTTATTAATCTTAGTGTATATTATATTTCTAGAATTTGGACTCATATCAAGATGCTCTGAAGAAGAACAACC
CTTTAGGATAGCCACTGCAACATCATGACCAAAGACAAAGAACCTATTGTTAAAAGCTTCCATTTTGTTTGCCTTATGAT
CATAATAGTTGGAACCAGAATCCAGTTCTCCGACGGAAATGAATTTGCAGTAGACAAGTCAAAAAGAGGTCTTATTCATG
TTCCAAAAGACCTACCGCTGAAAACCAAAGTCTTAGATATGTCTCAGAACTACATCGCTGAGCTTCAGGTCTCTGACATG
AGCTTTCTATCAGAGTTGACAGTTTTGAGACTTTCCCATAACAGAATCCAGCTACTTGATTTAAGTGTTTTCAAGTTCAA
CCAGGATTTAGAATATTTGGATTTATCTCATAATCAGTTGCAAAAGATATCCTGCCATCCTATTGTGAGTTTCAGGCATT
TAGATCTCTCATTCAATGATTTCAAGGCCCTGCCCATCTGTAAGGAATTTGGCAACTTATCACAACTGAATTTCTTGGGA
TTGAGTGCTATGAAGCTGCAAAAATTAGATTTGCTGCCAATTGCTCACTTGCATCTAAGTTATATCCTTCTGGATTTAAG
AAATTATTATATAAAAGAAAATGAGACAGAAAGTCTACAAATTCTGAATGCAAAAACCCTTCACCTTGTTTTTCACCCAA
CTAGTTTATTCGCTATCCAAGTGAACATATCAGTTAATACTTTAGGGTGCTTACAACTGACTAATATTAAATTGAATGAT
GACAACTGTCAAGTTTTCATTAAATTTTTATCAGAACTCACCAGAGGTTCAACCTTACTGAATTTTACCCTCAACCACAT
AGAAACGACTTGGAAATGCCTGGTCAGAGTCTTTCAATTTCTTTGGCCCAAACCTGTGGAATATCTCAATATTTACAATT
TAACAATAATTGAAAGCATTCGTGAAGAAGATTTTACTTATTCTAAAACGACATTGAAAGCATTGACAATAGAACATATC
ACGAACCAAGTTTTTCTGTTTTCACAGACAGCTTTGTACACCGTGTTTTCTGAGATGAACATTATGATGTTAACCATTTC
AGATACACCTTTTATACACATGCTGTGTCCTCATGCACCAAGCACATTCAAGTTTTTGAACTTTACCCAGAACGTTTTCA
CAGATAGTATTTTTGAAAAATGTTCCACGTTAGTTAAATTGGAGACACTTATCTTACAAAAGAATGGATTAAAAGACCTT
TTCAAAGTAGGTCTCATGACGAAGGATATGCCTTCTTTGGAAATACTGGATGTTAGCTGGAATTCTTTGGAATCTGGTAG
ACATAAAGAAAACTGCACTTGGGTTGAGAGTATAGTGGTGTTAAATTTGTCTTCAAATATGCTTACTGACTCTGTTTTCA
GATGTTTACCTCCCAGGATCAAGGTACTTGATCTTCACAGCAATAAAATAAAGAGCGTTCCTAAACAAGTCGTAAAACTG
GAAGCTTTGCAAGAACTCAATGTTGCTTTCAATTCTTTAACTGACCTTCCTGGATGTGGCAGCTTTAGCAGCCTTTCTGT
ATTGATCATTGATCACAATTCAGTTTCCCACCCATCGGCTGATTTCTTCCAGAGCTGCCAGAAGATGAGGTCAATAAAAG
CAGGGGACAATCCATTCCAATGTACCTGTGAGCTAAGAGAATTTGTCAAAAATATAGACCAAGTATCAAGTGAAGTGTTA
GAGGGCTGGCCTGATTCTTATAAGTGTGACTACCCAGAAAGTTATAGAGGAAGCCCACTAAAGGACTTTCACATGTCTGA
ATTATCCTGCAACATAACTCTGCTGATCGTCACCATCGGTGCCACCATGCTGGTGTTGGCTGTGACTGTGACCTCCCTCT
GCATCTACTTGGATCTGCCCTGGTATCTCAGGATGGTGTGCCAGTGGACCCAGACTCGGCGCAGGGCCAGGAACATACCC
TTAGAAGAACTCCAAAGAAACCTCCAGTTTCATGCTTTTATTTCATATAGTGAACATGATTCTGCCTGGGTGAAAAGTGA
ATTGGTACCTTACCTAGAAAAAGAAGATATACAGATTTGTCTTCATGAGAGAAACTTTGTCCCTGGCAAGAGCATTGTGG
AAAATATCATCAACTGCATTGAGAAGAGTTACAAGTCCATCTTTGTTTTGTCTCCCAACTTTGTCCAGAGTGAGTGGTGC
CATTACGAACTCTATTTTGCCCATCACAATCTCTTTCATGAAGGATCTAATAACTTAATCCTCATCTTACTGGAACCCAT
TCCACAGAACAGCATTCCCAACAAGTACCACAAGCTGAAGGCTCTCATGACGCAGCGGACTTATTTGCAGTGGCCCAAGG
AGAAAAGCAAACGTGGGCTCTTTTGGGCTAACATTAGAGCCGCTTTTAATATGAAATTAACACTAGTCACTGAAAACAAT
GATGTGAAATCTTAAAAAAATTTAGGAAATTCAACTTAAGAAACCATTATTTACTTGGATGATGGTGAATAGTACAGTCG
TAAGTAACTGTCTGGAGGTGCCTCCATTATCCTCATGCCTTCAGGAAAGACTTAACAAAAACAATGTTTCATCTGGGGAA
CTGAGCTAGGCGGTGAGGTTAGCCTGCCAGTTAGAGACAGCCCAGTCTCTTCTGGTTTAATCATTATGTTTCAAATTGAA
ACAGTCTCTTTTGAGTAAATGCTCAGTTTTTCAGCTCCTCTCCACTCTGCTTTCCCAAATGGATTCTGTTGTGAGCAAGA
GTTTATATGGCTTCATGGCAGCAAGGGAACAGTCAACTTCAGCATCATATGCACCAGTCCTCGGAGTGCCCTGTGAATCA
TATTGGTCTTTGGGTCAGTGTCATCATTCTCTTCAAGTCTGGGGCTTGGGGAAAAAATTAGATCAGCTACGGCATATAAA
AAAGTCTTTTGTTTCACATATGTGTAATAGCTTATTTAATTTTTTATCCTGCTACACAAATATGTAATTAACCAATGAGG
ACTCATGACTTGATAGTGTATGTATGTAAAGGGATATATGGACTTAATCATAAGCTGTTGAGGTGAAAGACGTGGATCCA
CCTGCTTTCCAAGAAAACTCGGCCAAATTTATTTGCAGCTGGATATTGAATGGGACTTTTCTGGTTGTCTTAGAATTCTG
GCTAAAGGCTCAAAGCTGACGAAAGACAGTAACTGCACCAACATGATACTAGACACAGCCAGTCTGGACTTATCAAAAGA
GCAGAAAGAGACCAATGACTCCCAGTCCGTATTATCCATCTCTAGAAGACTAGAGTCAAAAGCGTGATTAAAGAGTCATT
AAGCGGAGGTTCTAGGCCATAGGGAGATTGCTTTGAATTTCTTGCAGACAAGTGTGAGGGACTCAGCATGGTAGAAGGTA
GCCTGGCATCCCACTCCAAGACTGAAAGCTTGCAGAGTAACAGGAGCACACAGGTTCAGTGCAGCAGATGTGGTGTGGCT
TGAGAATTCTTGGAAGAGCTTGATGAGTGTTTGCTGGAGTCCGAGGGTGGGCACTGGGAACACAGAGACTGGTAAATAGT
GTTTGGCAAATACAAGTGCTTGATGAATATTTGTTGAATGAATAGATGAGTTCTTCCCCCCTGGGGAATTCAGGAGGTGA
AAGGTTGGCTTGAGCACCCAAAATGGCAGGATGAGAGAAGAGAAGCACTGATAGCAACCTGCCCTCCCATTATTGACATG
GTAAAAGGATGTGAATTTCTTCACATGGCTTTGACTATGGAAGAGTAGCTGGGCTTGCATTGTCATGACGGGATATCAGC
CAACAGGGTAGCCTGTTGTGCAAAGAAACTATAGCAGTAAGAGGACACGGGGTTAGGCAGAAGAGGGGTTTGGGGTGGAG
GTTGCTGCAAGAGGTCAGCCAGATAATGTGGCCCTGCATCATGGAACTGTGCAATGTGGGGTACACTCAAGGCCCTCCAA
TAACTCACAGATGTGCCCTATGAAAAAGCCAGCATTTGGACTCTGCCATAGCAGCTGGCAGGATCATGCTGGCCTGTCTG
CCTTATTCAATAGTTAACTACAGGAAGATCTGCTCCTCTTTGTGTAATACCCTCTTCCCTTGCAATGGCATAGGGACATC
TAGAATATAGAGAAGACAGAGACAATGGAGGAAGAGTAAAGAAACTGACTATATGCCTTCGTCATTTCACTGCAAGGAAG
GCCAAGCAGATTTTTGAATGAGGTGTGAGATTGCTGTTAAATTGGACTGGCCTGGACATTTTAATCCCTTAAATAGAGGT
GCAATGACTAAAGTGAGATTTGTCACTAAAATTTATGGTATCTGCCCAAGATTCAGGAGTGATGATGGGAGGAGATCCAA
CAGAACTTTGTTGTAAGGCAATGGTTAGAGAAAAATGAAGCCCTCGCTTTCTGGACTTAGTTCATTCAATAAACCAGTTT
CGGCCAGGCACGTTGGCTCACATCTATAATCCCAGTACTGTGGGAGGCTGAGGCAGGTGGATCACTTGAGGTCAGGAGTT
CGAGACCAGCCTGGCCAACATGGTGAAACCCTGTCTGTACTAAAAATACAAAAATTAGCCGGGTGTGGTGGTGTGCACCT
GTAGTCCCAGCTACTCGGGAGGCTGAGGCAGGAAAATCACTTGAACCTGGGAGACAGAGGCTGTAGTGAGCTGAGACAGC
GCTACTGTACTCCCCGCTGGGCAACAGAGTGAGACTCCATCTCAAAAAAGTTAAAAGAAAAAAAATCTGGTTTCATAATA
GCTGTAACGAAATAAGCCTTAATGATATTTTATTAGCATCATCTTCTGTCTGCATTAGCCCTTCCTTGCTCTTCAGGAGA
ACAACATTTGTTTTCCTCCCTAGGCTCTATCCCAAACGGCACATTCTTCCACAACCCCTGTTGAACAGATTTTTTAAACT
GTTGCCTAATCTAAAAACAATAAAAACAACAAACAACCACAGTAACAACAACGACAAAAAAAACTGCCACAGATTCTAAA
TAATCAGATCTTTTTAAATGGTATCAATGTTTCCCACAAAATATTGTTGACATTGAAAATATAGAATTTTAGCATTAATT
TTGTTAAACCTACATCCCCTCGGCAGAGGGGCCTCCCTGCATCCCAGTGGAAAGTAGGTTCCTCACAGTCCTCTCCGTCA
CATTCTTCCCATTTCTTTTCTTCACAGAACACATCACTGTCTAAAATTATCTTGTTTGCTTAGTTGCTTACTCATCTTCT
TCTTCTCTCCTCTGAAGTCTAAGCTCCAGGAAAAAGGGAGACTTCTCCACCTGTTCCCTGCCTCTCCCCAGTGCCGAGGG
GACACTGTGCACCCCATTGTAGATGCGCAGTAAAAACTCGTGGGATGAGCAAATGACTCTGAAACGGTCCCATGCGGGAA
ATGTCCATGAAGTCCTGGATTTTATCTAAAAAGCCCAGGCAGGGGGGGGCGGGGGCGGCGGGGCTACAGTTCCACGCTGA
GCTGCCTCCTGGCCGCTCGTCCCCGCCGCAGTGCCTGGGCGGCCCGGGCGCCCGACCTTGGCCGTGGACACCTTCGCGGT
GGGTGCTGCTCCTCCCCATCTGCCACTGGAAGATGCTGGGGCGACCCGGCTCCAGGTTTAGCAGGACACTGAGAAAAGGG
AATGGCTGCCTTTCGGAGGCTGGGTGAGCCCTTCTCTGTGCCTCACCTGCCCGCCCCACAGCGGCCCTGCACCTCGTCCC
ACGGGGCCCATTGCCCCGGTAGGATGCGCGCTTTTGTTTTGAGGGTCAGGCATCTTCCCTGCCGTCGTTTCTGGGAGGTT
GAAAAATTGATCCAGAAAGACCTAAAACAAAAAACAAAGAAAAACAAAAAAGCGAAAAGGAGGCAAAACAAAACAAAAAT
CCATACAAATAAAGTATCTCCTCCATCCATGTTTGCACTATTCCTTCTCATTCCCCCGTCCAATGCCACAATTTGGTAAG
TTTGCCTCAACACGTTTTCAGTTCTTAACACTGTGATAAGAATAAACAATAGATCCCGTAGTAAGATGACCTGTTAAAAG
GAATGGGGCATGGCAGAGATGAGTTTGGATGAGGAAAGGTGTCAGTCCGTTTGGGCTGCTCTAACTAAGTTCTGAGATGT
GTGTTAGGTAATTTTGACTTTGGTTGAACATCATAGAGTGCACTCACACAGACATAGATGACATAGCTTACTACACACCT
AGGCTTATGGTATAGCCTGTTGCTTCTAGGTGATTGTAACACAATGCTTAGTATTTGTGTATCCAAACATATCTAAACAT
AGCAAGGGTATGGTAAAAATATGGTAAAGAAGATAAAAAATGGTACACCTGTATAGGGCACTGATGTGGGTTGGCTGTGT
CCCCACCCAAATCTCATCTTGAATTCCCACGTGTTGTGGGAGGGACCCAGTGGGAGGTGATTGCATCATGGGGGCGGGTC
TTTTCCTGTGCTGTTCTCCTGTTAGCGAATGGGTCTCATGAAATCTGATGGTTCTTTAAGGGGGAGTTTCCCTGCACAAG
TTCTCTCTCTTCCCTGCTGACATGTAAGATGTGACTTGCTCCTCCTTGCCTTCTGCCATGATTGTGAGGCTTCCCCAGCT
CTGTGGAACTGTAGGTCCAATAAACCTCTTTCTTTTGTGAATTGCACAGTCTCAGATACGTCTTTATCAGCAGCATGAAA
ATGAACGAATACAGGCACTTACCATAAATGGAGCTTGCAGGATTGGAAGTTGCTCTGGGTGAGTCAGTGAGTGAGTGAGG
GGTGAGTGAATGTGAAGGCCTAGGACATTACCATATCCTACTGTGGACTTTACCATGTCCCACTGTGGACTTTATAAGTC
CCATTGTACATATAGGCCACACAAAAATTATTAAATTTGTTTTCTTTCTTTAGTAAGAAAAATTATTAAAATTGTTTTCT
TTCTTTAGTAAGAAGTCAACCTTAGCTTATTGTAATGTTTTTACTTTATCAACTTTTTGCCTTAAATTTTTTTTTGGCTC
TTTAGCAATAACACTTAGCTTAAAACACACATTCTACAGCTATATAAAAATATTTTCCTTCTTTACATCTTTATTTTGTT
TTTTCTATTTTTAAAATATTTTATTTTTATTTTTCACTTTTTAAACTTTTCTGTTAAAAACTGAGACACACACACAGACA
CACACACACTAGCCTAGGCCTACTCATAGGGTCATTATCAATATCACTGTCTTCTACTTCTACCTCTTGTTCCATTGGAA
GGTCTTTAGGGGCAATAACATGCATGAAGCCATCATCTCCTGTAACAACAATGCCTTCTTCTGGAAAACCTCCTGAAGGA
CCTGCTTGAGGATTTTTATAGTTAGCTTTTAAAAAAATAAGTAGGAGCACACTCTAAAATAATGATTAAAATTATAGTCT
ACTAGATACAGAAACCGGTAACATAGTTGCTTATTATCATTATCAAGTATTATGAACTGTACATAATTGTATGTGCTGTG
CTTTTATATGACTGTCAGTGTCGTATGTTTGTTTACAGCAGCATCACCACAAACATGTGAGTAATACATTGCACTGCAAT
GTTACTAGGCCATAGGAATTTTTCATCTCCATTATTATCTTATAGGGCCATGACTGTTGAGTGAAATATTGTTATGCACT
GCATGACTGTACCACAAATTGAGTAGCTTATAAACAATGGAAATTTATGGCTTATAGTTCTGGAGGCTGTAATGTCCAGG
GTCAGGCTGCTGACAGATTTGGTGCCTGGCGAGGGCCCACTTTCTGGTTCATAGATGGCTTGTCTTTTCACTGTATCCTC
ACCTGGGGGAGGGCACTAATGCCATTCATGAAGGCTCCACATTCATGACCTAATTGTCTCCCAACATCTCCAACTCTTAA
TACAGCTCTTTCTCCGTATCCACAGGGGATTGGTTCCAGGAGCCCTGTAGATTCCAAAGTCTGTGAATGCTCAAGTCCCT
TATGTAAAATGGCATACTTCCAACCTGAGCATCATAGTGAGACCCCACCTCCTCAAAAAAAATACATGCCTGGCGGCACA
TGCCTGTAGTCCCAACTACTCAGGAGGCTGAGGTGGGAGGATTGTTTGAGCCCAGGAATTCCAGGCTGCAGTGAGCTATG
ATCATGCCGTTGCACTATAGCCTGGGCAACAGAGCAAGACACTGACTCTGAAAAAAAAAGTGTACTATTTACATATAACC
TATGTACATCCTCTCATATACTTAAATCATCTCTAGATTTCTTACAATACCTAATACAATGTAAATGTTATGTAAATGTT
ATGTAGTTGTTAAACTGTATGGTCTTTTATTTTTATTATTTTGTATTATTATATTGTTATTATTTATTTATTCATTTATT
GAATATTTTCAATCCATGGTTGTTTGAATCTATGGATGTGGAACCCATGGATATGAAGGGCTGATTGTATTATCCCCTTG
GGGGTTAGGATTTCACCATAGGAATTTTGAGGGGCACACAAACATCCAGACCATAGCAAAAGAGATAAGAAACCTAGGTT
AGTGACCAGGGAGGGCTGCTAGGATGAGCCTTGATGAAAGATCTTTCATCAGGAGGATCCTGGGCATGTGGCTGGGAAGG
GTCCTGGGCATGTGGCTGGGAAGACCCTTCAAAGGGGAGGGCACAGTGGGTGCAAAGTTGGAAGTGAAAGAATACCATGA
GCGAAGAAAGCATCAAATTGGCAGTGTTGCTGGATGTGTTAGTTTCCTGTGGCTACCAAAACAAATTACCACAAACTGGG
TGGCTTAAAACAACAGAAATATATTTCTCCCAGTTCAGGAAGGCAGAAGTCAGAAATCAAGGTTGGCAGGGTTGTTCCTT
CCAGAGGCTGTCAGGGAGAAATCATCCCTTTCCTCTCTCCTAGCTTCTGGTAGTTGCTGGCAAACCTTGGCGTCCCTTGG
TTTGCAGATGCATCAGCTGCTTCTTAGAATGCCCTGGAGGACATGCCCCAGTAGTTAGCAGCTTTCTATAGAATGTGGTA
CATAATACTTCTTTGTCTTTAGTTTTGGACCCAAGTCATAAGAAAAGTTCCAGTCAACACCCTATTACGTAATCAGTAGA
ACCAACAGATGAGAAGAGAAACCAAAATTATTGGTAGGCTATTGACATACATGGCAGATGCGCAATCTAAAAAAATGTAT
CCCAAATTTGGGTTAAATAATCAATAATCTGTGCCTGTGTCTTCACATGGTGTTCTCCTCCAAGTCTCTGTCTTTTTTTT
CCTGTCTCTTACAAGGACACTCTAGTTGGATTTAGGAGCCTCCTTAATCTGATGCCATCACATCTTGGTCCTTAACTTAA
TTAGATCTACAAAGACTCTTTTTTCAATGAGGTCACATTCTGAGATTTCAGAATAAATCTGAAATAAATGGACATAAATG
TTTGGAGGTTGGGTGGAGGACACTATTCAACCCAGTCCTCTGGAGTTGGCATGACCAGCTCTGATGCCCTGGAGACTCAG
TTGAGTGTGGCAGCAGAAAAGAAATGTTATCCTGGAAAGAGTGAAGACTCTAAAAGAAGCTGCTTGGAAAGTCAGAGGTA
CAAAGAAAGGAGTAATAGGCAAAGAGAATAGAGTGGATGGGTGTAAGAGCACAGGAAAGAGACAGTGGATGGAGACCCGG
GATATAAACGGTGAGAGTGTGGGAGTTTTAAAGAGAATGAGACCACAAATATGGGGAAAGGAGGAGGTATTTCTGTCTAG
GTTTCTGGGGACTGGTTTGATTAGAAAGGAATGAGACTAAACTACAGGGAGTAAAAGACCTGAGGGGATTTCTAAAACCA
CAGGGACTTTCTGAGAGTTTCAATTACGGTTAGTTTATGAGGAATCTCAACATCATCTAGTCCAAATGTGAAGTAGTGTT
TATTTTTCTCAGTCATTCCATCAACTGGATTCTGAAATTCCCCTCTTGTCACTCCTTAGTGATATCTTGAGCAGATTTAA
TACTTCTGCAGTCCTTTTGAGATACTGATACTCCAATCTAGTTTAAAATTCTAAATCTCCATCTTCATCTAAAACTCATC
TTCTTTCTGCATGTCACAGGCTAGGTCTGTAGCTCAACCTTGGCATATATTTTGGAGGTAGGCATGGTTTATCTGAGATA
CACTCTTCTTGCCCCTCCCTCTCCCAGATCTAGCTCCCTGAAGTGGAGATCAAGGATGAGTGGTAAGAAGGCAGATATCC
TTTTCTATAATTGTGTCGATTAAGGTACAGTCTAAGCTGCAGGACTGGGAGACCTCAAATACAGAGGCTTAAATAAGATT
GAGGTTTATTTCTTTCTCATATAATAGAAAAAAGTATAGCTAAGAGGTCTAGAATAGGTAAGCTGTCTCTATTCCACAAA
ATCATTCAAGGGCTCAGGATGACCAGATGTCTCTGTTTCCTATAGCTCTGGTCTTATCCTATGGCCTCCATTCTCCCTTG
CATGGAGCCACAGGAGCTGATGAAGCCCTCCTGTGCCTTTTTGTGTGTGTGTGGCCCATGGGATGCTGAGGTGGAGATAC
ACCCCCTTTCCCAGGTACCTGATACCACAGTCTCTAATTGCCAGTTGGCAAGGGTTGGTCCAATGGAAATCTCCAACAGA
GTAGACACCAGTCGCTCTACTAATTTACACTCCTGAACCCCCAGGGGGCACGTGTAATGTTCTCTAATACTGCTCAGATG
GGCAGGGGGATCAACCTCAGAGGGTGATCCTACACATTGCTGCATCAAAACAAGAATCCTCTTGAGGAAAGAAATGCCTG
ATCTGCCTGTCCCGTGTCTAAGAGTAATTTTACTGTTATTTCCCCTTCACTTGGCTTGAGAAAGAGATGTCATCTCTTGC
ATAGGGAGAAGAGCTTGCAGGCTTACATCATTAACACTCATCTCCTGGTGCTGCTGACTCTCTTGTCACCCTCCCCAGCA
TTCTCAGGTGAATGGGGGAAGGTAGGGTGAGGCGATGCTGTGGACAGTGCTATCTGGTTCTACTGCTTCTTAGAATGCCC
CGGGGGACATGCTTCAGTTGCTAGCAGCTTTGTATAGAATGTGGTACATAATACTTCTTTGTCTTCAGTTTTGGACCCAA
GTCATAGGACAAGTTCCAGTCAACACCTTATTATGTAATCAGTACAACCAACAGATGAGAAGAGAAACCACATTGTTGAT
AGGCTATTGAATTATATGGCAGATGAGCAATCTAAAAAAAGTATCCTAAATTTGGGTTAAATAATTAATAAAAAAGACAT
TTCATCTATAGTAATAGAAAAAAAGGCAAAGAATAGTGGTATGGGTTTATGTCTATTGGTTCAAAAAGTCCACTTTATAG
AATATATATTAAAAAAATAATTGTTCAGCCAGGCACAGTGGCTCATGCCTGTATTCCCAGCACTTTGGGAGGTCGAGGTG
GGTGGATCACCTGAGGTCGGGAGTTCGAGACCAGCCCGACCAACATGGAGAAAACCTGTCTCTACTAAAAATACAAAATT
AGCCGGGCGTGGTGGTGCGTGCCTGTAATCCTAGCTATTCAGGAAGCTGAGGCAGGAGAATCTCTTGAACCTGGGAGGTG
GAGGTTGTGGTGAGCCGAGATCATGCCATTGCACTCCAGCTTGGGGAGCAAGAGCGAAACTCCATCTCAAAAAAAATAAA
TAAATAAAAATAAAAGTAATTGTTCATACACATCCCAAGGAAACATTGCTGCTTATTGCAGAATTGTTTCTAATTGTGAA
AATTGGAAGCAATTTAAATGTCCAACAACAGAAGATTGGTAAAATGCATTATAAACTAAAATATTAAAAATTGCATTATA
AATAAAAAGGAATATTATCCAGCCAATAATCATGAAATTGTAAATGGATAATTACTGACATGAAAAGATATTTATGATCG
ATTATAGGTTAAGAAAAGCATATTACAAACAGTATACGTAGTATGATGCTATGTGTGTGTGCATGCACTTGGGCATGTGT
AAGTAGATAAATTGTTATAAATTTTCTGAACCACAATTTGCAGTAGGAGCCAAGAATCTAAAGAATGTTAACATATAGAG
AAAAATGACTGCATGGTTGCTCTAAAAATGATATTGGTGTGATTCTATTTGGTGAGATTACTGCTTATTCAAATATTTTA
AAATTTATTTATATTTCCTGATTTTCCTACAGTAAACATTTATTACTTGGATAATTGAAAATTAAAAAAACTAAAAAAAT
ACATTTGCGAAGAACAGATACACGTATATCTATCTTATCTTGTTATGGAGAAAAACTTTCTAAATCTAAAACCAGGGTAG
CCATGGTGTGCTGGAGCTTGCTTGCACAGATTTGTTAAATCTGATTCTAAAGTATTCAGGGCCAGGCACGGTGGCTTACA
CTTGTAATCCCACCACTTTGGGAGGCGAAGGCGGGTGGATCACTTGAGGTCAAGAGTTTGAGACTAGCCTGACCAACATG
GTGAAACCCCATCTCTACTAAAAATACAAAATTAGCCAGGCATGGTGGCACATGCCTGCAATCCCAGCTACTAGGGAGGT
TGAGGCAGGAAAATCACTTGAACCTGGGAGGCAAAGGTTGTAGTGAACTGTGATCATGACATTGCACTCCAGCCTGGGCA
ACAAGAGCGAAACTCCATTTCAAAAAATAAAATTAAATATAAATAAATACATAAATAAAAATAAAATATTCAAGAGCTTT
GCCAAAAAATTAGTTGGTAGCTAGCCCTCAGCAATAGCGGGAATACTGACACTGCAGATATCAGCAAATGCTACAAATCA
GACTTTTCTTTTGAAACTGCTTTACCAGCACACATCTGAAAGACAGTAATAGCAAAGAAAAGGGGAACTTACTTGTTTAT
ATAAAAATGAATGTGGGGCCTGGTGAGAAGTAATTAGATCATGGGGGAGGAGTTCTCATGAATGGGTTGACATCATTGCC
TTAGTGCTGTTCTCGTGATAGTGAGTGAATGAGTTATTGTAAGATCTGGTTGTTTAAAAGTATGTAGCACCTCCCCACAC
TCTCTCTTGCTTCTGTTCCTGCTGTGGAAGACACCTTGCTCCCCCTTTGCCTTCCATCATGATTGAAAGCTCTGTGAGGA
CTCTCCAGAAACAGAAGCCACTATGCTTTCTGTACAGCCTGTGGAACTGTGAGCCAATTAAATCCCTTTTCTTTATAAAT
TAGTTAGTCTCAGGTATTTCTTTATAGCAGTGCAAGAATGAACTAATACAGAAAATTGGTACTGAGGAAGGGGGCATTGC
TATAAAAATGCCTGAAAATGTGGAAGCAGCTTCGGAATTGGGTAACAGGCAGGGGTTAGAAGAGTGTGGAGGGCTCAGAA
ATAGACAGGAAGATGAAGAAAAATTTGGAAATTCCTGGAGACTTGTTAAATTGTTTTGACCAAAATGCTGATAGTGACAG
GGACAATGAAGTCCAGGCTGAGGAGGTCTCATATAGAAATGAGGAACTTACTAGGAACTGGAGCAAAGGTCACTTTTGTT
AAGTGTTAGCAAAGAACTTGGAAACATTGAGTCCCTACCCTAGGAATCTGTGGAACTTTGAACTTGAGAGAGACCATTTA
GGGTATCTGGCAGAATGAATTTCTAAGCAGCAAAGCGTTCAACTTGTGGCCTGGCAGCTTCTAACAACCTATACTCCTAT
GTGTGAGCAAAGAAATGACCTGAAACTTGAACTTATATTTAAAAGGAAAGCAGAGTGTAAAGGTTTGGAAAATTTGCATC
CTGGCCATATGGTAGAAAAGAAAAGCCCATTTTCAGGAGAGGAATTAAGCAGGCTGCAGAAATTTTCATAACTAAAAGAA
AGGCAAATGCTGATAGCCAAGAAATGGGGAGGTGGCTTCAAAGGCATTTCAGAGACCTTAGAGGCAGCCACTTCCATCAC
AAGCCTAAGGAGGCTTAGGAAGGAAGAATGGTTTCCTGGACCAGACCCAGGGACACTGCTCCTCACATCCCAGCCACTCT
AGCTCCAACAATGGCTCAAAGTGGCCCAGGTACAGCTCAGGCCACTGCTTCGGAGAGTGTAAGCTATAAGCCTTGGCAGC
TTCCACGTGGTGTTAAGCCTTCAGGTGTGCAGAGTGCAAGAGTTGAGGCTTGGAAACCTCCACCTAGATTTCAGAAGAAG
TATGTAAAAGCCTGGATGTCCAGGCAGAAGCCTGCTGCAGGGCCAGAGCCCTCATGGAGAACCTCTACTAGGGCAGTGTG
GAGGGGAAATGTGGGGTTGGAGCCACCACACAGAGTCCCCACTGTGGCACTGCCTAGATGAGCTGTCAGAAGAGGGCCAC
TGTCCTCAAGACCCCAGAATAGTAGACCCACTGGCAACTTGCACCTTGTGCCTGGAAAAGCTGCAGGCAGTCAACACCAG
CTCTTGAGAGCAACTGTGGGGACTGAACCCTGCAAAGCCACAGAGGAGGAGCTGCCCAATGCTTTGGGAGCCCACCTCTT
GCACCAGTGTACCTTGGATGTGAGGCATGGAGTCAAGGGAGATTATTTTGGAGCTTTAAGATTTAATGACTGCCCTGCTG
GGTTTCAGACTAGCATGGGGCCTGTAGCCCCTTTCTTTTGGCTGATTTCTCCCTTTTGGAACAGTTGTATTTACCTAGTG
CCTGTACCCTTGCTGTTTAAGAGTAACTAACTTGCTTATGGTTTTACAGGCTCATAGGTGGAAGGGACTTGCCTTGTCTC
AGCTGAGACTTTGGACTTCTTTTGAATTAATGCTGGAATGAGATAAGACTTTGGGGGACTGTTGAGAAGGGATGATTGTA
TTTTGCAATATGAGAAGGACATGAAATTTGGAGGGGCTGGGGGTGGAATGATATAATTTGTATCTCTGTCCCCACTAAAT
CTCATGTCCAGTTATAATCCCCCATGTTGGAGGTGGGACCAAGTGGGAGGTGATTGGATGGGGGTAGAGTTCTCATGAGT
GGGTTAGCATCATCTTCTTGGTGATGTTCTCATGTTAGTGAGTAAGTGAGTTATCATATTATGAGATCTGGTTGTTTGAA
CGCATGTAGCCCTTCCTCTCTCTCTCTCTCTTGTTCCTGCTCCAGCCATGTTCCTGCTCTCTTGTTCACTTTTCACCACG
ATTGTAAGTTTCCTGAGACCTCGCCAGAATCAGAAGCCACTGTGTTTCCTGTACAGCTTGCAGAACTGTGAGCCAATTAA
ACCTCATTCCTTTATAAATTACCTAGTCTCAGGTATTTTTTTCATCAGTGCAAGAATGGACTATTACACAATTAAATAGT
AAAAAACAGAACCCTGAAAGAAAAATAGGTAAATGACATGAACAGGTAATTCACTAAAGAGATACATATTGCCAATAATA
AAAAAAAAATAAAACAATAATGAGATGTCACTCTACATCTATAAAATTGACAGCTCTCTATAATGTTATTTTTGTGGAGG
ATGTGTAGAATGTCTACCCCAGCACACTCTTGGAGCGCAGGTAAAGTGGAACAAACTTTATGGAGAGCACTTGGTGCTAT
GTAACATGACAATGCTCATGTCCTTCAACCTAGTCATTCTTTCCCAGAAATTTATCCTAAAGCAATAGTAAAAGATTCAG
ACCACGACTGTGTACAAAGGTGCTTATAACATTTTGTGAAATGCAAAGGGCAAATCTCTGCTCAGACAGAGCTGAGGGTG
GCTGGTCATGACCTAATATTTACTCAGGTACTGATATATCAGTGGCAATTTCAAAAATTTGACTGGAAAACTTTAGATCA
TTAAGGGAGAATTTATTAAAAGGTAAATGTAAATGTGATGACATTTCTCATTCCCAACACGTTTACAGGGTGCAGTTTTG
AAGTTCTAGGATCAGGTGGGTAAGAGAAACTATAGGATTGTGTAGGGGCAGTAGGGAGGAAAAGAGGGAAAGGAAGATCA
CTATTCTGCACAATTGATGTGAGTGCTAACCTCACTGCAAGAAGCACCTCACAAATTATGACACTGTGCCCACAGCATTT
GTTTCCTTCCTTATTGATTAAGAAACCATACTATCTACTCCTTCATGATAAAAACACCCAACAAGCTAGGAATAAAAGAG
GACTTTCTCAACTGATAAAAAGCATCTATGGAAAAACTCACAGTTAACATTGTACTTAATGGTAAAACATATACTTAAAG
CTTTTGCCCTAACATCAGCTACAAGATAAGGATGTCTGCTCCCACTACATTTTTTTGAAACTAGATCTCATTCTGTTACC
CAGGCTGGAGAGCAGTAGTGTGATTATAACTCACTGCAGCCTTGACCTCCAGGCTCAAGCCATGCCCCGACCTCAGCCTC
CCGGGTAGCTGGGACTTTAGGCACACATCACCATGCCTGGCTAATTTTGGTATTTTTTTTTTTTATTTTTGTAGAGACAG
AGTCTCCCTATGTTGCATATGCTGATCTTGAACCCCTGGCCTCAAGTGATCCTTCTGCCACAGCCTCCCACAGTTCTGGG
ATTACAGGTGTGACCGACTGTGCCTGGCCCTGCTCTCACCACTCCTATTTAACATTTTACTGGAGGTACTAGCCAGGGCA
ATTAGGAGTTAAGAATCCATATTGGAAAGGAAGAAGTAAAACTGCCTCTATTTGCAGATGACATTATTTTCTTTATAGAA
AATACTTAGAAATCTGCTTGTCTTAGTCAATTTGGGCTGCTATAAAAAGTGCTAGAAACTGAGTGGCTTATAAACAACAT
AAATGTATTTCTCATTGTTCTAGAGCCTGGAAGTCTGAGGTCAGAGTGTCAGCATGGTTGTCTTCTGGTGAGGGCCCTTC
TCTGGGTTGTAGACTTCTGTCTTTTCTTATACTCTCACATGGCAGAAAAAGGACTAGTGAGCTCTTTGGGTCTTTTTATA
AGGGCACTAATACGATTCATGAGGGCTCCACTCTCAAGACTCGATTACCTCCCAACTGATATTATCACATTGAGGGTTGG
GACTTCAACATATAAATTTTGGAGGAGCACACACATTTAGTCTGTAACACCACTAAAAAACCATTAGAACTAATAAACAA
GTTCAGCAAGATTGCAAGATACACATCACTATATAAAAATCTATGATATTTCTATACAGCAACAATGAACAAATCAAAAA
TTTGAAAAACAATGCATCAAAAGATACTTAGAAATAAACTGAACAAAAGAAATACAAAACATATACTCTGAAACTATATA
AGACCATTGAAAAAATTAAAGACCAAAATCAATAAAAGACATCCCATGCCCATGAATCAGAAGACTTTTTTTTTTTTTTT
TTTTTTTGAGACAGAGTCTTGTTCTGTTGCCCAGGCTGGAGTGCAATGGTGCAATCTCAGCTCACTGCAACCTCCACCTC
CCGGGTTCAAGCAGTTCTCCTGCCTCAGCCTCACAAGTTAGCAAGGATGTAGAGAAATTAGAACTCTTAACTATTGCTGA
TGGGAATGTAAAATGATATCAAGACAAAAGTGGCTCTATCTTGGGTGCAAATCCACCATGTTGACTTCTGATTAGCCTCA
GTCCCATGAATGCCTCCTGATTCCTACTTTATTTACTATCCTTAGTGTACTCACTATAGACAGTAGGAAGGGGTCTAAGC
TGTTGATCAAGAATCAGGTTTTTCAAAGGCCCACTATTCTGGCATTGGGTTGCTGATCTATTTTTTCTTTGCCTACTCCA
GCTGAGATCAGATCATACCACATTTGTTTCCTGATAATTCTAACCGGGCCTTTTAGGTATCCCTTTTTAAGTTCAGCAGA
ATTGGCTTCTCCCTTTGCCCAGGAAAGAATTCAAGCCAGAGGTAGAAGAAAACAGTTTTATTGAACAGGCAGTGTTATAT
CTCTGGTGGTATTATAGTTCCCTGACTGCTCCTGCAGAGCAGGGCTACCCCATAGACAGAGTAGCAACTCAGGGCAGTTT
TGCAGTCATATTTATACCCACTTTTAATTGCATGCAGATTATGGGGCAGTTTATGCAGAAATTTCTAGGGAAGGGGTAGT
AATCATTGGGTCATTGCCATGAAAAGGGGTGGTAATGTCTGGCTGTTGCCATGGCAATGGTAAATTGACGTGGCACGCTG
GTGGGTGTGTCTGATTGAAAGCTGCTTTCACCCCAGCCCTGTTTTAGTGAGTCCTCAATCTGGTCCAGTGTCTGAGCCCC
GCCCCTGGAGTCGAGTCCTGCCTCCTACCTCAAAGGCCTTAGAAACTTTAATTCAGTTGAGATAAATATACGGCTGGCAC
CACACCAACAACTCAGGACCACCGTAGAGGATGGTCCATTAACCACAGTGGCAAAGAAAGTCCAGCCAGACAAATTTCTA
CCAGAGCTGATAAAAGGCCAACAGCAAATATTATTTGGGACTCCCCAAGACCTTGAGGCAGAGGAGGGAACACTTGAATG
GAAGTTTGACTGGCAATCTCCCCCAGGTTGGATAAGGTATTTTTTTTTTTGCTACAGAATAGGGAATTCCTTGGCAACTA
AAGAGGTCTTCATTGATCCTGCTGGAGTCTGGACCAAGACGCTCCACATACCCATACACTGAACATGGCCCTTTTTAAAA
GGCACTCTGGTGGCTGCTTGGTATGGTCCTTTGCTGCCCCTGTGACCTTACCCTCGCCCCTCAGGCAACCCGTTTGGTGT
GCACCCTCAGCCCATAATCCTTGGGCTGCTTGCGTCATAACTAACAGAGATGAAGCTACCACAGCCATTTTGCTTGATGG
GGAAGAACTGCCCTGCCAAGTACCTACTCGAGACTTGTATTTCCTCCCATAGTCTTCTGTTCCTGCTGCTGCTCTGCCCT
GCAAAATGCCTCTTGGTTTGGATTCTGGAAAATATGGTGGCAAAGGCATGATTAATTCTGTGTCTCATACTTGACACAGG
TATCATTGCCTGTTGTTTGTATTGTTGCTGCAGCCTCTGCTTACAAGTAGAAAAGAAACTGATGCAATGTGTCACCCACA
TCCCCAAAATGACTGCAGCAGCCCCCTGGCTCAGGACCCATCATGGAAGAGGTGGGCGCTTGAGGTTGTAAGAGCCGGAT
TAGAGGGGTGGAGTGTGGAGACAAAAGGGGCTCTATCTTGGAAGCTAATCCACCATGCTGACTTCTGATTAGCTCCAGTA
TCCTGAATGCCTCCTGATTCCTACTTTATTTCCTGTCCTTAGTGTAAGAACATGAACTCACTACAGATCCTGATTTTAGA
TCAAAGCAACATTGATGTTATCACACATATTATAGGCTATGGTGCACATAGCAGTCTCGCCTGTTCTGGAAGGTGGCCTT
TAGCTGTCTCTATAGAGCATGTACTTCCTTTCCCTATATAAGGTGTCTATAAGTCCTGGGTCTGGATAGTAATCCTGCAG
AGATCTACCTGTATTGCTCCTGTTTTGACCATGCTTCTGTGGGTAAGTTCCCCAAATAAATCACCCTTTACTGACAAACT
GGATTTGTCTGCTTCGTTCTTTGGTTTCTCAGCTCCTAGGGCATTTGGGGGCTGCTTTTCCTATATGGCTGTTTCATAGA
ACAAATGGTACAGCTGCTTTCAGTCTGGCAGTTCCTCAAAAGGCCAAATGCAGAGTTACCATATGATGCAGCAATTCCAC
TCCTAGGTATATACCCAAGAGAAAATATGTCTACACAAAAACTTTTACGCGAATGTCAATGGCAGCATTATTCATAAGAG
CCATAATATCCATCAACTGATAAATGGATAAATAAAATATGGTACACACATACACTTGAAAATCATTTGGCAATAAAAAA
ATTAAAAATGAAATATTGAGTGGACGAACCATGGAAACACTATACTAAGTGAAAGAAGCCAGTCACAAAGGGCCACTTTT
TATATGATTGCATTTATGTGAAATATCCAGAATAGACAAATCTATAGAGACCAGTATGATCTATAGAGTAGATTGGTGGT
TGCCTAGGGCTTGGATACGTGGAGAAGTGGAGGTGATAGCTAAGGGGTATAGTGTTTCTTTTTAGTGTAATGAAAATGTT
CTAAATTGATTTGGTGATGGTTGCACCACTCTGTGAATATGCCTAGTCATTGAATTACACATTTTAAATGGGTGAAATTT
TTTTATGGTTTGTGAATTATAACTCAATAGAGCTTTGTTAAAAAAAACAAGCAACAACTAATTACAGATCACATCTAATT
GACAGCATATTTTTACTGCCCTGAATCCAAAAAGAAGTGCCCAGGTATGGGATAGGGGTGGGCGGAGAAACTGCTATGAA
GAGATGTATGGGGTTGCTTAGAGTATAAAGTATGAAGTCTTTATTTTGAATTAACTCTGTGGGCTGCTGTTAACTGGACT
TAGGCTTGTACTTCTTTGCATGGCTGTGATAATGCTATTCTAAACCTGCATTGTCCAATATGGTAGTCTCTGGCCAAACG
TGATTGAGTGCCTAAAATGGTATAACCCAGTTGAGATATTCTGTAAGTGTAGAACACACAGTGGATTTTGAAGACTTAGA
ATGAAAAAATATAAAATACCTCATTAATAATGTTTTATATTCATTGCATGTTTGGATGTATTAGGCTAAATAAAATATTT
TTAAAAATTTAAAAAGAAGCTGTATTCTCTGAGAATGGAAGTTTTCCCTGGGAAATGAGGCTGTGTCAACGCATGGTATA
CATGATTACAAGTGTGTGTGTTTTTTTTTTTTTTTTTGGCATTGTACATGGCAGAGACTGGAGCCCGGACTTAAACCTGG
GCAGTGTGGCTCACAGTCTGTGATTTTAATAACTGCAAATCTTAATTTCAGTATCCTATTTTTCAGTTCTGGAATTTCCA
TATGATCCTTTTTTATAGTTTCTAGTTTTCTGCTGACATTCTTGTCATGTTATTCCCTGAATGCATTTTTTTAAATTAAT
TAATTTATTTATTTTTATTGATCATTCTTGGGTGTTTCTCGCAGAGGGGGATTTGGCAGGGTCATAGGACAATAGTGGAG
GGAAGGTCAGCAGATAAACAAGTGAACAAAGGTCTCTGGTTTTCCTAGGCAGAGGACCCTGCAGCCTTCTGCAGTGTTTG
TGTCCCTGGGTACTTGAGATTAGGGATTGGTGATGACTCTTAATGAGCATGCTGCCTTCAAGCATCTGTTTAACAAAGCA
CATCTTGCACCGCCCTTAATCCATTTAACCCTGAGTGGACACAGCACATGTTGCAGAGAGCACAGGGTTGGGTTGGGGGT
AAGGTCACAGATCAACAGGATCCCAAGGCAGAAGAATTTTTCTTAGTACAGAACAAAATGAAAAGTCTCCCATGCCTACC
TCTTTCTGCACACACATGGCAACCATCCGATTTCTCAATCTTTTCCCCACCTTTCCTCCCTTTCTATTCCACAAAACCGC
CATTGTCATCATGGCCCGTTCTCAATGAGCTGTTGGGTACACCTCCCAGACGAGGAGGTGGCCAGGCAGAGGGGCTCCTC
ACTTCCCAGTAGGAGCAGCCGGGCAGAGGCGCCCCTCACCTCCCGGACCGGGCGGCTGGCCGGGTGGGGGGCTGACTCCC
CCACCTCCCTCCTGGACGGGGCGGCTGGCCGGGTGGGGGGCTGACCCCCCCATCTCCCTCCCGGACGGGGCGGCTGGCCG
GGCAGAGGGGCTCCTCACTTCCCAGTAGGGGCGGCTGGGCAGAGGCGCCCCTCACCTCCCGGACCGGGTGGCTGGCCAGG
TCGGGGGCTGACTCCCCCACCTCCCTCCTGGACGGGGCGGCTGGCCGGGCGGGGGACTGACCCCCCCACCTCCCTCCCGG
ATGGGGTGGCTGGCCTGGCGGGGGCTGTCCCCCACCTCCCTCCCGGACGGGGTGGCTGCCGGGCGGAGACACTCCTCACT
TCCCAGACGGGGTGGCTGCCTGGCGGAGGGGCTCCTCACTTCTCAGACAGGGCAGTTGCCAGGCAGAGGGTCTCCTCACT
TCTCAGACGGCGCGGCCGGGCAGAGACGCTCCTCACCTCCCAGACGGGGTCGCGGCCGGGTAGAGGCGCTCCTCACATCC
CAGATGGGGTGGCAGGGCAGAGGCGCTCCCCACATCTCAGACGATGGGCGGCCGGGCAGAGGCGCTCCTCACATCCCAGA
CGGGGCGGCGGGGCAGAGGTGCTCCCCACATCTCAGACGATGGGCGGCCGGGCAGAGACGCTCCTCACTTCCTAGATGGG
ATGGCGGCCGGGAAGAGGCGCTCCTCACTTCCTAGATGGGATGGCGGCCAGGCAGAGATGCTCCTCACTTTCCAGACTGG
GCAGCCAGGCAGAGGGGCTCCTCACGTCCCAGACGATGGGCGGCCAGGCAGAGACGCTCCTCACTTCCCAGACGGGGTGG
CGGCCGGGCAGAGGCTGCAATCTCGGCACTTTGGGAGGCCAAGGCAGGCAGCTGGGAGGTGGAGGTTGCAGCGAGCCGAG
ATCACGCCACTGCACTCCAGCCTGGGCAACATTGAGCACTGAGTGAACCAGACTCCGTCTGCAATCCCGGCACCTCAGGA
GGCCGAGGCTGGCGGATCACTCGCGGTTAGGAGCTGGAAACCAGCCCAGCCAACACAGCGAAACCCCGTCTCCACCAAAA
AAATACAAAAACCAGTCAGGCGTGGCGGCGTGCGCCTGCAATGGCAGGCACTCCGCAGGCTGAGGCAGAGAATCAGGCAG
GGAGGTTGCAGTGAGCCGAGATGGCAGCAGTACAGTCCAGCTTCGGCTCGGCATCAGAGGGAGACCGTGGAGGTAGAGGT
AGAGGTAGGGGTAGGGGTAGGGGTAGGGGTAGGGGTAGGGGTAGAGGTAGGTAGAGGTAGAGGTGTAGAGGTAGAGGGGA
AGTGTGTGTTTGTTGATTGATGGGTGTACATGATTACAAGTGTGTGTTTGTTCTGTGGTGACCCACTGGACAGCAGGGGG
AAAAAGAGGCAGGTAGGTGCCTCCGAATTAGACACTTTATTTTATTTATTTATTTATTTATTTATTTATTTATTTATTTA
TTTATTTTTTGAGATAGAGTCTTGCTCTGTCACCCAGGCTAGAGTGCAGTGGTGTGATCTTGGCTCACTGCAACCTCCGC
CTCTTGGGTTCAGGCGATTCTCCTGCCTCAGCCTCCTAAGTAGCTGAGATCACAGGCATGTGCCACCACGTCCGGCTAAT
TTTTTTTATTTTTAGTAGAGACGGGGTTTCGCCTTCTCTACTAGGCTGGTCTCAAACTCCTGACCTCAGGTGATCTGCCT
GCCTCAGCCTCCCAAAGTGTTGGGATTGTAGGCATGAGCTACCATGCCCGGCCTGAATTATACACTTTAAACGGGTGTAT
TTTTTATCGTTTGTGAAATTTTTTATGGAAGGTGAAATTTTTTATGGTTTGTGAATTATATATCAATAAGGTTTTGTTAA
AAAAAAACACAACTAACTACAGATTACATCTAATTGACAGCATATTTTTACTGCCCTGAATCCAAAAAGAAATGCCCAGG
TATGGGCTAGGGGTGGGCAGAGACACACACACTTGTAATCATGTACACAACTGGGAGTAGTGGGGATGAGTCTGTGGGGA
AAAACAGGAATTCAGAGTGCTGAGAAGCTTCCCTTACTGCCCTGCCCACTTCCCTCCCCTGGGCATTGCTCCCTCAGCCT
GGATTCACAAAGTCAATGCCTTTGGGAGCCAGGAACTAAATTCTTAGTCAAAGACACCTCCCTCCTGTCCAGTTATGGGT
CTCAGGGAGGAAATGGCAGGTGGGTGGTTTCATTCTTTCAGTTTCAGAAAATATCTGTAGACATAACATATTTCCTCTAT
TAAACAAATTCTGAGGTAAGGGGAAACAGAGAGAGACAGTCAGAACATTATAAGTCGAAAAGAATCTCAGCATTCTTAGT
TTAGGGAAGTTGGCCTTGCTCGCTGTGTGGTAGAAAAGCTGATTACGGGAGAGAACCGTGGAGGGAAGGAAGGGAGGCAG
TTTTGTCATCCCCACATCCGGTTTTGACTGGACACCATGCAGGCTGAGATCAACTTCAGAGGCAGTCAGCTGGAATCCCA
TAGCTTTATTTGCCCATTTATTGTGTTGTTATTTTGTTGTTGTCATTACTGGAAAAGGCAAGAGTAGAGCTATCAGAATG
TCTTCCTTGAACATGCTGTCAACAAACCCCAGGATTTTTATAAACCCTATGTGAAAAACACGCTAAGAATCTCTTTTCTT
CTTAGCATATTTTTCACTTAGGGTTTACAAAAATATGTTTTGAAGAAAGCTTGAAAAGATCAGTGGAAAAAAATTCAGCA
CCAAATTTTCTCTGAAAATCAACTTGTCAAAAAAGACGTGATTAACATCCATCCTGTAACCAGCACATGAATGATCTTCC
CTCTTTACGAGTGTGCTTCAGCAAAACTTTAATCTCGACCCCTCCCTCTTTCCACTTTTAATAGTGGATGGATGAAAAGG
CAAGAGGAAAAACAAAGCAGCCGAAACACACCCAGACGCACACTTCTGCCAAGCATAGCCACCACGGTAAGCATTTTGAA
TTCAGCCAATTATGATCTGTGGATTATTAACATTACCTCATTTACTTCATCAGTGTAGACAATAGTAAACTGACTGTAGT
GAAATTTGGAACCATAGAAACGTGATTCTATTTTAATACAGGCCTATCAGGTAACCCACAGACTATGCTACATATGAATA
AGAGAAAATAAGTTTATTCTTAACTATGTAATGTAATTCCAGTGGAATTCGGTAGGTGGCATAAAACAGCCTTTTTGTTT
TGTTTTGTTTTCAACACCAAATGTGGTTAACAGTGAAATAAATTCAAGGAAGTGCTTGAATTTCTTACAGACTGCCAAAT
GGAACAGACAAGCAGGTTGTCTTGGTAAGCAACACATTCTTTTCTTTTGTAAAAGAAAATAATTGTATAGCTAGTTATTA
AGTACAGAAGTCTCAAAAATCTGTGTAAGTCCTGGGTGTTTTTCTAAGTGGGTTATATTTCTGATATGTATATAGTTACT
GTGTAGTTTGTACTGGCATTTGTGTATCAGTTCTGAGTCCTAAATCAGAGAAAGTCCCCACACTCCTCTGGGAATAACAC
CTCGTGTGTGATTTGCTTATAGGAAATATTTTTGAGTTGGGAAATGAATATTTGGGTCACACACGTCTTCTGGTTTTCTT
CCAGAGCAGCTGCTAGTTGTTGATTTTGACAGCATTTCTCTTCACCTAATCCCGCCATTTGTATTCTCTTCTCAGTGTTA
AAGAAAATGAGATATAAGTCAGTTACTCCCGGAGGCAATGCTGCTGTTCAGCTCTTCTGTTTTTGTGGCCAGGTTTGTTT
CAGTTTTTCTCTCTGGGGCCTTCCCACTAGCTAGTCCTTCTGTTTCTCAGGCCACTAACACTGTTCTGGGGAGCTAGACG
TGAGGTAGAATTGCAGGTTTTGAAAATGTTTCCCAGGCTACATCCAATTTGGTCAAAAGACTTGAAAGTGAATTTGTTTT
ATAACAAAGCAAGAGTTCACAAAACCGCATAGAAAGCAAAGCAGAACAGTTCTTGAACTGTCTCAGATTCTTTTAACCTT
TGGTTAATAGCTATTTGGTCTTGTGCAGAGAAGGCCCTTAGTAAATATTTACAGGACTAAACTTAATGGGCCAGATAGAT
TGTATGGGTATTGTCCATTAAGACCAGTCAAAGCCTTGATTTGATGCCTCCAAAGTCTCCAAAAGAAGACAAATTAAATG
TATTGATTCATTGTATCATATAATGTGAGGCTAAACCTATCATAATAATGAAAATTCACAGAAGCTCACCTAGAGGCATT
TTACACTTTCAAATTAAAGCATCCTTTTCTGCTCAGCTTATTTTTTGGATAGTAAAAGAGTTTAAAGGTTCTAAAATAGA
TGAGGCTCAGTCATGGTCATTAAGACGTTGATCAAAAAATTCTTTGGTCTAGGAAACTGAGACATTGATTTTCATGGATT
TAGCAAGTTTGGTATATAAAATTCAAAGATCCTGGAAGTATTAAAAATCAGTTATTCCTGTGTATAACATTTTGATTTCT
TAATTATAGAAATAAGAATGGTTTCATAAACTGAGTACTTAAAATTAATGACTTTAAATGAAAGGGCAAGATGGAGGTTA
GGCAAATAGAAGATATTTGAGTCAATTAATTTAGCTCGACATAAAACTGAAGCTATGCTTTCATTAAATGCTTGCGTTAG
CAGTGGTGAATCCATATGGGTCGTAGAAACTTAATTCTTGCCTCCTCAGTGGAAAGAATTTGTGTGAGGGGCATAAGGCA
GAGTGAGAGACCGAGGCAAGTTTTAGAGTAAGAGAGAGAGTGTCACGCCCAGGGCCAGGTTCCAGCCCATACTGAGGTCT
GAGGGGAGGGGGTGGATGAGCAGATAGCTGAAAGAACACTCAGGGGGCCGTAGGCAGGTGAAAGGTGATTTTATTCAGCA
ACAGCTCTCATTAGCAGCTTACTTACAGTAGTTCTCTCAGACTGTCCGCCTTGTCCTGGCTGCTTAGTTCGGCAGCTTCC
ACACACAACTGTGCGTCCGGCTCTCCCTTGCCCTCAGGGTCAGCAGCTTAACTCTTTCTCTCTCTGGGTACAAGCAAGCC
GAGCTGTGTCCTGGCTCCCCTCAGTCCATCTGCAAAGATGGACAGCTTTGGCTCTCTCTCTCTCTCTTTCTCTGGCTGCC
AATGCACCTGTACAGCGTCAGCAGGGCAATTATACCATTTACGGACAATAGTGGCTTAGAGCCAAGGGATGAACCTTCCC
TATGTTATGGCTATGGTAAGCTTCTCTATGTTATGTCTACATGGCTATGATAAAAAGTGAGTTATACGCCTGCGCTCTAA
ACTCGCTGAGTCACTCTGGATGTTTACCTTGGCCTATCCTTGACCAAAGCACAGCCATATTCCTTACAGAGAGTTTATTA
AGAAGTTTTAGATCAGGAACGAAAGGAAATAAAGTACACTTGGAGGAGGGCCAAGTGGGCAACTTGAGAGATCCAAGTGC
CCAGTTTAACCTATTACTTGGGGCTTATTTGTTGGCAGGCTTCTGGAGTCTTGCGTCTCTTCTCCCCTGATTCTTCCCTT
GGGGTGGGCTGTCTGCATGTGCAGTGGCCTGCCAGCCCTTGGGATGGGCTGCACACACACTGTGTTTACTGAAGTTGTGT
ACATGCTCATTGGAGACATTTTTCCCTTGCCAGACAAGTCTTCCTAGAGGAAGGTCACATACCCGTTGAAGTCCGCCATT
TTGCCTCTTGGTATGCATGCTCAAGCCTGCTTGCCCAGCTCCTGAGAGTTTGTCAGGAGTCTGCTAACCACCAGCTTCAG
GTGTGTTTTCTATCTATTAGGAGACTGACTTTCCCTGATGCTGGCTGCAAACAATTATTATTCTAGAGAGACTGTTTTTT
TGTTTTTGTTTGTTTGTTTTTGAGACAGAGTCTAGCCCTGTTGCCCAGGCTGGAGTGCAATGGCATGATCTTGGCTCACT
GCAACCTCTGCCTCCTGGGTTCAAGCAATTCTCCTGCCTCAGCTTCCCCAGTAGCTGGGATTACAGGCACCTGCCATCAC
GCCCGGCTAATTTTTGTATTTTTAGTAGAGACAGGGTTTCACCATGCTGGCCAGACTAGTCTCAAACTCCCGACCTCAAG
TGATCCACCTATCTCAGCCTCCCGAAGTGCTGGAATTACAGGCGTGAGCCATGGCCCCCGGCTTAGAGAGACTGTTTAAC
AACTGCCTGATCATCACCTGATGGTCACCTGATATTCCTGGTTGGGGGGCCCCTCTCCTGTCCTGCTCATGTCTGCCTAA
GTACCTACTCTAACACTTGGGGGCAGGCAGTTATATAAATTTATATATTTTTGTGATGATAGCTGTTGTTAAGGGAAAAA
GGACAACTTTCATCTTGCGTGTGGTAGGTTCTGCGAGCCAAGCTCTCGCACTCATGCACGGGGTCCTGTGTGCCTCTGAT
CACTTTTGAATACCACGCCTGACTGTGCTGATCACTGCTTCCATGAACTCTGGAGTTGGCTAGGAGGTGGAAAGAGCCTT
GATCTGGATTCAGGCATAGGAATAAGCAGGGAACCCCACTTAGGCCTTAAGACCAGGCCACTTTTGCTTCGACCTAGCTT
GGCACTGCCCCAGGGTATTATTTTTATTCCCATTATTTTAGTCTGTTTAAATTGCTGTAACAAAGTACCAGAATGGGTGA
CTTATAAACAACAGACACTTACTTTTCACAGTTCTGGAAGGTAGAAGTCTAAGATCAGGGTGGTAGTGTTGGGTTCTGGC
GAAGGCCCTCTTCTGGTTTGCGGATGCCTCCTTCTTGTATTTTCATATGGCAGAATGACAGCCTGAGGCTCTCTGGGGTC
TTTTTTATAAAGGTACTAATCTGACTCATAAAGGCTCTATTCTTATGACCTAATTACTTCACAATTTTCTCCTGCTAATA
CCATCACCTTGGGGGTTAGGATTTCAACATAAGAATTTCACAGGGCACAAACATTCAAGCTATAACATCCATTGAAATAT
TGCTCCTTGTGTGAGTAGCTCTTTAAGGAAGCTGGAGAGGAAGAATTGGGAAATTTCCGGGTCTTTCAGCCAAATACAGT
TAATGTCATAAAAACACCACCTTGACTTTTAACATGAACATTAATGTCTGCATCATCTTCCTTGCTCAGGGTCTTCATGA
ACACTAATAGGGGTACCAGGCCCTCTTCCTCGTTAGAAGAAATCAGGATAACAAAGGCATATTGGGCACCCCTACAAAAG
GGTAAGTCCAAGGAAGGCTCCAAAACCCAGATAAGTTTGCACATCTAAATTGGGGAACTTTTGTAACCGAGGGTTGTTTG
TAGCAGCTAAATTATTTAGGTTCTCTGTGCTCATTGAGATATGGGAGGGGATATGTGAAGAGGAATTTGGTGCTTTGCTG
ATACTAATGCCCTGCTTGTCTAATGGTTCTAGGGGATTTTGAATAAGTTCCTTGTGAGAACTGGGTAGCTGTGTAATCAC
GGCTGAAAGGGTCTCTGGAGGTGAGGACTGCACACTCTAGAGTTCATCCCAACACAGCTCCCTCTGTTCTCATACGCCAC
TGGAATGCTGACCACAAAATGGTGTCTTAGCTAGAGCAGTTTGAATACTGGTACAATAGGCATAACCTAGAACAGTGCCT
ACCACTGCATGGAGAATAAATACATATTTGATGAATGAATGAAAAAAGTATTCTGAATAAGAGAGAGGAGGAAACAACTC
CCTTTCTCAAAACCTCCCAGTGTTCTCCACCCTCTCATCATTTTTGGAATAGAATCCCAACTCCTTGCAATGATCAACAA
AGCCTGAGTGATCTTGGCCCTAACTCCTCTCATCTTCCCTAACTCCTTACTAACATTGACCTTCCCGTTCCTTCTCAGAC
AAGCCAAGCTCAATTCCATCTCAGGTCCTTTATACTTGCCTTTTCTTTTGCCAGAAATGCTGTAGGTTACCCAGATCTTC
CCATAGGTTACTCATTCACTTCATTTTGATTTCTACTCTAGTGTCATCATCTCAAAAAGGCCTTCTCTAACTAATCACTC
AAAAATAGCCACCCCCAAGCACTGTCTCATACCTACAATTTACCCTGCTTTATTTTTCTCTACAGAACTTATCACTACAG
GAACTTGAGTTTAGTCATTCATTTATTTTCTGTCTCCCCACTAGATAAAAACTCCTTGAGAATTCGGACTTTATCTTGTT
CACTGCTCTATCTCCAGTTCTTAAAACATGGCCAGTAGGTGATCAATAAATACTGGTTGAATAAATAAATGGATGAATAA
ATGGATCTTCTGAGAGGCCTTTTATCAGGATCTAATTACCTTTTGAGTAAGACCAAAGTTAGACCCAAACCGTGTATAGG
TCATAAAGGGTAATAGAAGAGCAGTCCCAATACCACCATTAGGAACACCTCTTGAGAAAGTCATGGGGAGATGTACGTCA
AAGAGTGAAAGTACACCAGTTGAGTCTTAGCTGGAGTTCTAAATAATTTTGGGAAGCTTGGGGCCAGAAAACTTTCTATG
AACCCACCTGTATTAGTTTCCTGAGGCTGCTGTAACAGATGATCATAAACGTGGTGGCTTTGCCCTCAACGTTCTGGAGA
GCAGAAGTCGAAACTCAAGGTGTCAGCAGGGCCACCATCACTCCAGAGCCTCCTCTAGCTTCTGGCGCCTGTTGATGTTC
CTTGCCTTGTAGCTGCTGCACTCCAGTCTCTGCCTCCACTGTCACAAGGCCTTCTCCTCTGTGTCTCAATTGCCTCTTCT
CTTGTCTCTTTGTCAAATATCCCTCTGCCTTTCTCTTAAAAAAGCACTTGTCATTGGATTTATGGACCACCTGGATAATT
TGGGATGATCTCCTCATCTCAAGATCCTTAACTTTATTATCTCTGCAAAGACCCCTTTTCTAAGGAAGGTAACATTCATG
GTTTCTGGGGATTAGGACATGGTTAAGATGTAGACATATCTTTTGGTGAGCCACCATTCAACCTATTACACCATCCATAT
TAAAACTTATTTGATTAGAGCTGGCATCATTATTTACCAATATATATACTTCTCTAGTTTTAGGCCATGTAACCCAAAAG
AATGTAGCCTCCACTTTATAAGTCTGTAACAAAAAAAAGTTTAATGTCAGCCATGACTAATTTCGTGTATTTTAAAAATG
TATGTAATGTATTTCATCATTTCATTATTTTTTTTCTAGAATCTGTATCTGTATCAAGATGATCTGAAGAACAGCTTCTA
CCTTTAGGAATGTCTAGTGTTCCAAAATGACTAGCATCTTCCATTTTGCCATTATCTTCATGTTAATACTTCAGATCAGA
ATACAATTATCTGAAGAAAGTGAATTTTTAGTTGATAGGTCAAAAAACGGTCTCATCCACGTTCCTAAAGACCTATCCCA
GAAAACAACAATCTTAAATATATCGCAAAATTATATATCTGAGCTTTGGACTTCTGACATCTTATCACTGTCAAAACTGA
GGATTTTGATAATTTCTCATAATAGAATCCAGTATCTTGATATCAGTGTTTTCAAATTCAACCAGGAATTGGAATACTTG
GATTTGTCCCACAACAAGTTGGTGAAGATTTCTTGCCACCCTACTGTGAACCTCAAGCACTTGGACCTGTCATTTAATGC
ATTTGATGCCCTGCCTATATGCAAAGAGTTTGGCAATATGTCTCAACTAAAATTTCTGGGGTTGAGCACCACACACTTAG
AAAAATCTAGTGTGCTGCCAATTGCTCATTTGAATATCAGCAAGGTCTTGCTGGTCTTAGGAGAGACTTATGGGGAAAAA
GAAGACCCTGAGGGCCTTCAAGACTTTAACACTGAGAGTCTGCACATTGTGTTCCCCACAAACAAAGAATTCCATTTTAT
TTTGGATGTGTCAGTCAAGACTGTAGCAAATCTGGAACTATCTAATATCAAATGTGTGCTAGAAGATAACAAATGTTCTT
ACTTCCTAAGTATTCTGGCGAAACTTCAAACAAATCCAAAGTTATCAAATCTTACCTTAAACAACATTGAAACAACTTGG
AATTCTTTCATTAGGATCCTCCAGCTGGTTTGGCATACAACTGTATGGTATTTCTCAATTTCAAACGTGAAGCTACAGGG
TCAGCTGGACTTCAGAGATTTTGATTATTCTGGCACTTCCTTGAAGGCCTTGTCTATACACCAAGTTGTCAGCGATGTGT
TCGGTTTTCCGCAAAGTTATATCTATGAAATCTTTTCGAATATGAACATCAAAAATTTCACAGTGTCTGGTACACGCATG
GTCCACATGCTTTGCCCATCCAAAATTAGCCCGTTCCTGCATTTGGATTTTTCCAATAATCTCTTAACAGACACGGTTTT
TGAAAATTGTGGGCACCTTACTGAGTTGGAGACACTTATTTTACAAATGAATCAATTAAAAGAACTTTCAAAAATAGCTG
AAATGACTACACAGATGAAGTCTCTGCAACAATTGGATATTAGCCAGAATTCTGTAAGCTATGATGAAAAGAAAGGAGAC
TGTTCTTGGACTAAAAGTTTATTAAGTTTAAATATGTCTTCAAATATACTTACTGACACTATTTTCAGATGTTTACCTCC
CAGGATCAAGGTACTTGATCTTCACAGCAATAAAATAAAGAGCATTCCTAAACAAGTCGTAAAACTGGAAGCTTTGCAAG
AACTCAATGTTGCTTTCAATTCTTTAACTGACCTTCCTGGATGTGGCAGCTTTAGCAGCCTTTCTGTATTGATCATTGAT
CACAATTCAGTTTCCCACCCATCGGCTGATTTCTTCCAGAGCTGCCAGAAGATGAGGTCAATAAAAGCAGGGGACAATCC
ATTCCAATGTACCTGTGAGCTAGGAGAATTTGTCAAAAATATAGACCAAGTATCAAGTGAAGTGTTAGAGGGCTGGCCTG
ATTCTTATAAGTGTGACTACCCGGAAAGTTATAGAGGAACCCTACTAAAGGACTTTCACATGTCTGAATTATCCTGCAAC
ATAACTCTGCTGATCGTCACCATCGTTGCCACCATGCTGGTGTTGGCTGTGACTGTGACCTCCCTCTGCAGCTACTTGGA
TCTGCCCTGGTATCTCAGGATGGTGTGCCAGTGGACCCAGACCCGGCGCAGGGCCAGGAACATACCCTTAGAAGAACTCC
AAAGAAATCTCCAGTTTCATGCATTTATTTCATATAGTGGGCACGATTCTTTCTGGGTGAAGAATGAATTATTGCCAAAC
CTAGAGAAAGAAGGTATGCAGATTTGCCTTCATGAGAGAAACTTTGTTCCTGGCAAGAGCATTGTGGAAAATATCATCAC
CTGCATTGAGAAGAGTTACAAGTCCATCTTTGTTTTGTCTCCCAACTTTGTCCAGAGTGAATGGTGCCATTATGAACTCT
ACTTTGCCCATCACAATCTCTTTCATGAAGGATCTAATAGCTTAATCCTGATCTTGCTGGAACCCATTCCGCAGTACTCC
ATTCCTAGCAGTTATCACAAGCTCAAAAGTCTCATGGCCAGGAGGACTTATTTGGAATGGCCCAAGGAAAAGAGCAAACG
TGGCCTTTTTTGGGCTAACTTAAGGGCAGCCATTAATATTAAGCTGACAGAGCAAGCAAAGAAATAGATTACACATCAAG
TGAAAAATATTCCTCCTGTTGATATTGCTGCTTTTGGAAGTTCCAACAATGACTTTATTTTGCATCAGCATAGATGTAAA
CACAATTGTGAGTGTATGATGTAGGTAAAAATATATACCTTCGGGTCGCAGTTCACCATTTATATGTGGTATTAAAAATT
AATGAAATGATATAACTTTGATTTAAACAGTTCTGACACATAAGGGATCCACTTGTTTCTTTGCTATAACTGAGTTCTGA
ATTTATCATGAAGTCAAGGGAAGCACCTGTTTTTCTTCAGTGATGTGTGTTCTAGTGTATCATGGCCAATATTGGAAAAT
GCCGTGACTTTTGTGAATATAGCTGGGTTTGTATAGCAGATTTCTACAGCTTTTGATGCTTTCCTCATATCCATGGTGCA
TTCAACTCAACTTATTGATGGCTGCTCGTTGATAAAGCTGGAGACACTATGCCTGAAGGTTCCTCCCCAACCCCCCACCA
TCATGCCCACACAAAGGGCAGTTCAGAAGTGCTGGAAATTGTCCTTGGAAACAGCCCTCAACCAATGTCAGAAAGAGCTG
GTGGATAAATATCCCAACTTTCTCACTCATTTTTTGAGATAAATCAAAAAATGGGGTATACTTTACACTCTCCCTGAGTT
CCCCAGTAGGATAAAATTCTGGTTGTCCACAGTAGAAATGGCTTGATTAAGTATTCTCGACTGGCTTCCTTCCCATTTTT
TTCTCACTTACTTATTCCTCCATGTGTATTCCATGGGATCACCTCTGTGATAAACTACTTGCCACTGAATATTTGTCTCA
GGGGCTGCTTATGAGGGAGCCCAGACCAAGAAAGTGTTCCACCTCAGTCCAAATGAAAATTCTGAGTCTAGTTTTCCATG
ACTGGAGATCTCGCTGAGATTCTGGTTGTATGAACTAACATGCACAAGAGCCTTTGCTCTTTTGATTAGAAGGAAGAATA
TAGTTGATATACGGTGCTTCTATATATAGCATCTGTCTCTTTGGGGAACTATTAAAGTAGAGCATGAGCAGATGGGGCAG
TGTAAGGAATGGATATATCTGATGCTTTTCATACCCATCAAACATCCTGTCAGTCTACATTTTTACTCCAGCAGCCAGCT
CTGGGCAGGTGTAGTCTTGACAAAAACCTTGTCATAACTACACCATGTATCTTTCACTTTATGCCCCATGACTTCACTGA
TACCACTGCGTGGGATGCTTGTAGAAGCCTGTTCTACCATTCTGATACATGCACAAACCTGGAAGCACAAGGGATAACAC
TACGGGATGGGAAGCATTATATACATGCTCCTTTCTTCTATTCTTGGGTTAAAAAATGAGGTGTAATTGATACCTTTTCT
CAGACAATCCCTAGTGGCACTGAGGTTTACTTACCCACAGGGTAACCAGCTCAGTAACTTGGTTTTGCATTGACTTTCCA
TTCTTCCTTGGTCACTCTTCTCATTCGGGTCACTCTTGAGTCCTGAGATCGCTGCTCCAAACAAGCGACCTTTCCTTAAC
TCAAGGAGTCTAGAAAAAATAAATAAAATAAAAAGCAACAAAAACCAAAAAGCCTACAAATGACCAGCCCACAAAGCTCT
CATCTCAGTCTCTACTTTTTTGAGTGGGAAGGAACCCAACTAACACATACATATCTATTAGCTTCTTGGCCTAAAGAACT
TACAGTCAATCAGAGAACACTAAACATATCCAAATAACTAAAATAAAAATCAATGAAACAAAGCTATAAAATGGAAGTTC
AGAGGTTGCTTAAATTGAGTGAAAAAAAATCTTAGAAATGATGTGTCTTTTTTTTTTTTTTTCCATTAGGTCTTGAAGGA
AGACTTTAACTTTGGATTTTTCAATGTGTAATGATGAAGAATGGAAAGGCCATTCTAAACAGAAAGATAAAGAGAGCAAA
GGCAGGTATACCCAGGCCTATTCTTGGACTGGTATCCCAGTTTGACTGGACCGTGAGGTTCATGAAAAACAACAGTAGGA
TAGGAGTGTGGAAAGTTATATTAAGACTATATTGTCAAGGATCTTGAAAGTCAAACCACGGAGTTAGAGCTTGAAAATAG
AGGAAAGTCATTATTTAAACTTCTTTGAAGCATAATAGTGGGAGGCAAATTCAAACCATGAAATGTATGCTAAAAATAGG
AGAACTATTGTTCTGTAATGGAATTGCAAAAATTGTAAGAACTGGATTTTCCCATTTTCTCTAGGATGATTGCACTTTCT
GTATTGAACTTCAAGATTTCATAACCAAGTTTCAACTACTCAGCAAGCAGTTATGTATTCGTTTTCAAGGTCTATTTTAA
CAAAGTCTATTTTAACAAGCTGGGTGGCCTAAACAACTGAAATTTATTCCCATAGTTCTGGAGGCTGAAAGTCCAAAATC
AAGGTGTTGGCAGGGTTGGCTGCTTTTGAGAGGAGAGAGGGAAGGATCCATTTCTACTGTGGACAACCCAGTCTCTTTTC
TTAACTCATAGATGGCTGTCTTTTCTCTGTCTCTTCACATCGTCTTCCCTCTATGCCTGTCTGTCTCTGTGTCCAAAATT
CTTTTTTTTTTTTAATAAGGACACCAATTACATTGGATTAGGGCCCACTCTAATGACCTCATTTTAACTAAGTACATCTC
CCAACAACCTTATTTCCAAGGAAGGGGTAATTCTGAGGTTCTGGGGTTCAGGACTTCAACATATGAATTTTCGGAGACAC
AATTCAACTTGTTACACCCTGTAAATCCGAAAAAGAATTCCCTGAAAGGAACCTAAAGTCACTTAGAGTTTATTTAATAA
ACTCAAAATTTTTAGACGTTAGATCAGTCCTATACATAAATAGCTGATCTCATAAGTGGTTTTAAGGATGCTGGCGGGAG
GTTGAAATTGTATTGGTGGAGTCTCCTGCCAAAGAATTCAAAGTTGGAAGATGAAGAATTCAGGCCAAAGTATGATAAAT
ATCAATGAAAATAAGAGAGATTTCACAGATAAATGAGAGGAAGTATTACAGAATGGGTAAATATGCAAGCCCTTAATCAA
GTCTGGGTTTCAATTCTAGCTCCGTCATGTATTTGCAGATGATCTTGGACAAGTTACTTAAACTTTCTAAGCATAGATTT
CATTATTTGTAAAATATAGTCTTAGAGTAGCTAGCCAAGACCTTTAGATTTCCAAGTATAAATTAAAAGACTGGTCAAAT
ATGTCTCCCAAACTACAGTTTTCACATAGCGAGCTTTCCAAGTGGTACTGTTGAAGCCCTTTCCCTAGGACCTAGGTCAG
AGGGAGCACACCCCACCCATCCTTCTTGAGGGTAAATGAAACTCATAATATACCAAAACCTCTCTCCAAAATGCTCTTCT
AATGTATAACTCCTCTCAGTCTCTCCAATCCCCTTTATTGGCTAGAGATGGGTAGTTAGCAAAGGTCACAAAACCAGTTA
TCTAGTCTCACACCTCACTCGGGGGACCTCCACCAAAATTGAGACTAACCTAGTTCCATTTTATCAGCCTGCTATACTAG
CGAGACTGCAGTGGGATGGGCATTTCATATGGCAATAATAGGAGGGCAAAGTAATTTGGAGATATATATATATATATATA
TATAATTTGAAAATACTCATGCTCTTTGATCTGTCAGTTCCACTTTTAGGAATTTATCCTAAGGAAATAAGTTGAAATGT
GAACAAATATTTATTTATAAGTATGTTAACTGTGAGGTTATTAGCAGCCATGGAAAATTGGAGACATTCCAAATTTCTAA
CAATGGGGGAATGTGTTTAATGACATATTGTACAACCATTTAATATAATGTATAGGATTAAGATGGTAATTTTTATGTTA
TGTCTTTTTCACTACAATTTTTAAAAAAATGTATAGGGCCTGGTGTGGTGGCACAGTTTCTGTAATCCCAGCAACTCAGG
AGACTGAGGCAGGAGGATCGCTTGAGCCCAGGCATTCGAGGCTGCAGTGAGCTATGCTTATATTACAATATAAATAAAAA
GCATAGGATACAAAAGTATATGTACACTATGATTTCAACTAGGTTAAAATATGCATAGAAAATGGTCTAGATGAAATATG
AAAAAATTATCACTGCTTGCCTACAACTGGTGTTTGTTTTTGTTTTTATACTTTTCTGTACTTGAAAAATTTACAACAAA
CAAATAAGGAAATGGGGCTTAGGAGACCAAAAATAGGTCTCTCCAGTGGAACAATAGAGAATAAATAAAGGTTATAAAGC
TGGGCTTTGTAAACCGGCATGCTGGGATTAAATCCTGGCTCAATTTCTCTGTAGAAGAGAAAAGAGAGGGCACAAATCCT
CTGTCCATAGATGAATTCCTTCACCCTTCCTAGCCTCCTTTTTCTCATCTATAAAATGGGCATAATACTGGTACCTCTCT
CATATGGTTGTTTTAGAAATCGGTGGTACAATGTGTGTAACTGACTTATCTCAGGGTCATGGCTATTCACTCTAGAGGTG
GCAGACACAGTGGGGAAGAGAAGCATCTCGGACCTCCACCTGAATTTTGCTTCTTCCACCTTCTTCTAGTGACTTTGGGT
GGTTATGTACTCCTTCTCTGCTTTATTACTGTGGAGACGTAAAATGAGAGTAATGGAAATACCTGTCTCATACTATTGTT
ACGAGGATTAAGTGAGTTAATACATATAATGTCTATAGAACAGTTCCTGGCATTAAGCAAAAGCTCAATAAATGTTAGAG
TTATTATTATGTCATTATCATCATTCTTCTTGTCCTTATTATACCAACAAATATGGAACTGGAGCTGAAGTGCTGATCAT
GTTTCATCTAGATTATCCTGGGCTTTCTAAGATGAAATAAAATCAGAAAAGAGACAAAAGTTACAGAAAAACTGGGAACA
GCTCCATGTTAAACAAGCGAATGAACAAATGAGGCAAACAAAACAACTAAAAATGGCAAATGGGGAAATAAGAAATTGGG
AAGTAAACTGTTAGCAAATATCATAGATAAAATTCATGTATATCAAGAACTAATAGAATAAAATAGAAAAGCACTAAGAT
CTCCATTGATATGTGGGCAATAAGCATGGTCAGCCCAAAACAGGATCCAGTTAGAAAATGAATTTATGAAAAATTTCAAC
CTCATTAATGCAGCCCAAGTGTCCCCAAGATTTCTCATTTTTTGGGTTACCACTTCCTTTTCATTTTAAGTAAAATGACT
CAAGTCCCCAACCCCTGTGTTTACAGGAAAACAGTTCCTCCTGAGGATTTCAGAGAAGAGCTGGACTTCCTGGCTGACTC
TCACATTCTCTTCTTCTGAACTGCTTCTTCTGGTAGCTCCCTGTCACTTTGGACAAGCTCGCACTGCCCTCCTTGGCTGT
TCTGGAACTGTGTGATGGACTTCATCCTACTAGGACACAGTTGCTTCTTGAATTCCCCAGATAAACACCCACCCGTGTGA
ATGACAAGCCATAATAAAAATTAATTGTCAAAATTAATCAACAAGGTCCCAAACAAGATTGAGAGCCAGGGGACAGAAGT
GCCACCGGTAATTATATAGTCACAGGCAGACCTATGAGGATGTTCATCTTTCCTCTAAGACAACTTGAAGTTACAGACGT
TTCTTAGAACTTAAAAAGTTTGATAGCCCCTTACCCAAAGAAATAGAAGCATTGGAAAATATGTTAAAGAAATTTCTATG
AAATTAGAACAAAATGACTTATTTTGCAAGAGAAAAAATAAGTAAAAGCCTAACTCTGAGAAGTCCACATCCATCTAATA
GGTGTTCCAGAATCAGAAAAAGAGATGAGGTGAAGACATTATCAAAAAAAATAATAATAATAAGACGGAGAAATCCCACT
GAAGGACATGAATCTCCTAAAAGTACCCAGCAAAACGAACAGTTCAAAGACCCAAACCAAGGCACTTTCTCTTGAAATTT
TAGACCATCAGGAACAACAATAGTAAAAGCTTCCAGAGAGAGAGAAAGAAAAAAGAAACCAGGTTACATACAAAGAATCA
GAAATCAGAATAACCATGCATCTCTCAGTACAATGCTAGAAGCTAGAACTTACAGAACAATACCTTCAAAATTGTATAGA
AAAATCATTTGCAACATATAACTTTATATCCAGCCAAACAGTCAATCAAATATGAGGCATGACATGTAAGTTCTCCCTCC
AAATTAACCATCAATGCATTCTTTCGGGAAACTACTGGATGATGAATTCCACTATAACAGATAAAAACCAAGAAAGGAGA
TGCTGTGCATCCCAAGAAACGAGAGATCTAATACAGCAGAGAGGAGCAACAGCTTTCCAGGATAGCAACAACTCAAAGTC
AGAGGATGTCAGCTCTGCAGAAATCCAATGCATGCTGGAGCAAAATGACAGACAGTAATAATT
TLR7 gene (SEQ ID NO: 31)
>chromosome: GRCh38:X:12866483:12890980:1
          >Exon 1: 12,867,083-12,867,123
          >Exon 2: 12,867,481-12,867,581
          >Exon 3: 12,885,512-12,890,380
          >rs3853839: 12,889,539
TGGGGCCCACACTTTGAGAAGCAAATGCAGCTGAACTTTTTTAGAGGAAAGTGAGTGAACCAACTGGTAGCTTTGCCACT
GCTTAAAAACCAGCATCCTTTCCAGCTGGGTCTAAGACAGAATAAGGTAAATTTAGATATGTCTCTAATATATCTATAGA
ACAGTGGTTCTCAACCCGGGGTGTTTTTGCCCCTTAGGGGATAATTTGCAATGTCTGGAGACATCTGTGATTGTCATAAC
TGGAAGGGGGCAGTGCTATTGGCATCTAGTGGGTATAGAGCAAGGGTGCTACCAAATATCCTATGGTGCAACAGAGAATT
ATCTGGTCAAAAATGTAAATAGTGCTGAGGGTGAGAAACCCTGCTATAAAAACGAAAGAAATTTGGTCTACAGAGTTGTT
TGGATTTAGACAAGACGTTGCCCCAATAGTGGTGATAGAAATAAGAGGAACCCCGTGCTTTTGCAAAGCCCATATCTGGG
GTGGCTTAAATAATCATGCTCCTCCCCATCCCCCGACCTGATCTTTGTAGTTGGAAACTCCAGGGCTGGCTGCCTGTAGT
CTTTGTGACTACACTTCCTGCCTCCCATCACTTCATCTCAGAAGACTCCAGATATAGGATCACTCCATGCCATCAAGAAA
GGTATTTTAAACATTGGAACACATATAGATAATTTAAGTAGGTAGATGTATGTGCTGTTATAAGGAAGTGGGGAGGAGAG
AAGAGGGAACCGAAATCATATGCACAAAAATTTTTTTTAGAATATAAATAAAAAATGTGGTAGTCTAAAATGTCAATTCT
TCAAAGATAAAGTTAGGCTTTCAGTAACGTTAGAAATGGTTTTCTGGAATATGTCTCCAGTCTACCTAACTTTGAGGAAG
TAAATACTGTAAATAGATGTTTCAAACGCATTTTAAAGCAATGATCCTAGCATGTCTTTAAGCTACAGTATTGTGCTGTC
TTTGAAATGTAAACTTTGATGTCTTCTCTTTCTCTTAGTTGATGCTATTGGGCCCATCTCAAGCTGATCTTGGCACCTCT
CATGCTCTGCTCTCTTCAACCAGACCTCTACATTCCATTTTGGAAGAAGACTAAAAATGGTAAGAACAGCTCAGAGAACC
TTAAAAAGTGTTATCTGTAATCTTTGTGGAAACAACTGAAACCAGCTGGCAAGAGCAATATTGAAGAATCTGTACTTAGG
TTATTTGCTGGGGGAAAGTGCTTCCTGATATTTCACAATTGGCATTAATGAAGGGGGCATGTCACAATTTCAGATTAATC
AACGCTTGCTCTGTTCAACTTCCTACAAGAATTAAATATGTGCTGTGGGGAGGAGGAGCAGATGTTTGAATTGGGGACAT
AGCTTCTATGTATCTCATTTCTTCAGCCTACAATTTTGGCTTTAAAGCCATAACAAATCACTGAATTACTGAAGTTACTT
TGTGCTTTTTCCAGCATATGGTGTTGTCTTAATGACTGTGTGGATGAAAGTGTGTGGGCAGGCTCATAGCAATAAAATAC
GGGAAATCCCCGGGCTTGAGTGCTGTCAAAGAAAACTAAATTTGGACAGTAGATAAAGATACTATCAGGACTATTGCAAT
CGGCAGAAAGAGACCTCAGTATAGAAAGGGGCTCAATTCCAAATACAGCCAAAGACCAGTAAAGATTTCTGGCCAAGGAG
TAGAGTGGGGGTCAGTGGATGGAAAATTACTAAGAGGAAACATCAAGGGTAAAAGGATTCTGGCTAAACCGACCTGACAG
GATTCTTGCTGAAGACAGGCCAGGGTGATCAGACCTCACCTGTGGATGGTGGGAGATGAGGAATTTGATCAGATATTGAG
GGTGATCACATACCAAGAGGAGTGGATTATCAATAAAATGACTTAGCAGGATTCCTGCTTGAACTGGGCAATGCAAAGAT
GGACATGAAGCCAAAGGCCGAAGCCTAGGGGTGTAGTAGAGCCTGATTAAGTTGAATTAAGGAGAGTCTTTGTCAGCGCT
GGCTCTCCCAGTCACTAGTTGGGGGGGCCTTGTGCCTGTCATCAAAGTCCTCTGAAACTCAATTTCTCTGACTATGAAAT
AGGCATTAGAATCCCTCCCCTGTTGCCTTCCAGGGCCACTGTGAGGCTCAAATAATAGACTATTTTTCAAGTCCTTTGCA
AGTGGTATGATGCAAGTGTGAGTTATTAGGTATGCCAAAACTTAGTCGGAAAAAGACGTCAAGGGCCTTTTTCTGAAATT
ATTTTGTCACTTAAATCAGACACATTCTAGATCCGAATGTTAGCTCCTAGGCTCATTTTGTGTCAAAGTTCTAATGAAGC
ATTAACCATGGGGCTATTGTTACAAAGGAAACAACTGCTTACGGTTTCATTTCCTAGAAACCCAGATGTCTATTTTAATG
CAAACCTATGCCCACATCTGTCTTTGCCCCTTGATGGGTGGCATAATGGGAATGATAGTAATACAGAGAGCTCACATTTC
TTGACCACTCAACTATCATGCTGAGGGCTAGATAGACATGATTCTATTTTGGCCTCAAAGTAGCCCTATAAGGTAGAGAT
AACGAAACTGGGGCTTTGAGAGGTTAAGGAGCTTGGGTGGCTCTGAAAGCTGTGCTGAAGACTCTTCTGTTCTTCCTAGA
CCAAGCCCAGCACACACGCAATAAAGATGAGGTTGGATATGATGGCTTCCTACTCAAGTACAAAGGGGAAATAGTATATC
TTTTCTAAGAAAAGACGTGAAAATAATTTTCAATATAAGAAATTCAAAAGGCAAAAAAGCACAGGGAAAATATTCAACTG
TATTGAGTCATATGGCAGATCCTTTGATCTAGAGATTACACTTTTAGAAACTCTTCTTAAAGAAGTGACCATGAGACTGG
ATAAAAAAATGTGGCACATATACACCATGGAATACTATGCAGCCATAAAAAGGAATGAGATCATGTCCTTTGCAGGGACA
TTGATGAAGCTGGAAGCCATTATCCTCAGCAAACTAACACAGGAACAAAAAACCAAACACCGCATGTTCTCACTTATAAG
TGGGAGCTGAACAGTGAGAACACATGGACACAGGGAGGGGAACAACACTCACTGAGGCCTGTAGGAGGAGGGTGGGGCAG
GAGAGAGCATTAGGGTAAAAAGCTAATGCATGCTGGGCTTAATACCTAGGTGATGGGTTGATCTGTGCAGCAAACCACCA
TGGCACGTTTAACTATGTAACAAACCTGCACATCCTGCACATGTACCCCAGAACTTAAAAAAACAAGCAATAAAATAATT
TTAAAAAAACAAAAGAAGTGATCGTGGACATGGAAAACTATTTACCAAGATGGTCAGTGCAGCCAGGCAAAAAAAAAAAA
AAAAAAAAAATCATGTCCCATGTTGGGAAGGGGTGAATTAATTGTAGTAGACTCATTAAATGGAATATTATGTAATCATC
AAATCATGTTTTTTAAAATAATACTGAATGACCTAAGAAAGCACTCATGGTATAATGTTAAATGAAAAAAGCAAGCTAGA
AATGGATAAGTACCGTGTATTCCTCATGTTTTTACTGCACCTGCTAGGCAAATACTAGATGCTCACTAAATGTTGGATAA
TCTGTGATGATGGTTTACATAAACACATGTGTTGCATATTCTAATTTCATTCAACATCCCTACTTTATAACCATTTTACA
GTTGGCAAATCAGAGGCTCATGAGGTCAAGTGATTTATGAAAGTCAGAGAGCTCTTACATGACAGAACAAGGACTTAAAA
CCAAATTTTTGTACTGACAAAGCCTTGGCTGTTACTAGAATGCTTCTCACCATGTGAAATAGATGCAGGGATGGGAAATT
ACTATTAGAAGGGACCATCTCCCAAAATGTCAATAGTGGTTCAGCAAATTTAAAAGTAAAAATATTATTCTGCTCTTAAC
CTATAGGAAATTTCTTTATGGCTAAAAAAAGGTTATTAAGTAATCAATTTATTAAATTAATACAATCTGATTATTTAAAA
ATTTGGAACGCTGTACTAAAATTAAAAATCATCATTACAGATTAACCAGCCAGTACCTCTGCACCCCAAGAATAAATAAT
GTATATCCCCGAAACTCACCGAAGTTTAGGGCTGGGGTTGGCAAACTATGGCCCATGGGCTATATCCCACCTGCTGTACA
GCTCATGAGCTAAGGGGTTTTTTTTTAATTGTTGTTTTTAAAAGACTGAAAAATATCAGAGCAAAATTACTATTTTGTGA
CATATAAAAGTTACATTCAAGTTTCAGTGTTTACAAATGGTTTTATTGTTTGAGTATTTGTTTACTTATTGTTGATAAGT
GCTTTTGCACTACGATGGCAAACTATTCAAGGAGTTGGGTAGTGTGACAGAGAACCTGATGGCCTGCAAAGATTAAACCA
TTTACTAACTGGCCCTTTACAGAAAAAGTACGTCAGGCCGGGGCTTATAGAAAACAAAGGGATAAGGTATAAGGTCAAAT
AGGTTTGAGAGCCCTATGGTCTTTGGTGACTGTTGTGATGCATAATAGCTGTTGAGTTCCTAATTTATGTAAGACAACTT
TATATCCTTTTATTCTTTTAGTTTGAAAACTAAGTCTGTTGGGCTAAAATGATAGGAAGTAAATGATAACTCTCTCCTTT
TTTTAAAAAAAAGCAAGTGGTTTACAACCTTGTACTTAAACGTTTTGGTGACATAATGAAACTGATATTCATGGTATTTG
TACTTTACAGAGATTAAACTAAAATTAAAAATATTTCAAAATTCACAAATAGGGGATATTTGTTAATAAATCTATTTGGG
AAATTCCTAGCAGAGGCTCAGTCTATAAAATGAATAGCATTTCAGCAACTTCCCTTATTCACAGTGCTTGGTTATTCTCT
AGGGAGACATACACAACACATCTCTAGTTACCAAACAATTCAGTGTGATATAAACATGGCAAAAAGTCAATGAATTTGAG
GGCAAGGTTTCCAGCAATCGCCCCGGCCATTGCTTACTTCTTCCATGCCCTTTCTAAGTTTTCTTCAGCCAGGCAGCCAT
CCCCTCTGGTTTCTCCCAGACCCCCGCTGCAGGCTCCCCGCCATCACAGAAAGCCCCTCGCTCACACGTCTTGGCTCAAG
CAACTCTTTGTCTTAGAAATGCAGATCCCAACATTTCCTTTTAAACTCAGGCAACTTGGCTTTTTTCTGCTCTGTGATCT
TGAAAGTCGCTTGGAGGAACAGCTGAGTGCATGGGGCTGTTGTCCTCTCAGGGCTAACATGTTGTAGCCCAGGGGGTGCC
CAGGGGCCTTTCTGACTGGTTGGTTAGTTGGGTAAAAGAGTAGAGTCAGGAGAGCAGGAAATCCTTTCTTAACTCACTAT
AAAAATAAAAGCGTTCCCCAGGCCTCAAATAGTCTCATCTCAAGATAAATTTCCTTTTGCCAAGATTGCTGCTGAAAATA
ATCCATTGTAGCCAGATAATAGCTATGCAAAGAATATATAATAGACTGGCAGGGGCATGCCTACCGATTCAATACAGAAA
GGTGAGGGTTTCATTTGCTGGGGTGTAGTGGGTGGGAGAATTCCTTATTGCAATCACACTCTACTTCTCCATCCAGAAAA
CTCTCCAACCCTCCTGGAGGACTCTCCATTTTCTCCTCTTTCTCCTCCTTGTGTACCTACCTAGACCATCTGCTCCCATA
TGTCCTGTCTGACTTCCTGTTCCAGTTACCTATCACTGCGTAAGAGATCACCTCAAAATGCAATGGCTTCAAACAACAAC
AATCATATACTGCTTTCTATCATGGGTCCAGGAGTTGACTGGACTCATTAGGCAGCTCTCCCACAGGGTCTCTCTTGGGG
TGGCAGTCAGGCGGTGACTGCGACTGGAATCACCTGAAGACTCACTCTCCAGGTCTGATGCCTGGGCTAGGAGACTCAAC
AGCTAGGTGCCGAAGCAGCTGCAGCTCCTCAAGTGTCTCTGTCTCCATGTGGTCTCTCTAATATGGTGGTTGTCGTATAG
CCAGGCTTCTTACAAGGGTGATGACTCAGGACTCCAAAGCAAGTGGGTGAGAGAAAGGGAGAGAGGGAGAAACAGGGAGA
GAGAGAGAGAAAGTGTGTGTGTGCCAGTACGCGCGAGGTGAAAGCTGTATTGCCTGTGAACTACCCACCATGTCTTTCGT
CCTCTTGACAGGAAACCTCCTAGAAATGTTTGCTGTCTCCAAATCCCTCTCCTTACGTTCTTCCAAGAACTTTGAAGTCA
TATTTTATGTAGCTACTCCTTCAAAACATATCTGGTGTTCGGCCAGTTCTTACGCCCTCCAGCACTGCTACCTGGGACTT
CTGCTTGAATGACTGTAATAGCCTCTCAACTAGTCTCCCTGCTTTCACCCTTGCCCCTCACTGTCTATTCTCAACACAGC
AGCCAGCAGCATCCTTCTCAAATGTAAGTCAGACCAACTGATTGTCAGCTCAAAAATTTGCAATGCATCTGCATTCCACC
CAGAGCAGAGACCGCCATCCATGGAATGGTAGAGAAAGCCCAACATGCTCAGGGACACTCCCTCTCTGACTTCATCTCCT
ATTGTTCTCCTACACCCCCTGCTTCAGCAATATTGGCCCCGTTGCCATTTTTGTGAATATTCTAGCATGTTTTCACCTTG
GGGCCTTTGCTCCAGGCTAATCCATCTGTCTGGAATGCATTTCCCCTGGATGTCTGTTATGGATGACTTTGTCCTTTCCT
TGAGGTCTTTGTTTAGATATCAACTTCTTAATGATGCCTATCCAAGCTGCCCTATTTATCGTCACAATCCTACCCCACAT
TCCTGATCCTTTTCACTCTGCCCTGTTTTCTTTTTCAGTAACACTTATCACTTGACATGCAATATCATTTCTGACAGTTA
TATATTTTTGTGATTATTTAGAGAACATAAGCTATAGTTGAGTGGAAATCTTTTCTATTTTGTCCACTGATGTCCCAAAC
ACCTAGAGAAGTACCTGGCATGTTGCAGGCATCAATAAATACTTGTTGAATTTTTCCTTTTTCACAATTTCCTTCTACGT
TGTTATGATGAGATCTTATTTCCTCTGTAATTTGATTTTAAAAGTTTTAATAAAAAACAATACATATTATTTATGATAAA
AAGTCAAAGAGTAGAGAAGGGTATAACATAAAAATAGAAGTCCCCCTCTTCCCAGGGAAGGCCCCTTTATACCACTGCCC
AGAAGAAATTGCTATTAAAGGTTTCTTGTGTATTCTTTCCTACTTTTCTCTGCAAATACAAATATATGCATATATATTTA
TCATAAATGCATTATATGTTATATGTTATTTTAATGCTGCTTTAAAAATCCCCTTTATTTTTTGTAACTTAGTAGTAGAT
CATGCATAGCTTTTTATGTCGATACCCACAGCTCTACCACATTCTTTTTAAGGGACATTTGATATTTTACTATTGGTAGT
TTCCCATTTTTAACCATTCTCTCAAATCAATGGATTGTCATGTAATTCTTCCTATTCTTACTATTTCAGAAAGCTGAATC
AAACTAGCAAAATAGTTTTATCTAAAGACATATAAGGCCGGGCGTAGTGGCTCTTGCCTGTAATCCCAGCACTTTGGGAG
GCTGAGGCAGGCAGACCACCTGAAGTCAGGAGTTTGAGACCAGCCTGGCCAACATGGTGAAACCCCGTCTCTGCTAAAAA
TACAAAAATTAGCTGGGAGTGGTGGCGGCTGTCTGTAATCCCAGATACTCAGGAGGCTGAGGCAGGAGAATCACTTGAAC
CGGGTAGGCAGAGGTTGCGGTGATCCAAGATCGGGCCAGTGTACTCCAGCCTGGGCGACAGAGTGAGACTCTGTCTCAAA
ATAAATAAATAAATAAATAATAAAGACATATAATGCTTACTTTAAAGAAAAACAAAACAAAACATGTACTAGTTATTTTT
TTCCTCCCTCTGTGGAATTCTTAGAAGGTTTATGGTAGTTTGAAGCTTTGCATGGACCATTTTGAAACAGCAGCAGCCTG
AGGTTCCAGGGGGTTATGAAGACTCCCAGCTGAGGACAGACCCTGGCAGATAAGTTTCAGGGGGCTCTACACCAACCATT
AGAGTCATAGAATAAGCACAATAGAAAAGGACCATTAAGGTCAGTTAGCCAAACTCCAGAGTTTGTTGATGAGAAAGTCA
AGGTTCAGGATAATTCAGTTGGTAGCCCTGTAGCAGACAGAGAGACTGAAAACAAATCTGACTTTCAGTTCACGTGGTGC
TAACCCCTAGAATAAATAAACACGAGGAGAAATCAGACTAATCCCAGTCTTCTTCTAACTTGTCACAAGACACAAACCAC
TTACCTTCACTTCCTCATTTTTTCCATCTAATAGTTCCCAGTTATATACATGTCCTTCTCACTCCTCTGATTGCAACCAG
ACATCTCTTACAAGTTTACAAAGTTTTGAAGATAAAAACGCTATTTGGAAAGCGTAAAGTTAAAAACAGCTTGGTAAATG
TTTTTTTTTTTTTCTATTAGTAATTCGATCTCTACAACTGTAAATATTGTGGTAGGAATCTAATACAGATCTAAAATCAG
TAAAATTCAATCTTGAATATGGGCTTCAGTCCTGCCATCAAAATAGTGCATCCAGGTGGATAGGTTTTGCCACCTTGAAG
AGTTGTTTATTCAAACTTTTGTTTGAAGAGTAGGAAAGCAGTGTTACCTTTAGGCCTGACTTAGCCCTTGCCCCACAATC
TATTGTTTTTTCTCACCATAGATTTCCCTGACAGCAGAGAGAGAGTTCTGTGCTCAAGAGATACACACAGCTTCTGACAA
TAGAGCAGCAGAGTATTTGGTTCCTAATTGAGCAGGAATGGTGTTTGACTCATCATCATTTCCCTACTTTGTCTAGCACA
GTACCTTGCACAGAGTAGATTCTCAATAATGTTTGTTGAATGACTGTGGGAGCATATAATTCATAATGGAGACAAAGCTC
AATGAGGCTTTAAATTTCTAAATCCACAAAATGCCCTCATGTAACATTGCTGGATGATATGGTTTAGCTGTGTCCCCACC
TAAATCTCACCTTGAATTGTAGCTCCCATAATCCCCACGTGTTGTGGGAGGGACCCAGTGGGAGGTAATTGAATCATGGG
GGCGGGTTTTTCCCATGCTGTTCTCATGATAGTGGATAAGTCTCACAAGATCTGATGGTTTCATAAACGGCAGTTCCCCT
GCACATGCTCTCTTGCCTGACGCCATGTAAGACGTAATTTTGCTCCTCCTTCACCTTCCACCATGATTGTGAGGCCTCCT
CAGTCATGTGGAACTGTGAGTCCATTAAATCTCTTTTTCTTTATAAATTACCCAAACTCGGATATGTTTTTATTAGCAGC
ATGAGAACAGACTAATACAATGGACATTGGATGCAATTCATTTAAAAAATCATCTTAAAAATATCTTTCTTTTTTCTCCC
TCAAGTTGGTCCCACTCAAAACATAAACACACCATTTTTTTTTTTTTTTGTCTTGAGACAGAGTCTTGCTCTGTCACCCA
GGCTGGAGTGCAGTGGTATGATCGTGGCTTACTGCAACCTCTGCCTCCCGAGTTCAAGCAATTCTCCTGCGTCAGCCTCC
TGAGTAGCTGGGATTACAGGTGCATGCCACCATGCCCGGCTAATTTTGTATTTTTAGTAGAAATAGGGTTTCACCATGTT
GGCCATGCTGGTCTCAAACTCCTCACCTCAGGTGATCCTCCCGCCTTGGACTCCCAAAGTGCTGGGATTTCATGTGTGAG
CCAGTGTGCCCAGCCACCATTTTTTAATACTTGTAAATTTTTCCTATAAAAACAAACCAATTTCTCTATGCCCCAAAACC
GCTAAGTAGCACAAAATAGAAACATTAGAGTACCAAGAATACTTGAACTGAAAAGGAAATTAATCAAAATGCAGACACAC
ATTATACCAAGTGCATTTGCTGTAGCTGTGTAAGGCAACTTGAATAGAATTGGTCAACAATGAGTCTGAATCTTGGTTTG
AAATTGCCTGTCTGATCTCTGCTTCCTCATCAGTAAAATGAGAATATTTATATGGCCTTTCAACTTCAGTGTGAGGGATC
AATGATGTAATATAAACAACAAGTCTGCCTTAGAACCTGGCACACCATAAGTAATAAAAGGCAGCCAATATTTTAAAAAA
TACACAAATCATGGTCTGATGGCTGTCCAATATAAATTCTCTATTTTCCATTTTAACTAAAGAGACGATATATTGAGAAA
ATAGAAACACCTGTGTGTATGAAATCACCCATTCCCATTTTTACAATAATTAGTTTGCTAATTGAGCATCCAAATTTACC
CAGTGTATTTGCATGTGTAATTAGCTGTGATTCAATACCAAAGCCAGGCCTATCATGGTATACTATGCTATTTTACAAGT
CAAATTACTGAAAGATGCATGTCTTTAGGCAATCATTACAAATAAAAAAAAAAAAACCGAAGCAAAACAAAATAACATAG
ATTATTTGTATCAGATGGACAAAACAGACCTGGCTTGATGCCGAACCCTTAAATCTCAAAATAACGATAGTTGAAGCTAA
GGTTCCAGCTTAAGTCTGAAGCAGGTAGTTTCCAATGGCTTGAAAGGAGAAATTTCTACACTGAAGGAAATTTCCATTGG
AATAAAGGAATATTTCACACTTTTAAGTCATCTTCTCTAGATGGTCTTTTGGGTATACTTTCTCTTTAAATAACAGATTT
AGAAGCACTTTGTTCATTTGTTTAGAATTAATTCCATTCACAAGTTTAACACAGCCTAAGGTTTGGTCTAGACCAGGGGT
CTGCCAGCTATGACCTCTGGGCTAAATCTGTCCCTTCACCTGCTTTTTTTTTTTTTTTTTTTCCAACCTGTGAGCTAAGA
ATGGGTTTTACTATTCTAATAAATAGTGAGTTCATTTTTCTCCCTCACCTGCTTGATCAGAGCCCAACTTTCTCATTGCA
GTTAATCTTCCTTCTGGCATGGATCTTGGAATGCAAACTTGCTGGGATCTCCGAGTTCCAGGCTTCCCGTGCAGCCGGTG
TGGAGAGCCAAGAGATGTTTTGTTTGGCATAAAGCATTCCAAGGGTCAGTGGGCTTGGGCTCAACTATTGAGCATAGGAC
AAGGGCAGCCCCATCCTGACTGTGACTCTTCCCACAAGAGACAAACGAGCTCTGTGCTTTCACTGGGGTTTCAGGTTCAA
AGGGACAGAGCGTCTGAGAAAAAGGATTATGAAAGAGTCCGTCTGCAGCTCCACTTCCCGTGCCCTTCCAATGATACCAT
CCTCGTTTCTTCTGTGGCATGCTCCCCACTTCAATCCTTCCTTCAGAGGCCCCAAACCCTCCTGGTCTCTCCTTGTCACC
TTGTGAAAATCTGATCTTCAGGGAAAAATTCCTTACTATTTATACTAGTATAATGTGAATCTTCTATGGGATTTTAAGAA
AGTTCAAAGCCTTGGTTTACTCAGCAAATATTTAGCTTGCACTCACTATGTGGCGGGCATCCTAATGATGGAGTATATGT
AAAGACAAAAAAAGTTTCCGGACCTCAAAGTGTTCTCCATCTATAGGGGCAGATGACTGAGTTGACATCTCGAGAAGTAG
AATAGCAGAGTGGCTAAGAGTGCCAGCTCTGTCTCAATCACCTAGGTCTCACCTCAGCATTAATTTCACTTTCCTCATTG
TAAATGAGCATATCTCTTAGAATTGGGATAAGCATTAAATAATATAGACTTGGAATGAATTTGCTTAGAACTAATTCCAT
GCACAAGTTTATCACAGCCTAAGGTTTGGTCTAGACCAGAGGTCTGCCAAGTATGACCTGTGGGCTCAATCTGTCCCACT
ACCTATTGTTGTTGTTGCTGTTGTTTTTTAATGACCTGTGAGCTAAGAATGGGTTTTACTATTCTAATTAGTTACATTCT
CAATGGTTATTTAAGTACCTCCATAATATCCTCAATTTTGCCTAAAATATTTACCATCTGGCCCTTTACAGAATAAGTTT
GCTGACTTATTGGTCTGGACCAATGCTATCTAATAAAACTTTCTGCAATGATGAAAATGGTCTCTATCTGTACCCTTGAA
TACAGCAGCCACTAGCCTAATGTGGCTTTTTGAGCTCTTGAAATATAGTTAGTGTGACTAAGAGATTGAATTTTAATTAA
TTTAAATTTATGGAGCCACATGTGACTATGACATTAGAGCAGCTCTAGACAGCCTGAAGTCTAAAGACTCTATGCTTTGT
CGGTGCTCCCCTCTCTCAATTGAATCAACTACCCTGAGGCTGCATGAGTCAAGGGGAAGGCCACACTCTTCAATCAGATT
TTTTGCCCTGGACTGGCTTTCATTGTCTACTAGAAAATGCTTAATGGGAAGTGCTTAGAAAATGTACATGGGCATACACT
TAATTAATCTAAGTTGCTGCTTTGTCTGTATCCATTAAATCTGCTTTATTTTGGGGTAAACTACAGTAGAAGTTGGCTTT
TTCAACCCTGCAAAGCCTTAAAATTCAGGATGTCTTACTCAACTTAAAGTGTAGAGTTGCAGCCAGAGCACAACTGTATT
TCCTTCTAGCCCTGCTTGCAGAATGGCTAACTTCAGTCCTATTTCATTTCTCTTGTAAGACTGCTAAAAACAGTAAGAAG
CCACCAACATCATTATGAATATTGCCAAATCATTTCGCCTAAGAGTAAAGTCACAGTTGGCATGTGTTCTGCCCTCCAAG
ACAAGATAGCATAGGTGACAGTTTTATCAGATATCTTGTGATGGCATAATATAGGCCACCCAGCTTTCCAGCCTCTGATA
TCTGAGTCTTCCCAATAGCCTGATGACATCCGCATCACATATTTTAGGTTCGCTCATGGACAGTAACTTATTTCCAAATT
CTATACTGGTTAAAATTAGGTTTGCATTTGTGCAATAGAAAATCCAATTGACATTGGCTTAGCATAACAATTTTTTGATT
TCTCATAAACTCTTGGCAGTCAGCAGGTCCAAGCCATTATTTCTGCTCTGCTCTCTGAGGTCATATAAGGAAGGATCTGG
ATGCTCTGGGTCATCTACGTCATCTAACTGGTTGCTGTGCCATCCCTAGCTCATTTTTCTCATGTGCATTGCCCAAGATG
GCTGGCTACAACATCCACATTACAAGAAGCCAGGTGGAAGCAGACAGGAGAAAGAGGAGAAAGGGGAACTGCCCCACCGT
TTAAGGACATGTCCCAGAAACTGTACACCTCACTTCCTCCCAAATTTCACTGGCTATCACTTAGTCATATAGCCACACTT
AGCTGCAAGTGTGTCTGGGAGATATAATTATTTTTCACAGTGGTATATGCCCAACTACAAATGGAGGTTCTGTCATTATG
AGATGAGAGAAAGGCAGAAAACATGTTGAGAGATGTGTAGCAATCTCTGGACTCCACGGGGATAAAAAAGAATTGAGAGT
ATCAAAATTCAGGATCAAAATCAAAATTAAAGATAAAAAATATCAATAACTATCACCTGGAATAAGAACAACGTACAGTT
CAGCTACACATATACAAGTGGCAGCATCTTGTCTGGAAGGAACTAATGGTCTTTCTACATTGTATTTTAGATATGTATTT
TTTTTTCTCCCTTCCACAGGATTTTGAGCTCCTTAAGGGCAGAGACTTTGTGTCTCCTGCTCCTAGTAGGCATCCAACAC
GTATCTGTCAACTGAAAGAATGAATATGAGTCAGTAGATACATATTAGAATTCTAATATCCACTGGCTGGGTCCTTGGTG
TGTCCCATATTGTTGTTTCTGTGTCCATCATTCTTTTGCAGGGTATCTTCTACTGGGCACAGAACCTGCCTCAGAGGGGC
ATATGGGTAATGAACTACCAAGAAAGGAGTAGAACCCAGTTCTTCCAACCCTCCACCCAGAGTGCTTTTCACAACCTCAT
GTGTAATAAGTGCAGTAGGAGATGAGAGGAGGGAGTGATTACTTCTGTCTGGTTTGATCCCAGAAGGTTTTTTGAAGAAA
GTGTTTTTGAATGAGACATTATGAAAACAGAGCTTCTTAAACCTTTTCCCCCAAGGAATCCCTGGGCAGATAGAAGAGAC
AGAAATCTGACCTCTGCTTAGTCTGGGGGTATAGACTGAAGGAACCTACTCAAAGGAGAAATTTTTCTCATTTTTCTTTA
CTTCACGATTCATATATGCAGGCATTCATTCTTTCATTCATGTATCTCACAGACATAACGAGGTCCTAATTAAGTGCCAG
GCATTGTTTTACATGAGACCACAAGAGGCCCTACCCTCTTGCAGCTTACATTCTTGTACAGAATAGACATCATACGAATA
AGCAACATAAATCATCAAGATAATTTCTGACCGTGGTAAGGGCTATGACCGAAATCAAACAGGGTAGTCAGTTACAGAGT
GCATATACCTCTCTGTGCCTCAGTTGACTCATCTGTAAAATGGAGATAATAATAGAGGTCTAGGCTAGGCATGGTGGCTC
ATGCCTGTAATCCCAGCACTTTGGGAGGCCGAGGTGGGTGGTTCACTTGGGGTCAGGCATTCCAGACCAGCCTAACCAAC
ATGGTGAAACCCCGTCTCTACTAAAAATACAAAAATTAGCCAGGCCTGGTGGTGCATACCTGTAATCCCAGCTACTTGGG
AGGCTGAAGCAGGAGAATCGCTTGAACCCGGGAGGTGGAGGTTGCAGTGAACCGAGATTATGCCATTGCACTCCAGCCTG
GGCAATAAGAGCGAAACTCAGTCTCAAATAATAATAATAATAATAATAATAATAATAATAGTCTATAATTCCAAAACCCA
AAACTGAAAGCTTTGTCCTAACTCAGTTGATTGCAAACATAATATGATCTGAATGCATTTGGAGGTAGATCTTGACCTGA
ACTGAAGTTATTTATTCTTTTTAATAAATAAATGAGTTATTTATTCTTTTTAATAAATAAATGAGTCATTTATTCTTTTT
AATAAATGAGTTATTCTTTTTAATAAATAATAAACTGAGTTATTTATTCTTTTTAATAAATAATAAATAACTGAGTTATT
TATTCTTTTTAATAAATAATAAATGAGTTATTTATTCTTTTTAATAAATAATAAATAACTGAGTTATTTATTCTTTTTTT
TTTAATAATTCCACTTAGAGTGGACAATCCTATATGTCACTGCAGAAATTTTGTGTGTTTGATTATGGAATGCTGCCCCA
GGCCTCAATAGTTATTACATAATTTAGGGTACATGTAGCGTATTACCTTCTAAAATTTGAAAAATTCCGAATTCCAAAAC
ACATGTAGCACCAAAGGTTTCGGATAAGGGATTGAAGACCTGTAGTATCCATTATTGTGAGGATTAAATGAATGAATATA
TGGAAAACACTTAAAATGATGCCTGGCATGTGGTAAGTGCTACGTAAGTTAACTACTATTACTATTATTATCACTATTCT
TACATGAGAAGATATTTAGATAAGTTGGTCAGGGAAAGCCTCTCTGAGGATGTGTCACTTGAATAGGCAACTAAGGGGTG
GTAATGACCGGGCTGTGGGAAGAGGAGGAGAAAGATGATTTCAGGAATAGGAAACAGCAAGTGCCAAGACTGTGGTGGTT
ACAAGGCTGGCTTGAATGCAGAACAGAAAACAGACCAGATGGCTGATATGTGGTAAAGGAGGGGAAAGATGGCTCAAGGT
CAGAGAGGTAGGCTGAAGTCAGAACACCCTTGATATAAGCAATGGTAGAGACTTTGGATTTCATTTAAAGTGTAATAGGA
AGACATTATAGTTGATCTGATTCAGGTTTATAAAGAACGCTCTGATGCTGTTGGATGAATGAATTATAGAGGAGAAGGGG
GAGCAGGGAGAGCAATTTGGAGTCTAGCATAGTGGTCCAGATGAGACCTAATGACTAATTGGAGTTGGGAGGTGGTAATA
GTCAAAGAGAAAAGTGGACAGGTGCGAGAAAAAAGTTTAGAAATAAGTGGGGGGCGGGGGAGGTTTTCTGATTAATTTGC
ATTCTAATTTATAATATGTCACTGTGTAGAGGCTAAAAATTTCACAGTCATTGTCTCAGGTGTGTTAAGGCCAGTGGCGT
GCTGGACCCCACTTGAAATTGGCCATGGAGGGAATATTTACACTATAGAAATTGACAAATGCTACAAATCAAGACAACAA
ATCAGGCAAAGCTTCTTGTTAAACATTTACCATCACACCACTGGTGAAGGTGACTTGATTTTTCCACAACTAAACTTCCT
TCATTTCACAGCCTCCATTTTCCCTGATCACGAAAACACTTAAACTAGGCACATCCTCGGAAACGCAGTATGAGGACTGC
TGTGTCAATCACTTCATGTTTTTAACTCAATTCAGCGATCCTCCCACTTCTTCCCAGGCTCTCATTTAGGTACATGGGAA
TGGGATGGGAAGAGGGACCTGGTTCATGATTGTCATTTACCCACCTTGGCCCCCTCTGAAGTACAACTCCACTCTCTGCT
TTACAATATCACTCTGGGCAGCATTACCAATTGCCTCCTGATAGTGGGATCTATGAACCCATTATGTCTTTGGACAAAAG
CATAGCCAGGGGTTGGGTCCAGGGCCTGGGATCCTATAACCGTACAAATCCTATTATCAGGGACTATAAAATCCTATTAT
CAGGGACCATAGCCATCCCTCTATCTTGACTCAACTCCTCCTCCCTGAGTAGTGAACATTTTTCCTAAATCTCTGAGAAA
GACTGGTGCTCTAGAAAGATGTACCATATTTATTTAAGGGCTTCCTGTACCCACTGGCATATTGCCATATATTCTGAGGT
ATCTGAGTGCTCCTTTTGAGAAACATAGCCTTAAAGGATAAGTAGAAATCTGGTGGGTGAAAATGGTAGGGAAGAGGACT
TCTAACGGAGGGACTTGCAAGTCAGGGAACTTGGGTTTATCGACTAGTGAGGCTAGTAGAGGAATTCAATCAGGTAAGCC
GGACAAGTAGACAGGGTACAAATTATGGAAGACTTTGGATGCCATGATAAAAAGCTTCAGCTCATACTGTAAAAAATAAA
ATAAAATAAGAAGGTTGGGTGCAGTGGCTCATGACTGTAATTTCAGCACTTTGGGAGGCTGAGGTGGGACGATCGCTTGA
GCCTGGGAAACAATTTCAAGGAGTTCACAGCAAGAAACTGACTGATTAAGGTTTGGGAAGCTTGATAGATAGGGTAGACT
GGGAAAGTGAGAGAGGAGGCTTTGGAGTGGACCAAGGATAGAGGGATCTCAGCTGATATTATGTCAGCTAAAACCTCAAA
GCAAGGAGGATGTTAAGAACAATGAAGGAGGTCAGCTGGACTCTCAATGTTTTTAACGATAGGGAGGAAAAGATAGGGGG
GTGACAAGAAGAAGAGACAATTTTGTACCTCTAACTCCAACAAACTTTAGACCTGAAAAATCCCTTCTGAGCCATCTTGC
ATTGGAGAAAAAAAATTGCTTATTTACCTCCAATTAGAGGAATTAAGGGAAGTAGGATTTTTTTGTTTTTCTTTTGAGAC
AGGGTCTTGCTCTGTCACCCTGGCTGGGGTGCAGTGGTGTGATCACGGCTCACTGCAACCTCAAACTCTTGGGCTTAAGA
GGTCCTCCCAACTCAACCTCCCGAGTAGCTGAACTACAGTTGTGTGCCACCATGCCCAGCTAATTTTTTATTTTCTGTAG
AGAGAGGGGTCTCACGCTATGTTGCCCAGGCTAGTCTTGAACTCTGGCCTCAAGCGATCCGCCTGCCTTGTCCTCCCAAA
GCGTTGGTATTAGAGGCATGAGCCACCACATCTGGTGGAAGTAGGCATTTGGTTTCTTAGATAACAACATGATTGGTTGA
TTCAGTCACTTGGGAAGATAAAAGCATTAACTGAGCTAGATCCCTATGGTAGAGACACAGGCTGGACCACTCCATGCGTA
AGTACTAAACTAAAACCAGTGTTCTGGAGTAGACATTGCTAGAAATCCTGAAACTTGAGAGCCAGTCCACGGTTAAAGCA
TTCTGTAAGGCAGAGCCAGTGGAAGGTAATAAGGTGATTTTTAAAGCTCTTCTGCACTTCCCATATTCCCTTTTAGGGCC
TTTCTCCCTAGGGTCCCAGTGTCTGTCATGCTAAACCTAGATGCACAACAATCATCTTTATGGGTAGTTTCCCATATGTC
CCAGTTTGCCTGACAGACTCTTGGTTTATGCCTATAGTCTTGGTGTAATTATTACCAGCCCCACTTCATTCTTGTAAGTA
TACTAATGGATCAGTTATACGGTTCCTCTGATTATGTATCACCTAGGCAGTGCCCTGACTCTACTACTATCTCCTCTCCA
AATTTATGTAATGTAAACCCAATGTGTAGGGAAAATGCTCATCCTAAAATCTCCTTGGAGGGGATAATTTGCAAGATTCT
TTGCAAAAACAATCCAAGACAAGAGCCAGATTATGGAATGTCAGTGCCAGAATGGCAGGAATGTATGTTTTCTAATCAAA
TGCCACTTACTACTGGGTAACCTTGGGCTAATCAGTTAATATTGCTGAGCGATGTCTTCATTTGTAAAACGGGAATCTTA
GAATATTCTGAGACTCAAATACTATGAAAGACTCATGTAATGTGTACCAGGGCAGGTTTAGCAGGCCGACATAAATTGCA
CTAAAGTCTTCATGTGTTATTTTTCATGGGTGTATCCATATTCTAACATTTCTTCACCCTCCAAATTTCAGACTTTGGCA
GTGAATCTATGGCTCTGCAATTTTAGTGTTCCATGTAACAACGAATAGGAAAATGCTGCTTCTACCCTCTCGAAAGCTAT
TTTGCTAAAGAGCTAAGATGCTAAAAGCTAAATATGTAACTAAATAGTTGCAAATCTCAGTAACTGACAAATACAGTCAT
GGGGTTGGGGATGCTGTTTAGACAGCTGAAAATAAGACCTGAATTGTTTATTTTTAAAATGTTGCAAAAGAGAGGCAGCA
AATGGGAATTTTTAATTCTGATTCTTGGTATGTTTTAGAACAATGATTTGTTCTTTCTTATACTTTCAGGTGTTTCCAAT
GTGGACACTGAAGAGACAAATTCTTATCCTTTTTAACATAATCCTAATTTCCAAACTCCTTGGGGCTAGATGGTTTCCTA
AAACTCTGCCCTGTGATGTCACTCTGGATGTTCCAAAGAACCATGTGATCGTGGACTGCACAGACAAGCATTTGACAGAA
ATTCCTGGAGGTATTCCCACGAACACCACGAACCTCACCCTCACCATTAACCACATACCAGACATCTCCCCAGCGTCCTT
TCACAGACTGGACCATCTGGTAGAGATCGATTTCAGATGCAACTGTGTACCTATTCCACTGGGGTCAAAAAACAACATGT
GCATCAAGAGGCTGCAGATTAAACCCAGAAGCTTTAGTGGACTCACTTATTTAAAATCCCTTTACCTGGATGGAAACCAG
CTACTAGAGATACCGCAGGGCCTCCCGCCTAGCTTACAGCTTCTCAGCCTTGAGGCCAACAACATCTTTTCCATCAGAAA
AGAGAATCTAACAGAACTGGCCAACATAGAAATACTCTACCTGGGCCAAAACTGTTATTATCGAAATCCTTGTTATGTTT
CATATTCAATAGAGAAAGATGCCTTCCTAAACTTGACAAAGTTAAAAGTGCTCTCCCTGAAAGATAACAATGTCACAGCC
GTCCCTACTGTTTTGCCATCTACTTTAACAGAACTATATCTCTACAACAACATGATTGCAAAAATCCAAGAAGATGATTT
TAATAACCTCAACCAATTACAAATTCTTGACCTAAGTGGAAATTGCCCTCGTTGTTATAATGCCCCATTTCCTTGTGCGC
CGTGTAAAAATAATTCTCCCCTACAGATCCCTGTAAATGCTTTTGATGCGCTGACAGAATTAAAAGTTTTACGTCTACAC
AGTAACTCTCTTCAGCATGTGCCCCCAAGATGGTTTAAGAACATCAACAAACTCCAGGAACTGGATCTGTCCCAAAACTT
CTTGGCCAAAGAAATTGGGGATGCTAAATTTCTGCATTTTCTCCCCAGCCTCATCCAATTGGATCTGTCTTTCAATTTTG
AACTTCAGGTCTATCGTGCATCTATGAATCTATCACAAGCATTTTCTTCACTGAAAAGCCTGAAAATTCTGCGGATCAGA
GGATATGTCTTTAAAGAGTTGAAAAGCTTTAACCTCTCGCCATTACATAATCTTCAAAATCTTGAAGTTCTTGATCTTGG
CACTAACTTTATAAAAATTGCTAACCTCAGCATGTTTAAACAATTTAAAAGACTGAAAGTCATAGATCTTTCAGTGAATA
AAATATCACCTTCAGGAGATTCAAGTGAAGTTGGCTTCTGCTCAAATGCCAGAACTTCTGTAGAAAGTTATGAACCCCAG
GTCCTGGAACAATTACATTATTTCAGATATGATAAGTATGCAAGGAGTTGCAGATTCAAAAACAAAGAGGCTTCTTTCAT
GTCTGTTAATGAAAGCTGCTACAAGTATGGGCAGACCTTGGATCTAAGTAAAAATAGTATATTTTTTGTCAAGTCCTCTG
ATTTTCAGCATCTTTCTTTCCTCAAATGCCTGAATCTGTCAGGAAATCTCATTAGCCAAACTCTTAATGGCAGTGAATTC
CAACCTTTAGCAGAGCTGACATATTTGGACTTCTCCAACAACCGGCTTGATTTACTCCATTCAACAGCATTTGAAGAGCT
TCACAAACTGGAAGTTCTGGATATAAGCAGTAATAGCCATTATTTTCAATCAGAAGGAATTACTCATATGCTAAACTTTA
CCAAGAACCTAAAGGTTCTGCAGAAACTGATGATGAACGACAATGACATCTCTTCCTCCACCAGCAGGACCATGGAGAGT
GAGTCTCTTAGAACTCTGGAATTCAGAGGAAATCACTTAGATGTTTTATGGAGAGAAGGTGATAACAGATACTTACAATT
ATTCAAGAATCTGCTAAAATTAGAGGAATTAGACATCTCTAAAAATTCCCTAAGTTTCTTGCCTTCTGGAGTTTTTGATG
GTATGCCTCCAAATCTAAAGAATCTCTCTTTGGCCAAAAATGGGCTCAAATCTTTCAGTTGGAAGAAACTCCAGTGTCTA
AAGAACCTGGAAACTTTGGACCTCAGCCACAACCAACTGACCACTGTCCCTGAGAGATTATCCAACTGTTCCAGAAGCCT
CAAGAATCTGATTCTTAAGAATAATCAAATCAGGAGTCTGACGAAGTATTTTCTACAAGATGCCTTCCAGTTGCGATATC
TGGATCTCAGCTCAAATAAAATCCAGATGATCCAAAAGACCAGCTTCCCAGAAAATGTCCTCAACAATCTGAAGATGTTG
CTTTTGCATCATAATCGGTTTCTGTGCACCTGTGATGCTGTGTGGTTTGTCTGGTGGGTTAACCATACGGAGGTGACTAT
TCCTTACCTGGCCACAGATGTGACTTGTGTGGGGCCAGGAGCACACAAGGGCCAAAGTGTGATCTCCCTGGATCTGTACA
CCTGTGAGTTAGATCTGACTAACCTGATTCTGTTCTCACTTTCCATATCTGTATCTCTCTTTCTCATGGTGATGATGACA
GCAAGTCACCTCTATTTCTGGGATGTGTGGTATATTTACCATTTCTGTAAGGCCAAGATAAAGGGGTATCAGCGTCTAAT
ATCACCAGACTGTTGCTATGATGCTTTTATTGTGTATGACACTAAAGACCCAGCTGTGACCGAGTGGGTTTTGGCTGAGC
TGGTGGCCAAACTGGAAGACCCAAGAGAGAAACATTTTAATTTATGTCTCGAGGAAAGGGACTGGTTACCAGGGCAGCCA
GTTCTGGAAAACCTTTCCCAGAGCATACAGCTTAGCAAAAAGACAGTGTTTGTGATGACAGACAAGTATGCAAAGACTGA
AAATTTTAAGATAGCATTTTACTTGTCCCATCAGAGGCTCATGGATGAAAAAGTTGATGTGATTATCTTGATATTTCTTG
AGAAGCCCTTTCAGAAGTCCAAGTTCCTCCAGCTCCGGAAAAGGCTCTGTGGGAGTTCTGTCCTTGAGTGGCCAACAAAC
CCGCAAGCTCACCCATACTTCTGGCAGTGTCTAAAGAACGCCCTGGCCACAGACAATCATGTGGCCTATAGTCAGGTGTT
CAAGGAAACGGTCTAGCCCTTCTTTGCAAAACACAACTGCCTAGTTTACCAAGGAGAGGCCTGGCTGTTTAAATTGTTTT
CATATATATCACACCAAAAGCGTGTTTTGAAATTCTTCAAGAAATGAGATTGCCCATATTTCAGGGGAGCCACCAACGTC
TGTCACAGGAGTTGGAAAGATGGGGTTTATATAATGCATCAAGTCTTCTTTCTTATCTCTCTGTGTCTCTATTTGCACTT
GAGTCTCTCACCTCAGCTCCTGTAAAAGAGTGGCAAGTAAAAAACATGGGGCTCTGATTCTCCTGTAATTGTGATAATTA
AATATACACACAATCATGACATTGAGAAGAACTGCATTTCTACCCTTAAAAAGTACTGGTATATACAGAAATAGGGTTAA
AAAAAACTCAAGCTCTCTCTATATGAGACCAAAATGTACTAGAGTTAGTTTAGTGAAATAAAAAACCAGTCAGCTGGCCG
GGCATGGTGGCTCATGCTTGTAATCCCAGCACTTTGGGAGGCCGAGGCAGGTGGATCACGAGGTCAGGAGTTTGAGACCA
GTCTGGCCAACATGGTGAAACCCCGTCTGTACTAAAAATACAAAAATTAGCTGGGCGTGGTGGTGGGTGCCTGTAATCCC
AGCTACTTGGGAGGCTGAGGCAGGAGAATCGCTTGAACCCGGGAGGTGGAGGTGGCAGTGAGCCGAGATCACGCCACTGC
AATGCAGCCCGGGCAACAGAGCTAGACTGTCTCAAAAGAACAAAAAAAAAAAAACACAAAAAAACTCAGTCAGCTTCTTA
ACCAATTGCTTCCGTGTCATCCAGGGCCCCATTCTGTGCAGATTGAGTGTGGGCACCACACAGGTGGTTGCTGCTTCAGT
GCTTCCTGCTCTTTTTCCTTGGGCCTGCTTCTGGGTTCCATAGGGAAACAGTAAGAAAGAAAGACACATCCTTACCATAA
ATGCATATGGTCCACCTACAAATAGAAAAATATTTAAATGATCTGCCTTTATACAAAGTGATATTCTCTACCTTTGATAA
TTTACCTGCTTAAATGTTTTTATCTGCACTGCAAAGTACTGTATCCAAAGTAAAATTTCCTCATCCAATATCTTTCAAAC
TGTTTTGTTAACTAATGCCATATATTTGTAAGTATCTGCACACTTGATACAGCAACGTTAGATGGTTTTGATGGTAAACC
CTAAAGGAGGACTCCAAGAGTGTGTATTTATTTATAGTTTTATCAGAGATGACAATTATTTGAATGCCAATTATATGGAT
TCCTTTCATTTTTTGCTGGAGGATGGGAGAAGAAACCAAAGTTTATAGACCTTCACATTGAGAAAGCTTCAGTTTTGAAC
TTCAGCTATCAGATTCAAAAACAACAGAAAGAACCAAGACATTCTTAAGATGCCTGTACTTTCAGCTGGGTATAAATTCA
TGAGTTCAAAGATTGAAACCTGACCAATTTGCTTTATTTCATGGAAGAAGTGATCTACAAAGGTGTTTGTGCCATTTGGA
AAACAGCGTGCATGTGTTCAAGCCTTAGATTGGCGATGTCGTATTTTCCTCACGTGTGGCAATGCCAAAGGCTTTACTTT
ACCTGTGAGTACACACTATATGAATTATTTCCAACGTACATTTAATCAATAAGGGTCACAAATTCCCAAATCAATCTCTG
GAATAAATAGAGAGGTAATTAAATTGCTGGAGCCAACTATTTCACAACTTCTGTAAGCTTTATTGTGTTTCATAGTTTCC
GTTCTTCTTCTGTGAGAACAAGGATAATGGCATTAAAAAATCAGCTTTTGGTCATTATAAATTGTCTTCTATTAAAACAC
ATATACACATAAAATCACTTGAAGACAATTTAAACATCTTCTGAAATGGATCAAGAGGAAGGGAAACTGAAAATAATGCA
ACTCAGAAACCACAGAGTATTTTGACATGAGGTTAAGCACCGTGGTTTGTTGTAGGAAAATAACAGCACACCAACAGATG
GTTTTTATCTGAATTCTTTGGTAATCTTGACATGTCATTCTTCTAACTTTCTGAGGGCCCTCAGTGCAGTTTTGTAGGAC
TGGAGCTGTTCACAGACGGTCCCCACAAAGCTCTGAACGTGGGGCTTCTCTGCTGACTGGCCTCTGGTTGGCTCCACCCC
GGAAGGAACTCCCAGATTCTCCATGAATTCCGCTTCCACCATCAAGCCTTGGTCCAAGCCCCTTTCAACCTTGACTTGGC
CAGGAAGTGTCCTTTCTCTTCAGATAGATACTACACCTTAGCAAGACTTGGCATTTTTAGAATCCAAGCCAAGGGAGGCA
CTTGGCAAGGCAAATGTT
```

1. An in vitro method of detecting a polymorphism in a cancer patient or a patient suspected of having cancer, the method comprising screening a biological sample from the patient to detect the genotype of (G/G) for rs3853839.

2. A method for selecting a cancer patient for a therapy comprising administration of an effective amount of cetuximab, the method comprising performing the method of claim 1, and selecting the patient for the therapy if the genotype of (G/G) for rs3853839 is present in the sample.

3. A method for classifying a cancer patient as eligible for a therapy comprising administration of an effective amount of cetuximab, the method comprising performing the method of claim 1, and classifying the patient as eligible for the therapy if the genotype of (G/G) for rs3853839 is present in the sample.

4. A method for identifying whether a cancer patient is likely to experience a relatively longer or shorter progression free survival following a therapy comprising administration of an effective amount of cetuximab, the method comprising performing the method of claim 1, and identifying that the patient is likely to experience a longer progression free survival if the genotype of (G/G) for rs3853839 is present in the sample, relative to a corresponding cancer patient not having the genotype.

5. A method for treating a cancer patient selected for treatment based on the presence of the genotype of (G/G) for rs3853839 in a biological sample from the patient, comprising administering to the patient a therapy comprising a therapeutically effective amount of cetuximab or an equivalent thereof, and wherein the patient was selected by the method of claim 2.

6. A method for increasing the progression-free and/or overall survival of a cancer patient, comprising performing the method of claim 1, and classifying the patient as eligible for a therapy comprising cetuximab if the genotype of (G/G) for rs3853839 is present in the sample or not eligible for a therapy comprising cetuximab if the genotype of (G/G) for rs3853839 is not present in the sample.

7. The method of claim 6, further comprising administering a therapy comprising a therapeutically effective amount of cetuximab, or an equivalent thereof, or an cetuximab-free therapy in accordance with the classification.

8. The method of claim 1, wherein screening comprises contacting the biological sample or nucleic acid isolated from the biological sample with a labeled nucleic acid probe that specifically binds to a nucleic acid having the sequence of SEQ ID NO:1 and overlaps the rs3853839 polymorphic site.

9.-12. (canceled)

13. The method of claim 7, further comprising administering a therapeutically effective amount of folinic acid and/or a pyrimidine analog.

14. The method of claim 7, further comprising administering a therapeutically effective amount of leucovorin and/or fluorouracil (5-FU).

15. The method of claim 7, further comprising administering a therapeutically effective amount of bevacizumab or an equivalent thereof.

16. The method of claim 1, wherein the cancer patient or the patient suspected of having cancer is KRAS and BRAF wild-type.

17. The method of claim 1, wherein the cancer is selected from gastrointestinal cancer, colon cancer, rectal cancer, colorectal cancer, non-metastatic colorectal cancer, or metastatic colorectal cancer.

18.-26. (canceled)

27. An in vitro method of detecting a polymorphism in a cancer patient or a patient suspected of having cancer, the method comprising screening a biological sample from the patient to detect the genotype of (G/T) or (G/G) for rs5743618.

28. A method for selecting a cancer patient for a therapy comprising administration of an effective amount of irinotecan and bevacizumab, comprising performing the method of claim 27, and selecting the patient for the therapy if the genotype of (G/T) or (G/G) for rs5743618 is present in the sample.

29. A method for classifying a cancer patient as eligible for a therapy comprising administration of an effective amount of irinotecan and bevacizumab, comprising performing the method of claim 27, and classifying the patient as eligible for the therapy if the genotype of (G/T) or (G/G) for rs5743618 is present in the sample.

30. A method for identifying whether a cancer patient is likely to experience a relatively longer or shorter progression free survival following a therapy comprising administration of an effective amount of irinotecan and bevacizumab, comprising performing the method of claim 27, and identifying that the patient is likely to experience a longer progression free survival if the genotype of (G/T) or (G/G) for rs5743618 is present in the sample, relative to a corresponding cancer patient not having the genotype.

31. A method for treating a cancer patient selected for treatment based on the presence of the genotype of (G/T) or (G/G) for rs5743618 in a biological sample from the patient, comprising administering to the patient a therapy comprising a therapeutically effective amount of irinotecan and bevacizumab, and wherein the patient was selected by the method of claim 28.

32. A method for increasing the progression-free and/or overall survival of a cancer patient, comprising performing the method of claim 27, and classifying the patient as eligible for a therapy comprising irinotecan and bevacizumab if the genotype of (G/T) or (G/G) for rs5743618 is present in the sample or not eligible for a therapy comprising irinotecan and bevacizumab if the genotype of (G/T) or (G/G) for rs5743618 is not present in the sample.

33. The method of claim 32, further comprising administering a therapy comprising a therapeutically effective amount of irinotecan and bevacizumab or an irinotecan and bevacizumab-free therapy in accordance with the classification.

35.-39. (canceled)

40. The method of claim 28, wherein the irinotecan and bevacizumab therapy further comprises administering a therapeutically effective amount of folinic acid and/or a pyrimidine analog.

41. The method of claim 28, wherein the irinotecan and bevacizumab therapy further comprises administering a therapeutically effective amount of leucovorin and/or fluorouracil (5-FU).

42. The method of claim 28, wherein the cancer patient or the patient suspected of having cancer is KRAS and BRAF wild-type.

43. The method of claim 27, wherein the cancer is selected from gastrointestinal cancer, colon cancer, rectal cancer, colorectal cancer, non-metastatic colorectal cancer or metastatic colorectal cancer.

44.-52. (canceled)


SEQUENCE LISTING (31 sequences)

SEQ ID NO: 1 - DNA, 51 bp, Homo sapiens
tgcttcagtgcttcctgctctttttscttgggcctgcttctgggttccata

SEQ ID NO: 2 - DNA, 20 bp, Artificial Sequence
  Description of Artificial Sequence Synthetic primer
attgcttccgtgtcatccag

SEQ ID NO: 3 - DNA, 20 bp, Artificial Sequence
  Description of Artificial Sequence Synthetic primer
tccctatggaacccagaagc

SEQ ID NO: 4 - DNA, 51 bp, Homo sapiens
gctgtgactgtgacctccctctgcakctacttggatctgccctggtatctc

SEQ ID NO: 5 - DNA, 21 bp, Artificial Sequence
  Description of Artificial Sequence Synthetic primer
tggcacaccatcctgagatac

SEQ ID NO: 6 - DNA, 23 bp, Artificial Sequence
  Description of Artificial Sequence Synthetic primer
acccggaaagttatagaggaacc

SEQ ID NO: 7 - DNA, 21 bp, Artificial Sequence
  Description of Artificial Sequence Synthetic primer
tggcctgagaaacagaaggac

SEQ ID NO: 8 - DNA, 21 bp, Artificial Sequence
  Description of Artificial Sequence Synthetic primer
cccgccatttgtattctcttc

SEQ ID NO: 9 - DNA, 23 bp, Artificial Sequence
  Description of Artificial Sequence Synthetic primer
ccttgaggaacttgagattgatg

SEQ ID NO: 10 - DNA, 21 bp, Artificial Sequence
  Description of Artificial Sequence Synthetic primer
ccaaacattccacggaacttg

SEQ ID NO: 11 - DNA, 21 bp, Artificial Sequence
  Description of Artificial Sequence Synthetic primer
atggttctggagtctgggaag

SEQ ID NO: 12 - DNA, 23 bp, Artificial Sequence
  Description of Artificial Sequence Synthetic primer
ccaagggagcagtttattgtgag

SEQ ID NO: 13 - DNA, 23 bp, Artificial Sequence
  Description of Artificial Sequence Synthetic primer
ccttcgtcatgagacctactttg

SEQ ID NO: 14 - DNA, 20 bp, Artificial Sequence
  Description of Artificial Sequence Synthetic primer
ctcatgcaccaagcacattc

SEQ ID NO: 15 - DNA, 21 bp, Artificial Sequence
  Description of Artificial Sequence Synthetic primer
cccaggcagaatcatgttcac

SEQ ID NO: 16 - DNA, 21 bp, Artificial Sequence
  Description of Artificial Sequence Synthetic primer
ttggatctgccctggtatctc

SEQ ID NO: 17 - DNA, 26 bp, Artificial Sequence
  Description of Artificial Sequence Synthetic primer
gctaagatgagagtcaagacagagac

SEQ ID NO: 18 - DNA, 25 bp, Artificial Sequence
  Description of Artificial Sequence Synthetic primer
gctgagttaattctgacaaaaggac

SEQ ID NO: 19 - DNA, 25 bp, Artificial Sequence
  Description of Artificial Sequence Synthetic primer
tcctcaaattagacaaggaacagag

SEQ ID NO: 20 - DNA, 22 bp, Artificial Sequence
  Description of Artificial Sequence Synthetic primer
agaagcctaggccttaaaggtg

SEQ ID NO: 21 - DNA, 20 bp, Artificial Sequence
  Description of Artificial Sequence Synthetic primer
aagaatggacccctgccttc

SEQ ID NO: 22 - DNA, 22 bp, Artificial Sequence
  Description of Artificial Sequence Synthetic primer
gccttcatcattagcccttacc

SEQ ID NO: 23 - DNA, 122 bp, Artificial Sequence
  Description of Artificial Sequence Synthetic polynucleotide
attgcttccgtgtcatccagggccccattctgtgcagattgagtgtgggcaccacacaggtggttgctgcttcagtgcttcctgctctttttscttgggcctgcttctgggttccataggga

SEQ ID NO: 24 - DNA, 51 bp, Homo sapiens
tgcttcagtgcttcctgctctttttccttgggcctgcttctgggttccata

SEQ ID NO: 25 - DNA, 51 bp, Homo sapiens
tgcttcagtgcttcctgctctttttgcttgggcctgcttctgggttccata

SEQ ID NO: 26 - DNA, 51 bp, Homo sapiens
gctgtgactgtgacctccctctgcagctacttggatctgccctggtatctc

SEQ ID NO: 27 - DNA, 51 bp, Homo sapiens
gctgtgactgtgacctccctctgcatctacttggatctgccctggtatctc

SEQ ID NO: 28 - DNA, 122 bp, Artificial Sequence
  Description of Artificial Sequence Synthetic polynucleotide
attgcttccgtgtcatccagggccccattctgtgcagattgagtgtgggcaccacacaggtggttgctgcttcagtgcttcctgctctttttgcttgggcctgcttctgggttccataggga

SEQ ID NO: 29 - DNA, 122 bp, Artificial Sequence
  Description of Artificial Sequence Synthetic polynucleotide
attgcttccgtgtcatccagggccccattctgtgcagattgagtgtgggcaccacacaggtggttgctgcttcagtgcttcctgctctttttccttgggcctgcttctgggttccataggga

SEQ ID NO: 30 - DNA, 67341 bp, Homo sapiens
gtgttatttattattcctaaattaaaatctaaaccatttaataacaccaaatgttgatcagaatgtgggtatgattgctcgtacatatggctaaatatgtagtcataaatatgtatcaaccagtaaactttccttagaagttgatttttaaaaaaaaacccaaattgtggaactatacttattttattttaaactatctcatccagctaaaataatgctcaaaattgcttgataagggaaactgcaattattcttgaaatgttaaatatggtgataatagaaaacaggctgtttaaaataactttccactcaggtgacaaatgtaataaccaggctgcaaactcttacagagaaatattaccttagaaaaatatttttcttcacattgaattgtttgtaagttgcttcctcacacgaattagaaaattggattactggttttgtctgtagcccagaaaaaactgctaactcacctccggacctttgaaaggtaaaaatatacgtaggagtaattccagtgtgggccaacagatggcaccaatctgtttctaaggtaccgctttactcaactttaagaacttgtgtttgtcaagcattttcaattgtatttccgttcatttacaagttattttctcttcttctgaaaaagagatcttggtaagtagaaagacttctgtggctttttcacattatggcacaggtgcttcaagtaagcaagtttgctaaccattcattatataggaaataggtaatgtttccttgaaatttttgcaaaatgatttgaagtggttttttttgtagatgttgcaaatgtcctttgaatttcacgagcaggatgaagagcagcgtctttacagctgtcttgaaaagatagaaaacaactagcgcttgtggtgataattaaggaacaaaactaatgttatttatttcttctgatttggcccagcgaattttctcttttcgtctttctctagtttattgttttcagtaacatttaacagtaatttttatataccactgatatggcacctatttattatatgtttttacagcagacgtgtgtttgatctcccttgtcgtttgtcactgtaacagtttgctggtctgtgtctgtccctaattcataagctttttgaaagcaggaattatgtcagtcctattcatatttatatccctggtgcctagtgcgtagcatggcactggcagaaaaatagactctgaataaacatttattaaatggaggaagtggccgggaccaagccccttcacaagcttgaatctggcacagtgtagcaattgccttcctccaggtccttccaccttcagtttgacaaatgaaacaatgtttttcttgcctatttgttaacttgattatgtgatctttcctccacttatttcacttaattttttttctgctctcttcttccttgtagttttaattttcataggtcttgtctaccctttttttctgaaatgtagcttatttcatctatttcctcaccctttttatttgatgcatttaaaaaatcaagagcatttacctgtttatttttactttctttaaaaaactgtgaactctctaatcttccactctcatcttcccatttttccctccttttttcccaataaatagaaaagttactatcttgttcatttagtgtattccctaggaactgcctcagggtatgaacgcctctggaggaatattgcgtttttgtggcaggataccataaggacagctgaccagcttttccacctggtgactgattttcagtcccatgaacccagacaccgtgggtctggttgccggggacctgtagggtaggtcctcatctgtgaatgccagttatctactattatctgaaagttctccatctttatattttaatattccaaataaaacaagcacgtttctttaaatataaaatattttcacacagctataaagacaactcttaaattgccttacgataacttttttggattataaaggacattcaaaatgagacattttagggatatctgaaagtataagaaagaaaattaaattcatctataattcagagattcagagaaccactattattagtatttggtatatttcatcacattgtttgtcctctagcttttacgtatatgtcaggttttaatcatattttgcaaaattagagtcttactacatatataattttttttttttttttttgagacggagtcttgctctgtcgcccaggctggagtgcagcggcacgatctcggctcactgcaagctccgcctcccgggttcacgccattctcctgcctcagcctcccgagtagctgggactacaggcgcccgccaccacgcccggctaatttttttgtatttttttttagtagagacggggtttcaccgttagccaggatggtctcgatctcctgacctcgtgatccacccgccttggcctcccaaagtgctgggatgagaggcgtgagccactgcgcccggccataattttctattactgtattcatttaacattatcattttttcaaagtacagtatttgaggctattatttaattatacataactgatttaatcattttctacactagacattctgattttttcaatatttcattatgtcatgagtatccttatagtcatatcttctgtgtgttatttttgactatttctttagaaaattccttgtagtggaattgctaaaataaaggaaggtatatttttaagaaatttatgtaaattaccaaattaccctttagaaagcttgtactaatttttcattcctgcctaagtagtaaatgagggttcttattttagtttccacctaccaacctggatattatattttttaattttccctatttgataggtgaaaaaaaaatcttgccttttaaaaaatcttttaagaaacaagagtcctcctttgacccccttcctctttttatgcttaaatatcttatagtaaaaataacaggcattctggaaacctatttctaaaagctttattctctatctctaaaaaaattacattttcctaaaatagccaaataacattataaaaaattacaatttatttcttaatagcatctaatggctggtccatcatagaatctgcccctccttctgttactgaatatgttttatagcaagtctgtcttaactgagatctaatccaagatcatgagtttcatctagatgttatgtgcttttttgatttaacgtgctgttttattttttgtgagattgcacattattcatattattggcaattgatttttggcaaattattcagtaaatagtacagtaaacttaattttgaatgttaaaagtttgaaaaatatagataaaaactaagaggaaaataaagttccattataatcttactgtgcaaagataattactgttcatatcttggtatgtatcttattaatttttctacttgcataggtacatatttactttggtactttttaaataaactgctttttgtcacatatagtgaaaatattttcatgcaattcgatttttattatttttaaaggctacatagtactatttaattatacagagatactaaaatttatttgataacctgctatttttggataattgagggatttgttttcaaaatagaatttatttttgccaggagcagtggcttgtacctgtaaccccagctccgtgggaggctgaggtgagaggatggcatgagcccaggaccttaagctatgattgtgccactgcactccagcctgggcagtgggacaaaaccctgtctcttaaaaagaaagtgtttaagaaaatagtaatgataataatttattttgtgacggccactttattgtagtcttttattggtgctaacattttttcaatgacttctctttggctttccacattgacagtcttataatctaaaaataataatgattttctttcttcatttcagtatttcttcctgtcattttttttctttattacagtggttaaaacttccggaaagaacaatgtgtgcattcttttttttttattattatactttaagttctagggtacatgtgcacaacatgcaggtttgttatatatgtatacatgtgccatgttggtgtgctgcacccattaactcgtcatttacattacgtatatctcctaatgctttccctcccccctccccccaccccacgacaggccccagtgtgttatgttccctttcctgtgtccaagagttctcattgttcatttcccacctatgagtgagaacatgcggtgtttggttttttgtccttgcgatagtttacgagaatgatggtttccagcttcatccatgtccctacaaaggacatgaactcatccttttttatggctgcatagtattccatggtgtatatgtgccacattttcttaatccagtctatcattgatggacatttgggttggttccaagtctttgctattgtgaatagtgccacaataaacatgtgtgtgcatgtatctttatagcagcatgatttataatcctttgggtatatacccagtaatgggatagctgggtcaaatcgtatttctagttctagatccctgaggaatcaccacactgtcttccacaatggttgaactagtttacagtcccaccaacagtgtaaaagtgttcctatttctccacgtcctctccagcacctgttgtttcctgactttttaatgatcgccattctaactggtgtgagacggtatctcattgtggttttgatttgcatttctctgatggccagtgatgatgagcattttttcatgtgtcttttggctgcataaatgtcttcttttgagaagtgtctgttcatatccttcgcccactttttgatggggttgtttatttttttcttgtaaatttgtttgagttcattgtagattctggatattagccctttgttggatgagtagattgcaaaaattttctcccattctgtaggttgcctattcattctgatggtagtttcttttgctgtgcagaagctctttagtttaattagatcccatttgtcaattttgtcttttgttgccattgcttttggtgttttagacttgaagtccttgcccatgcctatgtcctgaatggtattgcctaggttttcttctagagtttttatggttttaagtctaacatttaagtctttaatccatcttgaattaatgtttgtataaggtgtaaggaagggatccagtttcagctttctccatatggctagccagttttcccagcaccatttattaaatagggaatcctttccctatttcttgtttttgtcaggcttgtcaaagatcagatggttgtagatgtgtggtattacttctgagagctctgttctgttccattgatctatatctctgttttggtaccagtaccatgctgttttggttactgtagccttgtagtatagtttgaaattaggtagcatgatgcctccagctttgttcttttggcttaggattgacttggcaatatgggctcttttttgaccccatatgaactttaaagtagttttttccagttctgtgaagaaagtcattggtagcttgatggggatggcattgaatctataaattactttgggcagtatggccattttcatgatattgattcttcctatccatgagcatggaatgtttgtttgtgtcctcttttatttcattgagcagtggtttgtagttctccttgaagaggtccttcacatcccttgtaagttggattcctaggtattttattctctttgaagcaattgtgaatgggagtccactcatgatttggctctctgtttgtctgttattgatgtataagaatgcttgtgatttttgtacattgattttgtatcctgagactttgctgaagttgcttatcagcttaaggagattttgggctgagacgatggggttttctaaatatactatcatgtcatctgcaaagagggacaatttgacttcctcttttcctaattgaataccctttatttctttcttttgcctgattgccctggccagaacttccaacactatgttgaatatgaataggagtggtgagagggggcatctctgtcatgtgccagttttcaaagggaatgcttccagtttttgcccattcagtatgctattggctgtgggtttgtcataaatagctcttattattttgagatacgtcccatcaatacctaatttattgagaatttttagcatgaagagctgttgaattttgtcaaaggccttttctgcatctattgagataatcatgtggtttttgtctttggttctgtttatatgctggattacatttattgatttgcgtatgttgaaacagccttgcatcccagggatgaagcccacttgatcatggtggataagctttttgatgtgctgctggattcagtttgccagtattttattgaggatttttgtattaatgttcatcagggatattggtctaaaattctctttttttgttgtgtctctgccaggctttggtatcaggatgatgctggcctcataaaatgagttaaggaggattccctctttttctattgattggaatagtttcagaaggaatggtaccagctcctagttgtacctctggtagaattcagctgtgaatccgtctggtcctggactttttttggttggtaggctattaattattgcctcaatttcagagcctgttattggcctattcagggattcaacttcttcctggtttagtcttgggagagtgtaggtgtcgaggaatttattcacttcttctagattttctagtttatttgcatagaggtgtttatagtattatctgatggtagtttgtatttctgtgggatcagtggtgatatcccctttatcattttttattgcgttgatttgatatttctctcttttcttctttattagtcttgctagaggtctatcaattttgttgatcttttcaaaaacccacctcctggattcattgattttttgaagggtgttttgtgtctctatctccttcagttctgctctgatcttagttatttcttgccttctgctagcttttcaatatgtttgctcttgcttctctagttcttttaattgtgatgttagggtgtcaattttagatctttcctgctttctcttgtgggcatttagtgctataaatttccctctacacactgctttaactgtgtccctgagattgtggtatgttgtgtctttgtcctcgctggtttcaaggaacatctttatttctgccttcatttctttatgaacccagtagtcattcaggagcaggttgttcagtttccatgtagttgagtggttttgagtgagtttcttaatcctgagttctagtttgattgcaccatggtctgagagacagtttgttataatttctgttcttttacatttgctgtggagtgctttacttctaactatgttatcaataagtgcagtgtggtgctgagaaaaatatatattctgttgatttggggtggagagttctgtagatgtctattaggttcgcttggtgcagagctgagttcaattcctggatatccttgttaactttctgtctcgttgatctgtctaatgttgacagtggggtgttaaagtctcccattattattgtgtgggagtctaagtctctttgtattgggtgcatatatatttaggatagttagctcttcttgttgaattgatccctttaccattatgcaatggccttctttgtctcttttgatttttgttagtttatagtttgttttatcagagactaggattgcaacccctgcctttttttgttttccatttgcttggtagatcttcctccatccctttattttgagcctatgtgtgtctctgcacgtgagatgggtctcctgaatacagcacactggtgggtcttgactctttatccaatttgccagtctgtgtcttttaattggagcatttagcccatttacatttaaggttaatattattatgtgtgaatttgatctgtcattatgatgttagctggttattctgctcgttagttgatgcagtttcttcctagcatcaatggtctttacattttggcatgttgttgcagtggctggtaccggttcttcctttccatgtttagtgcttccttcaggagctcttttagggcaggcctggtggtgacaaaatctctcagcatttgcttgtctgtaaaggattttatttctccttcacttatgaagcttagtttggctggatatgaaattctgggttgaaaattcttttctttaagaacgttgaatattggcccccactctcttctggcttgtagagtttctgccgagagatcagctgttagtctgatgggcttccctttgtgggtaacccaacctttctctctggctgcccttaacattttttccttcattcaactttggcgaatctgacaattatgtgtcttggagttgctcttctggaggagtatctttgtggtgttctctgtatttcctgaatttgagtgttggcctgccttgctaggttggtgaatttctcctggataacatcctgcagaatgttttccaacatggttccattctccccgtcaatttcaggcacaccaatcagacatagatttggccttttcacatagtcccatatttcttggaggctttgtttgtttctttttactctttttcctctaaacttctcttctcacttcatttcattcatttgatcatcaatcactgatactctttcttccagttgatcaaatcagctactgaaacttgtgcattcgtcacatagttctagtgccatggttttcagctccatcaggtcctttaaggacttctctacactgcttattctagctagccattcgtctaatcttttgtcaaggtttttagcttctttgcaatgggttcgaacttcctcctttagttggagaagtttgatcgtctgaagccatctctcaacatgtcaaagtcattctctctccagctttgttccgttgctggcgaggagctgcgttcctttggagtgggagaggtgctctgatttttagaattttcagcttttctgctctgttttttccccatctttgtggttttatccacctttggtctttgatgatggtgacgtacagatgggtttttggtgtggatgtcctttctgtttgttagttttccttgtaacagtcaggaccctcagctgcaggtctgttggagtttgctggaggtccactccagaccctgtttgactgggtatcagcagcggaggctgcagaacagcgaatattgctgaacagcaaatgttgctgcctgatcgttcctctggaagctttgtctcagaggggtacctggccatgtgaggtatcagtctgcccctactggggggtgcctcccagttaggctacatgggggtcagggacccacttgaggaggcagtctgtccattcccagatctcaaactctgtgctgggagaaccactactctcttcaaagctgtcagacagggacatttaagtctgcagaggtttctgctgccttttgttgggctatgccctgcccccagaggtggagtctacagaggcaggcaggcctccttgagctgtggtgggctccacccagtttgagcttcctggctgctttgtttacctactcaagcctcagcaatggctggcgcccctcccccagcctcactgccaccttgcagtttgagctcagactgctgtgctagcaatgagcgaggctccgtgggtgtgggaccctccaagccaggtgcgggatataatctcctggtatgccgtttgctaagaccgttggaaaagcacggtattagggtgggagtgacccaattttccaggtgccatctgtcaccccttcctttggctaggaaagggaattcctgaccccttgtgcttcccgggtgaggcaatgcctcaccgtgcttcggctcacactcattgagctgcacccactgtcctgcccccactgtccgacgggccccagtgagatgaacccggtacctcagctggaaatgcagaaatcacccatcttctgcatcacttatgctgagagctatagactggagctgttcctatttggccatcttggaaccaccccccctaatgtgtgcattcttgatgttgactttaacaggaatgccttggggtggaatattgattaaagttagatatttgaggtattctttataaagttttcaaagtattttttaaatacctatagttgtatttaatcaaagatgaggactgaattttatcaagtgaatttttcacctttatagagatgctcatctatttttcttgtttgacttactcaattgaattattgttatagacttgttaatgatagaccatttctacaatctcagaaacatcctaattactcattatattttttcttcaggacacagctagattgattggggtttctttattctgagctctatcccttccccataaatgactgaaatttatcccttttatttgtattttctcataattactcttaattttataatatatatacttacaaatatttttctaacaacatcaggagttaattggcatctatagcttcctttcaattagacaactatcttacatggttttagtgcactctctccctccctaccccaccttcagccaactcccttctcccttcttatcattggcagtttttagctcgagatttctattaagatttttgtcatcaatactttttggtacttagcactaaattttactacttttctttgctaaataatccttcttgtattttattattttagagttaatttctttttaaactctccatcctctgggaattcagatatggatctgtcttccatgtcttttagcattttagtatgttttctgtctctttggcctcccctggtgagtttgggaaaattctccttggtcttctctatcattaattttctcattagtggtgctatttgcaactcaagtcatccagtgaatttttcttttcaataattgtctataatttccatgattcttactctatttttaaaagaatctctcctctttctgaagatatctattataaatgcctattctgctcttttatttctgtttagttgagtattaacttctttcatatcttgagtttggctcctttctattatggtacttgctttctgcagtatcatgcttcttgattgtgcaactgtgtttaagccttcttgtgagagtgcttgttgatctattttcatagcctgcttgttgggatgctggatcaaaccagtctctactaattttcttttcttttctttttttttttttttttttgagatgtctcgctctgtggcccagcctggagtacagcggcacaatctcagctcactgcaacctccacctcccgggttcaagtgattctcctgcctcagcctcctgaatagctgggactgcaggcacccactgccacacccggctaatttttgtatttttaatggagacagggtttcaccatgttggccaggctggtctcgaacttctgacctgaggcaatccatccacctcggcctcccaaagtgctaggattacagaggtaagccaccatgcctggccccagtctctactaattttctgttgatgctgtaacaaattaccacaaacgttgtggcttaaaacaatatacatttattatcttacagttctgtagggtaggaatctgacacaggtcttgttggctaaaatcaagggactggcaaggctgcattctttctggaggctctaggagggaatccaggtacttgccttttcaaacttctagaggctgctcacatttcttgactcatagtccccttcctttatcttcagggccagcaaggatgggccaagttcttctcatatcacatcaccctgacatcctctctgccttcctcttccacttttaagggtgcttgtgattatactggggccacccaagtaattcaggataatcttcctattttaagatcagttgattagcaactataatttcatccacaacattaattttcctttgccatgtaacatagcatattcaccaagttgggggattagaagtagacatctttgggaggccagtattctgtcataggtgagtccctttctggatttcagaggcttctcggtttcagtcccctaaaggtccccttgccttttagtgtgttatggatctatcaatcgatcaatatagataaaactttaatagatagatagagatataacttaatatttttcaaaatatttggtcatttcaggaatttaaggcaagaagaggaagctggaacacatacaagccacccttttgactatcataatttgtaatgtttgtatttgaaaatgtggttttgtcacccaggctggagtgcagtggcacaatcttagctcactgcaacctccgcctcccaggttcgaacgattctcttgcctcagcccccaaagtagctgggaatacaggtgtgtaccaccacgcctggctaattttttttttgtttgtttagtagagacagggtttcaccatgttggcaaggctggtctcgaactccttacctcaggagatccacccaccttggcctcccaaagtgctgggattacaggtatgagccaccaggccaggtctcttagaacatttttaaatgtgattttaaaagttccttttatagtcagaaatatgacttttataatttctgttttgggatacttaatttaagatgtatgtatttgacctaaaaaacaattgattttagtaaatgctctatgaactctagagacttagacatatgatgttctctttttgtagggtacagcttatgtctctattaaatcctttattaatttctcattcaaatttccatatatgtttttattttttgctacttgatctaacagtgacccagagaaatatgctaaatacttttactcacaatgtctccatgtgttttgtaacattttttactttgtaaattttgatgcaatattacttgctgcataactgttcttatctgttacatcttcattgtgaactatttgttttgaccatataaccagtataaagtagtagcttctcctatcgttgcccaactctctagtactttgaccgatatatactttgtatatattgtgtataaaaactgcaattcctgatgaacaccgctaccagtattccagtcgctgtccatatcggtaagtattgtctgagttacacatctcagaatgcggtgtgctccctaaagtggttccctgccccatgtccagctcttacctggtagtgtccatcactaagtgtaaagtttggggagaatggagtgtggcagtccgtagaaggaaaactcagcatgctgatccactcatgtcagtaccattggggtaaaagggacactgcctagctactcattaagtactagttggaactcatcctcactgtttgaatcagagacagagagaggacaaatttctttctgcctttcatcagggagtctattttcctgagacaaagcttgagtgtggcaggtttgccctaacacagttccagttacatttttgtgagtgaagatgctcctggatcctgccattacatttctgtgcttctttgttttcagtggccaagtggatttttcaaatgtatgtgtgtctctgtgggtgttctgatgggcagttgggagaagttatatggatcagtgaagagcttttaaccatacgccattcagtaataaaagttccccaactcagagttttcactcttccaaagaaaggttggttgaaactctactcatctcttgcctgttttactatttccaatatatcctctgatgacgaaaagtggatcctttgtaaataaaggactcatctctttcctgttttactatttccaatatataccctgatggtgaaaagtggaccctttgtaaataaaggattcatctcttgactgttttactatttccagtatatcccctgatggtgaaaagtgggtcctttttaaataaaactgagcaatgtgcagtgctcattttggtaatctgctctcaattatccctgcaacacagagacctactttaaaaatgttggtggctgcttttgtcccccaggagctcatagatgctttcttgtgcagaacagagcctgaaaaggttctgagccagcctggcagttccagtcctttgtctcagtcatgaacatggggtttgtgggctcatgcacctgccgccctgcctcagtcaagtattcttttcttgtttgctcagcatcattgtcatgaacgtcaatgtctataatgtgttgtaatgcaacgaggctttctgggccctgagaaagtagttatctcttccatacggcagctttagcctggcaaactttttcattttgtcagtgtgttagctgttagaacttgatgttttcagattatcatgtgctgaatcctgtctctctaaatttgtggcttgcagtctaatgttggttggaaaaatacccattgtaatgggaattgtgtcaacaatgagattctataactaaaacactcctgggattaaaaaaaaaaagcacagtatatgtgtcattgctatggcccgaatgtttgtgtctcttcgaaattcatgtgttgaaacctaattaccaacgtgatggtattagatgtgtgagcctctggaaggtgattaggtcatgaggacaggcggccaggatgcccctgtcggagctgggtggccatggagacttgtctggccttgtgtgctggcacacagaggcattctggctttgccccccatgggggctcacagatgcttttctgtgcggatcagagcctaaggagagtagactcctaagcatatgtctgagtttctgagactggctccttgctttccctgtagaataattttagtcccacccctctttcccagtcacaaacatgggatctgtgggcttgtgtgcctgctaccagcccccagcaaggcacaaatgaccactttggcctgggttaattgcattttctcactggctccatgaatggtgatatcatttctgccaggccacttatggctagttttggtcaggtccaattcatgaaggtggagggagtaggtagttcctccttaagatataaatatagcctcattccagtcctcatctgccctcccaaaaggattgatccctctcatattccacagggctttggtcatcacattgcaggcagatccacctcctggccaggcctcaatgtcctgccaaccaccttcctccccttccctttcttttgatgtcaccttgcccaaattgggttggggatcagaaagtcgggtttgggtcagaatcctgaaagatgcaatcccaagtgccataatcctgaatgttgaaatcctggaagatcaaaatcctgaaaatataattctgaaaaaaaaatttttaaacattctttaaaagacattcatttacatttttgaaaggcgatttatttgagaaacatatgaaaacatgacaatattttataaatcactttacaaataaaataaacaataatgacgtacatatttttgcaagcatgaacactcagatatgctaaggacagttgcatgcgtaaacactcaggaacagatgaaccgtactcacaaataaatggcttacgtaactgctgccatccacactgtcaaaccatgatgggcaacctaagtcctttgatgcgattgatcaaaaactgtgatgggtcaccaccgcatatatatagttgcccagagagctgagagcttgagaaattttatctttcacaaatgcagatgggcaaaaaggacttctgttcatttattgagaaagtttcccttttttcttctctctttctctctctctctccttttaatctctgctcactgcaactacctctgtctcctgggctcaagccatcctccacttcagcctccctagtagctgggactacaggcaggcaccactatgcctggataatttttgtattttttgtagagacagggtttcactgtgttgcccgggttggtctcaaaccccagagctcaagccatctgcccgctgtggcctcccaaagtgctgggattacagagatgagcctctgctcccggcagaagtttcagtgttttcacatacacacacaatgcttgcacacaaagtcaatgttgtgataatgtacttttatggagtcaacgttgcataaaataaatcagaactctctaaatagtctttacaaaatttatacctccagtattggaaatgatgtgaagatgaaatacatagcatagcaaattgtaaaaaatagtgttgatgatttaaaataatgggacaaaggaaaagaaaagcaaaaaaattaaccatccaccccaccccccaaaaaaaattaagaagaaaaagtatatcacagggacagattatgggcaattgcatggaggtagtccataagagctggccaactttcaccattattaatattttaaagtcttgcatcacaacaaatagctgatttatttctttcagggcactgttctcctcagagaatacattcacatttattttcgatgtggcactgcttgtttggaaatgcttctctgcttctatttacatatttacaccaacatgagcattcctgttaaatgttctcatcttcagtgccgtgcttctgtgttgatttgggtacatgaaaatctattctgcatgcactcatatacagaccacacatttggcagaaacaatactggtgatcaatcaacaacagcattgtgtgtcttcttttttttttttttttgagagggagtcttgccctgtcacccaggctggagtgtaatggcgcgatctcggctcattgcaacctccacctcccgggtttaagcaattctcctgcctcagcctcccaagtagctgggattacaggaacccaccaccacgcccagctaattcttgtatttttttagtagagacggggtttcaccatattggccaggcaggtctcgaactcctgacctcgtgatccatgggcctcagcctcccaaagtgctggaattacaggtgtgagccaccacgccagtcctgtgtcttcttatcctaccatatgcattattattttcaaatcagtcagtaacttcactgggtgctttaggcaaatgcaactttaattcattgagaactcctggtatttcatcagccagaaggaatgccagtgcagacaaataatgtatttttaaactaaatttttcatcattgccacattatgtggccaatcctctcaactgaattttctgacaaatgcattgggctgaatggaagaaaacaaactttattggtaacaccttgaaattcccttttagaagccttgatcacacctaattccaaatcaagtgataggtcattatgatttgaggattcagttggacattagggattttagactttggggactttgatcttttaggatttcagccttcaggattgtgtctttcgggattataatcagcactgctgaaagtctagctcagcactggcaccactgtcgagtgcccatcggaggtttctcaggctcaactaaacattcatacccaaatgccccaataggccacactcagaagctcaattttctacctggtacttatagttcatacacagtcctttcatcctggatgcaatttaccagtcagggatcattgttacaataagtaatgttggctggtacacagctgactttctacatgtttcaggttttcagaatagggctaggaagttaacttgaggtcagatcagtccatggagaaagagagaaatggatttgttaacccttcagccgtgaagggtcactggttgcagcagatggctgagttggtgccaaactggatactggtataaactactgagtatgtcttggagtttttgggactggcttcttgcctgcttttttaaattatcaaaccggctggtctaatatttggtggtggggaaggataaaagaacttcttctttaaaagagaataactattttttgagtacagcacgtagttaactttcaatacagttgatttaaagcctttgcctagtaagtccaatgtctatgctttttccatttctattaattttttttaaaaaaaaatcaatagactttattttttagagcagtttttttttgtctatttagttttgcctttcctggaatatcatatagttgaaataaaatagtatttagctttttcagactgacttctttcacctagcaaaatggatttaaggatcttcccccccttccctcccctccctcccttccctcccttcctcccctccctttgcctcccctccctcccttccttccttcctctttctctctttctatttctttctctctttttctttctttctctctctttcttaactttctttctctctctccttctctctctccccctctctccctcctttctttccttcctccccttccttccttccttcctcccttccttccttccttccctccctccctcccttccttccttctctttttctctttctttcttctttccttccccacaaaataattatacatatttatggggtaactacaatatttgacacatacataccgtgtgtcaaatcagggtatttaggatatccaccatctcaaacatacatcattttgtgtgtgggtgggggagcatttcagatcttctcttctagctgttttgaaatatacaataaattattgttagctatagtcaccctactgtgttattgaacactagaactttttctttttatctaactgtatgtttttatccattaaccaacctctcttcattccctcactcacctcttagtctctggtaactattgttctactctctaccctcctgagatgcgtattttagctcccacagatgagtgagaacacaatatttgcctttctgtgcctggcttattttgctaatgtaatgacctccaggtccatccatattgctgcaaatgacaggatttaattattttaaatggctgaatggtattccatcgggtatatatatcacattttatttatccattcatctgttgatggactgttaagttgattccatattttggctattgtggatagtgctgcaatgaacatggaggtgcaggtatccctttgatacactgatttctgttcttttagataaacatccagaagtggggttgctggattgtatgttagttctatttttagttttttgagacaactctgtactgttctccataatggctgtactaatttacattcccaccaacagtgtataagagttcccttttctctgcattctcaccagcatttgctatttattgtctttttgatgacagccatttaaactagaatgagatgatatctcgttgtggttttgatttgcacttccttgatgattcatggtgagcatttttttcatataactcttggccatttgtacgtcttcttttgagaaatgtctattcaaatcctatgcctactttttcataggattattattatttttgctgttgagttgtttgagttctttgtatattgtggctattagtctttattggatgaatagtttgcaaatattttcgcccatttaaccctgttatctattcaccctgttgattgtttcctgtgctgtgtagaagtttttagtttaatgtagtcccatttgtctattcatattttcattgcctgtacttttgaaattttagccataagatctttacctagaccagtgtcctcaagcatttcttctgttttctcttagtagttttatactttcaagtcttacatttaagtctgtaatccattttgaattgatttttgtatatggtgagagataggggtctagtttcattcttacaggggtctagtttcattcttatacatatgagtgaatatgaagccagcaccatttattgaagagagtggtctttccccagtgtatgttgttggcatctttgttgaaaattgattggctgtaaatatgtggattaatttctgggttctgtattctgttctattggtctatgtatctgtttttgtaccaataccatgctacttttgctttgcagtatcgtttgaagtcaagtagtgtgatcccttcagctttgccccttttcctcagtactgatttggctatttggggtgttttctggttccatatgaattttgagattattttttatatttctgtaaataatatcattggtattttgatagggattgcattggatctgtagattgctttgagtagtgtggtcattttaacaatgttaattcttccagtccatgaacatgggatgtctttccatttttttgtgttctcaatttctttcatcagtattttgtagtttttgttgtaggggccttttgccttcttgattaagtttattcctaggtattgtaattgtcttgtagctatgcaaataagattgctttcttgatttcttttttctttttttttttttttgagactgagtctcactctgtcacccagtctggagtgcagtggcacaatctcagctcactacaacctctgcctcccaggttcaagtgattctcatgcctcggcctcctgagtagctgggattccaggtgtctgccaacacacctggctaattttttgtatttttagtagagataaggtttcaccatgttggccaggcttgtcttgaactcctgacctcaggtgatctgcccacctcagcctcccaaagttctgggattacaggtgtgagtcaccacacccagtccttgtctggttttggtatcaggaaaatgctggctttgtagaatgagttaggaggaataccttcatctttaatttttttgaatagtttgagaaaactttgtgttaattgtttgttaaaaattcagcagtaaagccatccagtcctgagattttctttgttgggaaatgttttattactgattcaaacttattacttgttattggtctgttcagatttttctatttcttccaggctcaaatttggtaggttgtatatatccagaaatttatacatttcctgtagggtttccaatttgttaacatataggtgttcataattgcctccaatgatcttttatatttctgtggtatcagttgtaatgtctccttttttatttctgattttatttattttggtcttctttcttttcttgtcttggttagtctagctaaaagtttattggttttgtttatcttttcaaaaaataatttttttgttttattgatcttttctatttttaaatatctcttttgtttagttctctgagctttagtatttctttctttctcctaacatttgaaagtgtttgttcttgcttttctatttgttcttgcttccttgaggttcgttgttaggttgtttatttcaaatctttctacttttttgatgtaggcatttattgctataaatttccatcttagcattgtttttgctgtgtctcctaggctttggtgtgctgtatttgcattttaatttttttcaagaaacttttttacctcctatttaatttcttcattcacccagtggttgttcaggagcatgttgtttaatttccatgtatttatacagtttcaaaagttcatcttgttattgatttgtagttttattccattgtgatctgagaaaatacttgatatgatttcaatttttaaaacttttgttgaaacttgttttgtggcctaagatatggtctattctgaacaatgttccatgcactgatgagaagaatgtgtactctgcagctatcagatgaaatgttctgtaaatatctgttaggtccattttgtctaaagtgcagtttaaatccaatgtttctttgctgatattctgtctaaatgatctgttcaatgctgagagtgggatgttgaagttcccaaccattattgtattggagtctatctctctcttttgatctaataatatttgctttatatatctggatgttcctgtgtggggcacatatatacttagaattgttatatccttttgatgaattgatccctttaacattatataatggctttttttgtcttttttaatagttttatacttaaagtctgttttatctgataagtatagctattcttgcttgcttttggtttccattagcaaggaatatctttttcctttccttcaatttcagtctatatgtgtctttgaaggtggggtgaatgtcttgtaggcagcatatagttgggtaagtaaaaaaaatctattcagctagtttgtatcttttaagtgaggaagttaatctttttacattcaaggttatcactgataggtaacgacttatttctgtcatttggcttattgttttctggttgttttgtatatcctttgttcctttcttcctctcattgtttatcattgaactttggtggttttctgtagtagtaacttcaactcttttctctttcatttatgtatttgctctactagtgagttttatactttcatgtgttttcataatggtagatataatcttttcacgtccagacgttctactctcttaagcatttcttgtagggctcgtctagtggtgatgaattccctcagttgtcgcttgtctgggaaagaatatttcttcttcattatggaaggacagctttgatgggtatagtcttcttggttgacagtttcttctttcagcactttgagtatattatcccattctctcctggcctatagtttctgctgaaaaatctgttagtctgatggggagtctcttctatatgacttgactttttttatttcttgctgtttttaggattctgtcttagtcttcaacttttgagagtttgactgtgatcttcctcagagagaacatttttgaattaaatctacttgggaatttttaagcttcttgtatctggatgtctatacctcttgtaagacctggaaagtttttagctattatttcattaaacatgttttctatccctttgctcatctcttctgcttctggaactcccaaaatgtgaattttgtttgcttaatggtgttccataggctttctttattcttttttattccttttttttttttttcgcctcactgggttatttcaaaagacctgtcttccagttcagaaattctttattttgctcaatctagtctactgttttttaatttttaattttatatatatattttttttttgagctggagttttgctcttgtcacccaggctggagcatagtggtgcaatctcagctcgctgcaacctctgcctcctgggttcaagcaattctcatgcctcagcctcgcaagtagctgggattacaggtgcctgccaccacgcccaactaatttttgtaatttttttttttttttagtacagatatggtttcaccattttggccaggctggtctcgaactcctgacctcaggagatccacccacctcagcctcccaaagtgctgggattacagttgtgagccactgcacctggcctagtctattgtttgtacaagctcttgattatatttttttacttcatttattgacctctgtagttccaaaatttctgttttgttctatttttaatatctatttctttgttgaatttctgactcagatcataaattatttttctgatttcttttattatttatctatgctctcttgtaactcactgagtttctttagtatcattattttgaattacttttcaggcattttatagattttctttttgttgagatctgttattagagaattattgtgttcctttggaggcttcatgtttccttgcacttttgtatttcttgtgtccttatgttgatatctatggatctgatgtaatagctgctgcttccaattttatggattggctttgatagggaaagacttttgcctctaggtgtttttatagtgttagttgagtatagggtgctttggctttgattctgagtgggcacagcagtgtagtttctgtatgatttcttcacctgtcatcagtgtcagtggcatttgggttcctcagtggcttaggttacagttgtcagcagacactatggcaaggctttgctggggatggggacaccaggcaggcctgttctcaggcaacagtggtggcagcagtggcctaggcttgccggttctcaggcccctgtgtgaaatatgtgggcactcgtggtgatgggtccttgagcctccaggagatttagttggatgcttgcagtggcagtaatggaccaagtgggagggcaggttcttgggcccctgcacaatttgtgtggcattaactgtagcagtaggaaaagtggaccaaccctctggcccccaagtggtagacacaggcagcagcaggctgggcaagccagtcctcaggaccccgtgtggcatgcatgtgccacaccctgtgtggcactgccctgccatggcagtggcaggcaggcactagcagcaaagggcatggtggacctgtcctcaggccccttggaggcatgtgtagatgtgtcgtagtcctgctgctagggaagatggggttgctgtaagtggcggtggccctgggcaggtaactctgaggctctggggaatgcacactttgactccctttgtcccagggaaagccactaggtgcactgtgcttcctgtttcctgggatgcggggcattgcatgggctagagtgctggggacctagctgcactgctgatcttagctggcattacaatgctgtagccctctaggtggacgtaggagaatatcagtggggcttcaggggtgtggagattcagggttgttgggccccagaacaggatgtgtatggtgagggctgggctcttaaaagagccttatgctgaagctgtttgggtctcagggtatggggtatgtgggacccaatgtgaagtcccattgtggaacaatgtgattgcatatattccagacagccccagggcatgtgaaggccaaggagctagctcatggctaggattacaggagtccatagtggaaacattgacctctgaagatctctcagttaaactttccctggaatgggaagttcctcctggttctgagctagtcccagctgggctggctgcttcacttccctcttctcccatgcctcagaggtgcactgtcacttctccgctgaattccagtgttctctcttagatgctcattcaatgtgtgattatctgctcactgttttggtccttctttgcagagggggtgagggctaggtgcctctattcagccattttaatgcttctccggctcattttttaattgttcaatagtattttattgtgtgaatgtaccagtttgttaatatttttatctattgagggatatcttgttgcttccaatgtttgacagttatgaataaagcttctataaacatccacatggaggcttttatgtggacataaattttcagttcttttggttaaatacccaggaatgtgatttgctggattgtgtgataagactatgcttagttttataagaaactgcattccatagtggctgcaccattttgcattcccacaaacagtgaaagagagttcttgttgctccacatcctcaccaacatttgatgttgtttttggattttacccattctcataagtgtaatgtaatttcattgctgtattaatctgcaaaaccatgatgaaatatgatgtcaaatagctatgtatatgtgtatttgctctttacatatcttctttggtgaggtgtctgtttagatttttgtgcccatttaaaaactaggttgtttgttttcttattgtggagcttaagagttctttgcatagtttggatacaagtccctatcatatatgggttttgcaaatatcttctctgagtctgtggtttgtctttttcactctcttgcagtgtcttttgcaaagcaaaaattttaattttaatgaagtccaacttaacagtgttttctttcttggattatgtttttgatgcgtatctcaaaagtcatcaccaaatccaaagtcacctagattttctcctatgttatcttctggaaattttatagttttgcattatacatttaggtctatggttaattttgagctgatttttgtgaaaactgtaaggtctgcgtctaaattctttctcttcttcttctttttttttttttgcatgtggaggtctaattgttccagcaccatttgttgaaaagactatccattctccactgagttgcctttgctcgtttgtcaaacagcagttgattatttttttgtgggtctatttctaagctctgtattctggtcatttgacctatttgtctattctttcaccaaaaccacactgccttgattgctgtagctttgtagcaagtctcaatgttaggtggtgtcagttatctggctttgttcttttaatttcttttttttttttttcctgtgaatgaacaatactttctgtttctttgcatggctaatttttttaaacaaataaattagagattcctttaatgccaagttacacaatcaaaaagataacaatctctttccatttgctttttgcaaggtttgtgttatttcagggcaaaacgtagagcaagtccactggcaaatctgggggctagtgattattttgttttgatttttatttttttaaatcagacattttaaacactacaatgaggtaactctagaaatcaaattctgtccccctttcccagaggtgttttatttatttatttatttattttttgacagggtctcattctgtcacccaggctggagtgcagtggtgtaatcacagctcattggagcctcgacttcctcggccccaaacaatcctcccactcagcctcccaagtagctgggactataggcacacgccaccatgcccagctaagttttgtattttttgtagagatggagtttcactatgttgcgcaggctggctttgaactcctggactcaagcaatccacctgccttgccctccccaagttctgggactataggcttaagccactgtgccgggcctccctttcccagggtgtattgttgcttcttgtggcttatagttgcttgtttaatgacttttctaaataatttttataaagattattctttgtcatgtgtggctattacattttctgtccagttagcttagtggtcagccagtaacttgagatttctttaaacatctggagcaaaaacaaaaaccaaataaactagcaaaagcaaaaacaaaaacacccctgatctttgcaggttggctgtgttggatcactttctcaatgcttagccagtctattcacagctctgctttagccttcactttctgcatgcatggaagctaaagatgagccagaggtgaagtttaggattttctcaggtctcctctgaacatgcgtccagccttgggcacgtgagtggacttctggactccctggtatatacagagatttcctcagccttttttctcccacatatctttctccatatatctttgcatatttcccagactttttgatgtgtctgctgcttgccccctctgttagcccttgctcaggtggctgcagctagtatgtttgcctttaattgtttacagcaaacaccagccagaaggctgttccagccctgagaaagttctaaggtgtacaaaacaaaggcaagcccctgagtgtgaatccctcaggcagcaaccagacaggtcaaaacacacaaccacaattctttgataacaaggcctatattgccccttctggcatgagcaagctatataactggttataaaaagtattacattaaattatggggacagaaatgactctactagtgaatgaagaaatcatttagattttatggttagtaagtggaggtttgagagtaaccatccgatctttattaatcttagtgtatattatatttctagaatttggactcatatcaagatgctctgaagaagaacaaccctttaggatagccactgcaacatcatgaccaaagacaaagaacctattgttaaaagcttccattttgtttgccttatgatcataatagttggaaccagaatccagttctccgacggaaatgaatttgcagtagacaagtcaaaaagaggtcttattcatgttccaaaagacctaccgctgaaaaccaaagtcttagatatgtctcagaactacatcgctgagcttcaggtctctgacatgagctttctatcagagttgacagttttgagactttcccataacagaatccagctacttgatttaagtgttttcaagttcaaccaggatttagaatatttggatttatctcataatcagttgcaaaagatatcctgccatcctattgtgagtttcaggcatttagatctctcattcaatgatttcaaggccctgcccatctgtaaggaatttggcaacttatcacaactgaatttcttgggattgagtgctatgaagctgcaaaaattagatttgctgccaattgctcacttgcatctaagttatatccttctggatttaagaaattattatataaaagaaaatgagacagaaagtctacaaattctgaatgcaaaaacccttcaccttgtttttcacccaactagtttattcgctatccaagtgaacatatcagttaatactttagggtgcttacaactgactaatattaaattgaatgatgacaactgtcaagttttcattaaatttttatcagaactcaccagaggttcaaccttactgaattttaccctcaaccacatagaaacgacttggaaatgcctggtcagagtctttcaatttctttggcccaaacctgtggaatatctcaatatttacaatttaacaataattgaaagcattcgtgaagaagattttacttattctaaaacgacattgaaagcattgacaatagaacatatcacgaaccaagtttttctgttttcacagacagctttgtacaccgtgttttctgagatgaacattatgatgttaaccatttcagatacaccttttatacacatgctgtgtcctcatgcaccaagcacattcaagtttttgaactttacccagaacgttttcacagatagtatttttgaaaaatgttccacgttagttaaattggagacacttatcttacaaaagaatggattaaaagaccttttcaaagtaggtctcatgacgaaggatatgccttctttggaaatactggatgttagctggaattctttggaatctggtagacataaagaaaactgcacttgggttgagagtatagtggtgttaaatttgtcttcaaatatgcttactgactctgttttcagatgtttacctcccaggatcaaggtacttgatcttcacagcaataaaataaagagcgttcctaaacaagtcgtaaaactggaagctttgcaagaactcaatgttgctttcaattctttaactgaccttcctggatgtggcagctttagcagcctttctgtattgatcattgatcacaattcagtttcccacccatcggctgatttcttccagagctgccagaagatgaggtcaataaaagcaggggacaatccattccaatgtacctgtgagctaagagaatttgtcaaaaatatagaccaagtatcaagtgaagtgttagagggctggcctgattcttataagtgtgactacccagaaagttatagaggaagcccactaaaggactttcacatgtctgaattatcctgcaacataactctgctgatcgtcaccatcggtgccaccatgctggtgttggctgtgactgtgacctccctctgcatctacttggatctgccctggtatctcaggatggtgtgccagtggacccagactcggcgcagggccaggaacatacccttagaagaactccaaagaaacctccagtttcatgcttttatttcatatagtgaacatgattctgcctgggtgaaaagtgaattggtaccttacctagaaaaagaagatatacagatttgtcttcatgagagaaactttgtccctggcaagagcattgtggaaaatatcatcaactgcattgagaagagttacaagtccatctttgttttgtctcccaactttgtccagagtgagtggtgccattacgaactctattttgcccatcacaatctctttcatgaaggatctaataacttaatcctcatcttactggaacccattccacagaacagcattcccaacaagtaccacaagctgaaggctctcatgacgcagcggacttatttgcagtggcccaaggagaaaagcaaacgtgggctcttttgggctaacattagagccgcttttaatatgaaattaacactagtcactgaaaacaatgatgtgaaatcttaaaaaaatttaggaaattcaacttaagaaaccattatttacttggatgatggtgaatagtacagtcgtaagtaactgtctggaggtgcctccattatcctcatgccttcaggaaagacttaacaaaaacaatgtttcatctggggaactgagctaggcggtgaggttagcctgccagttagagacagcccagtctcttctggtttaatcattatgtttcaaattgaaacagtctcttttgagtaaatgctcagtttttcagctcctctccactctgctttcccaaatggattctgttgtgagcaagagtttatatggcttcatggcagcaagggaacagtcaacttcagcatcatatgcaccagtcctcggagtgccctgtgaatcatattggtctttgggtcagtgtcatcattctcttcaagtctggggcttggggaaaaaattagatcagctacggcatataaaaaagtcttttgtttcacatatgtgtaatagcttatttaattttttatcctgctacacaaatatgtaattaaccaatgaggactcatgacttgatagtgtatgtatgtaaagggatatatggacttaatcataagctgttgaggtgaaagacgtggatccacctgctttccaagaaaactcggccaaatttatttgcagctggatattgaatgggacttttctggttgtcttagaattctggctaaaggctcaaagctgacgaaagacagtaactgcaccaacatgatactagacacagccagtctggacttatcaaaagagcagaaagagaccaatgactcccagtccgtattatccatctctagaagactagagtcaaaagcgtgattaaagagtcattaagcggaggttctaggccatagggagattgctttgaatttcttgcagacaagtgtgagggactcagcatggtagaaggtagcctggcatcccactccaagactgaaagcttgcagagtaacaggagcacacaggttcagtgcagcagatgtggtgtggcttgagaattcttggaagagcttgatgagtgtttgctggagtccgagggtgggcactgggaacacagagactggtaaatagtgtttggcaaatacaagtgcttgatgaatatttgttgaatgaatagatgagttcttcccccctggggaattcaggaggtgaaaggttggcttgagcacccaaaatggcaggatgagagaagagaagcactgatagcaacctgccctcccattattgacatggtaaaaggatgtgaatttcttcacatggctttgactatggaagagtagctgggcttgcattgtcatgacgggatatcagccaacagggtagcctgttgtgcaaagaaactatagcagtaagaggacacggggttaggcagaagaggggtttggggtggaggttgctgcaagaggtcagccagataatgtggccctgcatcatggaactgtgcaatgtggggtacactcaaggccctccaataactcacagatgtgccctatgaaaaagccagcatttggactctgccatagcagctggcaggatcatgctggcctgtctgccttattcaatagttaactacaggaagatctgctcctctttgtgtaataccctcttcccttgcaatggcatagggacatctagaatatagagaagacagagacaatggaggaagagtaaagaaactgactatatgccttcgtcatttcactgcaaggaaggccaagcagatttttgaatgaggtgtgagattgctgttaaattggactggcctggacattttaatcccttaaatagaggtgcaatgactaaagtgagatttgtcactaaaatttatggtatctgcccaagattcaggagtgatgatgggaggagatccaacagaactttgttgtaaggcaatggttagagaaaaatgaagccctcgctttctggacttagttcattcaataaaccagtttcggccaggcacgttggctcacatctataatcccagtactgtgggaggctgaggcaggtggatcacttgaggtcaggagttcgagaccagcctggccaacatggtgaaaccctgtctgtactaaaaatacaaaaattagccgggtgtggtggtgtgcacctgtagtcccagctactcgggaggctgaggcaggaaaatcacttgaacctgggagacagaggctgtagtgagctgagacagcgctactgtactccccgctgggcaacagagtgagactccatctcaaaaaagttaaaagaaaaaaaatctggtttcataatagctgtaacgaaataagccttaatgatattttattagcatcatcttctgtctgcattagcccttccttgctcttcaggagaacaacatttgttttcctccctaggctctatcccaaacggcacattcttccacaacccctgttgaacagattttttaaactgttgcctaatctaaaaacaataaaaacaacaaacaaccacagtaacaacaacgacaaaaaaaactgccacagattctaaataatcagatctttttaaatggtatcaatgtttcccacaaaatattgttgacattgaaaatatagaattttagcattaattttgttaaacctacatcccctcggcagaggggcctccctgcatcccagtggaaagtaggttcctcacagtcctctccgtcacattcttcccatttcttttcttcacagaacacatcactgtctaaaattatcttgtttgcttagttgcttactcatcttcttcttctctcctctgaagtctaagctccaggaaaaagggagacttctccacctgttccctgcctctccccagtgccgaggggacactgtgcaccccattgtagatgcgcagtaaaaactcgtgggatgagcaaatgactctgaaacggtcccatgcgggaaatgtccatgaagtcctggattttatctaaaaagcccaggcaggggggggcgggggcggcggggctacagttccacgctgagctgcctcctggccgctcgtccccgccgcagtgcctgggcggcccgggcgcccgaccttggccgtggacaccttcgcggtgggtgctgctcctccccatctgccactggaagatgctggggcgacccggctccaggtttagcaggacactgagaaaagggaatggctgcctttcggaggctgggtgagcccttctctgtgcctcacctgcccgccccacagcggccctgcacctcgtcccacggggcccattgccccggtaggatgcgcgcttttgttttgagggtcaggcatcttccctgccgtcgtttctgggaggttgaaaaattgatccagaaagacctaaaacaaaaaacaaagaaaaacaaaaaagcgaaaaggaggcaaaacaaaacaaaaatccatacaaataaagtatctcctccatccatgtttgcactattccttctcattcccccgtccaatgccacaatttggtaagtttgcctcaacacgttttcagttcttaacactgtgataagaataaacaatagatcccgtagtaagatgacctgttaaaaggaatggggcatggcagagatgagtttggatgaggaaaggtgtcagtccgtttgggctgctctaactaagttctgagatgtgtgttaggtaattttgactttggttgaacatcatagagtgcactcacacagacatagatgacatagcttactacacacctaggcttatggtatagcctgttgcttctaggtgattgtaacacaatgcttagtatttgtgtatccaaacatatctaaacatagcaagggtatggtaaaaatatggtaaagaagataaaaaatggtacacctgtatagggcactgatgtgggttggctgtgtccccacccaaatctcatcttgaattcccacgtgttgtgggagggacccagtgggaggtgattgcatcatgggggcgggtcttttcctgtgctgttctcctgttagcgaatgggtctcatgaaatctgatggttctttaagggggagtttccctgcacaagttctctctcttccctgctgacatgtaagatgtgacttgctcctccttgccttctgccatgattgtgaggcttccccagctctgtggaactgtaggtccaataaacctctttcttttgtgaattgcacagtctcagatacgtctttatcagcagcatgaaaatgaacgaatacaggcacttaccataaatggagcttgcaggattggaagttgctctgggtgagtcagtgagtgagtgaggggtgagtgaatgtgaaggcctaggacattaccatatcctactgtggactttaccatgtcccactgtggactttataagtcccattgtacatataggccacacaaaaattattaaatttgttttctttctttagtaagaaaaattattaaaattgttttctttctttagtaagaagtcaaccttagcttattgtaatgtttttactttatcaactttttgccttaaatttttttttggctctttagcaataacacttagcttaaaacacacattctacagctatataaaaatattttccttctttacatctttattttgttttttctatttttaaaatattttatttttatttttcactttttaaacttttctgttaaaaactgagacacacacacagacacacacacactagcctaggcctactcatagggtcattatcaatatcactgtcttctacttctacctcttgttccattggaaggtctttaggggcaataacatgcatgaagccatcatctcctgtaacaacaatgccttcttctggaaaacctcctgaaggacctgcttgaggatttttatagttagcttttaaaaaaataagtaggagcacactctaaaataatgattaaaattatagtctactagatacagaaaccggtaacatagttgcttattatcattatcaagtattatgaactgtacataattgtatgtgctgtgcttttatatgactgtcagtgtcgtatgtttgtttacagcagcatcaccacaaacatgtgagtaatacattgcactgcaatgttactaggccataggaatttttcatctccattattatcttatagggccatgactgttgagtgaaatattgttatgcactgcatgactgtaccacaaattgagtagcttataaacaatggaaatttatggcttatagttctggaggctgtaatgtccagggtcaggctgctgacagatttggtgcctggcgagggcccactttctggttcatagatggcttgtcttttcactgtatcctcacctgggggagggcactaatgccattcatgaaggctccacattcatgacctaattgtctcccaacatctccaactcttaatacagctctttctccgtatccacaggggattggttccaggagccctgtagattccaaagtctgtgaatgctcaagtcccttatgtaaaatggcatacttccaacctgagcatcatagtgagaccccacctcctcaaaaaaaatacatgcctggcggcacatgcctgtagtcccaactactcaggaggctgaggtgggaggattgtttgagcccaggaattccaggctgcagtgagctatgatcatgccgttgcactatagcctgggcaacagagcaagacactgactctgaaaaaaaaagtgtactatttacatataacctatgtacatcctctcatatacttaaatcatctctagatttcttacaatacctaatacaatgtaaatgttatgtaaatgttatgtagttgttaaactgtatggtcttttatttttattattttgtattattatattgttattatttatttattcatttattgaatattttcaatccatggttgtttgaatctatggatgtggaacccatggatatgaagggctgattgtattatccccttgggggttaggatttcaccataggaattttgaggggcacacaaacatccagaccatagcaaaagagataagaaacctaggttagtgaccagggagggctgctaggatgagccttgatgaaagatctttcatcaggaggatcctgggcatgtggctgggaagggtcctgggcatgtggctgggaagacccttcaaaggggagggcacagtgggtgcaaagttggaagtgaaagaataccatgagcgaagaaagcatcaaattggcagtgttgctggatgtgttagtttcctgtggctaccaaaacaaattaccacaaactgggtggcttaaaacaacagaaatatatttctcccagttcaggaaggcagaagtcagaaatcaaggttggcagggttgttccttccagaggctgtcagggagaaatcatccctttcctctctcctagcttctggtagttgctggcaaaccttggcgtcccttggtttgcagatgcatcagctgcttcttagaatgccctggaggacatgccccagtagttagcagctttctatagaatgtggtacataatacttctttgtctttagttttggacccaagtcataagaaaagttccagtcaacaccctattacgtaatcagtagaaccaacagatgagaagagaaaccaaaattattggtaggctattgacatacatggcagatgcgcaatctaaaaaaatgtatcccaaatttgggttaaataatcaataatctgtgcctgtgtcttcacatggtgttctcctccaagtctctgtcttttttttcctgtctcttacaaggacactctagttggatttaggagcctccttaatctgatgccatcacatcttggtccttaacttaattagatctacaaagactcttttttcaatgaggtcacattctgagatttcagaataaatctgaaataaatggacataaatgtttggaggttgggtggaggacactattcaacccagtcctctggagttggcatgaccagctctgatgccctggagactcagttgagtgtggcagcagaaaagaaatgttatcctggaaagagtgaagactctaaaagaagctgcttggaaagtcagaggtacaaagaaaggagtaataggcaaagagaatagagtggatgggtgtaagagcacaggaaagagacagtggatggagacccgggatataaacggtgagagtgtgggagttttaaagagaatgagaccacaaatatggggaaaggaggaggtatttctgtctaggtttctggggactggtttgattagaaaggaatgagactaaactacagggagtaaaagacctgaggggatttctaaaaccacagggactttctgagagtttcaattacggttagtttatgaggaatctcaacatcatctagtccaaatgtgaagtagtgtttatttttctcagtcattccatcaactggattctgaaattcccctcttgtcactccttagtgatatcttgagcagatttaatacttctgcagtccttttgagatactgatactccaatctagtttaaaattctaaatctccatcttcatctaaaactcatcttctttctgcatgtcacaggctaggtctgtagctcaaccttggcatatattttggaggtaggcatggtttatctgagatacactcttcttgcccctccctctcccagatctagctccctgaagtggagatcaaggatgagtggtaagaaggcagatatccttttctataattgtgtcgattaaggtacagtctaagctgcaggactgggagacctcaaatacagaggcttaaataagattgaggtttatttctttctcatataatagaaaaaagtatagctaagaggtctagaataggtaagctgtctctattccacaaaatcattcaagggctcaggatgaccagatgtctctgtttcctatagctctggtcttatcctatggcctccattctcccttgcatggagccacaggagctgatgaagccctcctgtgcctttttgtgtgtgtgtggcccatgggatgctgaggtggagatacaccccctttcccaggtacctgataccacagtctctaattgccagttggcaagggttggtccaatggaaatctccaacagagtagacaccagtcgctctactaatttacactcctgaacccccagggggcacgtgtaatgttctctaatactgctcagatgggcagggggatcaacctcagagggtgatcctacacattgctgcatcaaaacaagaatcctcttgaggaaagaaatgcctgatctgcctgtcccgtgtctaagagtaattttactgttatttccccttcacttggcttgagaaagagatgtcatctcttgcatagggagaagagcttgcaggcttacatcattaacactcatctcctggtgctgctgactctcttgtcaccctccccagcattctcaggtgaatgggggaaggtagggtgaggcgatgctgtggacagtgctatctggttctactgcttcttagaatgccccgggggacatgcttcagttgctagcagctttgtatagaatgtggtacataatacttctttgtcttcagttttggacccaagtcataggacaagttccagtcaacaccttattatgtaatcagtacaaccaacagatgagaagagaaaccacattgttgataggctattgaattatatggcagatgagcaatctaaaaaaagtatcctaaatttgggttaaataattaataaaaaagacatttcatctatagtaatagaaaaaaaggcaaagaatagtggtatgggtttatgtctattggttcaaaaagtccactttatagaatatatattaaaaaaataattgttcagccaggcacagtggctcatgcctgtattcccagcactttgggaggtcgaggtgggtggatcacctgaggtcgggagttcgagaccagcccgaccaacatggagaaaacctgtctctactaaaaatacaaaattagccgggcgtggtggtgcgtgcctgtaatcctagctattcaggaagctgaggcaggagaatctcttgaacctgggaggtggaggttgtggtgagccgagatcatgccattgcactccagcttggggagcaagagcgaaactccatctcaaaaaaaataaataaataaaaataaaagtaattgttcatacacatcccaaggaaacattgctgcttattgcagaattgtttctaattgtgaaaattggaagcaatttaaatgtccaacaacagaagattggtaaaatgcattataaactaaaatattaaaaattgcattataaataaaaaggaatattatccagccaataatcatgaaattgtaaatggataattactgacatgaaaagatatttatgatcgattataggttaagaaaagcatattacaaacagtatacgtagtatgatgctatgtgtgtgtgcatgcacttgggcatgtgtaagtagataaattgttataaattttctgaaccacaatttgcagtaggagccaagaatctaaagaatgttaacatatagagaaaaatgactgcatggttgctctaaaaatgatattggtgtgattctatttggtgagattactgcttattcaaatattttaaaatttatttatatttcctgattttcctacagtaaacatttattacttggataattgaaaattaaaaaaactaaaaaaatacatttgcgaagaacagatacacgtatatctatcttatcttgttatggagaaaaactttctaaatctaaaaccagggtagccatggtgtgctggagcttgcttgcacagatttgttaaatctgattctaaagtattcagggccaggcacggtggcttacacttgtaatcccaccactttgggaggcgaaggcgggtggatcacttgaggtcaagagtttgagactagcctgaccaacatggtgaaaccccatctctactaaaaatacaaaattagccaggcatggtggcacatgcctgcaatcccagctactagggaggttgaggcaggaaaatcacttgaacctgggaggcaaaggttgtagtgaactgtgatcatgacattgcactccagcctgggcaacaagagcgaaactccatttcaaaaaataaaattaaatataaataaatacataaataaaaataaaatattcaagagctttgccaaaaaattagttggtagctagccctcagcaatagcgggaatactgacactgcagatatcagcaaatgctacaaatcagacttttcttttgaaactgctttaccagcacacatctgaaagacagtaatagcaaagaaaaggggaacttacttgtttatataaaaatgaatgtggggcctggtgagaagtaattagatcatgggggaggagttctcatgaatgggttgacatcattgccttagtgctgttctcgtgatagtgagtgaatgagttattgtaagatctggttgtttaaaagtatgtagcacctccccacactctctcttgcttctgttcctgctgtggaagacaccttgctccccctttgccttccatcatgattgaaagctctgtgaggactctccagaaacagaagccactatgctttctgtacagcctgtggaactgtgagccaattaaatcccttttctttataaattagttagtctcaggtatttctttatagcagtgcaagaatgaactaatacagaaaattggtactgaggaagggggcattgctataaaaatgcctgaaaatgtggaagcagcttcggaattgggtaacaggcaggggttagaagagtgtggagggctcagaaatagacaggaagatgaagaaaaatttggaaattcctggagacttgttaaattgttttgaccaaaatgctgatagtgacagggacaatgaagtccaggctgaggaggtctcatatagaaatgaggaacttactaggaactggagcaaaggtcacttttgttaagtgttagcaaagaacttggaaacattgagtccctaccctaggaatctgtggaactttgaacttgagagagaccatttagggtatctggcagaatgaatttctaagcagcaaagcgttcaacttgtggcctggcagcttctaacaacctatactcctatgtgtgagcaaagaaatgacctgaaacttgaacttatatttaaaaggaaagcagagtgtaaaggtttggaaaatttgcatcctggccatatggtagaaaagaaaagcccattttcaggagaggaattaagcaggctgcagaaattttcataactaaaagaaaggcaaatgctgatagccaagaaatggggaggtggcttcaaaggcatttcagagaccttagaggcagccacttccatcacaagcctaaggaggcttaggaaggaagaatggtttcctggaccagacccagggacactgctcctcacatcccagccactctagctccaacaatggctcaaagtggcccaggtacagctcaggccactgcttcggagagtgtaagctataagccttggcagcttccacgtggtgttaagccttcaggtgtgcagagtgcaagagttgaggcttggaaacctccacctagatttcagaagaagtatgtaaaagcctggatgtccaggcagaagcctgctgcagggccagagccctcatggagaacctctactagggcagtgtggaggggaaatgtggggttggagccaccacacagagtccccactgtggcactgcctagatgagctgtcagaagagggccactgtcctcaagaccccagaatagtagacccactggcaacttgcaccttgtgcctggaaaagctgcaggcagtcaacaccagctcttgagagcaactgtggggactgaaccctgcaaagccacagaggaggagctgcccaatgctttgggagcccacctcttgcaccagtgtaccttggatgtgaggcatggagtcaagggagattattttggagctttaagatttaatgactgccctgctgggtttcagactagcatggggcctgtagcccctttcttttggctgatttctcccttttggaacagttgtatttacctagtgcctgtacccttgctgtttaagagtaactaacttgcttatggttttacaggctcataggtggaagggacttgccttgtctcagctgagactttggacttcttttgaattaatgctggaatgagataagactttgggggactgttgagaagggatgattgtattttgcaatatgagaaggacatgaaatttggaggggctgggggtggaatgatataatttgtatctctgtccccactaaatctcatgtccagttataatcccccatgttggaggtgggaccaagtgggaggtgattggatgggggtagagttctcatgagtgggttagcatcatcttcttggtgatgttctcatgttagtgagtaagtgagttatcatattatgagatctggttgtttgaacgcatgtagcccttcctctctctctctctcttgttcctgctccagccatgttcctgctctcttgttcacttttcaccacgattgtaagtttcctgagacctcgccagaatcagaagccactgtgtttcctgtacagcttgcagaactgtgagccaattaaacctcattcctttataaattacctagtctcaggtatttttttcatcagtgcaagaatggactattacacaattaaatagtaaaaaacagaaccctgaaagaaaaataggtaaatgacatgaacaggtaattcactaaagagatacatattgccaataataaaaaaaaaataaaacaataatgagatgtcactctacatctataaaattgacagctctctataatgttatttttgtggaggatgtgtagaatgtctaccccagcacactcttggagcgcaggtaaagtggaacaaactttatggagagcacttggtgctatgtaacatgacaatgctcatgtccttcaacctagtcattctttcccagaaatttatcctaaagcaatagtaaaagattcagaccacgactgtgtacaaaggtgcttataacattttgtgaaatgcaaagggcaaatctctgctcagacagagctgagggtggctggtcatgacctaatatttactcaggtactgatatatcagtggcaatttcaaaaatttgactggaaaactttagatcattaagggagaatttattaaaaggtaaatgtaaatgtgatgacatttctcattcccaacacgtttacagggtgcagttttgaagttctaggatcaggtgggtaagagaaactataggattgtgtaggggcagtagggaggaaaagagggaaaggaagatcactattctgcacaattgatgtgagtgctaacctcactgcaagaagcacctcacaaattatgacactgtgcccacagcatttgtttccttccttattgattaagaaaccatactatctactccttcatgataaaaacacccaacaagctaggaataaaagaggactttctcaactgataaaaagcatctatggaaaaactcacagttaacattgtacttaatggtaaaacatatacttaaagcttttgccctaacatcagctacaagataaggatgtctgctcccactacatttttttgaaactagatctcattctgttacccaggctggagagcagtagtgtgattataactcactgcagccttgacctccaggctcaagccatgccccgacctcagcctcccgggtagctgggactttaggcacacatcaccatgcctggctaattttggtattttttttttttatttttgtagagacagagtctccctatgttgcatatgctgatcttgaacccctggcctcaagtgatccttctgccacagcctcccacagttctgggattacaggtgtgaccgactgtgcctggccctgctctcaccactcctatttaacattttactggaggtactagccagggcaattaggagttaagaatccatattggaaaggaagaagtaaaactgcctctatttgcagatgacattattttctttatagaaaatacttagaaatctgcttgtcttagtcaatttgggctgctataaaaagtgctagaaactgagtggcttataaacaacataaatgtatttctcattgttctagagcctggaagtctgaggtcagagtgtcagcatggttgtcttctggtgagggcccttctctgggttgtagacttctgtcttttcttatactctcacatggcagaaaaaggactagtgagctctttgggtctttttataagggcactaatacgattcatgagggctccactctcaagactcgattacctcccaactgatattatcacattgagggttgggacttcaacatataaattttggaggagcacacacatttagtctgtaacaccactaaaaaaccattagaactaataaacaagttcagcaagattgcaagatacacatcactatataaaaatctatgatatttctatacagcaacaatgaacaaatcaaaaatttgaaaaacaatgcatcaaaagatacttagaaataaactgaacaaaagaaatacaaaacatatactctgaaactatataagaccattgaaaaaattaaagaccaaaatcaataaaagacatcccatgcccatgaatcagaagacttttttttttttttttttttttgagacagagtcttgttctgttgcccaggctggagtgcaatggtgcaatctcagctcactgcaacctccacctcccgggttcaagcagttctcctgcctcagcctcacaagttagcaaggatgtagagaaattagaactcttaactattgctgatgggaatgtaaaatgatatcaagacaaaagtggctctatcttgggtgcaaatccaccatgttgacttctgattagcctcagtcccatgaatgcctcctgattcctactttatttactatccttagtgtactcactatagacagtaggaaggggtctaagctgttgatcaagaatcaggtttttcaaaggcccactattctggcattgggttgctgatctattttttctttgcctactccagctgagatcagatcataccacatttgtttcctgataattctaaccgggccttttaggtatccctttttaagttcagcagaattggcttctccctttgcccaggaaagaattcaagccagaggtagaagaaaacagttttattgaacaggcagtgttatatctctggtggtattatagttccctgactgctcctgcagagcagggctaccccatagacagagtagcaactcagggcagttttgcagtcatatttatacccacttttaattgcatgcagattatggggcagtttatgcagaaatttctagggaaggggtagtaatcattgggtcattgccatgaaaaggggtggtaatgtctggctgttgccatggcaatggtaaattgacgtggcacgctggtgggtgtgtctgattgaaagctgctttcaccccagccctgttttagtgagtcctcaatctggtccagtgtctgagccccgcccctggagtcgagtcctgcctcctacctcaaaggccttagaaactttaattcagttgagataaatatacggctggcaccacaccaacaactcaggaccaccgtagaggatggtccattaaccacagtggcaaagaaagtccagccagacaaatttctaccagagctgataaaaggccaacagcaaatattatttgggactccccaagaccttgaggcagaggagggaacacttgaatggaagtttgactggcaatctcccccaggttggataaggtatttttttttttgctacagaatagggaattccttggcaactaaagaggtcttcattgatcctgctggagtctggaccaagacgctccacatacccatacactgaacatggccctttttaaaaggcactctggtggctgcttggtatggtcctttgctgcccctgtgaccttaccctcgcccctcaggcaacccgtttggtgtgcaccctcagcccataatccttgggctgcttgcgtcataactaacagagatgaagctaccacagccattttgcttgatggggaagaactgccctgccaagtacctactcgagacttgtatttcctcccatagtcttctgttcctgctgctgctctgccctgcaaaatgcctcttggtttggattctggaaaatatggtggcaaaggcatgattaattctgtgtctcatacttgacacaggtatcattgcctgttgtttgtattgttgctgcagcctctgcttacaagtagaaaagaaactgatgcaatgtgtcacccacatccccaaaatgactgcagcagccccctggctcaggacccatcatggaagaggtgggcgcttgaggttgtaagagccggattagaggggtggagtgtggagacaaaaggggctctatcttggaagctaatccaccatgctgacttctgattagctccagtatcctgaatgcctcctgattcctactttatttcctgtccttagtgtaagaacatgaactcactacagatcctgattttagatcaaagcaacattgatgttatcacacatattataggctatggtgcacatagcagtctcgcctgttctggaaggtggcctttagctgtctctatagagcatgtacttcctttccctatataaggtgtctataagtcctgggtctggatagtaatcctgcagagatctacctgtattgctcctgttttgaccatgcttctgtgggtaagttccccaaataaatcaccctttactgacaaactggatttgtctgcttcgttctttggtttctcagctcctagggcatttgggggctgcttttcctatatggctgtttcatagaacaaatggtacagctgctttcagtctggcagttcctcaaaaggccaaatgcagagttaccatatgatgcagcaattccactcctaggtatatacccaagagaaaatatgtctacacaaaaacttttacgcgaatgtcaatggcagcattattcataagagccataatatccatcaactgataaatggataaataaaatatggtacacacatacacttgaaaatcatttggcaataaaaaaattaaaaatgaaatattgagtggacgaaccatggaaacactatactaagtgaaagaagccagtcacaaagggccactttttatatgattgcatttatgtgaaatatccagaatagacaaatctatagagaccagtatgatctatagagtagattggtggttgcctagggcttggatacgtggagaagtggaggtgatagctaaggggtatagtgtttctttttagtgtaatgaaaatgttctaaattgatttggtgatggttgcaccactctgtgaatatgcctagtcattgaattacacattttaaatgggtgaaatttttttatggtttgtgaattataactcaatagagctttgttaaaaaaaacaagcaacaactaattacagatcacatctaattgacagcatatttttactgccctgaatccaaaaagaagtgcccaggtatgggataggggtgggcggagaaactgctatgaagagatgtatggggttgcttagagtataaagtatgaagtctttattttgaattaactctgtgggctgctgttaactggacttaggcttgtacttctttgcatggctgtgataatgctattctaaacctgcattgtccaatatggtagtctctggccaaacgtgattgagtgcctaaaatggtataacccagttgagatattctgtaagtgtagaacacacagtggattttgaagacttagaatgaaaaaatataaaatacctcattaataatgttttatattcattgcatgtttggatgtattaggctaaataaaatatttttaaaaatttaaaaagaagctgtattctctgagaatggaagttttccctgggaaatgaggctgtgtcaacgcatggtatacatgattacaagtgtgtgtgtttttttttttttttttggcattgtacatggcagagactggagcccggacttaaacctgggcagtgtggctcacagtctgtgattttaataactgcaaatcttaatttcagtatcctatttttcagttctggaatttccatatgatccttttttatagtttctagttttctgctgacattcttgtcatgttattccctgaatgcatttttttaaattaattaatttatttatttttattgatcattcttgggtgtttctcgcagagggggatttggcagggtcataggacaatagtggagggaaggtcagcagataaacaagtgaacaaaggtctctggttttcctaggcagaggaccctgcagccttctgcagtgtttgtgtccctgggtacttgagattagggattggtgatgactcttaatgagcatgctgccttcaagcatctgtttaacaaagcacatcttgcaccgcccttaatccatttaaccctgagtggacacagcacatgttgcagagagcacagggttgggttgggggtaaggtcacagatcaacaggatcccaaggcagaagaatttttcttagtacagaacaaaatgaaaagtctcccatgcctacctctttctgcacacacatggcaaccatccgatttctcaatcttttccccacctttcctccctttctattccacaaaaccgccattgtcatcatggcccgttctcaatgagctgttgggtacacctcccagacgaggaggtggccaggcagaggggctcctcacttcccagtaggagcagccgggcagaggcgcccctcacctcccggaccgggcggctggccgggtggggggctgactcccccacctccctcctggacggggcggctggccgggtggggggctgacccccccatctccctcccggacggggcggctggccgggcagaggggctcctcacttcccagtaggggcggctgggcagaggcgcccctcacctcccggaccgggtggctggccaggtcgggggctgactcccccacctccctcctggacggggcggctggccgggcgggggactgacccccccacctccctcccggatggggtggctggcctggcgggggctgtcccccacctccctcccggacggggtggctgccgggcggagacactcctcacttcccagacggggtggctgcctggcggaggggctcctcacttctcagacagggcagttgccaggcagagggtctcctcacttctcagacggcgcggccgggcagagacgctcctcacctcccagacggggtcgcggccgggtagaggcgctcctcacatcccagatggggtggcagggcagaggcgctccccacatctcagacgatgggcggccgggcagaggcgctcctcacatcccagacggggcggcggggcagaggtgctccccacatctcagacgatgggcggccgggcagagacgctcctcacttcctagatgggatggcggccgggaagaggcgctcctcacttcctagatgggatggcggccaggcagagatgctcctcactttccagactgggcagccaggcagaggggctcctcacgtcccagacgatgggcggccaggcagagacgctcctcacttcccagacggggtggcggccgggcagaggctgcaatctcggcactttgggaggccaaggcaggcagctgggaggtggaggttgcagcgagccgagatcacgccactgcactccagcctgggcaacattgagcactgagtgaaccagactccgtctgcaatcccggcacctcaggaggccgaggctggcggatcactcgcggttaggagctggaaaccagcccagccaacacagcgaaaccccgtctccaccaaaaaaatacaaaaaccagtcaggcgtggcggcgtgcgcctgcaatggcaggcactccgcaggctgaggcagagaatcaggcagggaggttgcagtgagccgagatggcagcagtacagtccagcttcggctcggcatcagagggagaccgtggaggtagaggtagaggtaggggtaggggtaggggtaggggtaggggtaggggtagaggtaggtagaggtagaggtgtagaggtagaggggaagtgtgtgtttgttgattgatgggtgtacatgattacaagtgtgtgtttgttctgtggtgacccactggacagcagggggaaaaagaggcaggtaggtgcctccgaattagacactttattttatttatttatttatttatttatttatttatttatttatttattttttgagatagagtcttgctctgtcacccaggctagagtgcagtggtgtgatcttggctcactgcaacctccgcctcttgggttcaggcgattctcctgcctcagcctcctaagtagctgagatcacaggcatgtgccaccacgtccggctaattttttttatttttagtagagacggggtttcgccttctctactaggctggtctcaaactcctgacctcaggtgatctgcctgcctcagcctcccaaagtgttgggattgtaggcatgagctaccatgcccggcctgaattatacactttaaacgggtgtattttttatcgtttgtgaaattttttatggaaggtgaaattttttatggtttgtgaattatatatcaataaggttttgttaaaaaaaaacacaactaactacagattacatctaattgacagcatatttttactgccctgaatccaaaaagaaatgcccaggtatgggctaggggtgggcagagacacacacacttgtaatcatgtacacaactgggagtagtggggatgagtctgtggggaaaaacaggaattcagagtgctgagaagcttcccttactgccctgcccacttccctcccctgggcattgctccctcagcctggattcacaaagtcaatgcctttgggagccaggaactaaattcttagtcaaagacacctccctcctgtccagttatgggtctcagggaggaaatggcaggtgggtggtttcattctttcagtttcagaaaatatctgtagacataacatatttcctctattaaacaaattctgaggtaaggggaaacagagagagacagtcagaacattataagtcgaaaagaatctcagcattcttagtttagggaagttggccttgctcgctgtgtggtagaaaagctgattacgggagagaaccgtggagggaaggaagggaggcagttttgtcatccccacatccggttttgactggacaccatgcaggctgagatcaacttcagaggcagtcagctggaatcccatagctttatttgcccatttattgtgttgttattttgttgttgtcattactggaaaaggcaagagtagagctatcagaatgtcttccttgaacatgctgtcaacaaaccccaggatttttataaaccctatgtgaaaaacacgctaagaatctcttttcttcttagcatatttttcacttagggtttacaaaaatatgttttgaagaaagcttgaaaagatcagtggaaaaaaattcagcaccaaattttctctgaaaatcaacttgtcaaaaaagacgtgattaacatccatcctgtaaccagcacatgaatgatcttccctctttacgagtgtgcttcagcaaaactttaatctcgacccctccctctttccacttttaatagtggatggatgaaaaggcaagaggaaaaacaaagcagccgaaacacacccagacgcacacttctgccaagcatagccaccacggtaagcattttgaattcagccaattatgatctgtggattattaacattacctcatttacttcatcagtgtagacaatagtaaactgactgtagtgaaatttggaaccatagaaacgtgattctattttaatacaggcctatcaggtaacccacagactatgctacatatgaataagagaaaataagtttattcttaactatgtaatgtaattccagtggaattcggtaggtggcataaaacagcctttttgttttgttttgttttcaacaccaaatgtggttaacagtgaaataaattcaaggaagtgcttgaatttcttacagactgccaaatggaacagacaagcaggttgtcttggtaagcaacacattcttttcttttgtaaaagaaaataattgtatagctagttattaagtacagaagtctcaaaaatctgtgtaagtcctgggtgtttttctaagtgggttatatttctgatatgtatatagttactgtgtagtttgtactggcatttgtgtatcagttctgagtcctaaatcagagaaagtccccacactcctctgggaataacacctcgtgtgtgatttgcttataggaaatatttttgagttgggaaatgaatatttgggtcacacacgtcttctggttttcttccagagcagctgctagttgttgattttgacagcatttctcttcacctaatcccgccatttgtattctcttctcagtgttaaagaaaatgagatataagtcagttactcccggaggcaatgctgctgttcagctcttctgtttttgtggccaggtttgtttcagtttttctctctggggccttcccactagctagtccttctgtttctcaggccactaacactgttctggggagctagacgtgaggtagaattgcaggttttgaaaatgtttcccaggctacatccaatttggtcaaaagacttgaaagtgaatttgttttataacaaagcaagagttcacaaaaccgcatagaaagcaaagcagaacagttcttgaactgtctcagattcttttaacctttggttaatagctatttggtcttgtgcagagaaggcccttagtaaatatttacaggactaaacttaatgggccagatagattgtatgggtattgtccattaagaccagtcaaagccttgatttgatgcctccaaagtctccaaaagaagacaaattaaatgtattgattcattgtatcatataatgtgaggctaaacctatcataataatgaaaattcacagaagctcacctagaggcattttacactttcaaattaaagcatccttttctgctcagcttattttttggatagtaaaagagtttaaaggttctaaaatagatgaggctcagtcatggtcattaagacgttgatcaaaaaattctttggtctaggaaactgagacattgattttcatggatttagcaagtttggtatataaaattcaaagatcctggaagtattaaaaatcagttattcctgtgtataacattttgatttcttaattatagaaataagaatggtttcataaactgagtacttaaaattaatgactttaaatgaaagggcaagatggaggttaggcaaatagaagatatttgagtcaattaatttagctcgacataaaactgaagctatgctttcattaaatgcttgcgttagcagtggtgaatccatatgggtcgtagaaacttaattcttgcctcctcagtggaaagaatttgtgtgaggggcataaggcagagtgagagaccgaggcaagttttagagtaagagagagagtgtcacgcccagggccaggttccagcccatactgaggtctgaggggagggggtggatgagcagatagctgaaagaacactcagggggccgtaggcaggtgaaaggtgattttattcagcaacagctctcattagcagcttacttacagtagttctctcagactgtccgccttgtcctggctgcttagttcggcagcttccacacacaactgtgcgtccggctctcccttgccctcagggtcagcagcttaactctttctctctctgggtacaagcaagccgagctgtgtcctggctcccctcagtccatctgcaaagatggacagctttggctctctctctctctctttctctggctgccaatgcacctgtacagcgtcagcagggcaattataccatttacggacaatagtggcttagagccaagggatgaaccttccctatgttatggctatggtaagcttctctatgttatgtctacatggctatgataaaaagtgagttatacgcctgcgctctaaactcgctgagtcactctggatgtttaccttggcctatccttgaccaaagcacagccatattccttacagagagtttattaagaagttttagatcaggaacgaaaggaaataaagtacacttggaggagggccaagtgggcaacttgagagatccaagtgcccagtttaacctattacttggggcttatttgttggcaggcttctggagtcttgcgtctcttctcccctgattcttcccttggggtgggctgtctgcatgtgcagtggcctgccagcccttgggatgggctgcacacacactgtgtttactgaagttgtgtacatgctcattggagacatttttcccttgccagacaagtcttcctagaggaaggtcacatacccgttgaagtccgccattttgcctcttggtatgcatgctcaagcctgcttgcccagctcctgagagtttgtcaggagtctgctaaccaccagcttcaggtgtgttttctatctattaggagactgactttccctgatgctggctgcaaacaattattattctagagagactgtttttttgtttttgtttgtttgtttttgagacagagtctagccctgttgcccaggctggagtgcaatggcatgatcttggctcactgcaacctctgcctcctgggttcaagcaattctcctgcctcagcttccccagtagctgggattacaggcacctgccatcacgcccggctaatttttgtatttttagtagagacagggtttcaccatgctggccagactagtctcaaactcccgacctcaagtgatccacctatctcagcctcccgaagtgctggaattacaggcgtgagccatggcccccggcttagagagactgtttaacaactgcctgatcatcacctgatggtcacctgatattcctggttggggggcccctctcctgtcctgctcatgtctgcctaagtacctactctaacacttgggggcaggcagttatataaatttatatatttttgtgatgatagctgttgttaagggaaaaaggacaactttcatcttgcgtgtggtaggttctgcgagccaagctctcgcactcatgcacggggtcctgtgtgcctctgatcacttttgaataccacgcctgactgtgctgatcactgcttccatgaactctggagttggctaggaggtggaaagagccttgatctggattcaggcataggaataagcagggaaccccacttaggccttaagaccaggccacttttgcttcgacctagcttggcactgccccagggtattatttttattcccattattttagtctgtttaaattgctgtaacaaagtaccagaatgggtgacttataaacaacagacacttacttttcacagttctggaaggtagaagtctaagatcagggtggtagtgttgggttctggcgaaggccctcttctggtttgcggatgcctccttcttgtattttcatatggcagaatgacagcctgaggctctctggggtcttttttataaaggtactaatctgactcataaaggctctattcttatgacctaattacttcacaattttctcctgctaataccatcaccttgggggttaggatttcaacataagaatttcacagggcacaaacattcaagctataacatccattgaaatattgctccttgtgtgagtagctctttaaggaagctggagaggaagaattgggaaatttccgggtctttcagccaaatacagttaatgtcataaaaacaccaccttgacttttaacatgaacattaatgtctgcatcatcttccttgctcagggtcttcatgaacactaataggggtaccaggccctcttcctcgttagaagaaatcaggataacaaaggcatattgggcacccctacaaaagggtaagtccaaggaaggctccaaaacccagataagtttgcacatctaaattggggaacttttgtaaccgagggttgtttgtagcagctaaattatttaggttctctgtgctcattgagatatgggaggggatatgtgaagaggaatttggtgctttgctgatactaatgccctgcttgtctaatggttctaggggattttgaataagttccttgtgagaactgggtagctgtgtaatcacggctgaaagggtctctggaggtgaggactgcacactctagagttcatcccaacacagctccctctgttctcatacgccactggaatgctgaccacaaaatggtgtcttagctagagcagtttgaatactggtacaataggcataacctagaacagtgcctaccactgcatggagaataaatacatatttgatgaatgaatgaaaaaagtattctgaataagagagaggaggaaacaactccctttctcaaaacctcccagtgttctccaccctctcatcatttttggaatagaatcccaactccttgcaatgatcaacaaagcctgagtgatcttggccctaactcctctcatcttccctaactccttactaacattgaccttcccgttccttctcagacaagccaagctcaattccatctcaggtcctttatacttgccttttcttttgccagaaatgctgtaggttacccagatcttcccataggttactcattcacttcattttgatttctactctagtgtcatcatctcaaaaaggccttctctaactaatcactcaaaaatagccacccccaagcactgtctcatacctacaatttaccctgctttatttttctctacagaacttatcactacaggaacttgagtttagtcattcatttattttctgtctccccactagataaaaactccttgagaattcggactttatcttgttcactgctctatctccagttcttaaaacatggccagtaggtgatcaataaatactggttgaataaataaatggatgaataaatggatcttctgagaggccttttatcaggatctaattaccttttgagtaagaccaaagttagacccaaaccgtgtataggtcataaagggtaatagaagagcagtcccaataccaccattaggaacacctcttgagaaagtcatggggagatgtacgtcaaagagtgaaagtacaccagttgagtcttagctggagttctaaataattttgggaagcttggggccagaaaactttctatgaacccacctgtattagtttcctgaggctgctgtaacagatgatcataaacgtggtggctttgccctcaacgttctggagagcagaagtcgaaactcaaggtgtcagcagggccaccatcactccagagcctcctctagcttctggcgcctgttgatgttccttgccttgtagctgctgcactccagtctctgcctccactgtcacaaggccttctcctctgtgtctcaattgcctcttctcttgtctctttgtcaaatatccctctgcctttctcttaaaaaagcacttgtcattggatttatggaccacctggataatttgggatgatctcctcatctcaagatccttaactttattatctctgcaaagaccccttttctaaggaaggtaacattcatggtttctggggattaggacatggttaagatgtagacatatcttttggtgagccaccattcaacctattacaccatccatattaaaacttatttgattagagctggcatcattatttaccaatatatatacttctctagttttaggccatgtaacccaaaagaatgtagcctccactttataagtctgtaacaaaaaaaagtttaatgtcagccatgactaatttcgtgtattttaaaaatgtatgtaatgtatttcatcatttcattattttttttctagaatctgtatctgtatcaagatgatctgaagaacagcttctacctttaggaatgtctagtgttccaaaatgactagcatcttccattttgccattatcttcatgttaatacttcagatcagaatacaattatctgaagaaagtgaatttttagttgataggtcaaaaaacggtctcatccacgttcctaaagacctatcccagaaaacaacaatcttaaatatatcgcaaaattatatatctgagctttggacttctgacatcttatcactgtcaaaactgaggattttgataatttctcataatagaatccagtatcttgatatcagtgttttcaaattcaaccaggaattggaatacttggatttgtcccacaacaagttggtgaagatttcttgccaccctactgtgaacctcaagcacttggacctgtcatttaatgcatttgatgccctgcctatatgcaaagagtttggcaatatgtctcaactaaaatttctggggttgagcaccacacacttagaaaaatctagtgtgctgccaattgctcatttgaatatcagcaaggtcttgctggtcttaggagagacttatggggaaaaagaagaccctgagggccttcaagactttaacactgagagtctgcacattgtgttccccacaaacaaagaattccattttattttggatgtgtcagtcaagactgtagcaaatctggaactatctaatatcaaatgtgtgctagaagataacaaatgttcttacttcctaagtattctggcgaaacttcaaacaaatccaaagttatcaaatcttaccttaaacaacattgaaacaacttggaattctttcattaggatcctccagctggtttggcatacaactgtatggtatttctcaatttcaaacgtgaagctacagggtcagctggacttcagagattttgattattctggcacttccttgaaggccttgtctatacaccaagttgtcagcgatgtgttcggttttccgcaaagttatatctatgaaatcttttcgaatatgaacatcaaaaatttcacagtgtctggtacacgcatggtccacatgctttgcccatccaaaattagcccgttcctgcatttggatttttccaataatctcttaacagacacggtttttgaaaattgtgggcaccttactgagttggagacacttattttacaaatgaatcaattaaaagaactttcaaaaatagctgaaatgactacacagatgaagtctctgcaacaattggatattagccagaattctgtaagctatgatgaaaagaaaggagactgttcttggactaaaagtttattaagtttaaatatgtcttcaaatatacttactgacactattttcagatgtttacctcccaggatcaaggtacttgatcttcacagcaataaaataaagagcattcctaaacaagtcgtaaaactggaagctttgcaagaactcaatgttgctttcaattctttaactgaccttcctggatgtggcagctttagcagcctttctgtattgatcattgatcacaattcagtttcccacccatcggctgatttcttccagagctgccagaagatgaggtcaataaaagcaggggacaatccattccaatgtacctgtgagctaggagaatttgtcaaaaatatagaccaagtatcaagtgaagtgttagagggctggcctgattcttataagtgtgactacccggaaagttatagaggaaccctactaaaggactttcacatgtctgaattatcctgcaacataactctgctgatcgtcaccatcgttgccaccatgctggtgttggctgtgactgtgacctccctctgcagctacttggatctgccctggtatctcaggatggtgtgccagtggacccagacccggcgcagggccaggaacatacccttagaagaactccaaagaaatctccagtttcatgcatttatttcatatagtgggcacgattctttctgggtgaagaatgaattattgccaaacctagagaaagaaggtatgcagatttgccttcatgagagaaactttgttcctggcaagagcattgtggaaaatatcatcacctgcattgagaagagttacaagtccatctttgttttgtctcccaactttgtccagagtgaatggtgccattatgaactctactttgcccatcacaatctctttcatgaaggatctaatagcttaatcctgatcttgctggaacccattccgcagtactccattcctagcagttatcacaagctcaaaagtctcatggccaggaggacttatttggaatggcccaaggaaaagagcaaacgtggccttttttgggctaacttaagggcagccattaatattaagctgacagagcaagcaaagaaatagattacacatcaagtgaaaaatattcctcctgttgatattgctgcttttggaagttccaacaatgactttattttgcatcagcatagatgtaaacacaattgtgagtgtatgatgtaggtaaaaatatataccttcgggtcgcagttcaccatttatatgtggtattaaaaattaatgaaatgatataactttgatttaaacagttctgacacataagggatccacttgtttctttgctataactgagttctgaatttatcatgaagtcaagggaagcacctgtttttcttcagtgatgtgtgttctagtgtatcatggccaatattggaaaatgccgtgacttttgtgaatatagctgggtttgtatagcagatttctacagcttttgatgctttcctcatatccatggtgcattcaactcaacttattgatggctgctcgttgataaagctggagacactatgcctgaaggttcctccccaaccccccaccatcatgcccacacaaagggcagttcagaagtgctggaaattgtccttggaaacagccctcaaccaatgtcagaaagagctggtggataaatatcccaactttctcactcattttttgagataaatcaaaaaatggggtatactttacactctccctgagttccccagtaggataaaattctggttgtccacagtagaaatggcttgattaagtattctcgactggcttccttcccatttttttctcacttacttattcctccatgtgtattccatgggatcacctctgtgataaactacttgccactgaatatttgtctcaggggctgcttatgagggagcccagaccaagaaagtgttccacctcagtccaaatgaaaattctgagtctagttttccatgactggagatctcgctgagattctggttgtatgaactaacatgcacaagagcctttgctcttttgattagaaggaagaatatagttgatatacggtgcttctatatatagcatctgtctctttggggaactattaaagtagagcatgagcagatggggcagtgtaaggaatggatatatctgatgcttttcatacccatcaaacatcctgtcagtctacatttttactccagcagccagctctgggcaggtgtagtcttgacaaaaaccttgtcataactacaccatgtatctttcactttatgccccatgacttcactgataccactgcgtgggatgcttgtagaagcctgttctaccattctgatacatgcacaaacctggaagcacaagggataacactacgggatgggaagcattatatacatgctcctttcttctattcttgggttaaaaaatgaggtgtaattgataccttttctcagacaatccctagtggcactgaggtttacttacccacagggtaaccagctcagtaacttggttttgcattgactttccattcttccttggtcactcttctcattcgggtcactcttgagtcctgagatcgctgctccaaacaagcgacctttccttaactcaaggagtctagaaaaaataaataaaataaaaagcaacaaaaaccaaaaagcctacaaatgaccagcccacaaagctctcatctcagtctctacttttttgagtgggaaggaacccaactaacacatacatatctattagcttcttggcctaaagaacttacagtcaatcagagaacactaaacatatccaaataactaaaataaaaatcaatgaaacaaagctataaaatggaagttcagaggttgcttaaattgagtgaaaaaaaatcttagaaatgatgtgtctttttttttttttttccattaggtcttgaaggaagactttaactttggatttttcaatgtgtaatgatgaagaatggaaaggccattctaaacagaaagataaagagagcaaaggcaggtatacccaggcctattcttggactggtatcccagtttgactggaccgtgaggttcatgaaaaacaacagtaggataggagtgtggaaagttatattaagactatattgtcaaggatcttgaaagtcaaaccacggagttagagcttgaaaatagaggaaagtcattatttaaacttctttgaagcataatagtgggaggcaaattcaaaccatgaaatgtatgctaaaaataggagaactattgttctgtaatggaattgcaaaaattgtaagaactggattttcccattttctctaggatgattgcactttctgtattgaacttcaagatttcataaccaagtttcaactactcagcaagcagttatgtattcgttttcaaggtctattttaacaaagtctattttaacaagctgggtggcctaaacaactgaaatttattcccatagttctggaggctgaaagtccaaaatcaaggtgttggcagggttggctgcttttgagaggagagagggaaggatccatttctactgtggacaacccagtctcttttcttaactcatagatggctgtcttttctctgtctcttcacatcgtcttccctctatgcctgtctgtctctgtgtccaaaattcttttttttttttaataaggacaccaattacattggattagggcccactctaatgacctcattttaactaagtacatctcccaacaaccttatttccaaggaaggggtaattctgaggttctggggttcaggacttcaacatatgaattttcggagacacaattcaacttgttacaccctgtaaatccgaaaaagaattccctgaaaggaacctaaagtcacttagagtttatttaataaactcaaaatttttagacgttagatcagtcctatacataaatagctgatctcataagtggttttaaggatgctggcgggaggttgaaattgtattggtggagtctcctgccaaagaattcaaagttggaagatgaagaattcaggccaaagtatgataaatatcaatgaaaataagagagatttcacagataaatgagaggaagtattacagaatgggtaaatatgcaagcccttaatcaagtctgggtttcaattctagctccgtcatgtatttgcagatgatcttggacaagttacttaaactttctaagcatagatttcattatttgtaaaatatagtcttagagtagctagccaagacctttagatttccaagtataaattaaaagactggtcaaatatgtctcccaaactacagttttcacatagcgagctttccaagtggtactgttgaagccctttccctaggacctaggtcagagggagcacaccccacccatccttcttgagggtaaatgaaactcataatataccaaaacctctctccaaaatgctcttctaatgtataactcctctcagtctctccaatcccctttattggctagagatgggtagttagcaaaggtcacaaaaccagttatctagtctcacacctcactcgggggacctccaccaaaattgagactaacctagttccattttatcagcctgctatactagcgagactgcagtgggatgggcatttcatatggcaataataggagggcaaagtaatttggagatatatatatatatatatatataatttgaaaatactcatgctctttgatctgtcagttccacttttaggaatttatcctaaggaaataagttgaaatgtgaacaaatatttatttataagtatgttaactgtgaggttattagcagccatggaaaattggagacattccaaatttctaacaatgggggaatgtgtttaatgacatattgtacaaccatttaatataatgtataggattaagatggtaatttttatgttatgtctttttcactacaatttttaaaaaaatgtatagggcctggtgtggtggcacagtttctgtaatcccagcaactcaggagactgaggcaggaggatcgcttgagcccaggcattcgaggctgcagtgagctatgcttatattacaatataaataaaaagcataggatacaaaagtatatgtacactatgatttcaactaggttaaaatatgcatagaaaatggtctagatgaaatatgaaaaaattatcactgcttgcctacaactggtgtttgtttttgtttttatacttttctgtacttgaaaaatttacaacaaacaaataaggaaatggggcttaggagaccaaaaataggtctctccagtggaacaatagagaataaataaaggttataaagctgggctttgtaaaccggcatgctgggattaaatcctggctcaatttctctgtagaagagaaaagagagggcacaaatcctctgtccatagatgaattccttcacccttcctagcctcctttttctcatctataaaatgggcataatactggtacctctctcatatggttgttttagaaatcggtggtacaatgtgtgtaactgacttatctcagggtcatggctattcactctagaggtggcagacacagtggggaagagaagcatctcggacctccacctgaattttgcttcttccaccttcttctagtgactttgggtggttatgtactccttctctgctttattactgtggagacgtaaaatgagagtaatggaaatacctgtctcatactattgttacgaggattaagtgagttaatacatataatgtctatagaacagttcctggcattaagcaaaagctcaataaatgttagagttattattatgtcattatcatcattcttcttgtccttattataccaacaaatatggaactggagctgaagtgctgatcatgtttcatctagattatcctgggctttctaagatgaaataaaatcagaaaagagacaaaagttacagaaaaactgggaacagctccatgttaaacaagcgaatgaacaaatgaggcaaacaaaacaactaaaaatggcaaatggggaaataagaaattgggaagtaaactgttagcaaatatcatagataaaattcatgtatatcaagaactaatagaataaaatagaaaagcactaagatctccattgatatgtgggcaataagcatggtcagcccaaaacaggatccagttagaaaatgaatttatgaaaaatttcaacctcattaatgcagcccaagtgtccccaagatttctcattttttgggttaccacttccttttcattttaagtaaaatgactcaagtccccaacccctgtgtttacaggaaaacagttcctcctgaggatttcagagaagagctggacttcctggctgactctcacattctcttcttctgaactgcttcttctggtagctccctgtcactttggacaagctcgcactgccctccttggctgttctggaactgtgtgatggacttcatcctactaggacacagttgcttcttgaattccccagataaacacccacccgtgtgaatgacaagccataataaaaattaattgtcaaaattaatcaacaaggtcccaaacaagattgagagccaggggacagaagtgccaccggtaattatatagtcacaggcagacctatgaggatgttcatctttcctctaagacaacttgaagttacagacgtttcttagaacttaaaaagtttgatagccccttacccaaagaaatagaagcattggaaaatatgttaaagaaatttctatgaaattagaacaaaatgacttattttgcaagagaaaaaataagtaaaagcctaactctgagaagtccacatccatctaataggtgttccagaatcagaaaaagagatgaggtgaagacattatcaaaaaaaataataataataagacggagaaatcccactgaaggacatgaatctcctaaaagtacccagcaaaacgaacagttcaaagacccaaaccaaggcactttctcttgaaattttagaccatcaggaacaacaatagtaaaagcttccagagagagagaaagaaaaaagaaaccaggttacatacaaagaatcagaaatcagaataaccatgcatctctcagtacaatgctagaagctagaacttacagaacaataccttcaaaattgtatagaaaaatcatttgcaacatataactttatatccagccaaacagtcaatcaaatatgaggcatgacatgtaagttctccctccaaattaaccatcaatgcattctttcgggaaactactggatgatgaattccactataacagataaaaaccaagaaaggagatgctgtgcatcccaagaaacgagagatctaatacagcagagaggagcaacagctttccaggatagcaacaactcaaagtcagaggatgtcagctctgcagaaatccaatgcatgctggagcaaaatgacagacagtaattt

SEQ ID NO: 31 - DNA, 24498 bp, Homo sapiens
tggggcccacactttgagaagcaaatgcagctgaacttttttagaggaaagtgagtgaaccaactggtagctttgccactgcttaaaaaccagcatcctttccagctgggtctaagacagaataaggtaaatttagatatgtctctaatatatctatagaacagtggttctcaacccggggtgtttttgccccttaggggataatttgcaatgtctggagacatctgtgattgtcataactggaagggggcagtgctattggcatctagtgggtatagagcaagggtgctaccaaatatcctatggtgcaacagagaattatctggtcaaaaatgtaaatagtgctgagggtgagaaaccctgctataaaaacgaaagaaatttggtctacagagttgtttggatttagacaagacgttgccccaatagtggtgatagaaataagaggaaccccgtgcttttgcaaagcccatatctggggtggcttaaataatcatgctcctccccatcccccgacctgatctttgtagttggaaactccagggctggctgcctgtagtctttgtgactacacttcctgcctcccatcacttcatctcagaagactccagatataggatcactccatgccatcaagaaaggtattttaaacattggaacacatatagataatttaagtaggtagatgtatgtgctgttataaggaagtggggaggagagaagagggaaccgaaatcatatgcacaaaaattttttttagaatataaataaaaaatgtggtagtctaaaatgtcaattcttcaaagataaagttaggctttcagtaacgttagaaatggttttctggaatatgtctccagtctacctaactttgaggaagtaaatactgtaaatagatgtttcaaacgcattttaaagcaatgatcctagcatgtctttaagctacagtattgtgctgtctttgaaatgtaaactttgatgtcttctctttctcttagttgatgctattgggcccatctcaagctgatcttggcacctctcatgctctgctctcttcaaccagacctctacattccattttggaagaagactaaaaatggtaagaacagctcagagaaccttaaaaagtgttatctgtaatctttgtggaaacaactgaaaccagctggcaagagcaatattgaagaatctgtacttaggttatttgctgggggaaagtgcttcctgatatttcacaattggcattaatgaagggggcatgtcacaatttcagattaatcaacgcttgctctgttcaacttcctacaagaattaaatatgtgctgtggggaggaggagcagatgtttgaattggggacatagcttctatgtatctcatttcttcagcctacaattttggctttaaagccataacaaatcactgaattactgaagttactttgtgctttttccagcatatggtgttgtcttaatgactgtgtggatgaaagtgtgtgggcaggctcatagcaataaaatacgggaaatccccgggcttgagtgctgtcaaagaaaactaaatttggacagtagataaagatactatcaggactattgcaatcggcagaaagagacctcagtatagaaaggggctcaattccaaatacagccaaagaccagtaaagatttctggccaaggagtagagtgggggtcagtggatggaaaattactaagaggaaacatcaagggtaaaaggattctggctaaaccgacctgacaggattcttgctgaagacaggccagggtgatcagacctcacctgtggatggtgggagatgaggaatttgatcagatattgagggtgatcacataccaagaggagtggattatcaataaaatgacttagcaggattcctgcttgaactgggcaatgcaaagatggacatgaagccaaaggccgaagcctaggggtgtagtagagcctgattaagttgaattaaggagagtctttgtcagcgctggctctcccagtcactagttgggggggccttgtgcctgtcatcaaagtcctctgaaactcaatttctctgactatgaaataggcattagaatccctcccctgttgccttccagggccactgtgaggctcaaataatagactatttttcaagtcctttgcaagtggtatgatgcaagtgtgagttattaggtatgccaaaacttagtcggaaaaagacgtcaagggcctttttctgaaattattttgtcacttaaatcagacacattctagatccgaatgttagctcctaggctcattttgtgtcaaagttctaatgaagcattaaccatggggctattgttacaaaggaaacaactgcttacggtttcatttcctagaaacccagatgtctattttaatgcaaacctatgcccacatctgtctttgccccttgatgggtggcataatgggaatgatagtaatacagagagctcacatttcttgaccactcaactatcatgctgagggctagatagacatgattctattttggcctcaaagtagccctataaggtagagataacgaaactggggctttgagaggttaaggagcttgggtggctctgaaagctgtgctgaagactcttctgttcttcctagaccaagcccagcacacacgcaataaagatgaggttggatatgatggcttcctactcaagtacaaaggggaaatagtatatcttttctaagaaaagacgtgaaaataattttcaatataagaaattcaaaaggcaaaaaagcacagggaaaatattcaactgtattgagtcatatggcagatcctttgatctagagattacacttttagaaactcttcttaaagaagtgaccatgagactggataaaaaaatgtggcacatatacaccatggaatactatgcagccataaaaaggaatgagatcatgtcctttgcagggacattgatgaagctggaagccattatcctcagcaaactaacacaggaacaaaaaaccaaacaccgcatgttctcacttataagtgggagctgaacagtgagaacacatggacacagggaggggaacaacactcactgaggcctgtaggaggagggtggggcaggagagagcattagggtaaaaagctaatgcatgctgggcttaatacctaggtgatgggttgatctgtgcagcaaaccaccatggcacgtttaactatgtaacaaacctgcacatcctgcacatgtaccccagaacttaaaaaaacaagcaataaaataattttaaaaaaacaaaagaagtgatcgtggacatggaaaactatttaccaagatggtcagtgcagccaggcaaaaaaaaaaaaaaaaaaaaaatcatgtcccatgttgggaaggggtgaattaattgtagtagactcattaaatggaatattatgtaatcatcaaatcatgttttttaaaataatactgaatgacctaagaaagcactcatggtataatgttaaatgaaaaaagcaagctagaaatggataagtaccgtgtattcctcatgtttttactgcacctgctaggcaaatactagatgctcactaaatgttggataatctgtgatgatggtttacataaacacatgtgttgcatattctaatttcattcaacatccctactttataaccattttacagttggcaaatcagaggctcatgaggtcaagtgatttatgaaagtcagagagctcttacatgacagaacaaggacttaaaaccaaatttttgtactgacaaagccttggctgttactagaatgcttctcaccatgtgaaatagatgcagggatgggaaattactattagaagggaccatctcccaaaatgtcaatagtggttcagcaaatttaaaagtaaaaatattattctgctcttaacctataggaaatttctttatggctaaaaaaaggttattaagtaatcaatttattaaattaatacaatctgattatttaaaaatttggaacgctgtactaaaattaaaaatcatcattacagattaaccagccagtacctctgcaccccaagaataaataatgtatatccccgaaactcaccgaagtttagggctggggttggcaaactatggcccatgggctatatcccacctgctgtacagctcatgagctaaggggtttttttttaattgttgtttttaaaagactgaaaaatatcagagcaaaattactattttgtgacatataaaagttacattcaagtttcagtgtttacaaatggttttattgtttgagtatttgtttacttattgttgataagtgcttttgcactacgatggcaaactattcaaggagttgggtagtgtgacagagaacctgatggcctgcaaagattaaaccatttactaactggccctttacagaaaaagtacgtcaggccggggcttatagaaaacaaagggataaggtataaggtcaaataggtttgagagccctatggtctttggtgactgttgtgatgcataatagctgttgagttcctaatttatgtaagacaactttatatccttttattcttttagtttgaaaactaagtctgttgggctaaaatgataggaagtaaatgataactctctcctttttttaaaaaaaagcaagtggtttacaaccttgtacttaaacgttttggtgacataatgaaactgatattcatggtatttgtactttacagagattaaactaaaattaaaaatatttcaaaattcacaaataggggatatttgttaataaatctatttgggaaattcctagcagaggctcagtctataaaatgaatagcatttcagcaacttcccttattcacagtgcttggttattctctagggagacatacacaacacatctctagttaccaaacaattcagtgtgatataaacatggcaaaaagtcaatgaatttgagggcaaggtttccagcaatcgccccggccattgcttacttcttccatgccctttctaagttttcttcagccaggcagccatcccctctggtttctcccagacccccgctgcaggctccccgccatcacagaaagcccctcgctcacacgtcttggctcaagcaactctttgtcttagaaatgcagatcccaacatttccttttaaactcaggcaacttggcttttttctgctctgtgatcttgaaagtcgcttggaggaacagctgagtgcatggggctgttgtcctctcagggctaacatgttgtagcccagggggtgcccaggggcctttctgactggttggttagttgggtaaaagagtagagtcaggagagcaggaaatcctttcttaactcactataaaaataaaagcgttccccaggcctcaaatagtctcatctcaagataaatttccttttgccaagattgctgctgaaaataatccattgtagccagataatagctatgcaaagaatatataatagactggcaggggcatgcctaccgattcaatacagaaaggtgagggtttcatttgctggggtgtagtgggtgggagaattccttattgcaatcacactctacttctccatccagaaaactctccaaccctcctggaggactctccattttctcctctttctcctccttgtgtacctacctagaccatctgctcccatatgtcctgtctgacttcctgttccagttacctatcactgcgtaagagatcacctcaaaatgcaatggcttcaaacaacaacaatcatatactgctttctatcatgggtccaggagttgactggactcattaggcagctctcccacagggtctctcttggggtggcagtcaggcggtgactgcgactggaatcacctgaagactcactctccaggtctgatgcctgggctaggagactcaacagctaggtgccgaagcagctgcagctcctcaagtgtctctgtctccatgtggtctctctaatatggtggttgtcgtatagccaggcttcttacaagggtgatgactcaggactccaaagcaagtgggtgagagaaagggagagagggagaaacagggagagagagagagaaagtgtgtgtgtgccagtacgcgcgaggtgaaagctgtattgcctgtgaactacccaccatgtctttcgtcctcttgacaggaaacctcctagaaatgtttgctgtctccaaatccctctccttacgttcttccaagaactttgaagtcatattttatgtagctactccttcaaaacatatctggtgttcggccagttcttacgccctccagcactgctacctgggacttctgcttgaatgactgtaatagcctctcaactagtctccctgctttcacccttgcccctcactgtctattctcaacacagcagccagcagcatccttctcaaatgtaagtcagaccaactgattgtcagctcaaaaatttgcaatgcatctgcattccacccagagcagagaccgccatccatggaatggtagagaaagcccaacatgctcagggacactccctctctgacttcatctcctattgttctcctacaccccctgcttcagcaatattggccccgttgccatttttgtgaatattctagcatgttttcaccttggggcctttgctccaggctaatccatctgtctggaatgcatttcccctggatgtctgttatggatgactttgtcctttccttgaggtctttgtttagatatcaacttcttaatgatgcctatccaagctgccctatttatcgtcacaatcctaccccacattcctgatccttttcactctgccctgttttctttttcagtaacacttatcacttgacatgcaatatcatttctgacagttatatatttttgtgattatttagagaacataagctatagttgagtggaaatcttttctattttgtccactgatgtcccaaacacctagagaagtacctggcatgttgcaggcatcaataaatacttgttgaatttttcctttttcacaatttccttctacgttgttatgatgagatcttatttcctctgtaatttgattttaaaagttttaataaaaaacaatacatattatttatgataaaaagtcaaagagtagagaagggtataacataaaaatagaagtccccctcttcccagggaaggcccctttataccactgcccagaagaaattgctattaaaggtttcttgtgtattctttcctacttttctctgcaaatacaaatatatgcatatatatttatcataaatgcattatatgttatatgttattttaatgctgctttaaaaatcccctttattttttgtaacttagtagtagatcatgcatagctttttatgtcgatacccacagctctaccacattctttttaagggacatttgatattttactattggtagtttcccatttttaaccattctctcaaatcaatggattgtcatgtaattcttcctattcttactatttcagaaagctgaatcaaactagcaaaatagttttatctaaagacatataaggccgggcgtagtggctcttgcctgtaatcccagcactttgggaggctgaggcaggcagaccacctgaagtcaggagtttgagaccagcctggccaacatggtgaaaccccgtctctgctaaaaatacaaaaattagctgggagtggtggcggctgtctgtaatcccagatactcaggaggctgaggcaggagaatcacttgaaccgggtaggcagaggttgcggtgatccaagatcgggccagtgtactccagcctgggcgacagagtgagactctgtctcaaaataaataaataaataaataataaagacatataatgcttactttaaagaaaaacaaaacaaaacatgtactagttatttttttcctccctctgtggaattcttagaaggtttatggtagtttgaagctttgcatggaccattttgaaacagcagcagcctgaggttccagggggttatgaagactcccagctgaggacagaccctggcagataagtttcagggggctctacaccaaccattagagtcatagaataagcacaatagaaaaggaccattaaggtcagttagccaaactccagagtttgttgatgagaaagtcaaggttcaggataattcagttggtagccctgtagcagacagagagactgaaaacaaatctgactttcagttcacgtggtgctaacccctagaataaataaacacgaggagaaatcagactaatcccagtcttcttctaacttgtcacaagacacaaaccacttaccttcacttcctcattttttccatctaatagttcccagttatatacatgtccttctcactcctctgattgcaaccagacatctcttacaagtttacaaagttttgaagataaaaacgctatttggaaagcgtaaagttaaaaacagcttggtaaatgtttttttttttttctattagtaattcgatctctacaactgtaaatattgtggtaggaatctaatacagatctaaaatcagtaaaattcaatcttgaatatgggcttcagtcctgccatcaaaatagtgcatccaggtggataggttttgccaccttgaagagttgtttattcaaacttttgtttgaagagtaggaaagcagtgttacctttaggcctgacttagcccttgccccacaatctattgttttttctcaccatagatttccctgacagcagagagagagttctgtgctcaagagatacacacagcttctgacaatagagcagcagagtatttggttcctaattgagcaggaatggtgtttgactcatcatcatttccctactttgtctagcacagtaccttgcacagagtagattctcaataatgtttgttgaatgactgtgggagcatataattcataatggagacaaagctcaatgaggctttaaatttctaaatccacaaaatgccctcatgtaacattgctggatgatatggtttagctgtgtccccacctaaatctcaccttgaattgtagctcccataatccccacgtgttgtgggagggacccagtgggaggtaattgaatcatgggggcgggtttttcccatgctgttctcatgatagtggataagtctcacaagatctgatggtttcataaacggcagttcccctgcacatgctctcttgcctgacgccatgtaagacgtaattttgctcctccttcaccttccaccatgattgtgaggcctcctcagtcatgtggaactgtgagtccattaaatctctttttctttataaattacccaaactcggatatgtttttattagcagcatgagaacagactaatacaatggacattggatgcaattcatttaaaaaatcatcttaaaaatatctttcttttttctccctcaagttggtcccactcaaaacataaacacaccatttttttttttttttgtcttgagacagagtcttgctctgtcacccaggctggagtgcagtggtatgatcgtggcttactgcaacctctgcctcccgagttcaagcaattctcctgcgtcagcctcctgagtagctgggattacaggtgcatgccaccatgcccggctaattttgtatttttagtagaaatagggtttcaccatgttggccatgctggtctcaaactcctcacctcaggtgatcctcccgccttggactcccaaagtgctgggatttcatgtgtgagccagtgtgcccagccaccattttttaatacttgtaaatttttcctataaaaacaaaccaatttctctatgccccaaaaccgctaagtagcacaaaatagaaacattagagtaccaagaatacttgaactgaaaaggaaattaatcaaaatgcagacacacattataccaagtgcatttgctgtagctgtgtaaggcaacttgaatagaattggtcaacaatgagtctgaatcttggtttgaaattgcctgtctgatctctgcttcctcatcagtaaaatgagaatatttatatggcctttcaacttcagtgtgagggatcaatgatgtaatataaacaacaagtctgccttagaacctggcacaccataagtaataaaaggcagccaatattttaaaaaatacacaaatcatggtctgatggctgtccaatataaattctctattttccattttaactaaagagacgatatattgagaaaatagaaacacctgtgtgtatgaaatcacccattcccatttttacaataattagtttgctaattgagcatccaaatttacccagtgtatttgcatgtgtaattagctgtgattcaataccaaagccaggcctatcatggtatactatgctattttacaagtcaaattactgaaagatgcatgtctttaggcaatcattacaaataaaaaaaaaaaaaccgaagcaaaacaaaataacatagattatttgtatcagatggacaaaacagacctggcttgatgccgaacccttaaatctcaaaataacgatagttgaagctaaggttccagcttaagtctgaagcaggtagtttccaatggcttgaaaggagaaatttctacactgaaggaaatttccattggaataaaggaatatttcacacttttaagtcatcttctctagatggtcttttgggtatactttctctttaaataacagatttagaagcactttgttcatttgtttagaattaattccattcacaagtttaacacagcctaaggtttggtctagaccaggggtctgccagctatgacctctgggctaaatctgtcccttcacctgctttttttttttttttttttccaacctgtgagctaagaatgggttttactattctaataaatagtgagttcatttttctccctcacctgcttgatcagagcccaactttctcattgcagttaatcttccttctggcatggatcttggaatgcaaacttgctgggatctccgagttccaggcttcccgtgcagccggtgtggagagccaagagatgttttgtttggcataaagcattccaagggtcagtgggcttgggctcaactattgagcataggacaagggcagccccatcctgactgtgactcttcccacaagagacaaacgagctctgtgctttcactggggtttcaggttcaaagggacagagcgtctgagaaaaaggattatgaaagagtccgtctgcagctccacttcccgtgcccttccaatgataccatcctcgtttcttctgtggcatgctccccacttcaatccttccttcagaggccccaaaccctcctggtctctccttgtcaccttgtgaaaatctgatcttcagggaaaaattccttactatttatactagtataatgtgaatcttctatgggattttaagaaagttcaaagccttggtttactcagcaaatatttagcttgcactcactatgtggcgggcatcctaatgatggagtatatgtaaagacaaaaaaagtttccggacctcaaagtgttctccatctataggggcagatgactgagttgacatctcgagaagtagaatagcagagtggctaagagtgccagctctgtctcaatcacctaggtctcacctcagcattaatttcactttcctcattgtaaatgagcatatctcttagaattgggataagcattaaataatatagacttggaatgaatttgcttagaactaattccatgcacaagtttatcacagcctaaggtttggtctagaccagaggtctgccaagtatgacctgtgggctcaatctgtcccactacctattgttgttgttgctgttgttttttaatgacctgtgagctaagaatgggttttactattctaattagttacattctcaatggttatttaagtacctccataatatcctcaattttgcctaaaatatttaccatctggccctttacagaataagtttgctgacttattggtctggaccaatgctatctaataaaactttctgcaatgatgaaaatggtctctatctgtacccttgaatacagcagccactagcctaatgtggctttttgagctcttgaaatatagttagtgtgactaagagattgaattttaattaatttaaatttatggagccacatgtgactatgacattagagcagctctagacagcctgaagtctaaagactctatgctttgtcggtgctcccctctctcaattgaatcaactaccctgaggctgcatgagtcaaggggaaggccacactcttcaatcagattttttgccctggactggctttcattgtctactagaaaatgcttaatgggaagtgcttagaaaatgtacatgggcatacacttaattaatctaagttgctgctttgtctgtatccattaaatctgctttattttggggtaaactacagtagaagttggctttttcaaccctgcaaagccttaaaattcaggatgtcttactcaacttaaagtgtagagttgcagccagagcacaactgtatttccttctagccctgcttgcagaatggctaacttcagtcctatttcatttctcttgtaagactgctaaaaacagtaagaagccaccaacatcattatgaatattgccaaatcatttcgcctaagagtaaagtcacagttggcatgtgttctgccctccaagacaagatagcataggtgacagttttatcagatatcttgtgatggcataatataggccacccagctttccagcctctgatatctgagtcttcccaatagcctgatgacatccgcatcacatattttaggttcgctcatggacagtaacttatttccaaattctatactggttaaaattaggtttgcatttgtgcaatagaaaatccaattgacattggcttagcataacaattttttgatttctcataaactcttggcagtcagcaggtccaagccattatttctgctctgctctctgaggtcatataaggaaggatctggatgctctgggtcatctacgtcatctaactggttgctgtgccatccctagctcatttttctcatgtgcattgcccaagatggctggctacaacatccacattacaagaagccaggtggaagcagacaggagaaagaggagaaaggggaactgccccaccgtttaaggacatgtcccagaaactgtacacctcacttcctcccaaatttcactggctatcacttagtcatatagccacacttagctgcaagtgtgtctgggagatataattatttttcacagtggtatatgcccaactacaaatggaggttctgtcattatgagatgagagaaaggcagaaaacatgttgagagatgtgtagcaatctctggactccacggggataaaaaagaattgagagtatcaaaattcaggatcaaaatcaaaattaaagataaaaaatatcaataactatcacctggaataagaacaacgtacagttcagctacacatatacaagtggcagcatcttgtctggaaggaactaatggtctttctacattgtattttagatatgtatttttttttctcccttccacaggattttgagctccttaagggcagagactttgtgtctcctgctcctagtaggcatccaacacgtatctgtcaactgaaagaatgaatatgagtcagtagatacatattagaattctaatatccactggctgggtccttggtgtgtcccatattgttgtttctgtgtccatcattcttttgcagggtatcttctactgggcacagaacctgcctcagaggggcatatgggtaatgaactaccaagaaaggagtagaacccagttcttccaaccctccacccagagtgcttttcacaacctcatgtgtaataagtgcagtaggagatgagaggagggagtgattacttctgtctggtttgatcccagaaggttttttgaagaaagtgtttttgaatgagacattatgaaaacagagcttcttaaaccttttcccccaaggaatccctgggcagatagaagagacagaaatctgacctctgcttagtctgggggtatagactgaaggaacctactcaaaggagaaatttttctcatttttctttacttcacgattcatatatgcaggcattcattctttcattcatgtatctcacagacataacgaggtcctaattaagtgccaggcattgttttacatgagaccacaagaggccctaccctcttgcagcttacattcttgtacagaatagacatcatacgaataagcaacataaatcatcaagataatttctgaccgtggtaagggctatgaccgaaatcaaacagggtagtcagttacagagtgcatatacctctctgtgcctcagttgactcatctgtaaaatggagataataatagaggtctaggctaggcatggtggctcatgcctgtaatcccagcactttgggaggccgaggtgggtggttcacttggggtcaggcattccagaccagcctaaccaacatggtgaaaccccgtctctactaaaaatacaaaaattagccaggcctggtggtgcatacctgtaatcccagctacttgggaggctgaagcaggagaatcgcttgaacccgggaggtggaggttgcagtgaaccgagattatgccattgcactccagcctgggcaataagagcgaaactcagtctcaaataataataataataataataataataataatagtctataattccaaaacccaaaactgaaagctttgtcctaactcagttgattgcaaacataatatgatctgaatgcatttggaggtagatcttgacctgaactgaagttatttattctttttaataaataaatgagttatttattctttttaataaataaatgagtcatttattctttttaataaatgagttattctttttaataaataataaactgagttatttattctttttaataaataataaataactgagttatttattctttttaataaataataaatgagttatttattctttttaataaataataaataactgagttatttattcttttttttttaataattccacttagagtggacaatcctatatgtcactgcagaaattttgtgtgtttgattatggaatgctgccccaggcctcaatagttattacataatttagggtacatgtagcgtattaccttctaaaatttgaaaaattccgaattccaaaacacatgtagcaccaaaggtttcggataagggattgaagacctgtagtatccattattgtgaggattaaatgaatgaatatatggaaaacacttaaaatgatgcctggcatgtggtaagtgctacgtaagttaactactattactattattatcactattcttacatgagaagatatttagataagttggtcagggaaagcctctctgaggatgtgtcacttgaataggcaactaaggggtggtaatgaccgggctgtgggaagaggaggagaaagatgatttcaggaataggaaacagcaagtgccaagactgtggtggttacaaggctggcttgaatgcagaacagaaaacagaccagatggctgatatgtggtaaaggaggggaaagatggctcaaggtcagagaggtaggctgaagtcagaacacccttgatataagcaatggtagagactttggatttcatttaaagtgtaataggaagacattatagttgatctgattcaggtttataaagaacgctctgatgctgttggatgaatgaattatagaggagaagggggagcagggagagcaatttggagtctagcatagtggtccagatgagacctaatgactaattggagttgggaggtggtaatagtcaaagagaaaagtggacaggtgcgagaaaaaagtttagaaataagtggggggcgggggaggttttctgattaatttgcattctaatttataatatgtcactgtgtagaggctaaaaatttcacagtcattgtctcaggtgtgttaaggccagtggcgtgctggaccccacttgaaattggccatggagggaatatttacactatagaaattgacaaatgctacaaatcaagacaacaaatcaggcaaagcttcttgttaaacatttaccatcacaccactggtgaaggtgacttgatttttccacaactaaacttccttcatttcacagcctccattttccctgatcacgaaaacacttaaactaggcacatcctcggaaacgcagtatgaggactgctgtgtcaatcacttcatgtttttaactcaattcagcgatcctcccacttcttcccaggctctcatttaggtacatgggaatgggatgggaagagggacctggttcatgattgtcatttacccaccttggccccctctgaagtacaactccactctctgctttacaatatcactctgggcagcattaccaattgcctcctgatagtgggatctatgaacccattatgtctttggacaaaagcatagccaggggttgggtccagggcctgggatcctataaccgtacaaatcctattatcagggactataaaatcctattatcagggaccatagccatccctctatcttgactcaactcctcctccctgagtagtgaacatttttcctaaatctctgagaaagactggtgctctagaaagatgtaccatatttatttaagggcttcctgtacccactggcatattgccatatattctgaggtatctgagtgctccttttgagaaacatagccttaaaggataagtagaaatctggtgggtgaaaatggtagggaagaggacttctaacggagggacttgcaagtcagggaacttgggtttatcgactagtgaggctagtagaggaattcaatcaggtaagccggacaagtagacagggtacaaattatggaagactttggatgccatgataaaaagcttcagctcatactgtaaaaaataaaataaaataagaaggttgggtgcagtggctcatgactgtaatttcagcactttgggaggctgaggtgggacgatcgcttgagcctgggaaacaatttcaaggagttcacagcaagaaactgactgattaaggtttgggaagcttgatagatagggtagactgggaaagtgagagaggaggctttggagtggaccaaggatagagggatctcagctgatattatgtcagctaaaacctcaaagcaaggaggatgttaagaacaatgaaggaggtcagctggactctcaatgtttttaacgatagggaggaaaagataggggggtgacaagaagaagagacaattttgtacctctaactccaacaaactttagacctgaaaaatcccttctgagccatcttgcattggagaaaaaaaattgcttatttacctccaattagaggaattaagggaagtaggatttttttgtttttcttttgagacagggtcttgctctgtcaccctggctggggtgcagtggtgtgatcacggctcactgcaacctcaaactcttgggcttaagaggtcctcccaactcaacctcccgagtagctgaactacagttgtgtgccaccatgcccagctaattttttattttctgtagagagaggggtctcacgctatgttgcccaggctagtcttgaactctggcctcaagcgatccgcctgccttgtcctcccaaagcgttggtattagaggcatgagccaccacatctggtggaagtaggcatttggtttcttagataacaacatgattggttgattcagtcacttgggaagataaaagcattaactgagctagatccctatggtagagacacaggctggaccactccatgcgtaagtactaaactaaaaccagtgttctggagtagacattgctagaaatcctgaaacttgagagccagtccacggttaaagcattctgtaaggcagagccagtggaaggtaataaggtgatttttaaagctcttctgcacttcccatattcccttttagggcctttctccctagggtcccagtgtctgtcatgctaaacctagatgcacaacaatcatctttatgggtagtttcccatatgtcccagtttgcctgacagactcttggtttatgcctatagtcttggtgtaattattaccagccccacttcattcttgtaagtatactaatggatcagttatacggttcctctgattatgtatcacctaggcagtgccctgactctactactatctcctctccaaatttatgtaatgtaaacccaatgtgtagggaaaatgctcatcctaaaatctccttggaggggataatttgcaagattctttgcaaaaacaatccaagacaagagccagattatggaatgtcagtgccagaatggcaggaatgtatgttttctaatcaaatgccacttactactgggtaaccttgggctaatcagttaatattgctgagcgatgtcttcatttgtaaaacgggaatcttagaatattctgagactcaaatactatgaaagactcatgtaatgtgtaccagggcaggtttagcaggccgacataaattgcactaaagtcttcatgtgttatttttcatgggtgtatccatattctaacatttcttcaccctccaaatttcagactttggcagtgaatctatggctctgcaattttagtgttccatgtaacaacgaataggaaaatgctgcttctaccctctcgaaagctattttgctaaagagctaagatgctaaaagctaaatatgtaactaaatagttgcaaatctcagtaactgacaaatacagtcatggggttggggatgctgtttagacagctgaaaataagacctgaattgtttatttttaaaatgttgcaaaagagaggcagcaaatgggaatttttaattctgattcttggtatgttttagaacaatgatttgttctttcttatactttcaggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggagccaccaacgtctgtcacaggagttggaaagatggggtttatataatgcatcaagtcttctttcttatctctctgtgtctctatttgcacttgagtctctcacctcagctcctgtaaaagagtggcaagtaaaaaacatggggctctgattctcctgtaattgtgataattaaatatacacacaatcatgacattgagaagaactgcatttctacccttaaaaagtactggtatatacagaaatagggttaaaaaaaactcaagctctctctatatgagaccaaaatgtactagagttagtttagtgaaataaaaaaccagtcagctggccgggcatggtggctcatgcttgtaatcccagcactttgggaggccgaggcaggtggatcacgaggtcaggagtttgagaccagtctggccaacatggtgaaaccccgtctgtactaaaaatacaaaaattagctgggcgtggtggtgggtgcctgtaatcccagctacttgggaggctgaggcaggagaatcgcttgaacccgggaggtggaggtggcagtgagccgagatcacgccactgcaatgcagcccgggcaacagagctagactgtctcaaaagaacaaaaaaaaaaaaacacaaaaaaactcagtcagcttcttaaccaattgcttccgtgtcatccagggccccattctgtgcagattgagtgtgggcaccacacaggtggttgctgcttcagtgcttcctgctctttttccttgggcctgcttctgggttccatagggaaacagtaagaaagaaagacacatccttaccataaatgcatatggtccacctacaaatagaaaaatatttaaatgatctgcctttatacaaagtgatattctctacctttgataatttacctgcttaaatgtttttatctgcactgcaaagtactgtatccaaagtaaaatttcctcatccaatatctttcaaactgttttgttaactaatgccatatatttgtaagtatctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaatctctggaataaatagagaggtaattaaattgctggagccaactatttcacaacttctgtaagctttattgtgtttcatagtttccgttcttcttctgtgagaacaaggataatggcattaaaaaatcagcttttggtcattataaattgtcttctattaaaacacatatacacataaaatcacttgaagacaatttaaacatcttctgaaatggatcaagaggaagggaaactgaaaataatgcaactcagaaaccacagagtattttgacatgaggttaagcaccgtggtttgttgtaggaaaataacagcacaccaacagatggtttttatctgaattctttggtaatcttgacatgtcattcttctaactttctgagggccctcagtgcagttttgtaggactggagctgttcacagacggtccccacaaagctctgaacgtggggcttctctgctgactggcctctggttggctccaccccggaaggaactcccagattctccatgaattccgcttccaccatcaagccttggtccaagcccctttcaaccttgacttggccaggaagtgtcctttctcttcagatagatactacaccttagcaagacttggcatttttagaatccaagccaagggaggcacttggcaaggcaaatgtt